(19) United States
(12) Patent Application Publication
(10) Pub. No.: US 2007/0065415 A1
(43) Pub. Date: Mar. 22, 2007

(54) Compositions and methods for the augmentation and repair of defects in tissue

(76) Inventors: Donald A. Kleinsek, Elkhart Lake, WI (US); Adriana Soto, Elkhart Lake, WI (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 11/229,237

(22) Filed: Sep. 16, 2005

(51) Int. Cl. A61K 35/12; C12N 5/08

(52) U.S. Cl. 424/93.7; 435/366; 435/371

(57) ABSTRACT

Compositions and methods of treating a defect in a patient are disclosed, including expanding a culture of autologous cells in vitro to form cultured cells, collecting the cultured cells for introduction into the patient, and depositing the cultured cells with ancillary proteins.

### FIELD OF INVENTION

The field of the invention relates to methods and materials for using the repair or augmentation of defects in human and/or animal tissues, for example, as caused by aging, tissue degeneration, diseases, medical disorders, trauma, surgery, or patient desire (e.g. augmentation). The field of the invention further includes compositions of proteins or other macromolecules in combination with living cells to treat the tissues.

### BACKGROUND

Repair and augmentation of a tissue by use of a non-living material is helpful to address cosmetic and medical concerns and injuries. In particular, the use of collagen to treat wrinkles has been shown to be a generally safe and effective procedure. Collagen implants, however, tend to be resorbed into the body so that its implantation is of only temporary usefulness. The use of living cells provides a long term solution, provided that the cells successfully adapt to the implant site.

### SUMMARY OF THE INVENTION

Materials and methods are described herein to improve the successful adaptation of living cells to an implant site in a patient. Examples of various defects that may serve as the implant site are provided. Some embodiments of improved methods comprise treating a defect in a patient with in vitro expanded cells (autologous or non-autologous) and implanting into the tissue defect the cells with associated protein or proteins. Some embodiments comprise treating a defect in a patient by expanding a culture of autologous cells in vitro and suspending the autologous cells in a nongellable physiological solution having an immunogenic amount of asoluble protein and depositing the cells and the protein at the defect in the patient to repair or augment a tissue at or near the defect. Various proteins are described, including immunogenic and/or cell adhesion mediating proteins.

Other embodiments of improved methods comprise treating a defect in a patient by expanding a culture of cells in vitro and depositing the cells with a predetermined apoptosis inhibiting factor in the patient to repair or augment a tissue at a defect, with at a defect meaning in or nearby the defect.

In other embodiments, an in vitro expanded culture of cells plus a purified serum protein is deposited at the defect to repair or augment a tissue. Other embodiments comprise treating a defect in a patient by expanding a culture of cells in vitro and depositing the cells with a predetermined protease inhibiting factor at the defect.

Other embodiments comprise treating a tissue in a patient by expanding a culture of cells in vitro and implanting the cells into the tissue to treat the tissue for a deficiency caused by aging.

Other embodiments comprise treating a tissue in a patient with cells that are not autologous, expanding a culture of cells in vitro and implanting the cells at the tissue defect.

Other embodiments comprise treating a defect in a patient by depositing an immunogenic amount of protein at the defect in the patient to repair or augment a tissue at or near the defect. Other embodiments may use non-immunogenic proteins to treat the defect. Additional embodiments are also described herein.

Additional embodiments comprise the use of gene therapy in which carriers for genes are implanted to treat the defect can be used. Various cell types containing the gene of interest can be the carrier. Other forms of carriers containing genes encoding proteins can be used.

Furthermore, 3 dimensional tissue can be synthesized in vitro for implantation in vivo.

### DETAILED DESCRIPTION OF THE INVENTION

Tissues are subject to the effects of aging, and become deficient over time. Fortunately, however, it has been discovered that many tissue defects may be treated by adding living cells to the tissue. One effect of aging is the loss of elasticity in tissue. This affects the appearance of the tissue and its function. Described herein are methods of treating a tissue in a patient by expanding a culture of autologous cells in vitro and implanting the autologous cells at the tissue to treat the tissue for a deficiency caused by aging. Aging and diseased tissue become dysfunctional in large part due to loss of appropriate numbers of cell types. This in turn results in lower cell populations and changing gene expression that alter ECM matrix, protein and enzymatic activities (proteases), cell adhesion, cell migration, cell proliferation, cell differentiation, hormone and growth factor production, signaling pathways, feedback mechanisms, tissue homeostasis and dystrophic tissue morphology, amongst other actions, as described in greater detail below.

In general, aging tissue that is connective or contains connective tissue cells displays less moisture or hydration content, less proteoglycan or ground substance content and less tone or turgor. In skin for example, this is true for all tissue layers, but in particular the dermal and subcutaneous layers. Aging tissue frequently contains less ECM and more protease activity. Cells (e.g. fibroblasts) and/or factors such as proteins (e.g. proteoglycans) that improve these changes can repair or restore aging tissue to specific young tissue parameters and function.

An abundance of living cells may be obtained from a relatively small tissue sample when modem cell culture techniques are used. It is thus possible to take a tissue sample from a patient or another source, obtain cells from the tissue, expand the number of cells, and reintroduce the cells into the patient to treat a defect in the patient's tissue. The implantation of cultured cells into a patient's tissue has the challenges of helping the implanted cells “take” to their new site and has not been adequately addressed in the past. Even when autologous cells from the patient's own body are used, the cells must still be integrated into the new site and use, or develop, means for receiving oxygen, sources of nutrition, and means for maintaining metabolic activity.

The living cells would typically have some amount of internal resources that can temporarily sustain them after implantation, but must quickly adjust after implantation. For example, the cells should respond appropriately to their new environment. Part of their response can depend on cues that the cell receives from its new environment. In the absence of appropriate cues, however, the cells may respond poorly or die. The adjustment process may thus be facilitated by providing proteins and other biomolecules to the cells during the implantation process so as to provide suitable cues to direct the cells. Such proteins may provide biochemical cues to stimulate a particular metabolic response, cause the production of useful proteins, or otherwise help the cell to adapt. Further, such proteins may provide mechanical advantages by giving support for cell anchorage or covering up undesirable cues in the implant site. And some such proteins may serve as reservoirs for other helpful biomolecules that are provided at the time of cell implantation or that are produced by the cells.

One set of helpful proteins is immunogenic proteins. While some previous scientists have emphasized the need for the cells and other materials associated with the implant to be essentially non-immunogenic, the use of immunogenic proteins in an appropriately controlled way may be helpful, as discussed in greater detail, below. In brief, one reason that the response can be helpful is that immunogenic agents can induce an immune response activating immune cells to cause inflammation to trigger macrophages and other cells to produce cytokines. Further, the immune response may create local site inflammation and erythema. Inflammation and erythema increases blood flow. Increased blood flow enhances delivery of oxygen and nutrients to the implant site. Moreover, increased proliferation of fibroblasts, deposition of extracellular matrix molecules, angiogenesis, and secretion of growth-inducing and survival-enhancing factors are all associated with the immune response. Moreover, an immune response may also result in the scarring of the surrounding local area of introduction. Scarring can, in itself, augment tissue. Since the response may be directed to the proteins introduced with the cells, and not the implanted cells, the implanted cells are not destroyed by the immune response.

Certain other embodiments include the introduction of a protein into a site at or near a defect to treat the defect, e.g., as in a defect in a tissue. In such cases, the protein may be immunogenic. Some embodiments are a method of treating a defect in a patient comprising depositing an immunogenic amount of protein at the defect in the patient to repair or augment a tissue at or near the defect. The protein may be, e.g., a cell adhesion mediating protein, a serum protein, a protease inhibitor, or other protein described herein. The term protein includes proteoglycans and also peptides having at least 3 residues. The residues may be amino acids found in nature, or synthetic residues, e.g., with altered backbones or side chains. Proteins may be obtained from various sources, e.g., natural sources, by chemical synthesis, recombinant DNA or from cell culture translation systems. Various proteins are described herein. It is recognized that fragments of the proteins may be used, that the proteins may be combined with, or decorated with, other chemicals, polymers, or proteins, and that alternatively spliced versions may be used.

An improved method of treating a defect in a patient involves expanding a culture of cells in vitro and suspending the cells, e.g., in a physiological solution that further comprises an immunogenic amount of cell protein, and depositing the cells (and/or the protein) at the defect in the patient to repair or augment a tissue at or near the defect. The cells may be, e.g., autologous. The protein may be an adhesion (adhesion to cells or to other proteins such as the ECM) mediating protein or proteoglycan, e.g., fibronectin or laminin. In certain embodiments, the solution is nongellable and/or the protein is not gelled, and the solution and/or protein does not gel upon introduction into the body. Instead, the protein is free to associate with the cells that are introduced and/or with cells or ECM and tissues at the implant site. Without being bound to a particular mechanism of action, the protein can generally be expected to diffuse a limited distance from the implantation site by virtue of having multiple specific or non-specific binding events that slow its diffusion from the site. As a result, the protein exerts its effects, in general, at or near the site of implantation. At the same time, because of its nongelled state, the protein has enhanced availability and diffusivity relative to a gelled protein, or one crosslinked to form a hydrogel. In the case of immunogenic proteins, these may serve to recruit an immune response to enhance the “take” of the implanted cells. A gel refers to a semisolid, jellylike state assumed by some suspensions or colloidal dispersions at rest. A gel that is crosslinked is insoluble. A gellable solution is a liquid that can form a gel, for example, a solution, suspension, or dispersion that gels with time, changes in pH, or changes in temperature.

Another set of helpful proteins or factors is a predetermined apoptosis inhibiting factor. Predetermined refers to the choice of a particular factor for introduction into the patient. It is recognized that some factors might, in theory, be incidentally introduced into patients from time to time with cells if the cells are in a complex mixture derived from a cell culture or tissue source. The incidental inclusion of such factors, however, is distinct from selecting a predetermined factor that can be intentionally introduced and/or adjusted to achieve a particular concentration, amount, or a desired effect. The prevention and/or inhibition of apoptosis advantageously enhances “take” of the implanted cells by extending their life during the time of adjustment after introduction into the patient. Factor is a broad term that refers to biologically active molecules, including proteins, molecules of natural or synthetic origin, proteoglycans, polysaccharides, glycosaminoglycans, hormones, and small molecule drugs.

The choice of an apoptosis inhibiting factor for implantation with a cell into a tissue depends, in part, on the cell and the tissue because some biological factors inhibit apoptosis only for particular cells or biological environments. The scientific literature is rich with studies that describe factors that inhibit apoptosis for particular cells so that the ordinary artisan can use such literature as a guide to select factors that are suitable for the application. The detailed discussion of apoptosis factors, below, provides additional information for choosing suitable factors.

One embodiment is a method of treating a defect in a patient comprising expanding a culture of cells in vitro and depositing the cells with a predetermined apoptosis inhibiting factor at the defect in the patient to repair or augment a tissue at or near the defect. The cells may be, e.g., autologous.

Another set of helpful proteins is serum proteins. One advantage of serum proteins is that they are readily available from an autologous or other donor source. Serum proteins have been proven to be important for maintenance of cells in vitro and, similarly, can be effective for maintaining cells in vivo at an implantation site. The effectiveness of serum proteins is not fully understood, but, in some aspects, it may relate to the presence of cell adhesion factors, growth factors, various transport proteins and/or procoagulation factors. In general, serum factors used in the culture of cells in vitro may be used to some advantage when applied in combination with the implanted cells. In some embodiments, the serum proteins are in solution or suspension and not gelled or cross-linked, so as to be fully available for interaction with cells and subject to cellular receptor interaction, transduction of signaling pathways, internalization and/or cellular down regulation, as needed. Serum factors are described in more detail, below. An embodiment is a method of treating a defect in a patient by expanding a culture of cells in vitro and preparing a composition that comprises the cells and a serum protein, and depositing the composition at the defect to repair or augment a tissue at or near the defect. The cells may be, e.g., autologous.

Another set of helpful proteins is protease inhibitors. Proteases are enzymes that degrade proteins. As such, they can damage cells and/or cellular factors that are needed by cells. Protease inhibitors, described in greater detail below, may be introduced into a site of implantation to advantageously limit the action of proteases. An embodiment is a method of treating a defect in a patient by expanding a culture of cells in vitro and depositing the cells with a predetermined protease inhibiting factor at the defect in the patient to repair or augment a tissue at or near the defect. The cells may be, e.g., autologous.

Another set of helpful proteins and factors is proteins or other factors that induce coagulation. The procoagulation proteins are helpful in reducing blood loss and bleeding at the implantation site. These proteins are also mitogenic for many cell types enhancing the introduction of cell numbers to the implantation site.

These proteins and others can be useful for the in vitro expansion of cells and/or treatment of the defect.

These proteins and others can be useful for the three-dimensional synthesis in vitro of tissue to be implanted in vivo. Preferably, the tissue components simulate the in vivo environment closely. Alternately, the tissue components are functional, yet distinct from the natural in vivo environment. This enablement includes the in vitro synthesis of organs or tissues.

Compositions for treating a defect may be formed by mixing a cell and a protein or factor that is described herein. Thus autologous cells or autologous cells may be combined with a helpful protein that assists in the introduction of the cell into the implant site.

Immunogenicity

Implantation of cells or proteins can cause an immune reaction. A major concern of implanting xenogeneic or allogeneic cells is that they will provoke an immune response from the host that destroys or compromises the implanted cells, thereby reducing or losing the therapeutic value of the cells. The use of autologous cells can reduce or eliminate the immune response so as to preserve the value of the therapeutic regimen. Immunogenic agents are often proteins or carbohydrate molecules that the host recognizes as foreign.

Immunogenic agents may be advantageously be added with implanted cells. Indeed, an immunogenic reaction towards immunogenic noncellular agents does not negate the effectiveness of the implanted cells unless the agents provoke a reaction against the cells, e.g., because the agents are surface markers for the cells that identify the cells to the immune system. Instead, the immune reaction is directed to the particular agents and the cells are only indirectly affected. In particular, autologous cells, unlike with non-autologous cells, can be combined with immunogenic proteins and can be expected to lack intimate association with immunogenic agents so that the cells will be free of specific attacks from the immune system.

The immune response can benefit treatment of the defect in the tissue while not causing a host rejection of the introduced cells. The immunogenic agents can induce an immune response that activates immune cells and many aspects of the inflammation response involving cytokines produced by the immune response. For instance, the immune response causes local site inflammation and erythema. The inflammation and erythema increases blood flow and delivery of nutrients to the site. Leukocytes, in particular macrophages and polymorphonuclear types, migrate to the site and produce cytokines or growth factors that regulate connective tissue matrix deposition by the fibroblasts or cells. This chain of events is followed by fibroplasias, reflecting an increase in the proliferation of fibroblasts and deposition of extracellular matrix. Angiogenesis takes place, and results in an increase in the local blood and nutrient supply to deliver nutrients to increase the survival and proliferation of the introduced cells. Macrophages and other immune cells secrete growth factors and cytokines that can increase the survival and proliferation of the introduced cells. When inflammation diminishes, angiogenesis ceases and the fibroplasia ends.

An immune response may also result in scarring of the surrounding local area of introduction. Scarring can, in itself, augment tissue. Scarring increases synthesis of collagen and other extracellular matrix proteins and molecules. Scarring can maintain the localization of cells preventing migration of cells from the intended site of the defect. Altered vasculature patterns are found in scars, granulation tissue and collagen and other extracellular matrix deposition and remodeling occurs along the pathways of neovascularization.

Immunogenic, serum and ECM proteins and molecules that enhance cell survival, growth and extracellular matrix production can be combined with living cells for tissue augmentation and repair of defects. For example, inclusion of growth factors with the cells can be beneficial and superior to cells alone. Often times the only practically available form, or most desirable form, of the protein is a recombinant xenogeneic or allogeneic form or a non-recombinant xenogeneic or allogeneic form. Xenogeneic refers to a source (tissue, cell, protein, macromolecule, molecule) from another species. Allogeneic or homologous refers to a source from another person within the species. Autogenic or autologous refers to a source from the same person.

Xenogenic bovine serum is an immunogenic protein that elicits a hypersensitive reaction (redness or erythema due to dilation of blood vessels around injection site) when injected into a host, e.g., in the subcutaneous region of the skin. Indeed, about 90% of patients develop antibodies in response to the implantation of commercially used bovine collagen. Similarly, lipolysaccaride (LPS) is composed of an O-antigen, core polysaccharide and lipid A. The O-antigen is very immunogenic and stimulates antibody production. Or, for example, bovine serum albumin implanted into a human host is immunogenic and could be used in certain embodiments.

Immunogenic Reactions

A number of types of immunogenic reactions can occur. Neutralizing antibody, binding antibody or hypersensitivity responses are among the types of immunogenic reactions. The response may be without T-cell help, transient have altered pharmacokinetics and not result in sample antibodies.

Hypersensitive reactions have been shown to occur with xenobiotic products from microbes and animal proteins (e.g. aprotinin) and with human origin products. Hypersensitive responses occur when a therapeutic protein is administered to patients in which the endogenous factor is mutated, nonfunctional, altered, absent or present at physiologically insignificant levels. Binding antibodies are a sensitive indication that the protein is antigenic and can elicit an immune response. Binding antibody may foster epitope spreading and neutralizing antibody development.

Some of the cells that are involved in connective tissue immune reactions are the macrophage (mononuclear phagocytes) which are adherent cells in contrast to B and T lymphocytes. Langerhan's cells are dendritic cells made from bone marrow, circulate to the upper epidermis and are antigen presenting cells that communicate with the lymph nodes. A histiocyte is a macrophage in tissue such as in connective tissue. Histiocytes are actively phagocytic and may be derived from monocytes in the circulating blood. Histocompatibility antigens or differential antigen processing can differ among individuals and can differentially produce antibody responses to the same protein. Additional immune cells present as fixed or transient are lymphocytes including T and B cells, monocytes, eosinophils, neutrophils, and mast cells.

There are a number of immune cells in blood and tissue. Various immune cells besides T and B cells circulate in the blood. Granulocytes constitute 58 to 71% of leukocytes in the blood circulation and refer to 3 types of polymorphonuclear leukocytes that differ mainly due to staining properties of their cytoplasmic granules. Basophils, neutrophils and eosinophils are all mature myeloid-series cells with different functions. Lymphocytes are B or T cells 7 to 12 u and contain a round to ovoid nucleus. Macrophages are mononuclear phagocytic and tumoricidal cells derived from monocytes in the blood that are produced from stem cells in the bone marrow. They grow as adherent cells in cell culture. Monocytes make up 3-5% of the leukocytes in the blood. Macrophages are also known as Kupffer cells in the liver and histiocytes in connective tissues. They produce IL-1, proteases, lipases, acid hydrolase, complement components C1 through C5, factors B and D, properdin, C3b inactivators, and β-1H. Mast cells are in connective tissue and play a role in immediate type I hypersensitivity and inflammatory reactions by secreting a variety of chemical mediators from storage sites in their granules. Mast cells become sensitized and have membranes containing IgE antibody receptors that bind IgE made by plasma cells on first exposure to an allergen (e.g. foreign serum). Mast cells have a function in type I anaphylactic reactions, inflammation and allergic reactions.

Dendritic cells enhance immunostimulatory functions and are antigen presenting cells. Langerhan's cells in the epidermis of the skin are antigen presenting cells. These cells develop delayed-type hypersensitivity through the uptake of antigen in the skin and transport of it to the lymph nodes.

Macrophages produce growth factors for fibroblasts and vascular epithelium that promote the repair of injured tissues. Macrophages produce cytokines which recruit other inflammatory cells, especially neutrophils and are responsible for many of the systemic effects of inflammation, such as fever. Macrophages phagocytose foreign particles, such as microbes, macromolecules including antigens and self-tissues that are injured or dead, such as senescent erythrocytes. They also display foreign antigens to be recognized by antigen-specific T lymphocytes and thus are antigen-presenting cells. Macrophages are among the principal effector cells of cell-mediated immunity and opsonize or get rid of foreign antigens by humoral immune responses.

Cell-mediated immunity is an immune response that does not involve antibodies. Instead it involves the activation of macrophages and NK-cells for the destruction of intracellular pathogens, the production of antigen-specific cytotoxic T-lymphocytes that lyse cells showing the antigenic epitopes on their surface, and the release of various cytokines from antigenic stimulated cells that alters the function of other cells involved in adaptive and innate immune responses. Besides its role in removing microbes and virus infected cells, cell-mediated immunity plays a major role in transplant rejection.

Humoral mediated immunity involves antibodies, primarily produced by B cells characterized by the adaptive immune response.

An immunogen elicits a B or T cell response and interacts with the products of that response. Immunogen is a term often interchanged with antigen, but an antigen really means a substance that an antibody reacts with. Thus an immunogen is not limited to being an antigen because an immunogen can elicit more than an antibody response. Proteins and polysaccharides with at least a molecular weight of 1000 are typical immunogens. Antigen is a substance that binds with the antibodies and/or T lymphocyte receptors of the immune response that is stimulated by a specific immunogen. Antigens can be proteins, carbohydrates (complex and simple sugars), lipids and phospholipids, nucleic acids, hormones, intermediary metabolites, and autocoids. An incomplete antigen or hapten does not induce an immune response alone but can react with the products of it e.g. antibodies. Haptens are rendered immunogenic by covalent linkage to a carrier molecule. Principal immunogens are proteins and polysaccharides, while lipids can serve as haptens. To be immunogenic a substance needs to be recognized as foreign to the recipient. Significant molecular size and complexity, host factors such as genetic factors for responsiveness (immunocompetence), and previous exposure to the immunogen are factors that determine immunogenicity.

Immunological reaction is an in vivo or in vitro response of lymphoid cells to an antigen never encountered before or to an antigen for which they are primed or sensitized to. An immunological reaction may consist of antibody formation, cell-mediated immunity or immunological tolerance. Protective immunity or hypersensitivity may come from humoral antibody and cell-mediated immune reactions.

Allergy refers to altered immune reactivity to a spectrum of environmental antigens. Allergy is also referred to as hypersensitivity and normally describes type I immediate hypersensitivity of the atopic/anaphylactic type. Sensitization is when the reaction induced is more of a hypersensitive or allergic nature than of an immune protective type of response to an antigen. Isoallergens are allergenic determinants with similar size, amino acid composition, peptide fingerprint and other characteristics. They are molecular variants of the same allergen and are able individually to sensitize a susceptible subject. Isoantigen is an antigen found in a member of a species that induces an immune response when injected into a genetically different member of the same species. Isoantigens of two members may have identical determinants. If not, they are allogeneic with respect to each other and are called alloantigens. Tolerogen is an antigen that is able to induce immunologic tolerance (tolerance involves lymphocytes as individual cells whereas unresponsiveness is an attribute of the whole organism.). The production of tolerance vs. immunity in response to antigen depends on the physical state of the antigen (soluble or particulate), route of administration, level of maturation of the recipient's immune system and immunogenic competence. Soluble antigens administered intravenously can favor tolerance in many situations but particulate antigens injected into the skin favor immunity.

Detection of Immunogenic Reactions.

A number of analytical methods exist to detect immunogenic reactions in sera. Assays should be specific, sensitive and able to detect low affinity antibodies. The biosensor assay, bioassay for the identification of neutralizing antibodies, the radioimmune precipitation (RIP) and enzyme-linked immunosorbent assay (ELISA) binding assay are a few of the assays available. Each type of assay has advantages according to the nature of the sample and antigen, among other factors. The biosensor immunoassay can determine antibody isotypes, subclasses and dissociation rates and is a preferred method to detect lower-affinity antibodies. Assays for binding of antibody to protein include ELISA (coat protein, add antibody sample, add a detector such as a labeled protein to the antibody [traditional method] or the labeled protein [bridge method]), RIP (in which radioactive labeled protein is precipitated with antibody sample), immunoblotting and BIA core method (surface plasmon resonance). In the BIA core method the protein is immobilized and protein is added to inhibit binding of antibody sample. The concentration, isotype, relative affinity and specificity of antibody can be determined. The bioassay can determine if the antibodies are able to neutralize the biological effect of the therapeutic immunogenic protein. The bioassay can be formed in cultured cells in which biological response, such as proliferation, can be measured after the addition of the protein plus and minus the antibody sample to the protein. Other endpoints to measure antibody effects can be cytokine release (measure by ELISA), mRNA expression, or apoptosis (caspase or other apoptosis assays). The cells can be natural primary cells, cell lines, or engineered cell lines in the bioassay.

Immunogenicity can sometimes be predicted based on T cell epitope identification, use of computer algorithms and reaction with patient sera. Assessment in other animals can be used as well that examines titer, cross-reactivity, neutralization and kinetics of development and duration. As mentioned above a number of factors determine immunogenicity of proteins including the molecular structure of new epitopes, aggregation, glycosylation, degradation, oxidation, deamidation; immunoregulatory features of the protein; how the protein is formulated; what impurities are present; the administrative route, doses, frequency and duration of treatment; the immune status and genetic background of the patient and the disease or defect. However, the immune system is the ultimate system that can detect alterations in the protein that are immunogenic that current analytic methods can not.

Immunogenicity Factors

Many factors can cause a protein to be immunogenic. Naturally occurring, synthesized, purified or recombinant proteins share many such factors in their immunogenicity potential. One factor may be the route of administration. Inmune response of an antigen can vary according to the portal of entry of that antigen. Subcutaneous or intradermal administration usually creates immune responses to antigens more consistently than intravenous or intramuscular routes. The reason may be due to the preponderance of antigen presenting cells in these tissues. Macrophages in the dermis and Langerhans cells in the epidermis are antigen presenting cells that present antigen to T cells. Another factor is the depot effect of subcutaneous injection in which adjuvants or substances facilitate the slow release of antigen at the injection site over an extended period to attract macrophages to the site of antigen deposition. A third reason may be the aggregation of proteins (either to form or to maintain an aggregate) in a more confined space. Dosage, levels and length of treatment of the therapeutic may affect the immune response.

Frequent administration can increase the immune response. Lower dosages can increase the immune response, whereas high doses of protein (intravenously) can induce tolerance. Product origin is significant. Immune response to foreign proteins (xenogeneic or allogeneic) are expected and can also be anticipated for some self-proteins. Recombinant cytokines such as thrombopoietin, present at only picomolar levels, elicit an immune response.

The rapidness of the immune response and its strength and persistence depends on many factors including the administration route and previous or ongoing environmental exposure. Product related factors, such as the presence of an adjuvant or the type and level of aggregation and any inherent immuno-modulatory activity (primary structure, e.g., sequence) can increase the immunogenicity in a given dosing regimen. Alterations in molecular structure can also elicit an immune response. Neodeterminants, such as those created by the fusion of a therapeutic protein with a partner antigen, a signal or lead peptide, an amino acid modification or those created by improper or incomplete glycosylation are a source of immunogenicity.

Glycosylation may strongly modulate immunogenicity of the proteins to which they are attached. Glycosylation is a complex post-translational modification that can result in extensive heterogeneity in autologous, allogeneic, xenogeneic sources and for recombinant glycoproteins produced by eukaryotic systems. Differences in host cell type, the physiologic status of the cell, and protein structural constraints result in variation in post-translational modifications that affect its immunogenicity, stability, susceptibility to proteolysis and bioactivity. Such microheterogeneity can affect the protein's immunogenicity. There can be batch to batch inconsistencies and instability. Glycosylation has an effect on secretion efficiency, extracellular stability and solubility. N-glycosylation of heterologolous proteins has a major affect on intracellular proteolytic processing (decreases protease attack), secretion efficiency and post-translational ability of proteins secreted from eukaryotic host cells. E. coli host cells do not have the eukaryotic glycosylation ability and thus therapeutics derived through this type of manufacture will exhibit a non-native profile of glycosylation, as well as a host of other post-translational modifications affecting the immunogenicity of the protein. The effects of eukaryotic cells may be mediated by a number of possible pathways, such as to mammalian xenogeneic sugars, yearst mannans, or plant sugars. Some of these can be attached through the recombinant protein pathway. The absence of properly glycosylated amino acid residues may create neodeterminants that increase the immunogenicity of the protein. Modification of the glycosylation residues on proteins can induce antibody formation. Antibodies that develop against proteins that have unprotected glycosylation sites, that occur for example with recombinant human GM-CSF or by making the protein more soluble (e.g. INF-β).

The presence of a carbohydrate moiety on a recombinant glycoprotein, proteoglycan or protein can cause immunogenicity of the protein. For example, the addition of glycosylated sites not normally present on a protein can also cause an immune reaction. Proteins in pig organs contain sugar residues (galactose α 1,3 galactose) on the vascular endothelium that interact with host antibodies and cause immune rejection of the organ. About 1% of serum antibodies in humans is directed to this sugar residue. It is present on the cell glycoconjugates of all mammals except man, chimp and gorilla. A tissue or cell with this sugar residue will elicit a rapid rejection involving complement and leukocytes. Other post-translational modifications can also affect immunogenicity of a protein, including phosphorylation or dephosphorylation, addition or loss of lipid moieties, methylation, ADP-ribosylation, oxidation, conformation changes, amongst others and yet are needed for activity and stability.

The widespread use of recombinant proteins stems from their biological safety compared with products of animal or human origin. But recombinant proteins, which are typically xenogeneic proteins or allogeneic proteins can generally produce an immunogenic response. Antibodies develop to varying degrees with human proteins that belong to the human species and is homologous to the natural form. Examples include insulin, growth hormone, granulocyte-macrophage colony-stimulating factor, factor VIII, erythropoietin, interleukin-2 and the interferons α and β. Some studies show an incidence of forming antibodies of greater than 80% with human interferon preparations. There are reports of 100% incidence with erythropoietin. And 44% of diabetics elicited an antibody production with recombinant human insulin. In human growth hormone, 63% incidence was observed with methionine recombinant human growth hormone.

The mechanisms that generate antibody or an immune reaction to recombinant proteins are the subject of ongoing scientific investigations. Impurities or contaminants can foster an immune reaction. Additional factors are listed below.

Size Small proteins or peptides are less likely than large or complex proteins to elicit an antibody response.

Autoantibodies A self-antigen is administered as a protein in which the patient already has an immune response.

Denaturation Protein denaturation present neodeterminants of the primary structure of proteins or an altered conformation to the immune system.

Aggregation This is a significant mechanism of inducing an immune response.

Homologous proteins often induce antibodies due to aggregation. The antibody production may be slow and binding antibodies appear after treatment and disappear with time. Protein aggregates can induce an immune response to the monomeric form of the protein. This may take place by the cross-linking of a sufficient number of B-cell receptors causing efficient B-cell activation and enhance antigen processing and presentation, thereby efficiently recruiting the T-cell repertoire critical for generating a high-affinity IgG antibody. The ability of protein aggregates to generate antibody (such as neutralizing) may depend on the preservation of the native conformation of the molecule within the aggregate. Aggregates of denatured protein generate antibody (binding) but can be less potent in generating neutralizing antibody. Antibodies to linear determinants in the protein, contact sites or epitope spreading could account for the neutralizing activity. Protein aggregates have been shown to occur in many therapeutic proteins including type I interferons, rHu (recombinant human) interleukin-2 and human growth hormone. For example, IFN-α contains 10 to 5000 times more human serum albumin (HSA) and both IFN-α-IFN-α and even more likely HSA-IFN-α aggregates form during formulation and storage.

Proteins made by recombinant means in bacterial systems are normally aggregated in inclusion bodies. It is required for functionality to refold and re-nature the proteins to make them soluble. Not all are disaggregated. Filtration can cause aggregation or denaturation. It has been shown that aggregated human growth hormone, insulin and IgG are more immunogenic than the monomer. Physical or chemical protein modifications are added causes for aggregation.

There will often be an immune response against recombinant animal or human proteins. Factors in the immune response against human recombinant proteins can be classified into 3 major categories: 1) source of the recombinant protein that includes the host cell production in bacteria, yeast, plant or mammalian cells; the presence of any contaminating proteins, glycosylation differences, and factors as described above; 2) formulation factors including the use of excipients, chemical and physical protein modifications, including denaturation and aggregation; 3) clinical factors such as the route of administration, the dose and duration treatment, presence of autoantibodies, disease state and age of patient.

Formulation Components in the formulation of the protein product are included to maximize the in vivo activity by preserving the native conformation of the proteins that may be lost otherwise to hydrophobic interactions among protein molecules and surfaces such as air or glass. Also components are added to prevent protein degradation due to oxidation or de-amidation. Large proteins, like albumin, can be included as excipients in the formulation, but can contribute to an increase immunogenic response. Although the purpose of large proteins is to inhibit hydrophobic interactions, they may co-aggregate with product or form protein adducts. For example, as described above, interferon-α-human serum albumin aggregates foster immune responses to interferon α. IFN-α formulations contain HSA due to its good solubility, thermal stability and ability to prevent surface absorption of active proteins. HSA also interacts with other proteins.

Other excipients such as non-ionic detergents can cause micelle formation or leach organic molecules and metal ions, which can have adjuvant activity.

Adjuvants Adjuvant activity can arise from other sources than formulation. Adjuvants may be present in microbial host-cell proteins, oligonucleotides or polysaccharides which can exert direct adjuvant activity with toll-like receptors (e.g. macrophages) or other recognition molecules present on B cells and other antigen presenting cell populations. The protein product itself may be an adjuvant. For example, type I interferon, interleukin 2 and GM-CSF upregulate immune responses to themselves. This is true with other biological therapeutics, endogenous (self) proteins and small drug molecules.

Most proteins are sensitive to heat, light and mechanical agitation and these conditions cause aggregation and denaturation. Also storage conditions and time can affect these parameters. Handling conditions, can cause protein changes that result in immunogenicity, including protein to protein interactions. Proteins aggregates can also be induced by stress conditions, such as exposure to temperature and pH extremes, introduction of a high air/water or solid/water interface and addition of pharmaceutical additives.

Product origin An established example of the effect of product origin is insulin, a polypeptide hormone, m.w. 5,900, composed of 2 chains joined by disulfide bonds. The A-chain has 21 amino acids and B-chain has 30. Bovine insulin differs from human insulin by 3 amino acid changes. Structural differences between porcine and beef insulins and human insulin result in the antigenicity of the animal-source insulins. Porcine insulin differs from human insulin by 1 amino acid change. Bovine insulin was more immunogenic than the porcine source (60% incidence) which was more immunogenic than the human source (recombinant). The recombinant was still immunogenic to 44% of diabetics. The majority of recombinant proteins have amino acid sequences almost identical to the corresponding human proteins, but when individual polymorphisms, for example, are taking into account, there can be quite a number of amino acid differences between what the host tolerates and what the recombinant protein contains. Local reactions to insulin are due to immediate hypersensitivity (type I allergy) with formation of skin-sensitizing IgE antibodies and delayed hypersensitivity after T-lymphocyte stimulation. Others report both IgG and IgE insulin-specific antibodies in diabetics treated with recombinant insulin. In summary the immune response to insulin is a B lymphocyte production of humor antibodies, an immediate hypersensitivity characterized by skin sensitizing antibodies (IgE) and a rarer insulin resistance with neutralizing antibodies (IgG). In addition, the T lymphocytes display a delayed hypersensitivity, a local delayed allergy. The skin may show lipoatrophy or hypertrophy with an uncertain role of the immune response. Local cutaneous reaction to insulin is noted as a mild reaction consisting of a stinging, burning or itching sensation at the site of injection within hours after insulin administration. In others, the reaction is shown as local swelling, erythema (due to dilation of blood vessels around immunogen injection site), induration and occasional allergic wheal formation at the injection site.

Different classes of protein therapeutics can by immunogenic such as animal derived proteins, human derived proteins, human recombinant proteins of homologous sequence, variant sequence, chemical modification, fusion or hybrid proteins and antibody therapeutics, either fully human antibodies or humanized, murine or chimeric antibodies. Proteins introduced to patients can induce antibodies that either have no effect on the protein's efficacy or that can alter the pharmacokinetics of the therapeutic. Most biopharmaceuticals, primarily proteins made through recombinant DNA, induce antibody formation, usually through reaction to new antigens or immune tolerance breakdown mechanisms and thus are immunogens.

More examples of immunogenic protein therapeutics are antibodies that can neutralize the effects of the therapeutic such as observed with factor VIII, IFNα2a and GM-CSF or can cross-react with native proteins resulting in adverse effects as seen with EPO and MGDF. Example of recombinant proteins homologous to native proteins and yet are immunogenic by binding or neutralizing antibodies are IFN-α2a, GM-CSF, G-CSF, IFN-β, Epo, IL-2, GnRH, HCG; recombinant proteins that are sequence variants and are immunogenic are IFN-β and IFN-α Con 1; recombinant proteins that are chemically modified (pegylated MGDF) or hybrids (GM-CSF/IL-3 hybrid or TNFR2-Ig are immunogenic; proteins made by natural cells and yet are immunogenic are non-human proteins calcitonin and insulin, the human proteins glucocerebosidase and factor VIII. Antibodies are found against non-product related proteins derived from the expression system (e.g. E. coli proteins). Antibodies can be just binding or binding and neutralizing. Patient variability and environmental influences can be found in patients that differ in antibodies to the same therapeutic such as with GM-CSF.

Primary Structure Polymorphisms predominate in the genetic coding of proteins and account for immunogenic reactions even among different individuals of the same species. In general there is a 1% difference in coding sequence among individuals and a much larger difference between species. Thus recombinant or purified proteins from different individuals of the same species will have 10 base mutations for every 1000 base pairs of coding region or a 33 amino acid or 3,300 dalton protein or polypeptide will have one amino acid replacement due to mutation. Such a replacement can cause an immunogenic reaction since the antigen site will be different from one's own protein counterpart that is immunotolerated. Purified or recombinant proteins that are used in conjunction with implantable cells will thus carry such immunogenic potential. Responding T cells are often specific for one or a few linear amino acid sequences of the antigen. Thus differences in the primary structure due to polymorphisms can result in the protein being immunogenic. Xenogenic proteins can cause fast antibody production, after a single injection and last for long periods of time.

Other changes to amino acid sequence, not another amino acid, but to a modified amino acid (e.g. deamidation, oxidation) can cause an immunogenic response.

Proteins in the serum can be immunogenic by virtue of the age of the patient. The older the patient the higher the amounts of AGEs, advance glycosylated end-products. These proteins crosslink with sugar moieties and increase with age. AGEs include many types of proteins in the serum, such as amyloid, hemoglobin, albumin, and β2-microglobulin. AGEs also are present in the ECM and inside cells. Examples of ECM that is crosslinked as an AGE product is collagen, elastin, β-amyloid, neurofibrillary tangles and other aggregates present in Alzheimer's and other diseased tissue. Lipoproteins, such as LDL, can be immunogenic. Immune complexes isolated from human sera contain autoantibodies reacting with modified LDL such as malondialdehyde-modified LDL, N(carboxymethyl)lysine-modified LDL, oxidized LDL, and advanced glycosylation end product (AGE)-modified LDL.

Synthetic antigens are derived exclusively by laboratory synthesis, not living cells. Synthetic polypeptide antigens have a backbone consisting of amino acids that can include lysine (poly-L-lysine). Side chains of different amino acids are attached to the backbone and then elongated with a homopolymer or attached via the homopolymer. The specificities are determined by the number, nature and particular arrangement of the amino acid residues of the molecule and can be made more complex by further coupling to haptens or derivatized with various compounds. The size is less critical than with natural antigens. Thus p-azobenzenearsonate-N-acetyl-L-tyrosine, 451 molecular weight, or p-azobenzenearsonate coupled to three L-lysine residues, molecular weight 750, can be immunogenic. Polylysine can be used as an attachment molecule for cells in vitro and in vivo.

Materials from the cell culture can be immunogenic. For example, proteins used in cell culture that remain in the cell implant, trypsin digestion used to release cells from the cell culture vessel, serum proteins used for cell proliferation, ECM molecules or serum molecules used for cell attachment, such as fibronectin and other cell adhesion proteins can carry into the cell implant. Alternatively, many molecules or proteins can be added to the cell implant for improved safety or effectiveness of the treatment.

When serum is utilized autologous serum is the preferred embodiment to culture cells and may be present in the implantation of cells or by itself. Autologous family serum can be substituted and used in which a family member's serum is obtained. Autologous family serum from younger family members for superior growth and implantation characteristics may advantageously be used. Serum from family members contains less allogenic proteins that are immunogenic than the use of non-autologous human serum which contains more and a higher degree of allogenic proteins that are immunogenic.

To treat defects, immunogenic agents can vary in concentration from more than 0% to 100% v/v or 0% to 100% w/w if used alone and more than 0% to less than 100% w/w if part of the cell composition.

With respect to autologous cells, the inclusion of immunogenic agents (molecules) including polymers, polypeptides, amino acid sequences, proteins, serum proteins, extracellular matrix proteins and non-protein molecules can be introduced with the cells of the subject into the subject. Additionally, immunogenic agents without cells can be introduced to treat defects.

Cell and Serum Types

A variety of cells may be used with these methods, including, for example, fibroblasts, muscle cells, endothelial cells, epithelial cells, mesenchymal cells, and embryonic or adult stem cells. For example, stem cells or autologous cells may be used to correct the defects, or other cell types from different (non-autologous) and various (human and animal) sources.

Cells typically progress through stages of differentiation from uncommitted pluripotent cells into differentiated end cells. Differentiation is a process of cells becoming increasingly specialized and is marked by a transition from a first state to another, stable state. The pathway of differentiation and its progression through various cell types is the lineage of the cell. Examples of complete undifferentiated cells are totipotent embryonic cells or germ cells. The implanted cells may be terminally differentiated or non-differentiated. Non-differentiated cells represent those cells that have not undergone terminal differentiation and are thus totally undifferentiated or only partially differentiated. Terminal differentiation of a cell is normally found in adult tissue and represents the last normal differentiation state of a non-differentiated cell. For example, the reticular or papillary skin fibroblast is an example of a terminally differentiated cell whereas dermocytes or other progenitor cells prior to complete differentiation into a reticular or papillary fibroblast are a non-differentiated or partially differentiated precursor cell in the skin fibroblast lineage.

Embryonic stem (ES) cells can be totipotent if obtained at the morula stage. Totipotent cells can differentiate into any cell type in the body, including germ cells. Germ stem cells are in the totipotent class. Pluripotent cells, taken from the embryonic blastocyst stage, have already undergone some differentiation, so that these cells, derived from embryonic stem cells, have the capacity to differentiate further down the ectoderm, mesoderm or endoderm lineage into a variety of cell types, but can not differentiate into a germ cell. Thus, almost all cell types can be expected to be differentiated from pluripotent cells of embryonic stem cell origin. Pluripotent cells that differentiate further into one of three particular cell lineages are often referred to as multipotent cells. These cells have a limited number of differentiations remaining to convert into a specific cell type. The proliferation potential of stem cells are almost indefinite.

Adult stem cells are in the multipotent class and are present in many tissues and perhaps in all. Stem cells from umbilical cord and fetal stem cells can be in the multipotent or pluripotent class.

Cell types that can be used are from adult, fetal, neonatal, umbilical cord, embryonic tissue or somatic nuclear transfer and can present themselves as stem cells. Cells can be isolated directly from the living sources as primary culture or developed into cell lines. Stem cells can be totally undifferentiated (totipotent) so as to have the potential to generate any cell type lineage including germ cells or can be partially differentiated (pluripotent, multipotent) so as to have the potential to form a limited cell type or a set of multiple lineages. Stem cells can be from an autologous or heterologous or xenogeneic source. Some examples of adult stem cells are hematopoietic stem cells, bone marrow stem cells, unfractionated bone marrow stem cells, mesenchymal stem cells, neural stem cells and multipotent adult progenitor cells. Bone marrow cells can contain four cell lineages, hematopoietic stem cells, mesenchymal stem cells, multipotent adult progenitor cells and progenitor endothelial cells.

ES and other non-autologous stem cells, as they differentiate or grow in vitro and in vivo, express non-autologous immunogenic proteins and molecules. The embryonic stem cell established from a blastocyst, embryonic germ cell line established from the reproductive cells of the fetus, stem cells from embryoid bodies, and downstream intermediate stem cells established from these sources can be used as heterologous cells, unless modifications to cells are done to overcome donor/recipient incompatibility and graft rejection, such as embryonic stem cells derived by somatic nuclear transfer.

Transdifferentiation refers to cells that can be converted from one cell type into another. Transdifferentiation can be the conversion of terminally differentiated cells into another cell type.

The conversion of one cell type to the desired cell type, either cell transdifferentiation or differentiation of precursor cells of this invention can be accomplished in vitro or in vivo. In vitro, before, during or after expansion of cells the addition of extracellular matrix (especially ECM from the desired cell type) can convert the cells into the desired cell types. In an alternate method, cell extracts from the target cell phenotype desired can be added to the cells to produce the desired cell type. In a third method, co-culturing of the cells with the desired cell type can produce the conversion to the desired cell type. Alternately, the addition of specific hormones and/or growth factors in a temporal fashion to the cells can produce the cell type desired. Maintenance of the specific cell phenotype can be accomplished by the continued presence of the desired cell types' ECM, cell extracts, co-culture with the desired cell type, and other factors such as growth factor or hormones. The extracellular matrix or cell extracts in vitro can be obtained from the tissue the cell type resides or from the culturing and/or expansion of the cell type desired. In a preferred embodiment, differentiation of precursor cells, transdifferentiation of cell types and maintenance of a specific cell phenotype can be accomplished in vitro by incubation of a cell type in the desired cell type ECM and can be obtained from the desired cell type in vitro or desired tissue ECM. In vivo, implantation into the desired cell type environment (the extracellular matrix or specific in situ cell type(s)), can convert the implanted cell into the desired cell types.

ECM synthesized in three or two dimensions can be used. The ECM can be included in the implantate to further ensure cell phenotype maintenance, cell survival and inhibition of anoikis. Xenogenic, allogenic or autologous ECM or its constituents can be used with autologous or non-autologous cells. Matrices that can be used include natural and synthetic, are preferably biodegradable and can contain immunogenic determinants that with time are removed by degradation or other mechanisms. Matrices can contain a variety of physical forms of molecules. They can be scaffolds, nano-fibers, sponges, foams, and a number of polymer types, bipolymers, proteins, charged or hydrophobic surfaces, etc. can be used as components. Matrices can be multilayered with different proteins, molecules and polymers in each layer. Matrices can contain in whole or in part various proteins that are advantageous for implantation. Matrices can contain matrikines, motifs or domains of ECM proteins, MMPs or inhibitors of, ECM receptors such as integrins, growth factors, cytokines, chemokines, pro-coagulation sequences, plasmin degradation sites, proinflammation sequences, amongst many other possibilities, that can promote wanted cell proliferation, differentiation and other functional outcomes. Cells in culture can produce dense 3-D matrices (e.g. via proper serum supplementation that overcome contact inhibition) and cells within these 3-D matrices form a distinct class of adhesion. Fibrillar adhesions containing long fibrils of fibronectin or 3D matrix adhesions are dependent on integrin α₅β₁ and fibronectin. Cells adhere more rapidly to the 3D matrix and have more rapid migration, proliferation and morphological changes than 2D matrices or 3D collagen gels.

When serum is used, autologous serum is a preferred embodiment to culture cells and implant cells. Autologous family serum, especially in which there is a close genetic match can be substituted and used in which a family member's serum is obtained. Family serum from younger family members for superior growth and implantation characteristics are preferred. Younger serum, instead of older serum, contains factors that promote better cell growth and proliferation, cell adhesion and migration, and maintenance or differentiation of cell phenotype. Younger serum promotes the expansion of stem cells and differentiated cells. Younger serum, as opposed to older serum, contains factors related to the young phenotype including different concentrations and/or types of growth factors and hormones. Younger serum, from unrelated humans, can be used in a preferred embodiment, especially for the culturing of ES or stem cells.

Cell types described in this invention, including human cells such as embryonic stem cells, stem cells and other cell types, such as those incorporated by reference, can be grown in cell culture medium that is serum free or contains human or autologous serum. These serum medium conditions can be used for maintaining undifferentiated cells or for differentiating the undifferentiated cells to a partially or fully differentiated cell type state. For ES cells to grow fetal bovine serum and mouse feeder cells are now used—both animal derived requirements. Typically ES cells are grown on a mouse fibroblast feeder layer to maintain an undifferentiated state. In a preferred embodiment use of the subject's own fibroblasts as a feeder layer can be used to prevent differentiation of ES or other stem cells to other cell types. Other human cell types and non-autologous human cells can be used as an alternate method for a feeder layer. Also ECM and growth factors from the above cell types can be used instead of cells or in combination with cells for the feeder layer. For example, serum free medium containing growth factors, that fibroblasts secrete, such as the fibroblast growth factors (e.g FGF 2, epidermal growth factor, platelet-derived growth factor, the insulin growth factors, transforming growth factor family β), among others can be used in combination or by itself to maintain the non-differentiated state.

Animal serum has the disadvantage of contaminants that can transmit disease or make the cells immunogenic and rejected by the host. Bovine serum contains N-glycoslylneuraminic acid that is absorbed onto the ES cells and causes cell rejection. This sialic acid evokes an immune response with sialic specific antibodies present in human serum. Animal sera contains contaminants thus can alter the immunogenicity of stem cells resulting in increased immunogenicity of the stem cells and subsequent rejection by the host. Animal serum can also contaminate normal non-stem cells in a similar manner with similar consequences.

Human serum can prevent this problem present in embryonic stem cells, other stem cell types and somatic cell types. The preferred serum for ES, other stem cell and somatic cell culturing is the implanted subject's serum or younger human serum for enhanced cell growth. In a preferred embodiment autologous serum from the subject is used to culture the cells that are to be implanted into the subject. These cells can be non-autologous as well as autologous cells, including stem cells, differentiated adult cells, fetal and juvenile cells. In another preferred embodiment, serum from genetically matched or individuals genetically closer to the subject than the general population, such as family members, can be used to culture cells.

In another preferred embodiment serum from younger aged humans are used instead of the subjects serum or older serum to culture cells. This can result in better survival and proliferation of the cells, including the promotion of tissue stem cells. This can be especially true for stem cell types in which young serum contains the proper quality and/or quantity of growth promoting substances. Thus in vivo young serum can stimulate stem cell growth and gene expression to survive. Cell culture can use young serum for similar reasons. Another source of serum can be any human's serum. Amniotic fluid may be a source of human sera for cell culturing. Serum free medium can be used as well. Other serum that can be used is umbilical cord serum or blood and follicular fluid or serum. Serum free conditions using growth factors (e.g. insulin, selenium, transferrin), milk, sugar substitutes like dextrins, agarose, in serum free medium can be used. Benefits of these alternate serum sources include increased cell proliferation ability, decreased senescence and apoptosis of cultured somatic cells.

In another preferred embodiment younger whole blood, fractionated blood, plasma, and/or serum is implanted or infused into the subject's tissue or entire body. Younger whole blood or fractionated blood can contain progenitor cells, as well as other factors that are also found in (younger) plasmas or serum, such as hormones, growth factors, and other factors that enable relatively older tissue or diseased tissue to regain or improve its function. Preferably donor whole blood, fractionated blood, plasma, or serum is compatible (e.g., histocompatible, ABO type, Rh compatible) with the host or does not cause any adverse reactions (e.g. immune reactions). Younger whole blood, fractionated blood, plasma, or serum refers to whole blood, fractionated blood, plasma, or serum from a person that is younger than the patient that receives the blood, plasma, or serum, including, for example, younger by at least 5, 10, 15, 20, 30, 40, or 50 years. Embodiments include serum and/or cells taken from the patient and stored until a later date, e.g., 20 years later. Embodiments include selecting the donor to be a younger person, and selecting the donor based on their familial relationship; while blood donations are made between persons of different ages, it is believed that such donations are made by chance and not by intentional selection. The intelligent choice of donors of a younger age and/or close familial relationship advantageously makes stem cells, multipotent cells, and other factors available to the patient. Choice of the degree of the familial relationship include, for example, at least 10%, at least 25%, at least 50% genetic similarity, e.g., as between siblings, parents and children, nieces or nephews and their uncles or aunts, grandparents and grandchildren, and as between cousins of at least 10% genetic similarity. By way of example, a child is 50% genetically similar to a parent and an uncle is 25% genetically similar to a nephew. Multiple or repeat infusions may be used, for example every week, every month, or otherwise on a repeat basis. Without being limited to a particular theory, blood, plasma, and serum factors infused into a patient can be directed by the patient's body to tissues that are need repair, e.g., of defects, pathologies, or aging. Repetitive treatments may be performed until tissue function is enhanced as determined by observation or diagnostic testing.

Animal serum or animal feeder cell types (e.g. cell co-culture) can be used for certain cell applications.

Serum concentration used for cell expansion in vitro can vary depending on cell type and type of cell expansion (e.g. matrices) from greater than 0% to 100%, with a preferred range of less than 20%. Serum can be included with cells for implantation ranging in concentration ranging from greater than 0% to less than 100%. Serum used without cells during implantation can be used in concentrations from greater than 0% to 100%.

Serum-derived proteins can be used in the cell culture medium singly, in combination, as a constituent of the whole serum added or as an addition to whole serum added to the cell culture medium. Serum-derived proteins can be added to the culture medium in the cell expansion process or to the implantate. Serum-derived proteins can be obtained from xenogenic, allogenic, autogenic and/or recombinant, peptide sources, amongst other sources. Serum-derived proteins can be optionally immunogenic. Serum-derived protein(s) can be implanted singly or in tandem with cells into the subject to treat the tissue defect. Singly they can represent in content from greater than 0% to 100% v/v or w/w of the implantate composition. In tandem with cells serum-derived protein(s) can vary in concentration from more than 0% to less than 100% v/v or w/w. Persons of ordinary skill in these arts will appreciate that all ranges not explicitly articulated are contemplated, e.g., from 0.1%-50%, 0.2%-20%, or 1%-20% v/v or w/w.

The culture medium can be included with cells in the implantate in concentrations ranging from greater than 0% to less than 100%. Without cells culture medium can be used up to 100% concentration in the implantate. The culture medium (e.g. last cell passaged medium) contains proteins and other factors produced by the cells in vitro and can be considered conditioned medium that can be used in the implantation procedure. The conditioned medium can contain serum-derived proteins that are produced by the cells themselves. In addition, conditioned medium can contain additives of whole serum or serum-derived proteins. Conditioned medium may increase the effectiveness of the cells to treat the defect.

Techniques for culturing cells in vitro are known for many types of cells. The culture of differentiated or mostly differentiated cells has been studied at length so that ordinary artisans can perform a routine investigation of the cell culture literature to determine the necessary conditions for isolating cells from a sample or from a commercial source, maintaining the cells, and expanding them to increase their number. The culture of stem cells and pluripotent cells is the study of intense scientific investigation at this time, so that culturing techniques for many such cell types are known, although new techniques and stem cell types are continually being discovered. Materials and methods are described herein that can be adapted to take advantage of all of the cell types that are known and that are being discovered. The reference section of this patent application includes a variety of publications that illustrate some of the relevant cell culture techniques, but is not intended to be an exhaustive list of the voluminous cell culture literature.

Human cells including embryonic stem cells, stem cells and other cell types, such as those described in articles incorporated herein by reference, can be grown in cell culture medium that is serum free or contains human or autologous serum. These serum medium conditions can be used for maintaining undifferentiated cells or for differentiating the undifferentiated cells to a partially or fully differentiated cell type state. Human feeder cell types and ECM can be used instead of animal feeder cells also for the above reasons. Desired differentiated cell types and the ECM of the desired cell type can be used for these differentiation purposes. Animal serum can be used for certain cell applications as well as animal feeder cell types.

Autologous cells are preferred in the invention. Younger, rather than older, autologous cells are preferred and can be cells obtained and stored (e.g. cryopreservation) from previous chronological biopsies of the subject. Other non-autologous cells can be used that can act more efficaciously or in the case where autologous cells could be detrimental, as is the case with genetic diseases that confer dysfunctional characteristics. In another preferred embodiment, genetically similar cells can be substituted for autologous cells. Thus younger cells for example, can be preferably be used rather than older cells. These cells can be obtained from family members that do not induce rejection (e.g., cells with matching histocompatibility molecules). Alternately, non-genetically similar, non-autologous cells that do not induce rejection can be used. Younger cells include for example, younger adult, pre-adolescent, neonatal, fetal and embryonic cells. Younger cells are particularly important when older cells do not have the same functional profile as younger cells and an increase in their numbers are still insufficient to correct the accompanying tissue dysfunction.

Additionally, non-sun, chemical or radiation exposed cells are preferred for use in the invention for most purposes. The cell phenotype needs to be appropriate for the tissue site it is implanted into and taken into account with the ease of isolation of the cells from the patient and for expansion usefulness.

Some cell types that are useful for augmentation and/or repair of defects include cells that can be cultured in an adherent state. These include, for example, fibroblasts derived from connective tissue, dermis, fascia, or lamina propria tissue. Other cells are pre-adipocytes or adipocytes. Chondrocytes and osteoblasts may be used in some cases but are not suited for tissues wherein calcification would be disadvantageous, which is often the case for soft tissues. Chondrocytes and osteoblasts, however, are suitable for cartilaginous or bony tissue. Other cell types include epithelial, endothelial, muscle, (smooth muscle, skeletal and cardiac) amongst many others. The cells may be obtained from tissue samples, including samples from the patient to receive the cells (autologous), samples from others of the same species as the patient (allogeneic), and samples from other species (xenogeneic). A biopsy or other excision of tissue may be used to obtain samples.

In general, suitable cell and tissue culture techniques are available for the isolation and expansion of the cells, including primary cells, stem cells, and pluripotent cells e.g., Culture of Animal Cells: A Manual of Basic Techniques, Freshney, R. I., ed., (Alan R. Liss & Co., New York 1987); Animal Cell Culture: A Practical Approach, Freshney, R. I. ed., (IRL Press, Oxford, England (1986); and Methods in Molecular Biology Volume 290 Basic Cell Culture Protocols 3^{rd} Edition Cheryl D. Helgason and Cindy L. Miller Human Press Inc., Totowa, N.J., 2005, each of which are hereby incorporated herein by reference. Certain techniques for isolating and culturing some cell types, including fibroblasts, papillary and reticular fibroblasts are set forth in U.S. patent application Ser. No. 09/632,581 (filed Aug. 3, 2000) and Ser. No. 10/129,180 (filed May 3, 2002), which are hereby incorporated by reference herein. Isolation refers to obtaining a purified group of cells from a tissue sample. Expansion refers to increasing the number of cells. In general, expansion and differentiation are inversely related to each other, so that culture conditions that tend to differentiate the cells tend to suppress expansion.

Proteins and Macromolecules

A variety of proteins or other macromolecules may be used with these methods, for example, proteins from the extracellular matrix, serum-derived factors, or growth factors to improve or restore the functionality of defective tissue or tissue. The proteins or other macromolecules may be combined with cells or administered without the cells.

The proteins can be obtained by purification from xenogeneic, allogeneic or autologous sources. The proteins can be obtained by recombinant means or chemically synthesized in xenogeneic, allogeneic or autologous forms. The total protein, domains or motifs, fragments, or specific sequences can be the source of the protein added.

Recombinant proteins reduce the risks of prion contamination and plasma derived impurities for serum proteins that are available from animal sources. Examples of the many recombinant forms available include, but are not limited to human serum albumin, fibronectin and its fragments, fetuin, transferrin, and many other proteins, including those listed in this document.

Any of the factors listed above and/or present in serum, ECM, growth factors, cytokines, mitogens, hormones and others can be used in the invention singly or in combination with cells as an addition to the tissue of interest or entire organ or body of interest. Inhibitors of these factors, when beneficial to the tissue defect can be used. Also all forms can be used, the entire protein, fragments, domains, motifs and peptides that represent the protein's function. These forms can be obtained from natural sources, recombinant, chemical synthesis, proteolysis and a number of other man-made means. Autologous, allogenic or xenogenic sources of the proteins can be used.

Additional helpful proteins and factors are set forth in U.S. patent application Ser. No. 09/632,581 (filed Aug. 3, 2000) that claims priority to 60/037,961; Ser. No. 10/129,180 (filed May 3, 2002) that claims priority to 60/163,734, each of which are hereby incorporated by reference herein.

Extracellular Matrix

The extracellular matrix (ECM) is a structural entity surrounding cells in mammalian tissues. The extracellular matrix has numerous functions for supporting cellular activity and organization into tissues. Some extracellular matrix functions are related to mechanical properties, for example, elasticity, resilience, or osmotic properties. Other extracellular matrix functions are related to cell signaling cues that it provides. And, in some aspects, extracellular matrix serves as a scaffolding for other molecules that are useful to cells, for example, when it serves as a reservoir for growth factors that are released over time or in response to cell contact or cellular proteolytic action. The extracellular matrix, in general, is made from structural proteins such as collagen and elastin, specialized proteins such as fibrillin, fibronectin, or laminin. Proteoglycans (also termed mucopolysaccharides) have a protein core decorated by chains of repeating disaccharide units termed of glycosaminoglycans (GAGs) forming complex high molecular weight components of the extracellular matrix.

In general, cells require a suitable extracellular matrix for ultimately making a successful adaptation to a locale, and most tissues are characterized by a particular arrangement of extracellular matrix. Extracellular matrix provides biochemical cues and structural underpinnings for cellular survival, proliferation, and integration with other cells and organs. Therefore the introduction of extracellular matrix or extracellular matrix molecules with cells into an implant site is helpful to enhance the “take” of the implant, as well as helping to provide reproducible results by avoiding the unpredictable effects of excessive cellular mortality.

Introduction of extracellular matrix molecules as a solution or suspension is helpful to assure their availability for interaction with the cells and the implant site. A solution of an extracellular matrix molecule refers to a condition where in the extracellular matrix molecule is apparently dissolved. This use of the term is consistent with the state of these arts. It is recognized, however, that extracellular matrix molecules might be characterized in the terminology of other arts as being suspended or colloidally dispersed. Further, in a soluble form, extracellular matrix molecules may be more readily available to cells for metabolization as energy or building blocks for other molecules.

In fact, extracellular matrix has been recognized as a useful adjunct for culturing cells in vitro in some circumstances, e.g., to promote cell adhesion and/or differentiation of some cell types. And certain cells have been implanted within gels or hydrogels of particular types of extracellular matrix so as to enhance survival or create the desired amount of bulk at the implantation site. A difficulty of such approaches, however, is that the gels are a barrier that impedes oxygen and nutrient diffusion to the cells and also ultimately interferes with the remodeling of the site by the cells. Further, such gels impede movement and interaction of the implanted cells with surrounding cells. For example, a gelled collagen or hyaluronic acid impedes the flow of oxygen and factors from other cells into the locale of the implanted cells, and impedes cellular movement in or through the gel.

In contrast, the use of a soluble form of protein allows the protein to freely associate with the cells that are introduced and/or with cells and tissues at the implant site. And a soluble form of a protein is fully available for interaction with cells and subject to cellular internalization and/or cellular down regulation, as needed. And, without being bound to a particular mechanism of action, the protein can generally be expected to diffuse a limited distance from the implantation site by virtue of having multiple specific or non-specific binding events that slow its diffusion from the site. As a result, the protein exerts its effects, in general, at or near the site of implantation. In the case of immunogenic proteins, the immune response is provide in the general vicinity of the introduced cells, and serves to evoke a reaction beyond the immediate vicinity of the cells. These reactions may serve to recruit an immune response to enhance the “take” of the implanted cells.

Absorption of proteins onto cell surfaces mediates cellular responses as well as cell interactions with other cells, proteins, and biomaterials. Both in cell culture and implantation protein absorption can dictate the activity of the cells.

Extracellular matrix has multiple functions in tissue. Extracellular matrix provides strength and physical support for tissues and organisms. In vitro and in vivo it is useful for the survivability of cells, e.g., as with fibronectin for culturing of fibroblasts and other cell types. In vivo and in vitro extracellular matrix, as a whole or as a specific component, is involved in control of cell proliferation, adhesion, spreading, migration, differentiation, survivability, hormone interactions, and other interactions between the cell and its surroundings. The tissues of the body typically each have their own extracellular matrix characterized by its own mix of extracellular matrix molecules arranged in a characteristic pattern. In general, highly differentiated and specialized types of cells actively secrete extracellular matrix. And, in connective tissues, a significant portion of the extracellular matrix macromolecules in connective tissue are secreted by fibroblasts. Thus in skin, the fibroblasts tend to create a large proportion of the extracellular matrix. And, in bone, osteoblasts tend to create a large proportion of the extracellular matrix. Extracellular matrix also contains many serum proteins, growth factors, cytokines, chemokines and hormones.

Two main classes of extracellular matrix molecules make up the extracellular matrix. The first are the fibrous proteins, such as collagen, elastin, fibronectin, and laminin which have both structural and adhesive functions. The second are polysaccharide chains called glycosaminoglycans (GAGs) that are covalently linked to protein in the form of proteoglycans making a highly hydrated, gel-like “ground substance” in which the fibrous proteins are embedded. Glycosaminoglycans are, in general, long unbranched polysaccharides containing a repeating disaccharide unit. The disaccharide units typically contain either of two modified sugars: N-acetylgalactosamine (GalNAc) or N-acetylglucosamine (GlcNAc) and a uronic acid such as glucuronate or iduronate. Glycosaminoglycans are typically negatively charged and impart high viscosity to a solution. Glycosaminoglycans typically also impart low compressibility that provides structural support. Glycosaminoglycans include hyaluronic acid, dermatan sulfate, chondroitin sulfate, heparin, heparan sulfate, and keratan sulfate. Gels rich in glycosaminoglycans resist compressive forces, allow diffusion of molecules, are highly hydrated and imparts elasticity to the tissue. Rubberlike elastin fibers, in particular, impart elasticity and resilience.

Examples of extracellular matrix molecules below include annexin, cartilage matrix protein (chondronectin), chondroadherin, collagens, dentine extracellular matrix protein, elastin, fibrillins, fibrin, fibrinogen, fibronectins, fibulins, gelatin (denatured collagen), certain glycoproteins, certain glycosoaminoglycans (GAGs), growth factors, hylauronans, laminins, latent transforming growth factor-β binding proteins, link proteins, matrix GLa protein, microfibril-associated glycoproteins, lipids, monosaccharides, nidogen, oligosaccharides, osteocalcin, osteonectin, osteopontin, certain polysaccharides, prolargin, procollagen, many proteoglycans, certain serum proteins, tenascin, thrombospondin, vitronectin, and von Willebrand factor, amongst others.

The extracellular matrix molecules can be obtained from autologous or heterologous (allogeneic, xenogeneic) sources by purification. The extracellular matrix molecules can be autologous, allogeneic or xenogeneic and made as such by synthesis chemically, biologically (e.g. cell-free translation systems), and by recombinant DNA means. The extracellular matrix proteins can be diverse within its own class. Alternative spliced forms, isoforms, post-translational modifications, fragments, motifs, domains, functional segments and added or subtracted features of the protein, such as through recombinant DNA manufacture are examples of the diversity that can be utilized.

Extracellular matrix constituents of various cell types include anchorins, ankyrin, fibronectins, osteonectins, vitronectins, procollagen, collagen types, laminins, fibrillins, elastins, proteoglycans, annexins, integrins, growth factors and serum proteins (e.g. albumin) that can be extracelullar matrix macromolecules by virtue of being associated with the extracellular matrix.

Basement membrane is a sheet-like ECM separating epithelial tissues and some mesenchymal cells from connective tissues, such as the epidermis from the dermis in skin. Its ECM is made of a number of proteins including collagen type IV, laminin and heparin associated proteins and is often attached to connective tissue by collagen type VII and microfibril bundles. Basement membrane is present in most tissues. In skin it connects the epidermal-dermal junction via laminin, collagen type IV, in arteries the endothelial layer with the subendothelial layer and smooth muscle layer, and in other tissues the epithelial layer with the respective connective tissue layer. The architecture involves two enmeshed networks of collagen IV and laminin. The laminin network is maintained primarily by laminin interactions with laminin and its aggregation depends on Ca++. ECM proteins such as entactin bridge the two networks. Laminin's adhesive properties involve binding of the basement membrane protein to cell surface glycolipids.

The majority of the extracellular matrix proteins are made by most cells and are present to some degree in most tissues. Below is a description of some tissues that show a predominant expression of the protein.

Cell Adhesion Mediating Proteins

Many extracellular matrix molecules are cell adhesion mediating proteins. Cell adhesion mediating proteins are proteins, peptides, proteoglycans, and glycoproteins, including cell-adhesive fragments thereof that mediate cell adhesion by specifically interacting with cell surface adhesion receptors. Specifically interacting is a term that refers to interactions involving recognition between two molecules, as in a receptor-to-ligand or ligand-to-ligand binding event. Examples of specific interactions are lock-and-key interactions of enzymes with substrates and binding of integrin receptors to an RGD sequence. An example of a nonspecific interaction is cell adhesion to a polycation by charge-charge interactions (e.g., polylysine).

In general, adhesion proteins mediate cell spreading when present in an effective concentration. An important class of cell surface adhesion factors are integrins. Cell adhesion molecules such as CAMs and cadherins bind cells to each other, and can serve as cell adhesion mediating proteins. Receptors and cell surface molecules that take part in cell adhesion and spreading and also include anchorins and ankyrin Substrate adhesion molecules (SAMS) are extracellular molecules that share a variety of sequence motifs with other adhesion molecules. Most prominent of the SAMS are segments similar to the type III repeats of fibronectin and immunoglobulin-like domains. SAMS can link and influence the behavior of one another and do not have to be made by cells that bind them.

Tissue is comprised of cells and extracellular matrix mainly produced by cells. Cell numbers in a tissue, as well as in culture, are determined by a balance between apoptosis and proliferation and survival factors. Cell morphology has impact on cell growth, cell division, cell survival, and the cell phenotype. Cell shape changes as it spreads out and migrates on the substratum, be it the extracellular milleau or a surface like plastic, glass or metal. Fibroblasts, epithelial cells and other adherent cell types do not proliferate in vitro in suspension, in which their morphology is rounded up. These cells are anchorage dependent for cell proliferation. When the cells adhere to a substrata, the cells form focal adhesions at the attachment sites and begin to grow and proliferate. The attachment sites are places where extracellular matrix interacts with cell-surface matrix receptors, such as integrins. Integrins are then linked to the cytoskeletal network that controls the above parameters of cell morphology, cell growth, cell division, cell survival, gene expression and cell phenotype.

Cell adhesion is involved in tissue morphogenesis, cell spreading and migration, cell proliferation on a substrata, preventing anoikis, cell-ECM interactions, transmission of ECM information to the cell, cell activation (e.g. leukocytes), transmigration of cells to different locations in the body, differentiation, embryogenesis, cancer metastasis, gene expression, amongst other functions.

Major classes of cell adhesion molecules are the CAMs (immunoglobulin superfamily cell adhesion molecules), integrins, cadherins, lectins, selecting, ECM, and serum proteins amongst other macromolecules. Among the different groups of CAMs are the integrins. Cell adhesion occurs in 3 steps: attachment, spreading and focal adhesion and stress fiber formation. In attachment integrins and ancillary receptors such as syndecans interact with ECM ligands, which activates the integrins into clustering and increased affinity for the ECM ligand. Through formation of microfilaments and cell spreading cells increase the surface contact with the ECM ligand. This constitutes a state between weak and strong adhesion. The stage of strong adherence appears when appropriate ECM signals then promote cells to organize their cytoskeleton (e.g. talin, vinculin, α-actinin) as shown by focal adhesion consisting of ECM protein receptors and actin-containing stress fibers formation that links the termini of these fibers to the membrane and the ECM. The adhesive state undergoes modulation or reversibility during cell proliferation and metaplasia, tissue remodeling during wound healing and morphogenesis and tumor cell metastasis.

Cell adhesion can trigger ligand-independent activation of growth factor receptors resulting in the biological action of these receptors. Growth factors can induce adhesion molecules to promote adhesion-independent signals.

Attachment factors, such as fibronectin and vitronectin, increase cell mobility among other functions in vitro and in vivo. Attachment, cell spreading, cell migration and cell proliferatioin are the sequential steps of cell behavior upon cell adhesion.

Integrins are transmembrane proteins that mediate interaction between adhesion molecules located on adjacent cells or in the ECM. This process affects cell adhesion, spreading, migration, proliferation, survival, anoikis, differentiation, gene expression, wound healing, and many other processes. Integrins can be part of multimolecular signaling complexes through focal adhesions. Integrins exhibit both inside-out (intracellular integrin activation to change binding affinity for ligands) and outside-in signaling properties that occur after an integrin receptor binds its ligand and a signal is transmitted into the cell. Basal avidity, low avidity and high avidity are the three activation states of integrins. The link of structural ECM with the cytoskeleton also contains intracellular kinases adding regulatory and signaling capacity to the transmembrane protein complex, such as the mitogen activated protein kinases (MAPK) and its pathway.

At least 16 different α and 8 highly homologous β subunits combine into 22 different heterodimers each havng specific recoginiton and affinities for various ECM components or other cell bearing adhesion molecules. Focal adhesion kinase (FAK) or other intracellular tyrosine kinases can confer integrin mediated survival and resistance to anoikis. FAK is more active in fibroblasts plated on fibronectin (specific adhesion) than plated on polylysine (non-specific adhesion). While nonspecific adhesion can be used to mediate cell attachment to a surface or matrix, specifically-mediated adhesion often advantageously promotes specific cellular responses.

Most ECM proteins are involved in matrix-matrix and matrix-cell interactions, both of which promote cell adhesion. Examples of ECM and serum protein ligands for integrins are the collagens, laminins, nidogen/entactin, fibronectins, tenascins, fibrillins, fiblins, bonesialoproteins, proteoglycans, perlecan, vitronectin, fibrinogen, fibrin, thrombospondin, Von Willebrand Factor, gelatin, denatured collagens, other denatured ECM or serum proteins, blood clotting factor X, ICAM (intercellular adhesion molecule) and its isoforms, VCAM (vascular cell adhesion molecule), MAdCAM (mucosal addressin cell adhesion molecule) and osteopontin. Specific domains obtained by recombinant or proteolytic fragments can contain the binding sites to integrins as well as to other ECM sites.

Examples of ligand selectivity and integrin subtypes are: α₁β₁ and α₂β₁ binds collagen (including types I, II and IV), laminin, E1X or E8 domain of laminin. α₃β₁ binds laminin 5, other laminin isoforms, fibronectin, collagen and nidogen/entactin. α₄β₁ binds fibronectin, the IIICS region (peptides CS1 and CS5) of fibronectin, the second heparin binding region HepII, and VCAM-1. α₅β₁ binds fibronectin, the RDG sequence in the III10 region of fibronectin, denatured collagen, the RDG sequences in collagen, L1 cell adhesion molecule, vitronectin and insulin-like growth factor binding protein 1. α₆β₁, α₇β₁, α₆β₄ bind laminins 1, 2, 4, 5 and the E8 region of laminin. α₈β₁ binds fibronectin that is RGD dependent, vitronectin and tenascin. α₉β₁ binds collagen, laminin, and tenascin. αᵥβ₁ binds vitronectin, fibronectin and osteopontin. α_{IIb}β₃ binds fibronectin, the RDG sequence in the III 10 region of fibronectin, vitronectin, fibrinogen, von Willebrand factor, thrombospondin, laminin and fibulin-2. αᵥβ₃ binds fibronectin, vitronectin, von Willebrand factor, thrombospondin, tenascin, thrombin, osteopontin, fibulin, fibrillin, gelatin, denatured collagen, PECAM-1 or CD31 (cellular counter-recpetor platelet endothelial cell adhesion molecule-1), perlecan, L1 cell adhesion molecule, MAGP-2 (microfibril-associated glycoprotein 2) and cyr61. αᵥβ₅ binds osteopontin and vitronectin. αᵥβ₆ binds fibronectin and vitronectin. αᵥβ₈ binds vitronectin. α₄β₇ binds MadCAM-1, VCAM-1, fibronectin and the IIICS region of fibronectin. α_{E}β₇ binds E-Cadherin. α_{L}β₂ binds ICAMs-1, -2 and -3. α_{M}β₂ binds iC3b (inactivate complement factor 3b), blood clotting factor X, fibrinogen, I-CAMs-1 and -2. α₅β₃ and α₅β₃ are RGD dependent integrins.

Native conformation of ligands can be important for integrin binding (not for denatured collagens). Activation of integrins by antibodies enable avid binding to the ligands, e.g. α₂β₁ binds collagen and laminin when in an active conformation. After ligand binding both α₁β₁ and α₂β₁ trigger cell responses such as collagen gel contraction, MMP-1 gene activation and decreased expression of α1 chain of type I collagen.

Some of the cell types that express integrins are: α₂β₁ is expressed in fibroblasts, keratinocytes, and many other cell types. α₁β₁ is found in smooth muscle cells, hepatocytes, cells in close contact with basal membrane like endothelial cells of blood capillaries, astrocytes, neural crest cells, neural cells and many other cell types. α₃β₁ is expressed in most cell types. It binds laminin-5 which associates with laminin-6 to form epiligrin, which is found in epithelial basal membranes in organs of endodermal or ectodermal origin and lymph nodes. α₆β₁ is expressed in most tissues. α₇β₁, a laminin-1 receptor, is found in myoblasts and myotubes of skeletal and cardiac muscle. α₆β₄ is found on perineural fibroblasts of peripheral nerves, Schwann cells, endothelia, epithelium and immature thymocytes. The α₆β₄ integrin is located within hemidesmosomes. The cyclic peptide CRRETAWAC binds to α₅β₁. The 9^{th} fibronectin type III repeat contains the sequence PHSRN that confers selectivity and increases the affinity of fibronectin to the integrin. Fibronectin mediates anchorage dependent growth, thus upregulating cell proliferation genes when cells adhere to fibronectin. Also MMP-1, 3 and 9 have increased expression and secretion. αᵥβ₈ is found in brain, sensory neurons, placenta, ovary, uterus, kidney and melanoma cells. αᵥβ₆ is found in epithelial cells and β₅ is expressed in may cell types. αᵥβ₃ is found in osteoclasts and involved in bone remodeling and resorption, angiogenesis and tumor growth. α₈ is found in smooth muscle, other contractile cells in adult tissues, mesenchymal cells and neural cells during development. α₅ is ubiquitously expressed in tissues.

β subunits confer tissue specificity. α₅β₁ and αᵥβ₁ binding to fibronectin triggers cell spreading. α₅β₁ can migrate on fibronectin and produce a fibronectin matrix and the integrin remains diffusely distributed on the cell surface. αᵥβ₁ and αᵥβ₃ bind vitronectin and these integrins form focal contacts, evoke endocytosis (removal from the blood) of complement factors and serum proteins involved in blood coagulation. For example iC3b is opsonized by the integrins. This opsonization by vitronectin or other proteins is useful in the invention to rid the injection site of blood clots. α_{IIb}β₃ binds fibrinogen, triggering platelet activation and aggregation resulting in clot formation. This action is useful to limit the bleeding caused by the injectate or implantate in the invention. The integrin recognizes soluble fibrinogen, fibronectin, vitronectin, von Willebrand factor and insoluble fibrinogen and the HHLGGAKQAGDV sequence of γ chain of fibrinogen. β₃ are members of the cytoadhesin family that bind proteins present in both blood and ECM, the leukocyte integrins with the common β₂ subunit are involved in cell-cell interaction and bind primarily cell surface-anchored counter-receptors in immune processes. β₁ containing integrins are primarily receptors for ECM proteins. β₇ is involved in the immune processes. β1, 2, 3 integrins form focal contacts in which the integrins gather and anchor actin fibers to the membrane of the cell.

Some of the ECM proteins that contain an RGD intergrin-binding sequence, some of the integrin receptors and some of the cells that express the integrins are: Fibronectin binds α₅β₁ made by fibroblasts, platelets, macrophages, keratinocytes, and memory T cells. Fibronectin and vitronectin bind αᵥβ₁ made by endothelial cells. Fibronectin and tenascin bind α₈β₁ made by fibroblasts, smooth muscle cells and neural cells. Fibronectin, vitronectin, thrombospondin and von Willebrand factor bind αᵥβ₃ made by macrophges, endothelial cells, platelets and B lymphocytes. Fibronectin, vitronectin and tenascin bind αᵥβ₆ made by carcinoma cells. Fibronectin, laminins and thrombospondin bind α₃β₁ made by kidney glomerula cells and B lymphocytes. Fibronectin and VCAMs bind αᵥβ₁ made by macrophages, lymphocytes, NK cells, eosinophils and thymocytes. Fibronectin, vitronectin, collagens, fibrinogen, thrombospondin and von Willebrand factor bind α_{III}β₃ made by platelets. Fibronectin, collagens and laminins bind αᵥβ₈ made by renal tubular epithelial cells. Vitronectin binds αᵥβ₅ made by fibroblasts and hepatoma cells. Collagens and laminins bind α₂β₁ made by fibroblasts, endothelial cells, platelets, B and T cells. Laminins bind α₇β₁ made by skeletal and cardiac cells and cancer cells. Fibrinogen binds αₘβ₂ and αₓβ₂ found on leukocytes (macrophages, monocytes, granulocytes). Tissue transglutaminase (tTG) functions as a co-receptor for beta 1 and beta 3 integrins and stabilizes ECM proteins by isopeptide cross-linking.

RGD dependent integrins are α₅β₁, α₈β₁, αᵥβ₁, αᵥβ₃, αᵥβ₆, α_{IIb}β₃, α₃β₁ and bind to the 10^{th} type III repeat of fibronectin containing the RDG loop. The 9^{th} type III repeat domain of the protein plays an auxiliary role. α₄β₁ integrin binds cell contacts to the LDV of CS1, REDV of CS5 region and IDAPS of the 14^{th} type III repeat domain of fibronectin. The first type III repeat domain of fibronectin (III 1-C) binds β1 integrins and cell surface HPSGs as receptors. These peptides induce cell adhesion and spreading.

A number of integrin ligands can be reduced to proteolytic or recombinant fragments which can further be reduced to short peptides retaining the adhesive activity of the parent protein. Thus RGD of fibronectin and vitronectin, QAGDV of fibrinogen, LDV of fibronectin and IDSP of VCAM-1 are some of these peptides. Other peptides that bind integrins consist of the sequence GRGDS that is in vitronectin and binds to αᵥβ₃, the sequence of KGWTVFQKRLDGSV contained in fibrinogen and the shorter peptide KYGQKRLDGS that bind the αₘβ₂ integrin. The IETP and LETS sequences in ICAMs and for example from ICAM-2 the derived peptide GSLEVNCSTTCNQPVEGGLETS bind integrins.

Proteins containing the cell adhesive RGD sites within their peptide seqeunce include fibronectin and VTGRGDSPA, HVPRGDVDH, vitronectin and QVTRGDVFT, fibrinogen and (the α₁ EILRGDFSS, α_{2 G} DSRGDSAT, α₃ SYNRGDSTF and γ chains GNSRGDN), von Willebrand factor and MDERGDCVP, GSPRGDSQS, osteopontin and YDGRGDSVV, bone sialoprotein-2 and GEPRGDNYR, tenascin and ISRRGDMSS, thrombospondin-1 and GDGRGDACK, fibulin-2 and SVPRGDLDG, fibrillin-1 and IRPRGDNGD, fibrillin-2 and FANRGDVLT, FGPRGDGSL, laminin and α₁-chain FALRGDNPQ, VEKRGDREE, Collagens and XGXRGDREE, nidogen/entactin and IGFRGDGRT, perlecan and ASFRGDKVT, L1 adhesion molecule and ITWRGDGRD, LQERGDSDK, metargidin (a metalloprotease disintegrin protein) and RPTRGDCDL, thrombin and EGKRGDACE, insulin growth factor binding protein-1 and PEIRGDPNC, echinoidin and VPSRGDIDS, tiggrin and SKDRGDQPP, HIV-Tat Protein and SQPRGDPTG, VP1 of Foot-and-Mouth Disease Virus and PNLRGDLQV, VP1 of Coxsackievirus A9 and SRRRGDMST, VP-1 of Echovirus 22 and RALRGDMAN, Pertactin and TIRRGDALA, Penton base protein of Adenovirus type 2 and HAIRGDTFA, Filamentous Hemagglutinin and LAARGDGAL, disintegrins and neurotoxins from snake venom, decorsin and matins from leech proteins and cyclic RGD peptides.

Other peptide sequences for cell binding include YIGSR, RNIAEIKDI, and SIKVAV. Other sequences such as, collagen-like peptide, are homologue sequences found in collagen IV and XVIII, that can promote cell adhesion. The linear sequence GWTVFQKRLDSV of fibrinogen is the recognition site in which RLD is essential. Laminin has different cell binding fragments such as P1, E3, E8 and EX.

Synthetic RGD peptides, chemically synthesized, enzymatically derived, or from peptide libraries (randomized) can be made such as the sequences of X⁻³X⁻²X⁻¹R⁺¹G⁺²D⁺³X⁺⁴X⁺⁵X⁺⁶. Position +4 is critical for fibronectin cell adhesion and spreading. Without an amino acid in the position, cell adhesion and spreading activity is lost. With asparagine in the position fibronectin cell adhesion is inhibited, with threonine in its place vitronectin cell adhesion is inhibited. A hydrophobic or trytophan subsititution increases specificity to integrin α_{II}β_{b3} and asparagine-proline in positions +4, +5 increase activity towards αᵥβ₃ and α₅β₁. A series of sequences in proteins not containing RGD sequence can bind to integrins in a RGD heritiable fashion. Examples include the sequence KQAGDV of the γ chain of fibrinogen, KGD of the disintegrin barbourin, proteins containing RYD motifs such as stretavidin, OPG-2 and PAC-1 antibodies and the gp63 surface glycoprotein of leshmania. Thus the RGD loop binding to integrins is determined by a series of structural criteria including remote effects, loop shape, length and flexibility, and neighboring residues.

Some proteins bind receptors with greater affinity when in a natural conformation, other proteins interact stronger when denatured. For example, serum fibronectin can bind cells with even greater affinity when immobilized or denatured than in a more extended conformation. Fibronectin small fragments bind to cells. Limited proteolysis of laminin-1 liberates the RGD motif for high cell attachment and integrin binding. Similarly proteolytic degradation of collagens expose the numerous RGD sequences found in the triple helical regions. RGD containing proteins are potential cell adhesive ligands. It appears some RGD sites require the ancillary binding sites and conformation of the ligand for binding, while other integrins may bind a linear RGD site.

Integrin-ligand binding can be dependent on divalent cations. Usually it is promoted by magnesium or manganese ions and inhibited by calcium or divalent cation chelators such as EDTA. Both the binding affinity and avidity is affected. For example α₅β₁ has a high affinity site for manganese, a low affinity site for magnesium and calcium, and a high affinity site for calcium. At low concentration, calcium binds to the high affinity site and promotes magnesium binding, inducing cell adhesion. However, at high concentration, calcium binds to the low affinity magnesium site and inhibits cell adhesion.

Integrins can be activated by the presence of activating antibodies to the integrin receptor that in turn induces binding to ligands. Integrin mediated adhesion to ECM proteins are responsible for cell anchorage and migration on ECM proteins. Integrin mediated cell-cell contacts also elicit cell responses such as migration, cell shape changes, gene expression and secretion. These responses can be found on cells of the immune system that are involved in inflammatory and immune processes such as leukocyte movement.

Integrin actions are induced often by growth factors. For example, α₅β₅ and α₅β₃ integrins, in the presence of TGF-β1, are involved in the differentiation of fibroblasts into myofibroblasts in the mouth and skin and with α₅β₅ in kidney tissue. Angiogenin supports endothelial and fibroblast cell adhesion and spreading. Cell adhesion and growth factor binding to their receptors can mediate resistance to DNA damage from chemo or radiotherapy.

Disintegrins are polypeptides or proteins that contain the RGD sequence and competitively inhibits integrin-ligand interactions by binding to the integrin receptors. For example, VLO4, VB7, VA6 and EOA from snake venom and domains of proteins (proteases) that contain the RGD motif inhibits cell adhesion to the αᵥβ₁ integrin that binds to fibronectin. VLO5 and EO5 contain MLD and VGD motifs and block the adhesion of α₄β₁ integrin to VCAM-1. EMS11 inhibits both integrins. Different disintegrin subfamilies contain ADAMs or are related to ADAMs (a disintegrin and metalloproteinase-like) matrix. Thus adhesive functions are blocked and disintegrins acts as platelet aggregation inhibitors. Echistatin inhibits bone resorption and platelet aggregation as does falvoridin and kistrin. Other antagonists of RGD integrin function include the peptides Gly-Arg-Gly-Asp-Ser, Gly-Arg-Gly-Asp-Ser-Pro-Lys, and Gly-Arg-Gly-Asp-Thr-Pro.

Most ECM glycoproteins promote cell adhesion and cause cytoskeletal reorganization that lead to cell migration, proliferation, cell survival and differentiatoin. Another class of ECM proteins, matricellular proteins function as adaptors and modulators of cell-matrix interactions. These include TSPs 1 and 2, the tenascins and osteonectin (SPARC). The matricellular proteins function as both soluble and insoluble proteins. As soluble proteins these can have de-adhesive effects on cells in an adhesive state. Cell adhesion by TSP1, tenascin and osteonectin (SPARC) is dependent on the cell type and protein solubility.

De-adhesion can occur when ECM-integrin interactions are disrupted by proteolysis, the matricellular proteins TSP1, tenascin-C and osteonectin or integrin antagonists. De-adhesion can be used to remove cells from in vitro cell culture.

Poly (2-hydoxyethyl methacrylate) reduces adhesion of cells to growth surfaces while polylysine enhances electrostatic interaction between the negatively charged cell membrane and positively charged surface. This represents an example of opposite non-specific adhesion site effects on the cells.

CAMs. Among the different groups of CAMs are the integrins, immunoglobulin-cell adhesion molecules, cadherins, selectins, CD44-related molecules and transmembrane proteoglycans. CAMs are transmembrane glycoproteins, that bind integrins or other Ig superfamily CAMS.

Members of the Ig superfamily include ICAMs (intercellular adhesion molecules), VCAM-1 (vascular adhesion molecule), PECAM-1 (platelet-endothelial-cell adhesion molecule), and NCAM (neural cell adhesion molecule). Other CAMs are ALCAM (activated leukocyte cell adhesion molecule), BCAM (basal-cell adhesion molecule), BOC, CDO, CEACAM-1, the L1 family of CAMs (including L1 CAM-2 that promotes neuronal survival, integrin-mediated cell migration to ECM proteins and neurite outgrowth. Contactins (-1 to -6) are members of the CAM family. Contactin-1 interacts with L1, NCAM, neurocan, phosphacan and tenascin. Contactin-2 and -4 contain fibronectin type III-like repeats. EpCAM (epithelial cellular adhesion molecule) is expressed in kidney liver, skin, epithelia, pancreas, germ cells and carcinomas. Additional members are cadherins such as 4, 6, 8, 11, 12, 17 and the desmogleins-1 to -3. Other members include ESAMs, Kirrel 2, Nectins (e.g. -2,-4), OCAM, ICAMs (e.g. -1 to -5) that bind leukocyte integrins, JAM-A (junctional adhesion molecule A) that is expressed at intercellular junctions of epithelial and endothelial cells, JAM-B which is located in endothelial venules, heart and placenta, and JAM-C, an adhesive ligand for T, NK and dendritic cells. CAM members LAMP (limbic system-associated membrane protein) is involved in neuronal growth and guidance. MadCAM-1 (mucosal addressin cell adhesion molecule-1) is involved in lymphocyte homing to mucosal sites. NCAM and NrCAM are involved in neural development. RAGE (receptor for advanced glycation end products) ligands are AGEs (advanced glycation end products), amyloid-beta peptide, HMG-1 and several members of the S100 protein superfamily. RAGE can mediate neuronal outgrowth, survival, regeneration and pro-inflammatory reactions. RAGE is involved in diabetes, Alzherimer's disease systemic amyloidosis, apoptosis, tumor growth and aging tissues. TROP-2 is expressed in carcinomas. Polysialylation of N-CAM and other CAMS are part of the glycosylation pattern of these proteins. VCAM-1 binds integrins VLA-4, α₄β₁ and α₄β₇. It is a cell surface protein expressed by leukocytes, such as macrophages and endothelial cells. VCAM-1 is induced by IL-1β, IL-4, TNFα and IFNγ. Activated integrins stop rolling leukocytes during the inflammatory adhesion mechanism and attaches them to the vascular endothelium by binding to VCAM-1 ligands on the endothelium. Extravasation of white blood cells through the blood vessel wall to inflammation sites is mediated by VCAM-1/VLA-4/α₄β₇ interactions. Soluble VCAM-1 exists in serum and fluids. PECAM-1 (CD31) is expressed on endothelial cells, T cells, platelets, leukocytes such as monocytes and neutrophils and present in plasma. It binds α₅β₃ leukocyte integrin. PECAM-1 is needed for transendothelial migration of leukocytes via intercellular junctions in vascular endothelial cells and is modulated by the circulating form.

ICAMs and VCAMs are intercellular adhesion ligands for α_{L}β₂. VCAM-1 binds α₄β₁ integrin with the sequence QIDSL. ICAM-1, 2, 3 are counter-receptors. ICAM-1 is found on many cell types such as endothelial cells, fibroblasts, leukocytes, epidermal keratinocytes and epithelial cells. The immunoglobulin superfamily member is stimulated by IFNγ, TNFα, IL-1β and LPS. Soluble ICAM-1 and other ICAMs are found in the serum resulting from cleavage by proteases on the cell surface. ICAM-2 is found on lymphocytes, monocytes, vascular endothelium and ICAM-3 is found on leukocytes and epidermal Langerhan's cells. ICAM-1 binds leukocyte intergrins LFA-1 and Mac-1. ICAM-2 mediates adhesion to provide a co-stiumlatory signal for T cell aggregation, NK cell migration and NK cytotoxicity. ICAM-3 is involved in T cell stimulation by Langerhans cells. VCAM-1 and MadCAM-1 are expressed on endothelial cells of venules.

Cadherins are a family of transmembrane calcium dependent glycoprotein cell adhesion molecules involved in cell-cell and cell-ECM contacts. Cadherins have an extracellular domain containing several Ig-like intrachain disulfide-bonded loops with conserved cysteine residues, a transmembrane domain and an intracellular domain that interacts with the cytoskeleton. The cell-cell junctions are formed by interaction between the extracellular domains of identical cadherins that are located on the membrane of neighboring cells. The adhesive binding is stabilized by binding of the intracellular domain of cadherin with the catenins α, β, and γ and the actin cytoskeleton. In conjunction with desmocollins, desmoglein isoforms form the adhesive components of desmosomes found in epithelial cell-cell adhesive structures. Classical cadherins contain an extracellular domain of the transmembrane protein containing DXD and DXNDN repeats for mediating calcium-dependent adhesion.

Cadherins are present in all solid tissues and regulate a number of processes including cell migration, cell polarization, tissue morphogenesis, maintenance and regeneration. Cadherins, or the extracellular cell binding domain of cadherins can be used to cause cell-cell binding of implanted cells within themselves and to in situ cells.

Lectins are carbohydrate-binding proteins in which the carbohydrate portion comprise polysaccharides, glycoproteins, glycolipids and other similar moeities. Lectins can agglutinate cells. Lectins comprise the C-type lectins and receptors, galectins, Ig-type lectins, collectins and selectins, among other subclasses (R, M, L, M-lectins and calnexin). C-type lectins have various ligands and are involved in cell adhesion (selectins) and glycoprotein clearance and innate immunity (collectins). C-type lectins can be calcium dependent for ligand binding.

Some of the members of the lectin family are CD 72, CD94, chondrolectin, CL-P1, CLEC-1, -2, DC-SIGN (dendritic cell-specific ICAM-3 grabbin non-integrin), DC-SIGN related protein, DCI receptor, Dectin-1, -2a, DLEC, Fc epsilon RII, Ficolins, Langerin, Layillin and LOX-1 (lectin-like oxidized low-density lipoprotein receptor 1). These members are located on activated endothelial cells, vascular smooth muscle cells, macrophages, intestinal and dendritic cells, among other cell types. MBL (mannan binding lectin) belongs to the collectin family of innate immune defense proteins. MBL-1,-2, MDL-1, NKG2s (A, C, D) have an extracellular C-type lectin-like domain. Other lectins include NKps (80, 30, 44, 46) that are expressed on NK killers, Reg 2, Regs (e.g. I, II, III, IIIa, IV), SCGF, SIGN receptor 1, receptor 4 and SP-D.

Selectins are involved in cell adhesion and have 3 family members that are carbohydrate-binding proteins (e.g. fucosylated carbohydrates such as sialytated Lewis and mucins). The extracellular domain contains an EGF like motif, motifs to complement-regulatory proteins and a carbohydrate binding motif. E-selectin (endothelial leukocyte adhesion molecule-1 or ELAM-1) is expressed on vascular endothelial cells in the presence of IL-1β or TNF-α. L-selectin (leukocyte selectin or LAM-1) is expressed on leukocytes. P-selectin (GMP-140) is expressed by activated platelets and endothelial cells. PSGL-1 (P-selectin glycoprotein ligand-1) is the ligand for P-selectin and is present on all leukocytes. Selectins L, E and P are involved in cell-cell adhesion and have a C-type lectin domain at the extracellular amino termini, followed by an EGF like domain and then several complement regulatory domains, a transmembrane domain and a short cytoplasmic tail. L-selectin (LECAM-1) mediates the tolling and arrest on high endothelial venules by interaction with sulfosialyl lewis x antigens on HEV cells and is the basis of lymphocyte recirculation. It allows the migration of lymphocytes into peripheral lymph nodes, sites of chronic inflammation and entry of neutrophils into acute inflammatory sites. In combination with P and E-selectins, L-selectin mediates the initial interaction of endothelial cells with circulating leukocytes to produce a rolling of the leukocytes on the endothelium. E-selectin is upregulated by endothelial cells during inflammation by the proinflammatory cytokines IL-1 and TNFα. Selectin E, ˜115 kDa cell surface glycoprotein, is expressed on vascular endothelial cells in response to IL-1β and TNF-α and sLex antigens expressed by immune cells mediates the rolling and arrest of inflammatory cells to the site of inflammation. E-Selectin mediates attachment of flowing leukocytes to blood vessel wall during inflammation through binding to E-selectin ligands on leukocytes. The initial interaction is followed by ICAM-1 and VCAM-1 interactions in which white blood cell extravasation into the ECM of the vessel occurs. E-selectin ligands are present on monocytes, neutrophils, a subset of memory T cells which are silayted, fuscoylated molecules bound to the lectin domain of E-selectin. Thus adhesive (integrin mediated) and signaling events (chemokines, cell-cell contact) during leukocyte extravasation results in inflammation and lymphocyte homing. P-selectin is involved in leukocytes and neutrophil adhesion to the endothelium.

Sialoadhesins comprise MAG, CD22, CD33 and Schwann cell myelin protein. Sialoadhesins are cell surface glycans containing sialic acid residues defining this I-type sialyl lectin subgroup. Soluble forms are present in plasma and tissues.

Collectins (collagen-like lectins) include mannan-binding protein (MBP), conglutinin, lung surfactant proteins SP-A and D. They play roles in innate immunity without antibodies. Collectins rid the body of microorganisms. MBP can activate the complement system via the lectin pathway.

Galectins are a family of carbohydrate-binding proteins with N-acetyl-lactosamine-containing glycoprotein specificity and bind to cell-surface glycoproteins. Galectins bind to glycoconjugates on the cell plasma membrane and in the ECM. Galectins facilitate glycan crosslinking in the ECM and have β-galactosides as ligands. Extracellular galectins have the role of sugar binding proteins and intracellular galectins as non-sugar binding proteins. Inflammation induces galectin expression. Some of the members are galectins-1 to -13 and galectin-3 BP (binding protein). Galectin-1 is abundant in most tissues, is proapoptotic, blocks cell adhesion, is anti-inflammatory, suppresses autoimmunity and is antiproliferative. Galectins (e.g. -3) can oppose cell adhesion of fibronectin or laminin, are mitogenic, cytostatic, anti-apoptotic, increases proinflammatory cytokines production, such as IL-1, in immune cells (e.g. leukocytes, epithelial cells, cancer cells). Galectin-7 is made in skin and galectin-8 blocks integrin interaction with ECM in liver, kidney, heart muscle, brain and other tissues. Galectin-12 is made by adipocytes and induces apoptosis and cell cycle arrest.

Ig-type or I-lectins include MAG (Siglec 4) and other Siglecs (sialic acid binding Ig-like lectins). Siglecs (e.g. 1-11, F, L1) are members of the immunoglobulin superfamily. Sialic acids mediate cellular interactions and are often involved in the immue system. Siglecs are involved in cell adhesion. Siglecs have a large extracellular domain, the sialic acid binding domain, a transmembrane domain and a cytoplasmic domain (except Siglec-1). MAG is expressed by Schwann cell and oligodendrocytes whereas the other Siglecs are expressed by various immune cell types (1,11 by macrophages; 2, 6, 10 by B cells; L1 by luminal epithelium; 3, 5, 7, 9, 10 by monocytes; 8,10 by eosinophils; 5, 9 by granulocytes; 3 by myeloid precursors; 6 by placenta).

The extracellular domains can connect two glycoproteins to each other in the ECM or an ECM protein to a cell can be processed with the above cell adhesion molecules. Many of the lectins, CAMs in general, sugar-carrying polymers, polyelectrolyte polymers or hydrogels can be used for cell adhesion.

Some other adhesion molecules, the AMIGOs (amphoterin-induced Gene and ORF) are transmembrane proteins. CD2 (cluster of differentiation) is a transmembrane glycoprotein expressed on T cells and a target for CD58. CD58 (lymphocyte function-associated antigen or LFA-3) is a receptor on fibroblasts, endothelial and epithelial cells, leukocytes, erythrocytes, amongst other cells.

Certain other lectins induce mitogenic activity (e.g. lymphocytes), such as concanavalin A, pokeweed lectin, variety of agglutins such as leucoagglutinin PHA-L and phytohemagglutinin PHA-P.

Cells in culture can produce dense 3-D matrices (e.g. proper serum supplementation that overcome contact inhibition) and cells within these 3-D matrices form a distinct class of adhesion. Fibrillar adhesions containing long fibrils of fibronectin or 3D matrix adhesions are dependent on integrin α₅β₁ and fibronectin. Cells adhere more rapidly to the 3D matrix and have more rapid migration, proliferation and morphological changes than 2D matrices or 3D collagen gels.

Normal attachment and proliferation of cells are dependent on attachment factors and ECM components. Some examples of ECM proteins and cell type adhesion are described above and select examples are: collagen type I and mesenchymal cells such as fibroblasts, muscle cells, and others such as hepatocytes, Schwann cells, neurons, amongst many other cell types. Another example is collagen type II and chondrocytes as well as collagen type IV and epithelial, endothelial, fibroblasts, muscle and nerve cells. Fibronectin (plasma, cellular, recombinant fragment III-C, recombinant fragment III-C and plasma fibronectin complex, small fibronectin fragments, fibronectin-like engineered protein, superfibronectin, the fibronectin domains of heparin binding fragment, 30 kDa, gelatin binding fragment, 45 kDA, heparin and gelatin binding fragment, 70 kDa, fibronectin adhesion promoting peptide Typ-Gln-Pro-Pro-Arg-Ala-Arg-Ile or Lys-Asn-Asn-Gln-Lys-Ser-Glu-Pro-Leu-Ile-Gly-Arg-Lys-Lys-Thr) attaches to mesenchymal cells, fibroblasts, epithelial, endothelial and neuronal cells. Gelatin binds many cell types. Vitronectin binds mesenchymal cells (fibroblasts), endothelial cells and platelets. Laminin interacts with epithelial, endothelial, hepatocytes, muscle and tumor cells. Tenascin binds mesenchymal, epithelial, and neuronal cells. Thrombospondin binds fibroblasts, smooth muscle cells, endothelial cells, neurons and osteoblasts.

Hormones, growth factors, cytokines, chemokines and other molecules (drugs) can influence cell adhesion of specific cell types to other cells and ECM. For example, Deprenyl increases adhesion of neuronal and non-neuronal cells (fibroblasts) and is an enzyme (MAO B) inhibitor involved in Parkinson's, Alzheimer's diseases, atherosclerosis and tumor formation.

Mechanical stimuli such as stretching or pressure can alter ECM expression such as collagens, tenasinc-C, MMPs, etc. Cell-matrix adhesion sites can serve as a mechanosensory switch transmitting forces from the ECM to the cytoskeleton and in the reverse direction in which the integrins are key to signalling cascades. Regulatory gene expression of ECM, cytoskeletal, signal-transduction and stress-response genes occur. ECM is the primary means in which mechanical information is transmitted to cellular and tissue levels of function. Important links to signaling pathways, such as integrin localization are altered.

Extracellular Matrix Adhesion Proteins.

Extracellular matrix proteins have binding sites that interact with other extracellular proteins and themselves. Most ECM proteins that bind cells also have other sites for binding other extracellular proteins. Thus, cell adhesion proteins also have domains for binding to the extracellular matrix.

Intact, fragmented, recombinant, moieties of or other forms of the cell adhesion proteins can be used to facilitate binding of cells to ECM matrix of the implantate. Additionally, the in vitro culturing of the cells can be facilitated by a similar action.

Domains of ECM proteins and other proteins can bind specific cells or proteins, and can have physiological roles. For example, the cell adhesion domain of fibronectin III repeats or hemopexin domains can block angiogenesis and tumor growth. ECM proteins have domains that interact with growth factor receptors. Collagen has domains that interact with the discoidin domain receptors and increases cell proliferation, migration, ECM turnover and decreased MMP production for fibroblasts and epithelial cells. Matrikines (small peptide fragments of ECM proteins) or domains within tenascin-C, laminin-5, collagen and decorin interact with the EGF receptor that effect EGF actions. Matrikines can have potent tissue repair activities. For example, GHK (glycyl-histidyl-lysine) binds to collagen, triggering increased cell proliferation and anti-oxidant enzymes and wound contraction. Other ECM proteins have EGF like repeats that interact with EGF receptors. ECM proteins such as fibronectin, hyaluronic acid and other protein types (e.g. heat shock proteins) can be Toll-like receptor (TLR) family ligands (e.g. on macrophages). This initiates inflammatory responses and induces innate immunity against pathogens. Also regulatory T lymphocytes activated by TLRs exert enhanced immunosuppressive functions and also can activate fibroblasts for cell proliferation, etc. ECM made by fibroblasts and other cell types affect cellular immune responses. ECM such as the proteoglycans (e.g. testicans, CSPGs) can modulate the cell attachment of cells.

Any ECM proteins that have cell binding sites can be used for cell adhesion in vivo and in vitro. The binding sites can be the RGD domain as well as other known domains or sites that are not limited to the examples give above. Proteins with binding sites for other proteins that assist in adhesion to limit migration of the injected protein or cells can be used. Similarly, other functions such as nutrient delivery, transport protein, protease inhibitor, apoptosis inhibitor, amongst others, can encompass those ECM proteins demonstrating such properties.

Glycoproteins

Glycoproteins are biomolecules that can contain about 1% to about 60% carbohydrate. The term glycoprotein includes proteoglycans which can have a higher % carbohydrate. Many of the matrix glycoproteins contain distinct and functionally active peptide domains that interact with cell surface receptors as well as other matrix molecules. This heterogeneous group of proteins contain carbohydrate covalently attached to the protein core through O and/or N linkages. Glycoproteins can influence cell behavior by promoting attachment and migration of cells.

Cell adhesion mediating proteins, most of which are glycoproteins, include thrombospondin, von Willebrand factor, fibronectins, vitronectins, chondronectins, procollagen and collagen types I, III, IV, V, and many of the types II-XIX, laminins, fibrillins, fibrinogen, entactin, MAGPs, LTBPs, osteopontin, procollagen C proteinase, dentine extracellular matrix, phosphophorins and annexins. Fibronectin type III and EGF repeats are common to many adhesive glycoproteins. Many possess the RGD (Arg-Gly-Asp) sequences and RGD containing polypeptides, which mediate cellular adhesion through the integrin family of receptors.

Cell attachment regions include the cell-binding domains of fibronectin (III repeat regions) and other proteins. Cell-binding domains contain the cell-binding short amino acid sequences. This includes the RGD (Arg-Gly-Asp), RGDS (Arg-Gly-Asp-Ser), RGDSP (Arg-Glp-Asp-Ser-Pro), LDV (Leu-Asp-Val), REDV (Arg-Glu-Asp-Val) and Pro-His-Ser-Arg-Asn amino acid sequences. These cell attachment sequences can be used alone or as part of a molecules such as a synthetic molecule, peptide, polypeptide or protein.

RGD sequence containing proteins include fibronectins, entactins, laminins, collagens and fibrinogen. Peptides containing this RGD sequence can be useful in the development of anti-clotting drugs that mimic these peptides. RGD sequences are recognized by several members of the integrin family of cell-surface matrix receptors.

With respect to the cell adhesion mediating protein collagen, there are 19 distinct genetic types encoded by at least 34 genes that constitute the collagen types I through XIX (1-19). About 25 α chains have been identified. Collagen fibers both strengthen and help organize the matrix. The main types of collagen found in connective tissues are types I, II, III, V, VII and XI. Many types of collagen promote cell attachment and proliferation. Type I is the principal collagen of reticular portion of skin and bone and Type III is the principal collagen of the papillary portion of the skin.

Predominant tissue location and collagen types are: type I is the major structural component of ECM in connective tissue and internal organs and found in skin, tendon, bone, cornea and bone; type II is found in cartilage and vitreous; type III is found in skin, aorta, gut, uterus; type IV is the major component in basement membranes, which underlie epithelial and endothelial cells, surround muscle, fat and nerve cells and overlie connective tissue. It can promote cell attachment and proliferation; type V is found in skin, bone and placental tissue; type VI is found in skin, cornea, cartilage and uterus; type VII is found in skin, esophagus, amniotic membrane; type VIII is found in endothelial cells and Descemet's membrane; type IX is found in vitreous and cartilage; type X is found in calcifying cartilage; type XI is found in cartilage and intervertebral disc; type XII is found in skin, tendon and cartilage; type XIII is found in epidermis and endothelial cells; type XIV is found in skin, tendon and cartilage; type XV is found in kidney, heart, ovary, testis and placenta; type XVI is found in smooth muscle, heart and kidney; type XVII is found in hemidesmosomes of specialized epithelia in skin and at photoreceptor synapses and outer segments in the retina. It is expressed in cones and rods.; type XVIII is found in kidney, liver and lung; and type XIX is found in fibroblast cell lines. Specific collagen types for tissue placement is preferred with its natural tissue location. However, other collagen types normally present in different tissues can be used in a heterologous tissue placement. Type I collagen improves the attachment and adherence of cells in vitro, including but not limited to osteoblasts, chondrocytes, and fibroblasts (e.g. tendon and ligament), lung type II epithelial cells, smooth, striated and cardiac myocytes, aortic, venous and capillary endothelial cells.

Collagen contains a number of domains. Collagen interacts with a number of other proteins. Some of collagen's properties are listed below: Collagen I has a DGEA cell adhesion site, N-linked and hydroxylysine glycosylation sites, COL2 (collagen 2) domain, collagenase, N-proteinase and C-proteinase cleavage sites. Collagen I is associated with collagen III or V. Collagen II interacts with protein cores of the proteoglycans fibromodulin and decorin via crosslinking to collagen IX, and has stromelysin and collagenase cleavage sites. Collagen III contains the collagen 2 domain and collagenase cleavage site. Collagen IV interacts with laminin, nidogen, heparin sulphate proteoglycan, heparin and cell-binding sites, and contains N-linlced glycosylation sites. Collagen type V interacts with type I and III collagens, contains a MMP-9 cleavage site, N-linked glycosylation sites and a collagen 2 domain. Collagen VI interacts with hyaluronan, type II and XIV collagens, biglycan, and chondroitan sulphate proteoglycan NG2 receptor. It also contains N-linked and hydroxylsine glycosylation sites, fibronectin type III repeats, NC2, NC1 and helical domains. Collagen VII contains a N-glycosylation site, fibronectin type III repeats, NC1, NC2 and helical domains. Collagen VIII contains collagenase cleavage sites, NC1, NC2 and helical domains. Collagen IX interacts with type II collagen and links collagen fibrils to other extracellular matrix proteins. It contains stromelysin cleavage and N-glycosylation sites, collagen 1, 2, 3 and NC 1, 2, 3, domains. It may be considered a proteoglycan since its α2 (IX) chain can contain a glycosaminoglycan chain. It can serve as a bridge between collagen fibrils or with the aggrecan networks. Collagen X has collagenase cleavage and N-glycosylation sites, NC1, NC2 and helical domains. Collagen XI interacts with collagen V and has C-proteinase and N-linked glycosylaton sites, collagen 2 and helical domains. Collagen XII interacts with the glycosaminoglycan chain of decorin and the protein core of fibromodulin, contains N-linked glycosylation and glycanation sites, fibronectin type III repeats, collagen 1, 2, NC1, 2 and 3 domains. Collagen type XIII contains collagen 1, 2, 3, 4 and NC 1, 2, 3, 4 domains. Collagen type XIV interacts, as does types IX and XII, with proteoglycans or exist in a proteoglycan form. It associates with glycosaminoglycan chains of decorin, type VI collagen and procollagen I N-proteinase. It contains a N-linked glycosylation site, collagen 1, 2 and NC1, 2, 3 domains and fibronectin type III repeats. Collagen XV contains N-linked glycosylation sites, O-linked glycosaminoglycans, NC1-10 and collagen 1-9 domains. Collagen XVI contains N-linked glycosylation sites, COL 1-10 and NC1-11 domains. Collagen XVII contains N-linked glycosylation sites, antigenic sequences, an immunodominant site, COL 1-15 and NC 1-16 domains. Collagen XVII binds laminin and BPAG1 (dystonin) as part of a hemidesmosome complex needed for basal keratinocyte adhesion in skin and as part of the retinal rod photoreceptor cytomatrix attachment complex to the ECM. Collagen XVIII contains N-linked glycosylation sites, O-linked glycosaminoglycans, an RGD adhesion site, COL1-10 and NC 1-11 domains. Collagen XIX contains N-linked glycosylaton sites, COL1-5 and NC 1-6 domains.

Fiber forming collagens are types I, II, III, V and XI, nonfibrillar collagens that form sheet like networks are types IV, VIII, X, microfibrils are comprised of collagen type VI and short filaments are comprised of collagen type VII. The fibril associated collagens with an interrupted triple helix (FACIT) (collagens IX, XII and XIV) are associated with fibrils formed by collagen I and II. Collagen VI bridges cells to ECM. Collagen fibril density assists in regulating local cell-ECM biomechanics and fibroblast function under mechanical stimuli. For example, fibroblast proliferation is increased under low collagen-fibril density ECMs.

In aged and photodamaged tissue, such as skin, reduced interaction of the fibroblast with collagen and other ECM occurs. Most collagen types have intracellular cross-linking sites to stabilize the protein. Collagen increases stiffness and tensile strength of healing tissues in large part to appropriate cross-linking. Cross-linking also occurs between collagen and other ECM molecules. Cross-linking of collagen and other ECM proteins increases in tissue aging that may be deleterious.

Collagen-like peptide, are homologue sequences found in collagen IV and XVIII, that can increase the synthesis of other ECM proteins such as laminin 5, collagen I, III, IV and β1 integrin. Other short ECM peptides can also have similar effects. Collagen-like peptide promotes cell adhesion, differentiation, ECM sythesis and anti-apoptosis.

Collagen-like domains or peptides are present in many proteins and in the triple helical region of collagen. For example, in serum mannose-binding protein the collagen-like domain contain serum protease binding sites. The Gly-X-Y repeat pattern is present in some collagen like peptides or domains. Some of these peptides contain proline-hydroxyproline-glycine residues that are repeating in sequence.

Collagen can induce cell adhesion, migration and proliferation as well as cell aggregation, amongst other cellular properties.

Cyclophilin-C (CyCAP) associated protein, along with FN fragments 45 and 70, can induce MMP-13 expression. CyCAP affects ECM and MMP expression altering collagen, fibronectin and other ECM protein expression.

The CCN family of proteins are regulatory proteins present in the ECM and plasma. The family proteins are represented by CYR61, CTGF (CCN2), NOV (CCN3), WISP-1 (CNN4), WISP-2 (CCN5) and WISP-6 (CCN6). Members of this family have cell adhesion properties, amongst others. CYR61 (CCN1, cysteine-rich heparin-binding protein, IGFBP-10) and FISp-12 (murine homolog of connective tissue growth factor) are ECM proteins that promote ECM synthesis, cell adhesion, migration and proliferation of stromal and epithelial cells, endothelial cells and fibroblasts. The protein has pro-angiogenic activity for example, by binding αᵥβ₃ and α₆β₁ integrins in endothelial cells. Cyr61 can serve as a mechano-switch acting through the cytoskeleton network in cells. Mechanical stimuli can cause cyr61 to mediate appropriate ECM production, growth factor production (e.g. VEGF in smooth muscle cells) and integrin interaction (e.g. αᵥ) to accommodate cells with an altered phenotype, morphology and function as a result of a change in the physical microenvironment. Aberrant expression can result in atherosclerosis and restenosis. Connective tissue factor (CCN2, CTGF) is a secreted protein that contains domains mediating interactions with growth factors, integrins and other ECM components. CTGF promotes connective tissue production. CTGF promotes procollagen synthesis, collagen deposition, neovascularization, angiogenesis, chondrogenesis, wound healing, cell proliferation (e.g. fibroblasts, endothelial cells, chondrocytes) and differentiation (e.g. chondrocytes). CTGF is a chemoattractant for cells (e.g. fibroblasts). CTGF is induced by TGFβ in fibroblasts and keratinocyte production of IL-1α suppresses CTGF production by fibroblasts. CTGF promotes apoptosis in vascular smooth muscle cells. Excess production of CTGF can produce tissue fibrosis. CTGF can activate the NF-κB pathway in tubuloepithelial cells. CTGF promotes matrix contraction by fibroblasts. NOV/CCN3 (Nephroblastoma overexpressed gene), is expressed highly in smooth muscle of the arterial wall. CCN3 supports cell adhesion, migration and cell survival. It interacts with integrins α₅β₃, α₅β₁, α₆β₁ and heparan sulphate proteoglycans. It binds to the integrins lacking an RGD site. CCN3 acts upon endothelial cells to stimulate angiogenesis.

Dystroglycan (DG) is an adhesion molecule formed by two subunits, an extracellular α and transmembrane β that forms a continuous link from the ECM to the intracellular cytoskeleton in cells. DG affects cell adhesion, growth and proliferation. DG is needed for receptor cluster stabilization (acetylcholine receptor via laminin interaction), spans the membrane linking cytoskeleton to basement membranes such as in sarcolemmal membrane cytoskeleton linking to the basement membrane surrounding each muscle fiber. Perlecan, laminin, acetylcholinesterase (basement membrane protein e.g. in neuromuscular junction) interact with DG. DG through binding to perlecan assist in the synaptic basement membrane via acetylcholinesterase localization. The neuromuscular junction transmits signals from motor neuron to muscle via the nerve terminal, the synaptic basement membrane and the postsynaptic membrane. αDG is a laminin receptor. Laminin and αDG coassemble on the cell surface and bind other ECM such as collagen, entactin and perlecan. DG is O-mannosylated. DG can link dystrophin to the ECM. Dystroglycans and sarcoglycans are present in cardiac myocytes.

Soluble tropoelastin is the biosynthetic precursor of elastin, secreted into the extracellular space and assembled into elastic fibers close to the plasma membrane. Elastin is present in most tissues and is abundant in the aortic artery. Elastin can prevent the excessive proliferation of smooth muscle cells in the arterial wall. Elastic fibers are covered with a sheath of microfibrils containing a number of glycoproteins including fibrillin which binds to elastin and is needed for the integrity of elastic fibers.

Elastin (m.w. 54,000) is the major protein of the elastic fibers that form a randomly oriented and inter-connected network in many tissues. It comprises from 2% of dry weight in skin to 50% in aortic artery. Its high hydrophobicity makes the protein one of the most protease and chemically resistant proteins in the body. Elastin primarily provides elasticity and resilience to tissues and promotes cell adhesion. Its peptides have been shown to be chemotactic. Alternate splicing of tropelastin, a single polypeptide chain produces a number of different isoforms. Splicing, as with most proteins, is regulated in a tissue-specific and developmental manner. In association with microfibrillar components individual chains assemble to form elastic fibers. Deamination of specific lysines by lysyl oxidase allows covalent cross-links to stabilize the elastic fibers. Elastin contains hydrophobic, cross-link and alternatively spliced repeats, and β spiral motif Elastin improves the attachment of cells in vitro including endothelial and smooth muscle cells. Elastin and tropoelastin can advantageously be added to defects, with or without cells, to enhance elasticity and resilience of the treated defect site. Cells (e.g. fibroblasts, smooth muscle cells, chondrocytes, endothelial cells, etc.) have an elastin receptor that binds elastin. Cells recognize elastin (α elastin) through interactions with elastin-binding proteins (EBPs) and the VGVAPG hexapeptide sequence present in elastin. Elastin together with fibrillin-1 binds to cells via the RGD site of fibrillin-1. Elastin can play a role in cell attachment as well as cell migration and alteration in the phenotypic properties of cells (e.g. smooth muscle cells). Elastin is expressed by fibroblasts, endothelial cells, smooth muscle cells, and most other cell types.

Extracellular matrix protein-1 (ECM-1), 85 kDa glycoprotein, is expressed in many tissues including skin, cartilage and bone. It functions in promoting cell proliferation (e.g. endothelial cells), inducing angiogenesis, regulating bone formation and differentiation (e.g. keratinocytes). ECM-1 is an adhesive protein in the dermis, regulates collagen assembly and growth factor binding. ECM-1 binds to perlecan, the major heparin sulphate proteoglycan. ECM-1 has roles in wound healing, scarring and skin aging. ECM-2 is made by adipose tissue and other cells including those in female organs. There is homology to the proteoglycans keratocan and decorin. ECM-1 contains functional domains that are involved in protein-protein interactions, such as with von Willebrand factor and osteonectin, domains containing a leucine-rich repeat region, the RGD sequence, and the N-terminal fifth of the protein stimulates cell proliferation (e.g. lymphocytes). ECM-2 has a role in lymphopoiesis and hematopoiesis.

Fibulin-1 (m.w. 61,000) is a glycoprotein in the ECM and in plasma (33 ug/ml). It is secreted by fibroblasts and has fibronectin, entactin, itself, other extracellular matrix proteins and calcium binding capacity, contains Type I and EGF repeats, and has N-linked glycosylation and alternate splicing sites. There are at least 3 alternate spliced forms. Fibulin-1 and 2 have a broad binding spectrum for other extracellular proteins or ligands. Fibulin-2 (m.w. 126,000) plays also a role in the formation of matrices and is found in heart, placenta and ovaries. It contains Type I and EGF repeats, an RGD site and has a N-linked glycosylation site. Fibulin-3 (487 aa) and fibulin-4 (443 aa) are closely related to fibulin-1C. Both contain a central EGF-like calcium binding domain, and a C-terminal globular domain shared by the fibrillins. Fibulins can advantageously be added to defects, with or without cells, to enhance retention of fibronectin, provide mechanical support and enhance cell adhesion.

Fibronectin contributes to survival of cells in vitro and in vivo. It is expressed by fibroblasts in skin, in most other tissues and may other cell types (e.g. endothelial cells) and made in the liver. Fibronectin, (m.w. 440,000) is a dimer of two large subunits joined by disulfide bonds at one end. The single large gene contains about 50 exons of similar size. Some of the type III fibronectin repeats bind to integrins. The cell-binding domain with the RGDS sequence is located in the 10^{th} type III repeats of fibronectin. The synergy cell binding sequences in the 9^{th} type III repeats is a key attachment site for cell-surface receptors and the EDA spliced sequence of fibronectin, the connecting segment I (CS-1). The first type II repeat of fibronectin has a cell adhesion domain with cell surface heparan sulfate proteoglycans and integrins of cells. Fibronectin contains fibrin, heparin, gelatin, collagen and factor XIIIa transglutaminase cross-linking binding domains. Fibronectin can bind to itself. The central cell binding domain is recognized by most adherent cells via the integrin receptors. In addition it is involved in fibronectin matrix assembly. There are some 50 alternate spliced variants of fibronectin. Many of the different isoforms of the protein are due to alternative spliced forms of the ED-A, ED-B and II-CS regions and subsequent post-translational modification. Soluble serum fibronectin enhances blood clotting, wound healing, inflammation and phagocytosis. Insoluble fibronectin is deposited in the ECM and assembles on the surface of cells. Fibronectin binds many proteins in the ECM. Fibronectin can serve as a template for collagen deposition. Interaction with the integrin receptor can change the gene expression and behavior of the cells. The integrin interacts with actin filaments, cytoskeleton and myosin proteins that ultimately affect the nucleus, nuclear matrix and gene expression. Fibronectin controls matrix assembly of binding proteins such as latent TGFβ binding protein-1. Plasma and cellular produced fibronectin influence cell adhesion, cell migration, cell shape, cell survival, cell proliferation and differentiation.

Cancer cells produce less fibronectin and typically adhere poorly to culture substratum and fail to flatten out or develop stress fibers or organized intracellular bundles of actin filaments. With fibronectin, cells attach to extracellular matrix to grow and proliferate. The dependence of cell growth, proliferation and survival on attachment to a substratum is known as anchorage dependence. It is mediated by integrins and the intracellular signals they generate. Fibronectin binding to the cell prevents anoikis or subtrate detached apoptosis. Fibronectin has RGD, IDAPS, LDV and REDV cell adhesion sites. The protein has binding sites for fibrin, heparin, collagen, DNA and cells. Fibronectin contains Type I, II, III repeats, ED-A, ED-B and IIICS alternately spliced domains, N and O-linked glycosylation sites, and a factor XIIIa transglutaminase cross-linking site. Anchorage-dependent cells are recognized as being distinct from non-anchorage-dependent cell types in many respects, and especially with regards to their culture in vitro.

RetroNectin is a cell adhesion mediating protein that is a commercial recombinant DNA version of fibronectin cell binding domains made in E. coli. RetroNectin is comprised of 574 amino acids (63,000) with three functional domains of the central cell-binding domain (type III repeat (8-10)), a heparin-binding domain II (type III repeat 12-14) and a connecting segment CS-1 site with the alternatively spliced IIICS region. Other fibronectin variants are available commercially, including those manufactured by recombinant DNA means. For example, a 31,000 m.w. cell-binding domain fragment of fibronectin (C279) consists of three type III repeats (III8-10). Pronectin F uses the fibronectin 10 amino acid sequence containing the RGD cell binding domain and the sequence (GAGAGS)₉ from genetically engineered silk fibroin that crystallizes in a beta-sheet conformation. It has multiple cell attachment sites from human fibronectin. Pronectin F is used for improved plating efficiency, better cell growth, faster and stronger adherence and more in vivo like morphology. It has been demonstrated to work with fibroblasts, cells of bone, embryonic, endothelial, epithelial, eye-derived, glial, hematopoietic, muscle, neuronal, parenchymal and tumor cells. Pronectin F PLUS combines elements of the functionality of fibronectin, collagen, and polylysine. Pronectin L is a reagent presenting IKVAV epitopes from the laminin alpha chain. The recombinant C-terminal portion of the first type III repeat (protein III 1-C) assists in fibronectin matrix assembly and can be used for cell attachment and spreading. This cell binding domain stimulates ERK1/2 activation in cells (e.g. smooth muscle) and acts through integrin and HSPG receptors.

The fibrillins-1 and -2 are cell adhesion mediating glycoproteins (m.w. 311,00 and 314,000 respectively) that are a major subset of connective tissue microfibrils. They are made in connective tissue cells (e.g. fibroblasts) and other cell types. In skin, microfibrils extend from elastic fibers in the dermis to the basement membrane of the dermal-epidermal junction approximating the epidermal layer. Smooth muscle cells attach to isolated microfibrils. It provides the scaffold on to which elastin is assembled to form elastic fibers. The proteins contain EGF, TGF-β1 receptor repeats, an RGD cell adhesion and N-linked glycosylation sites.

Fibrinogen is a soluble plasma cell adhesion mediating protein cleaved by thrombin to produce an insoluble fibrin clot. It is a hexamer of two sets of α, β and λ chains, each with a m.w. of about 50,000. The α chain can be cross-linked to fibronectin. Two types of λ chains are alternately spliced. The α chain has RGD cell adhesion, α2-plasmin inhibitor binding, acceptor cross-linking and thrombin cleavage sites; the β chain has a thrombin cleavage and N-linked glycosylation site; and the λ chain has a calcium binding, N-linked glycosylation, cross-linking and QAGDV cell adhesion site.

Frem 1 (Fras1-related extracellular matrix protein) is an extracelular matrix protein involved in epithelial/mesenchymal interaction and epidermal remodeling, a dermal mediator of basement membrane adhesion, epidermal differentiation and epidermal adhesion. Fras1-related ECM protein 2 and 3 are members of the protein family.

Laminins are cell adhesion mediating glycoproteins (e.g. 820 kD) present in all basement membranes of tissues. It is expressed by skin fibroblasts, macrophages, endothelial cells, epithelial cells, Schwann cells, in the lung and is ubiquitous in most connective cell types. Laminin is an adhesion molecule for epithelial cells, for example. These proteins interact with cells via cell-surface receptors (integrins) and other basement membrane components such as type IV collagen, entactin/nidogen, heparin, glycosaminoglycans and heparan sulphate proteoglycan to promote cell attachment to the basement membrane components. They are involved in development, differentiation and migration, cell attachment, cell maintenance, cell proliferation, metastasis and cell outgrowth. For example, laminin-1 increases cell adhesion, migration, growth and differentiation of cells in vitro and in vivo. Laminins are important in dermal adhesion and synaptic development in the nervous system (CNS, PNS) such as astrocyte sialic acid residues control the laminin matrix assembly regulating neurogenesis. In skin, laminins, collagen XVII and dystonin (BPAG1) form part of a hemidesmosome complex needed for keratinocyte adhesion. In the retina these proteins anchor the rod photoreceptor cytomatrix to the ECM. Laminin's actions are mediated often by protein-protein and protein-carbohydrate interaction involving the integrin family, α distroglycan and HPSGs such as perlecan.

Laminin contains heparin and cell binding sites. The alchain of laminin (m.w. 337,000) contains EGF and G repeats, N-linked glycosylation sites and IKVAV, RGD and GD-6 cell adhesion sites. The α2 chain, m.w. 343,000, has EGF and G repeats and N-linked glycosylation sites. The a3a chain, m.w. 189,000, and the α3_{b} chain, m.w. 202,000, have EGF and G repeats and N-linked glycosylation sites. The α5 chain, m.w. 393,000 has EGF and G repeats, N-linked glycosylation sites and RGD and LRE cell adhesion sites. The β1 chain, m.w. 198,000, has EGF repeats, N-linked glycosylation sites, and LGTIPG, RYVVLPR, PDSGR and YIGSR cell adhesion sites. The β2 chain, m.w. 196,000, has EGF repeats and N-linked glycosylation sites. The λ1 chain, m.w. 177,000, has EGF repeats and N-linked glycosylation sites and the RNIAEIIKDI (p20) cell adhesion site. The λ2 chain, m.w. 131,000, has the EGF repeats and N-linked glycosylation sites. There are at least 12 different types of laminins as extracellular matrix proteins (1-10, I, S). Laminin-derived YIGSR peptides can improve the attachment of cells in culture for glial cells, neurons and cells grown on Type I collagen or Pronectin F.

Latent transforming growth factor-β binding proteins (LTBPs) are cell adhesion mediating proteins that are members of the TGFβ binding proteins. The platelet protein versions are smaller than the fibroblast versions. Alternate spliced variants or proteolytic variants exist. Motifs similar to the fibrillin family of proteins exist. The proteins contains EGF and TGFβ1 repeats and N-linked glycosylation sites. LTBP1 has a RGD cell attachment site.

Microfibril-associated glycoproteins-1 and 2 (MAGPs), m.w. 20,000, are components of the 12 nm microfibrils found in elastic and non-elastic tissues. In elastic tissues these proteins become incorporated into elastic fibers. MAGP2 has a N-linked glycosylation site and a RGD cell binding motif Microfibril-associated protein-1, m.w. 52,000, and microfibril-associated protein-2, m.w. 40,000 are proteins/glycoproteins associated with elastic-fiber microfibrils. MAGPs (e.g. MAGP-2) bind to fibrillins and can induce collagen expression (type I) and stabilizes the procollagen form.

Mystique is an IGFI regulated PDZ-LIM domain protein that promotes cell attachment and migration via cell adhesion to collagen and fibronectin. It is located at the actin cytoskeleton.

Nidogen or entactin, m.w. 136,000, is a cell adhesion mediating sulphated glycoprotein that is an integral component of the basement membrane and associates with laminin and type IV collagen. It has EGF, thryoglobulin and LDL receptor repeats, EF-hand-type divalent cation binding, O-linked sulphation, N-linked glycosylation, transglutaminase cross-linking and RGD cell adhesion sites. Nidogen 1 and 2 are basement membrane proteins. Nidogen binds to basement membrane collagen type IV.

Osteonectin or SPARC (secreted protein acidic and rich in cysteine), m.w. 35,000, is synthesized by bone, endodermal, epidermal and soft connective tissues. It is involved in bone formation and mineralisation, tissue differentiation and remodeling, wound healing, angiogenesis, tumorigenesis, signanl transduction and cell communication. Osteonectin promotes wound healing by enhancing fibroblast migration and thus granulation tissue formation. It is expressed in bone, skin, vitreous and aqueous humor among many other tissues. It is a matricellular protein that regulates endothelial function, endothelial cell proliferation and cell-ECM interactions. It inhibits VEGF production and is anti-angiogenic. SPARC is an anti-cell adhesion protein for certain cell types. Cell adhesion is dependent on the cell type and protein solubility.

Osteopontin or bone sialoprotein I, m.w. 36,000, is a cell adhesion mediating glycoprotein found in bone matrix, placenta, distal tubules and blood vessels of the kidney, the central nervous system and tumor tissue. It attaches osteoclasts and binds to hydroxyapatite. It has an RGD adhesion site and binds cells through integrins as well as through non-integrin interactions. OPN is chemotactic for macrophages, smooth muscle, endothelial and glial cells. OPN is modified by the proteases thrombin, enterokinase, MMP-3 and -7.

Procollagen C-proteinase, m.w. 115,000, is a cell adhesion mediating that removes the C propeptides of fibrillar procollagens type I, II, III, V and XI. This removal catalyzes the rate limiting step in the extracellular self-assembly of collagen into fibrils and larger fibers and is important in the assembly of all connective tissues. The proteins responsible for C-proteinase activity are related to the bone morphogenetic protein-1 and mammalian tolloid, as an alternative spliced form in some tissues. The protein has the EGF repeat and BMP-1 specific sequence. Procollagen I N-proteinase cleaves the amino-propeptides of type I and type II procollagens into collagens. It has a RGD cell adhesion site and a properdin repeat.

Spondins 1 and 2 are ECM cell adhesion proteins. Spondin 1 promotes cell (e.g. neuron, smooth muscle cell) outgrowth and attachment. The protein is present in many tissues such as lung, brain, heart, kidney, liver, testis, pancreas, skeletal muscle and ovary. Spondin 2 promotes adhesion of neuron cells and binds to bacteria as an opsonin for macrophage phagocytosis. The protein is needed for initiation of the innate immune response.

Tenascin-C, m.w. 241,000, is a cell adhesion mediating glycoprotein present in many developing organs and the stroma of tumors. It functions in cell-matrix adhesion (anti-adhesive activity for certain cell types) such as inhibiting adhesion and spreading of fibroblasts on fibronectin substrate in cell culture. It functions in cell migration, growth regulation, wound healing, tissue remodeling and differentiation during morphogenesis. The protein has EGF, fibronectin type III and alternatively spliced repeats, N-linked glycosylation and RGD cell adhesion sites. Tenascin-R, m.w. 150,000, is found in the central nervous system and contains similar domains as tenascin-C and other members of the tenascin family. It is a repulsive substrate for fibroblasts, astrocytes and neurons but adhesive for retinal cells. Tenascin-X, m.w. 386,000, is not as well glycoslyated as other members of the family and does not contain an RGD sequence. It is present in most tissues and in developing fetal tissues. It is an organizer and stabilizer of the ECM. Reduced collagen density and fragmented elastic fibers occurs in skin without the protein present. Tenascin-Y, m.w. 207,000, is present in embryonic, heart and skeletal muscle tissues. It has EGF and fibronectin type III repeats. Tenascin is also produced by embryonic mesenchymal cells and assists epithelial tissue differentiation.

Thrombospondin-1 (TSP-1) m.w. 129,000, is a cell adhesion mediating protein made by platelets, fibroblasts and smooth muscle cells, and is involved in cell adhesion (integrins, CD36, proteoglycans and suphatides), growth, embryogenesis and the regulation of cell migration and proliferation during wound healing, angiogenesis, development and tumorigenesis. The protein binds collagens, laminin, fibronectin and fibrinogen. It has EGF, Type 3, and properdin repeats, and N-linked glycosylation, heparin-binding, RGD cell adhesion, VTCG cell adhesion, and platelet adhesion istes. The protein has alternate spliced variants. Thrombospondin-2, m.w. 129,000, has EGF, Type I and III repeats, and a N-linked glycosylation and RGD cell attachment sites. Thrombospondin-3, m.w. 104,000, is present in the brain, lung and cartilage. It has EGF and Type III repeats and N-linked glycosylation sites. Thrombopsondin-4, m.w. 106,000, is present in heart and skeletal muscle and has EGF and Type III repeats and N-linked glycosylation and RGD cell adhesion sites. Thrombospondin-5, m.w. 83,000, is present in all cartilages and the vitreous of the eye. Tenascins and thrombospondins can promote or inhibit cell adhesion, depending on the cell type. Thrombospondins can interact with other ECM components such as fibronectin, fibrinogen, proteoglycans, laminin and collagen. TSP 1 and 2 in addition to modulating cell-matrix interactions, also have anti-angiogenic properties. The three type 1 repeats (3TSR) of thrombospondin-1 are natural domains that can be an angiogenesis inhibitor. As with the tenascins, thrombospondins can promote or inhibit cell adhesion, depending on the cell type.

Vitronectin (VN), m.w. 54,000, is a cell adhesion mediating present in the plasma and ECM. It is involved in cell adhesion (integrin receptors), as a spreading factor, in cell migration, in enhanced cell proliferation, in hemostasis, tissue repair and remodeling, phagocytosis, immune function, binds to proteins in the complement and coagulation pathways and inhibits cytolysis. It is present in the liver, platelets, macrophages and smooth muscle cells. It has hemopexin repeats, a somatomedin B and heparin-binding domain, an RGD cell adhesion, protease cleavage, factor XIIIa transglutaminase-catalyzed cross-linking and N-linked glycosylation sites. VN regulates pericellular porteolysis and cell motility. Recombinant VN and a fusionprotein (GST) consisting of VN's 40 amino acid heparin binding domain support cell (fibroblast) adhesion.

Vitronectin and insulin-like growth factors can stimulate enhanced cell migration and proliferation in skin and bone. The facilitation of wound healing requires the presence of skin cells, growth factors to enhance migration and proliferation of these cells and scaffolds to support them when required. Vitronectin and growth factors like insulin-like growth factors augment the activity of cells and can assist the culture of autologous cells in animal product-free media.

von Willebrand factor, m.w. 309,000, is a multimeric plasma cell adhesion mediating glycoprotein (5-10 ug/ml) important in the maintenance of hemostasis by promoting platelet-vessel wall interactions at the site of vascular injury. It promotes platelet adhesion to the subendothelium and binds collagen and heparin. The protein contains A, B, C, and D repeats, an RGD cell adhesion site, GPIb and N-linked glycosylation sites. The factor promotes cell adhesion.

One characteristic of bone, cartilage and dentine adhesive glycoproteins is their anionic nature thst is present in osteopontin, bone sialoprotein, osteocalcin and matrix Gla protein. Skeletal glycoproteins have the ability to influence ion concentrations and bone cell metabolism directly. These glycoproteins may advantageously be added to defects for cellular nutrition and ionic properties, and may be combined with cells, including cells found in bone or cartilage, for placement at a defect site, including a bony or cartilaginous defect.

Chondronectin or cartilage matrix protein, m.w. 54,000, is a major component of non-articular cartilage. It can bind to and bridge type II collagen fibrils and is involved in the cell adhesion of chondrocytes to the ECM, such as collagen amongst others. It contains von Willebrand factor A repeats and an EGF repeat and a N-linked glycosylation site. It functions as a cell adhesion protein for at least chondrocytes.

Chondroadherin is a leucine repeat-rich glycoprotein, m.w. 41,000, in cartilage. It can mediate cell attachment of chondrocytes to plastic, for example as does collagen type II, laminin, vitronectin, fibronectin and other ECM proteins.

Dentine extracellular matrix protein contains a number of non-collagenous proteins including phosphophoryns, dentine sialoprotein and dentine matrix protein (DMP1), all distinct from that in bone. DMP1, m.w. 53,000, is expressed in calvaria and preameloblasts, and has a RGD and N-linked glycosylation site. Dentine sialoprotein, m.w. 95,000, has a high carbohydrate content of 30% and sialic acid 10% and is made by differentiating odontoblasts and pre-secretory ameloblasts. It is similar in composition to osteopontin and bone sialoprotein and has a N-linked glycosylation site. Phosphophoryns, m.w. 95,000, play a role in dentinogenesis and has an RGD cell adhesion site. The proteins in this class that comprise RGD or other known cell adhesion mediating motifs are cell adhesion mediating proteins.

Matrix extracellular phosphoglycoprotein (MEPE) is present in bone and dental tissue. Dentonin, a 23 aa (amino acid) peptide derivative of MEPE stimulates dental pulp stem cell proliferation and differentiation. Enhanced cell proliferation requires RGD and SGDG motifs in the peptide. Dentonin down regulates p16, and up regulates ubiquitin protein ligase E3 and human ubiquitin-related protein SUMO-1.

βig-h3 (TGFβ-induced gene product) is a ECM adhesion protein inducible by TGF-β. It is prominent in skin, cornea and many other connective tissues. The 683 amino acid secreted protein contains a carboxyl-terminal RGD sequence and four homologous domains of 140 amino acids. It is homologous to other cell adhesion proteins such as osteoblast specific factor 2 (OSF-2), Drosophila fasciclin-1 and Mycobacterium bovis MPB70. The protein promotes cell adhesion, migration and proliferation (e.g. epithelial).

Additional ECM Proteins:

Enzymes exist in the ECM. For example lysyl oxidase and transgluaminases are needed for crosslinking and stabilization of ECM collagens, elastin and other proteins.

BMP-1 cleaves ECM precursor proteins to the mature ECM proteins. Metalloproteases, ADAMTS, superoxide dismutase, amongst many other enzymes are present in the ECM.

Superoxide dismutase (SOD) exists is several forms including extracellular SOD-3 which is attached to heparin sulfate proteoglycans in the interstitium of tissue. It is located also in between the plasma and endothelium of the vessels. SOD is in extracellular fluids including lymph, plasma, synovial fluid and serves as an anti-oxidant to destroy free radical that are produced by cells. Many tissue express SOD3 including heart, lung, skin, pancreas, placenta, kidney, skeletal muscle and liver. Other SOD forms are Mn, Cu and Zn SODs.

Tissue transglutaminase (tTG) functions as a co-receptor for beta 1 and beta 3 integrins and stabilizes ECM and serum proteins by isopeptide cross-linking, such as collagen, vitronectin, fibronectin and fibrinogen. Coagulation transglutaminase factor XIIIa is present in serum. Transglutaminases are one of many enzymes present in the ECM and serum.

Lysyl oxidase (LOX) catalyze lysine-derived cross-links in the ECM, in particular in the dermis. This copper and lysyl-tyrosyl cofactor containing amine oxidase cross-links collagen, elastin, amonst other proteins that help stabilize the particular proteins and the ECM. LOX is a multi-functional protein with regulatory and activation mechanisms. Fibronectin binds lysyl oxidase with high affinity for its proteolytic activation. LOX is involved in age pathologies and in wound healing, fibrosis, hypertrophic scarring, (e.g., keloids), diabetic skin and scleroderma.

Chitinase 3—like 1 is a 39 kDa glycoprotein expressed in articular chondrocytes, synovial cells, liver, bone marrow, spleen, brain but not in fibroblasts. It is involved in macrophage maturation.

Heparan sulfate (HS) sulfotransferases, such as HS6ST2 (heparan sulfate 6-O-sulfotransferase 2), are needed for the interaction between HS and a number of proteins that result in cell adhesion, migration, proliferation, differentiation, inflammation, blood coagulation and other diverse processes.

Other proteins such as endostatin (collagen XVIII), prolactin, fibronectin, angiostatin and hepatocyte growth factor are angiogenesis inhibitors derived from the plasma. Angiostatin is an amino-terminal fragment of plasminogen. Endostatin is a cleaved product of the carboxyl-terminal domain of collagen XVIII. Endostatin promotes apoptosis in HUVE and HMVE cells.

Endoglin is expressed at the surfaceof endothailial cells. It is a component of the TGFβ receptor complex and plays a role in cardiovascular development and vascular remodeling. Endoglin has extracellular, transmembrane and cytoplasmic domains. Endoglin regulates the actin cytoskeletal organization.

Ephrins are a family of proteins that are ligands for the class V receptors that are protein-tyrosine kinases. Ephrins type A are linked to the membrane via glycosylphosphatidylinositol linkage and ephrins type B are type-I membrane proteins. Ephrins, such as ephrin-A1, are angiogenic inducing proteins elevating angiopoietin-1 and thrombospondin-1 acitivities, induces cell cycle genes such as p21, affects cell-cell interactions (integrins, MMPs, Rho), and involved in the nervous system (development and guidance). EphA1 are receptors contain two fibronectin type III domains, a globular and cysteine-rich domain. Ephrins are expressed in neural tissue.

Extracellular matrix histone H1 binds perlecan, amongst other ECM proteins, and stimulates cell proliferation (e.g. myoblasts).

Fibstatin is a fragment containing the type III domains 12-14 of fibronectin. It is endogenous to the basement membrane and serum and is an inhibitor of angiogenesis and tumor growth.

FP-1 is an extracellular matrix protein, 549 amino acids, expressed by follicular papilla cells in a hair cycle-dependent manner e.g. in anagen and not other phases of the hair cycle.

Matrillins are adaptor proteins of the ECM that form collagen-dependent and independent filamentous networks. Matrillin 1, 2, 3, and 4 are known. Like some other ECM binding proteins collagen I, laminin-nidogen complexes, fibrillin-2 and fibronectin, matrillins bind other ECM proteins.

Matrix Gla protein, m.w. 12,000, is expressed in many tissues such as cartilage and visceral organs and acts as an inhibitor of calcification in arteries. It is a vitamin K-dependent protein. In bone, but not in kidney, 1,25 hydroxyvitamin D3 up-regulates matrix GLA protein expression. It may be added with cells or at a defect site to inhibit calcification in some cases.

Mindin has multiple functions in the immune response.

Keratins are a class of fibrous structural proteins present in epidermis, hair, nails, horny tissues and tooth enamel organic matrix. The two major conformational groups are α and β keratin.

Osteocalcin, m.w. 11,000, is the most abundant protein in bone and made by osteoblasts and odontoblasts. The protein binds to hydroxyapatite and assists in the assembly of mineralized bone. The protein is vitamin K dependently synthesized and 1,25 hydroxyvitamin D3 stimulated.

PRELP or prolargin, m.w. 44,000, is present in many types of tissue such as cartilage, aorta, sclera, kidney, liver, skin and tendon and has leucine-rich repeats.

To treat defects, ECM and growth factors can vary in concentration from >0% to 100% if used alone and >0% to <100% if part of the cell composition.

Proteoglycans

Proteoglycans include, but are not limited, to aggrecans, agrin, bamacan, BEHAB (brain enriched hyaluronan), biglycan, brevican, decorin, fibromodulin, heparan sulfate proteoglycans, keratocan, lumican, neurocan, perlecan, syndecans, and versican. Proteoglycans can be placed in a tissue with or without other cells or factors set forth herein, and can serve a mechanical support function, as a reservoir for other factors, provide cues to cells, hydrate and bulk tissues, serve as receptors, amongst other extracellular matrix functions.

Proteoglycans contain a core protein to which is attached one or more glycosaminoglycan (GAG) side-chains. Proteoglycans have highly acidic and hydrophilic glycosaminoglycan (GAG) chains that have a major influence on tissue hydration and elasticity. Proteoglycans provide mechanical support and also control the availability of growth factors to cells and permits the rapid diffusion of nutrients, metabolites and hormones between the blood and the tissue cells. The glycosaminoglycan group of complex carbohydrates include chondroitin sulphate, dermatan sulphate, keratan sulphate, heparan sulphate, and hyaluronan. The carbohydrate group can bind to other extracellular matrix proteins. Proteoglycans contain as much as 95% carbohydrate by weight.

The GAG chains can form gels of varying pore size and charge density and thus can serve as selective sieves to regulate the traffic of molecules and cells according to their size, charge or both. Proteoglycans can serve in chemical signaling between cells. They bind various secreted signal molecules, such as protein growth factors and can enhance or inhibit their signaling activity. For example, heparan sulfate chains of PGs bind to fibroblast growth factor (FGFs) which stimulate a variety of cell types to proliferate by oligomerizing the growth factor molecules so that they can cross-link and activate their cell-surface receptors, the transmembrane tyrosine kinases. Other signal molecules bind to the GAG chains, but others bind to the core proteins of the PG, such as transforming growth factor β (TGF-β) to decorin. Binding to decorin inhibits the activity of the growth factors.

Proteoglycans also bind and regulate the activities of other types of secreted proteins such as proteases and protease inhibitors. Such binding could immobilize the protein close to the site where it is produced to restrict its range of action; block the protein's activity; provide a reservoir of protein for delayed release; prolong the action of the protein by protecting its degradation; and alter the protein concentration for presentation to the cell-surface receptors. For example, heparan sulfate proteoglycans immobilize chemokines on the endothelial surface of a blood vessel at an inflammatory site. This prolonged period of chemokine immobilization stimulate white blood cells to leave the bloodstream and migrate into the inflamed tissue. Proteoglycans interact with ECM components that include cell adhesion and growth factors. Proteoglycans (e.g. CSPGs) can modulate cell attachment.

Some proteoglycans (syndecans, betaglycans) are also integral components of plasma membranes inserted across the lipid bi-layer or attached to the bi-layer by a glycosylphosphatgidylinositol (GPI) anchor.

Transmembrane proteoglycans are important cell adhesion molecules to interact with matrix components such as other proteoglycans and collagen. Both soluble and transmembrane proteoglycans act as low-affinity growth factor receptors that can stabilize, activate, or translocate the growth factor to the high-affinity receptor.

Most, if not all of the proteoglycans, especially the larger protein cores have alternately spliced variants or isoforms. And most of the proteoglycans listed are made by most cells and present to some degree in most tissues. Below are described some tissues and cell types that contain the predominant expression of the protein and other tissues or cell types not listed can also express many of these proteins.

Aggrecan interacts with hyaluronan via a hyaluronan binding domain and a link protein. Aggrecan has about 100 proteoglycan molecules per hyaluronan molecule and results in a high osmotic pressure in tissue. The core protein has a mass of 220 kilodaltons and the complex, 2.6×10⁶ daltons. Aggrecan has about 87% chondroitan sulphate, 6% keratan sulphate, 7% protein and contains immunoglobulin, link protein, EGF, lectin and CCP repeats. It contains keratan sulphate and chondroitan sulphate attachment domains and has N-linked glycosylation sites. It binds many other ECM proteins including tenascin-C. Aggrecan and a number of isoforms or alternately spliced variants exist in cartilage, spinal cord and skin extracellular matrix. It imparts a turgor to tissue. Transcription factors such as SOX9 and vitamin derivatives such as retinoic acid, upregulate aggrecan gene expression. Alternately spliced versions of domains and altered reading frames of proteoglycans including aggrecan can be used in the invention.

Agrin is a major heparan sulphate proteoglycan present in embryonic chick brain in muscle fiber basal lamina at the neuromuscular junction. Agrin has a mass of 225 kilodaltons and exists as at least 8 different isoforms. It contains EGF, G and Kazal-like repeats, nine follistatin-like repeats, three laminin globular G domains and has N-linked glycosylation sites. It is a component of the synaptic basal lamina and promotes acetylcholine receptor clustering on cultured myotubes. The N-terminal half of the molecule is responsible for the tight interaction with the ECM. Membrane and soluble forms of agrin exist.

Bamacan is a chondroitan sulfate proteoglycan and present in basement membranes. It has a m.w. 138,000, has structural features with proteins that stabilize the chromosomal scaffold at mitosis and contains O-linked glycosylation sites.

BEHAB is identical to the N-terminal half of brevican and functions in the brain as a link protein to stabilize interactions between hyaluronan and proteoglycans. It contains about 371 amino acids with the proteoglycan tandem repeat family of hyaluronan-binding proteins and immunoglobulin repeat and has N-linked glycosylation sites.

Betaglycan, m.w. 36,000, contains chondroitan sulfate and dermatan sulfate and is located on the cell surface and matrix. It binds TGF-β.

Biglycan, molecular weight of about 41,000, is a member of a family of the small chrondroitan/dermatan sulphate proteoglycans in which the protein chains contain leucine rich repeats, and is highly homologous with sequences in other proteoglycans such as decorin and fibromodulin. Biglycan is the primary small proteoglycan in aorta and cartilage. It can bind to fibronectin, TGFβ and collagen type I and VI.

Brevican is a chondroitan sulphate proteoglycan and is a member of the hyaluronan-binding family of proteoglycans, aggrecan, versican and neurocan. It has a molecular weight of 96,000 and is presenting in brain. It contains immunoglobulin, link protein, EGF, lectin and CCP repeats, a hyaluronic acid binding domain and N-linked glycosylation sites.

Decorin, m.w. 38,000, is a member of the family of small chrondroitan sulphate/dermatan sulphate and its protein cores contain leucine-rich repeats and N-linked gylcosylation sites. It is relatively abundant in bone, tendon, sclera and cornea. It is needed for collagen fiber formation. It can bind to TFG-β and collagen type I and II fibrils. Decorin is expressed by stromal cells and is involved in cell proliferation. Overexpression of decorin can inhibit growth in many cell types. It can suppress neoplastic cell growth. Decorin interacts with TGFβ neutralizing its action, binds to the EGF receptor, interacts with and induces p21, a strong inhibitor of cyclin-dependent kinases.

Fibromodulin is a member of the small chondroitin sulphate/dermatan sulphate proteoglycans with leucine-rich repeat core proteins which share homology with serum protein LRG and platelet surface protein GPIb. It can modulate collagen fiber formation and is present in most tissues, including skin, tendon, sclera, cornea and cartilage. Like decorin, fibromodulin binds types I and II collagen fibrils in vitro and plays a role in collagen fibril assembly. Fibromodulin is substituted with keratan sulphate glycosaminoglycan chains. It has sites for tyrosine sulphation and N-linked glycosylation.

Heparan sulphate proteoglycans (HSPGs) are present in many tissues and has the ability to bind and release growth factors to cells. It contains O-linked glycosylation sites. HSPGs comprise perlecan and the syndecan family of proteoglycans. These proteoglycans function in cell growth, differentiation and the transport of growth factors. Glypican-1 has a possible role of growth factor transport into the nucleus from the cytoplasm. Other HSPGs transport growth factors in the extracellular matrix between cells. Heparan sulphate proteoglycans may serve as reservoirs for growth factors that are, for example, added before or generated after the HSPG introduction to a tissue. HSPG binds to many components of the ECM such as laminin, fibronectin, collagen type IV, VEGF, VEGF receptor through it sugar moiety, FGFs (FGF2), MMPs as a docking molecule, amongst others.

Keratocan, m.w. 40,000, is one of three keratan proteoglycans in cornea. It has leucine-rich repeats and N-linked glycosylation sites.

Lumican is a small keratan sulphate proteoglycan, m.w. 39,000, whose core protein is homologous to the leucine rich proteoglycans decorin, biglycan and fibromodulin. It is present in the cornea, muscle, aorta and intestine. Lumican has N-linked glycosylation sites.

LYVE-1 (lymphatic vessel endothelial hyaluronan receptor) is a high molecular weight polymer composed of alternating units of D-glucuronic acid and N-acetyl-D-glucosamine. Hyaluronan is in the ECM of most tissues and modulates tissue development, remodeling, homoestasis, and other functions.

Neurocan is a chondroitin sulphate proteoglycan, m.w. 137,000, with immunoglobulin, link protein, EGF, lectin and CCP repeats, a hyaluronic acid binding domain, an RGD cell adhesion site and N-linked glycosylation sites. It is present in the brain.

Perlecan, m.w. 468,000, is a specific and integral component of all basement membranes and a heparin sulphate proteoglycan. It interacts with laminin, collagen type IV in the basement membrane and serves as an attachment substrate for cells. Perlecan filters molecules passing into the urine from the bloodstream in the basal lamina of the kidney glomerulus. Thus it has functions structurally and for filtering in the basal lamina. The heparin sulfate affects filtration of macromolecules. It contains LDL receptor, immunoglobulin, EGF and G repeats, and N-linked glycosylation sites. The core proteins interact with themselves, nidogen and other basement membrane components. Cell binding can occur through an RGD site as well as RGD independent sites. Perlecan is widely distributed in developing and adult tissues playing multiple physiological roles. Heparan sulfate chains bind and potentiate various growth factor activities such as FGF-2. Heparin sulfate proteoglycans and heparin sulfate bind and interact with collagen I fibrils.

Syndecan, about m.w. 30 kDa, contains chondroitin sulfate and heparan sulfate, is located on the surface of many cell types including fibroblasts and epithelial cells, where they serve as receptors for matrix proteins. For example, they modulate integrin function by interacting with fibronectin on the cell surface and with cytoskeletal and signaling proteins inside the cell. Syndecans bind FGFs and present them to FGF receptor proteins on the same cell. Syndecan is involved in cell adhesion. The syndecans are a family of heparin sulfate proteoglycans. There are syndecans 1 through 4. Syndecan 2 is known as heparin sulfate proteoglycan 1, cell-surface-associated HPSG or fibroglycan. Syndecan 3 is known as N-syndecan. Syndecan 4 (amphiglycan, ryudocan) and functions as a receptor in intracellular signaling.

Testicans are extracellular multi-domain chondroitin sulfate proteoglycans, highly expressed in the brain, modulates cell attachment and neurite outgrowth in vitro. Testican 1 and 3 inhibit MT1-MMP and MT3-MMP activities and testican 2 suppresses the inhibitory activity of other testican family members.

Versican, m.w. 264,000, is a large chondroitin sulphate proteoglycan secreted by fibroblasts. Versican contains domains highly homologous to aggrecan for the hyaluronan-binding domain, lectin, complement control protein and EGF repeats. Versican also has immunoglobulin, link protein repeats and N-linked glycosylation sites. Like other members of the chondroitan sulfate proteoglycan family, versican has unique N- and C-terminal globular regions, each containing multiple motifs. Versican has diverse binding partners that include other ECM constituents such as collagen type I, fibronectin, tenascin-R, fibrillin-1, fibulin-1 and -2, hyaluronan, P- and L-selectins, chemokines, and the cell surface proteins integrin β1, EGF receptor, CD44, and P-selectin glycoprotein ligand-1. Versican is involved in intracellular signaling, cell recognition and connecting extracellular matrix components and cell surface glycoproteins.

Hyaluronan is a long backbone of repeating disaccharide sugar units that can facilitate cell migration during repair and tissue morphogenesis, can serve as a lubricant in the joints and is produced in large quantities in wound healing. Many of the functions of hyaluronan depend on specific interactions with proteoglycans and other proteins. CD44 is the hyaluronan receptor on cell surfaces.

Link protein, m.w. 40,000, binds proteoglycan and hyaluronan to form supermolecular assemblies in the ECM. It is in cartilage and other connective tissues interacting with aggrecan, versican, neurocan and other proteoglycans. It has link repeats, the immunoglobulin repeat and N-linked glycosylation sites. Link modules are hyaluronan-binding domains in proteins involved in ECM assembly, cell adhesion and cell migration. TSG-6, a 35 kDA secreted glycoprotein in the ECM, contains a link module domain and interacts with hylauronan and aggrecan. TSG-6 is inducible in fibroblasts, chondrocytes, synovial cells and mononuclear cells by the proinflammatory factors TNF, IL-1, or LPS. TSG-6 is anti-inflammatory through its binding and enhancement of plasmin inhibitor IαI.

Defects can be treated with a proteoglycan or proteoglycans, the core protein portion glycoslyated or not, domains of the protein core, alterante spliced versions of the protein core and PG, the hyaluronic acid chain, the link protein, fragments or motifs of PGs, or the GAG side chains, saccharide resdues (mono, di, oligo, poly).

To treat defects, proteoglycans, ECM and growth factors can vary in concentration from >0% to 100% if used alone and >0% to <100% if part of the cell composition.

Extracellular Matrix Content

Extracellular matrix production or increased ECM content increase in the area of the implantation can be beneficial to the defect treatment. This is especially true for connective tissue defects, but also important for most tissues that rely on ECM present for function. Thus the addition of cells or macromolecules such as proteins, growth factors, cytokines, chemokines, hormones, ECM proteins, serum proteins, immunogenic proteins and other proteins or molecules that increase the synthesis of ECM is these cases are desired. ECM proteins and components can be added to the implantate area to immediately increase the ECM content of the region. Separate sections throughout the document describe those proteins and molecules that affect ECM content and are detailed throughout the document. Furthermore, other proteins and molecules and cell types that increase the ECM content but not described are included in which an ordinary artisan in the field would recognize. Additionally, other treatments known in the art, such as physical or mechanical therapy are amongst others that can be included as available therapies to increase the ECM content in the implantate area.

Serum Proteins and Molecules

Another set of helpful proteins is serum proteins. An advantage of serum proteins is that they are readily available from an autologous or other donor source, e.g., by drawing blood, allowing the blood to clot, and recovering supernatant from the unclotted portion. Serum proteins have been proven to be important for maintenance of cells in vitro and, similarly, can be effective for maintaining cells in vivo at an implantation site. While gels of certain proteins have been used as cellular scaffolding, the use of serum proteins in soluble form is not conventional. In general, serum factors used in the culture of cells in vitro may be used to some advantage when applied in combination with implanted cells. The serum proteins are preferably in solution or suspension and not gelled or cross-linked, so as to be fully available for interaction (absorption) with cells and subject to cellular receptors/transduction pathways/internalization and/or cellular down regulation, as needed.

Plasma is about 50 to about 55% of blood volume. It is about 92% fluid and 7% protein and 1% hormones, lipids, sugars, inorganic salts and gases. Serum is the part of plasma that remains following removal of fibrinogen and clotting factors (although smaller amounts of these, hemoglobin, complement system and other plasma proteins remain in the serum). Serum also contains extracellular matrix proteins and molecules such as laminin, tenascin, fibronectin and collagens. Serum provides a variety of macromolecular proteins, carrier proteins for water-insoluble molecules, nutrients and protein factors, attachment factors, hormones, growth factors, cytokines, chemokines, lymphokines, proteins to neutralize toxic components or to buffer the medium, amongst others.

Proteins in serum can be included in the composition of cell introduction to the defect for improved correction of the defect. Proteins introduced into the patient may also be the same proteins involved in the process of culturing of the cells. The proteins by themselves or in combination with other proteins may be used for the same reasons. The effectiveness of serum proteins is not fully understood, but, in some aspects, it may relate to the presence of cell adhesion factors, growth factors, and/or various transport proteins. The functions of many proteins in serum remain, in some aspects, obscure, though proteins are a major component of serum. Major functions of serum and its proteins for cells are cell attachment, cell spreading, cell mobility, cell migration, nutrition, trace element, vitamin and energy metabolism, ECM production, hormone transport, cell stimulation, cell proliferation, cell differentiation, cell protection and cell survival, among others. Adhesion proteins, such as fibronectin, enhance the binding of the cells to the local extracellular matrix and to integrins present on the cell surface. Fibronectin can also promote differentiation or maintain differentiation of the cells implanted. Fibronectin is useful for cell survival and for protecting cells from apoptosis or anoikis. Fibronectin also acts as an opsonin which assists in phagocytosis. Other adhesion proteins not only prevent migration of cells away from the injection or implantation site but may serve in other similar capacities, as for fibronectin listed above. Some other adhesion proteins (see below) include vitronectin and laminin. Serum proteins can serve as carrier proteins for lipids and trace elements such as albumin (fatty acid, hormone, growth factor and vitamin transport), transferrin (iron transport), ceruloplasmin (copper transport) ferritin (iron transport), lipoproteins (HDL, LDL, VLDL, apoA1, apolipoprotein A-II, apolipoprotein B) for cholesterol and fatty acid transport.

Serum proteins can also serve in cell implantation as an immediate nutrient source for survival and growth. Some serum proteins are transport proteins. Other proteins are needed as cell attachment factors, growth factors, protease inhibitors, cytotoxic quenchers or a host of other diverse activities. For example, fibronectin promotes cell attachment and fetuin present in fetal serum promotes cell attachment. Growth factors and hormones can be mitogenic for a number of cell and tissue types.

Proteins can be purified or made by chemical, recombinant or cell-free translation systems. The chemical approach for smaller proteins under m.w. 30,000 allows rapid preparation and prevention of biological contaminants such as viruses, prion and endotoxins.

The concentration range of greater than 9,500 proteins in plasma or serum begins at almost millimolar for albumin (up to 6700 mg/100 ml) down to femtomolar for proteins such as tumor necrosis factor (TNF) and lower for “leakage” proteins from dying cells that release their contents into the circulation. Albumin is greater than 50% of the protein mass in plasma. In addition immunoglobulins, transfernin, fibrinogen, complement components, apolipoproteins and a few other proteins are responsible for 99% of the protein mass in plasma. Immunoglobulins concentrations are about IgA, 70 to 400 mg/100 ml; IgG, 700 to 1,600 mg/100 ml; IgM, 40 to 230 mg/100 ml.

Serum proteins can include certain growth factors, cytokines, extracellular matrix molecules, cell adhesion factors, and transport proteins that are described elsewhere herein. Cells may be combined with serum proteins for implantation into a patient, e.g., at a defect site, and cells may be combined with serum factors or factors can be used by themselves, including any combination thereof.

Approximate concentration of various proteins in serum are: proteins and polypeptides 40-80 mg/ml; albumin 20-67 mg/ml; fetuin 10-20 mg/ml; globulins 1-15 mg/ml; α-1 globulin, 1 to 3 mg/ml; α-2 globulin, 6 to 10 mg/ml; β globulin, 7 to 12 mg/ml; and γ globulin 7 to 16 mg/ml; α1 acid glycoproteins (orosomucoid), 0.5 to 1.2 mg/ml; transferrin 2-4 mg/ml; protease inhibitors α₁-antitrypsin and α₂-macroglobulin 0.5-2.5 mg/ml; fibronectin (cold-insoluble globulin) 1-10 ug/ml; vitronectin or S protein binds to complement 20 ug/ml; EGF, FGF, IGF I and II, PDGF, IL-1, IL-6, insulin, VEGF, angiogenin, other growth factors 1-100 ng/ml and less; IgE 50 ug/ml; linoleic acid 0.01-0.1 uM; haptoglobin 0.3 to 2.0 mg/ml; ceruloplasmin 0.3 mg/ml; α2-microglobulin, 2.5 mg/ml; haptoglobulin, 2 mg/ml; hemopexin, 1 mg/ml; pre-albumin or transthyretin, 200-350 ug/ml; β2 glycoprotein, 20-25 mg/ml; α2+beta-lipoproteins (LDL) 4-7 mg/ml; α-high density lipoproteins, 0.6 to 1.5 mg/ml; high density lipoproteins, 2-4 mg/ml; fibrin, 2-5 mg/ml; C3, 0.9 to 1.8 mg/ml; C4, 0.1 to 0.4 mg/ml; C-reactive protein is present in trace amounts in the plasma of <8 ug/ml, but inflammation, trauma, tissue necrosis or malignant tumors can increase the levels 2,000 fold; oleic acid, ethanolamine, phosphoethanolamine are bound to proteins such as albumin. Trace elements and iron, copper and zinc can be bound to serum proteins.

Proteins in serum include fetuin (A&B), asialofetuin, complement C1-C9, ACE (angiotensin converting enzyme), angiotensin II, antithrombin III, antichymotrysin, β2-microglobulin, carboxypeptidase, CRP C-reactive protein, gelsolin, protein C, glycophorin, fraction IV globulin, HS alpha 2 glycoprotein, TPA tissue plasminogen A activator and inhibitor (PA1-1), alkaline phosphatase, lactate dehydrogenase and many other enzyme activities, parathyroid hormone, troponin, annexin V (a member of the calcium and phospholipids binding family of proteins with vascular anticoagulant activity), vasoactive angiotensin, PAP1, PP4, CPB-1, CaBP33, VACa, anchorin CII, lipocortin-V, endonexin II, thromboplastin inhibitor, haptoglobulins, macroglobulins, S100 proteins, α1 acid glycoproteins (orosomucoid), α1 glycoproteins, β2 glycoprotein, cold agglutinins, cryoglobulins, cryofibrinogen, platelet factor 4, coagulation and complement proteins, ghrelin (a secretagogue of growth hormone), cholesterol metabolism proteins such as serum lecithin cholesterol acyltransferase, cholesterol ester transfer protein, and lipoprotein lipase, adipocyte production of aP2, lipoprotein lipase, adipsin, adiponectin, leptin and resistin, plasma endocrine hormones such as insulin and parathormone, IGFBP3, growth factors such as TNF, α fetoprotein, serum binding proteins such as mannose, sex hormone globulin and other binding proteins.

Some of the supplements and active concentrations in serum-free culture that have been tried are listed below. Some correspond to in vivo serum concentrations and some do not for cell activity in vitro.

Many tissue growth factors, cytokines, chemokines, hormones and supplements are active for cell culture in the 0.1 to 100 ng/ml range. Some examples of tissue growth factors for cell culture can be: EGF, 0.1-10 ng/ml; heregulin (HRG), 10-100 ng/ml; β-cellulin, 1-50 ng/ml; αFGF, βFGF, 1-10 ng/ml; IGF-I, IGF-II 1-50 ng/ml; keratinocyte growth factor (KGF), 1-50 ng/ml; PDGF, 1-50 ng/ml; the TGF family of TGF-β1, 2, 3, 4, 5, 0.1-10 ng/ml; activins A,B,C, 1-100 ng/ml; inhibins A,B, 1-100 ng/ml; the neurotropins or NGF, 1-10 ng/ml; GDNF, 10-100 ng/ml; NT3, 10-100 ng/ml; NT 4/5, 10-100 ng/ml; SMDF, 0.01-20 nM; BDNF, 1-50 ng/ml; CTNF, 1-50 ng/ml; serotonin, 0.05-0.2 ug/ml; cytokines of T-cell growth factor, 0.01-1 ug/ml; TNFα, 0.1-100 ng/ml; TNFβ, 0.01-1 ug/ml; G-MCSF, 0.01-1 ug/ml; GCSF, 0.01-1 ug/ml; interleukins, 1-100 ng/ml; binding proteins or transport proteins, 1-5 ng/ml; ceruloplasmin, 1-5 IU/ml; BSA, 1-25 ug; α₂-macroglobulin 0.1-5 mg; follistatin 10-100 ng; IGF-1 binding proteins, 0.01-10 ug/ml; retinoid binding proteins, 0.01-10 ug/ml; hormones of insulin, 0.1-10 ug/ml; follicle stimulating hormone, 1 ng-1 ug/ml; leutenizing hormone, 1 ng-1 ug/ml; leutenizing hormone releasing hormone, 1-10 ng; glucagons, 10-100 ng/ml; parathryroid hormone, 2-100 ng/ml; growth hormone (somatotropin), 50 ng/ml; somatostatin, 10-500 ng/ml; TSH, 1-10 ng/ml; TRH, 1-10 ug/ml; T3, 20 nM; T4, 100 nM; calcitonin, 0.4-25 ng/ml; caerulin, 250-430 ug/ml; GLP, 20-100 pg/ml; gastrin, 100-200 pg/ml; substance P, 0.1-20 ug/ml; hydrocortisone, 10⁻⁸M; testosterone, 10⁻⁹ to 10⁻⁷M; estradiol, 10⁻⁹ to 10⁻⁸M; progesterone, 10⁻⁹ to 10⁻⁷M; prostaglandin-E1, E2a, and F, 10-100 ng/ml; some of the attachment factors can be fibronectin, (Clg) 10 ug/ml or coat; laminin, 1-5 ug/ml; lamin, 10 ug/ml or coat; collagen coat; polylysine coat; some of the other additives can be trace element mixtures; thrombin, 10-1000 ng/ml; aproteinin, 10-100 ug/ml; vitamins, fatty acids, 0.1-1 uM; linoleic acid, 0.01-0.1 uM; phospholipids, ˜2 mg/ml; and cholesterol, 10 uM. Lower or higher concentrations of the above factors and others can be used in cell culture and in cell implantation.

In culture, some of the most common required additives can be insulin (1-10 ug/ml) (which improves plating efficiency as one of its functions), transferrin (1-100 ug/ml), hydrocortisone (which improves cloning efficiency as one of its functions) and selenium (10-30 nM). Some cells have added lipid requirements in the form of bovine lipoprotein or lipid-rich bovine serum albumin. HDL and LDL can be used in serum free media formulation or as an additive to serum rich media. In vivo VLDL and LDL deliver cholesterol to cells from the liver whereas HDL transports cholesterol from cells to the liver. Heat inactivation can remove complement in the serum and reduces the cytotoxic action of immunoglobulins in the serum.

Functions, such as cell survival, differentiation, maintenance of differentiation and proliferation, can be carried out by a growth factor, cytokine, chemokine or hormone in the serum (e.g. epidermal growth factor, PDGF, TNF, Interleukins, etc.) present in cell culture or cell implantation in different forms, including as a recombinant protein.

The acute and chronic phase response increases concentrations of various serum proteins. After an infection, physical injury or inflammatory stimuli (acute or chronic), acute phase liver-derived plasma proteins are made: C-reactive protein (CRP), serum amyloid P component (SAP), serum amyloid A or serum amyloid associated protein (SAA), alpha 1-acid glycoprotein (AAG or orosomucoid) and fibrinogen. They provide enhanced protection against invading micro-organisms (helpful when doing injections and implantation), limit tissue damage (helpful when doing injections and implantation) and are involved in tissue repair and regeneration, the clearance of host and foreign debris, and promote a rapid return to homeostasis. CRP specifically, reacts with cell surface receptors that result in opsonization, enhanced phagocytosis and passive protection. This also results in activation of the complement pathway, scavenging of chromatin fragments, inhibition of the growth and metastasis of tumor cells and modulation of polymorphonuclear functions. SAA is a precursor of protein AA in secondary amyloidosis. AAG may play an immunoregulatory role and binds a number of diverse drugs. Fibrinogen, in addition to clot formation, binds with fibrin to complement receptor type 3. Fibrinogen is important in wound healing. The concentration of each protein varies in noninfectious, infectious and connective tissue disease states. CRP and SAA may increase in concentration by as much as 1000-fold, the AAG and fibrinogen about 2 to 4 fold. These proteins can also be produced in extrahepatic tissues by fibroblasts, adipocytes, endothelial cells and monocytes. Cytokines such as IL-6, IL-1, TNFα, interferon gamma and other stimulatory factors are involved. SAP binds fibronectin, heparan sulfate and dermatan sulfate. AP deposition can be an elastase inhibitor. SAAs (SAA 1, 2, 3, and 4) are small apolipoproteins that associate with the third fraction of high-density lipoprotein (HDL3) during the acute phase response. It displaces apoA1 thus interfering with cholesterol metabolism and perhaps promoting vascular disease.

Acute phase serum proteins increase during acute inflammation. They are α-1 antitrypsin, α-1 glycoprotein, amyloid A & P, antithrombin III, C-reactive protein, C1 esterase inhibitor, ceruloplasmin, haptoglobin, orosomucoid, plasminogen and transferrin. Other serum proteins involved in the acute phase response are complement proteins C2, C3, C4, C5, C9, Factor B, C1 inhibitor, C4 binding protein, the coagulation proteins, fibrinogen and von Willebrand factor.

Amyloidosis is produced during inflammatory states. Amyloid P, 180 kD, is a soluble serum protein and a minor component of amyloid deposits. It is a normal α1-glycoprotein and is closely homologous to C-reactive protein. It has an affinity for amyloid fibrils. Amyloid AA, 8.5 kDa, is a nonimmunoglobulin and makes up to 90% of amyloid deposits in amyloidosis due to chronic inflammation. Chronic inflammation leads to increased SAA levels. SAA is the serum precursor of AA amyloid. It constitutes the protein constituent of a high-density lipoprotein and acts as an acute-phase reactant. Amyloid AL consists of immunoglobulin light chains, their N-terminal fragments, or a combination of the two. Amyloid production increases with age.

SAA is a 12 kDa protein in serum and a precursor of the AA class of amyloid fibril protein. Formed in the liver, SAA associates with the HDL3 lipoproteins in the circulation. Conversion to AA is accomplished by cleaving amino and carboxy terminal peptides to yield an 8.5 kDa protein that forms fibrillar amyloid deposits. During inflammation, there may be a 1000 fold increase in SAA levels.

SAP is a 180 to 212 kDa serum protein and a minor second component in all amyloid deposits. It does not increase during inflammation and makes up 10% of amyloid deposits. It is indistinguishable from normal α1 serum glycoprotein and is closely homologous to C-reactive protein.

α-fetoprotein bears homology with serum albumin and is normally present in fetal serum. It induces immunesuppression by facilitating suppressor T lymphocyte function and diminishing helper T lymphocyte action.

α1-microglobulin (1) is a 30 kDa protein of the lipocalin family, has a role in immunoregulation and functions as a mitogen. The protein blocks antigen stimulation and migration of granulocytes. It can prevent any granulation formation from implantation of cells. It can dampen an immune response after use with immunogenic agents.

Angiotensin-renin system. Angiotensin II is a fibroblast mitogen and inducer of alveolar cell apotosis. ACE I is an angiotensin-converting enzyme inhibitor. Angiotensin II affects cardiac fibroblast proliferation and cardiac myocyte and fibroblast differentiation. Angiotensin II controls the renin-angiotensin system, which is the main regulator of blood pressure, intravascular volume and electrolyte balance. Angiotensin II is a vasoconstrictor and stimulator of aldosterone release. Angiotensin II promotes ECM deposition. XPP, a tripeptide, wherein X is C, M, S, T or K, is an inhibitor of angiotensin-converting enzyme. There at least seven different angiotensins (1-7).

β2 microglobulin promotes maturation of T lymphocytes and is a chemotatic factor. It is a component of the major histocompatibility complex class I proteins.

C-reactive protein (CRP), 115 kDa, is present in trace amounts in the plasma at <8 ug/ml. Inflammation, trauma, tissue necrosis or malignant tumors can increase the levels 2000 fold. IL-6 regulates its production. CRP may activate the complement pathway.

Ceruloplasmin binds copper and contains 8% carbohydrate.

Complement is a system of 20 soluble plasma proteins and plays a critical role in assisting phagocytosis of immune complexes, which activate the complement system. Complement system proteins include C1, C1 esterase inhibitor, C1 inhibitor, C1q, C1r, C1s, C2, C2a, C2b, C3, C3a, C3b, C3bi, C3c, C3 convertase, C3d, C3dg, C3e, C3f, C3g, Ce proactivator, C4, C4a, C4A, C4b, C4B, C4b binding protein, C4bi, C4b inactivator, C4c, C4d, C5, C5a, C5b, C5 convertase, C6, C7, C8 and C9. Small peptides and proteins released and involved in the coagulation cascade affect cell immune responses. C1 esterase inhibitor counteracts activated C1, thereby diminishing the generation of C2b and preventing the development of edema. C1 esterase inhibitor can be useful in the invention to prevent excess swelling or water retention that could occur from the implantation.

Cytokeratins are apoptotic proteins present in serum.

CRISP-3 is a 28 kDa cysteine-rich secretory protein 3 that is present in exocrine secretion and in secretory granules of neutrophil granulocytes and plays a role in innate immunity. CRISP-3 is present in high concentration in plasma and is bound to the plasma protein alpha1B-glycoprotein (A1BG).

Cytokines are immune system proteins that coordinate antibody and T cell immune system interactions and amplify immune reactivity. Cytokines include monokines made by macrophages such as interleukin 1, tumor necrosis factor, α and β interferons, colony-stimulating factors and lymphokines such as interleukins, interferons, GM-CSF, lymphotoxin, TCGF, T cell growth factor 1 (IL-2) or T cell growth factor 2 (IL-4). Growth factors can be cytokines that facilitate the growth and proliferation of cells such as PDGF, erythropoietin, IL-2 (T cell growth factor), amongst others. Cytokines are protein mediators that can be short-range with a wide range of actions. They have roles in all biological processes including immune regulation, inflammation, hematopoiesis, T cell subset differentiation, tumor, ECM production and tissue repair. Cytokines are involved cell proliferation such as in T cell growth (IL-2, IL-4, IL-7, IL-15, IL-21), inflammation (IL-1, IL-6, TNF, IFN-γ) and inhibition of inflammation (IL-4, IL-10, TGF-β). Cytokines, as ECM or serum molecules, are accessible to therapeutic proteins such as antibodies or soluble receptors. PMNs and the cytokines IL-8, IL-6, IL-1α and IL-β, and TNF-α are involved in inflammation and tissue damage. Cytokines are involved in differentiation. Stromal cells and stem cells in the presence of IL-3, GM-CSF and EPO progress through the erythroid lineage, while stem cells in the presence of IL-3 and TPO differentiate to megakaryocytes or platelets. Stem cells in the presence of Flt-3L, IL-3, GM-CSF and IL-6 give rise to myeloid progenitor cells, subsequent exposure to G-CSF or GM-CSF give rise to granulocytes, or exposure to IL-3, IL-6, GM-CSF, M-CSF give rise to monocytes. Stem cells proliferate in the present of IL-3 and SCF, in the presence of IL-3 stem cells give rise to lymphoid progenitor cells which when exposed to IL-2, IL-4, IL-7 differentiate into T cells, or IL-7, IL-4, IL-5, IL-6 differentiate into B cells, and in the presence of IL-2 differentiate into NK cells. Cytokines in immune regulation include IL-10, TGF-β, and cells such as T regulator cells, APC (antigen presenting cells) and effector T cells. Cytokines in T cell differentiation include IL-4, IL-15 in TH0 to TH1 cell conversion and IL-27, IFN-γ, IL-12 p70, IL-15, TRANCE, IL-23 in TH0 to TH2 cell conversion. Cytokines in tumor control include autocrine IFN-γ produced by T cells, IFN-γ production by T and NK cells. IFN-γ acts on macrophages and dendritic cells to produce IL-12 that affects T and NK cells, such as NK cell release of perforin.

Endothelins are 21 amino acid peptides made by vascular endothelial cells and are vasoconstrictors. Endothelin-1 is a fibroblast and myoblast mitogen.

Fibrin is formed through the degradation of fibrinogen into fibrin monomers. Fibrinogen may be degraded by plasmin. Fibrinogen is 340 kDa. Fibrinogen B chain and soluble partially degraded fibrinogen can be a fibroblast mitogen. Fibrin/fibrinogen can be useful to promote blood clotting and limit bleeding at the site from the injectate.

Fibronectin is an adhesion promoting dimeric glycoprotein. Over 50 alternately spliced variants exist. The tetrapeptide, Arg-Gly-Asp-Ser, assists cell adhesion. Fibronectin has fibrin, Clq, heparin, transglutaminase, collagen types I, II, III, V, VI and sulfated proteoglycans binding sites. Fibronectin functions in cell-substrate adhesion, contact inhibition, cell migration, cell differentiaton, inflammation and wound healing. Plasma fibronectin is soluble and differs from insoluble cellular fibronectin by the absence of the two commonly spliced domains EIIIA and EIIIB. A further description of fibronectin is listed above in the ECM section.

Serum contains glycated serum proteins (GSPs), such as albumin and serum protein. Many of these AGEs (advanced glycation end-products) can be sequestered in the invention by incubation with RAGE soluble receptor or the binding domain of the extracellular portion of the RAGE receptor that has the affinity for AGEs.

Serum contains anti-oxidant enzymes such as superoxide dismutase, catalase, glutathione peroxidase and glutathione transferase that keep cells from oxidative damage. Albumin bound bilirubin is also cytoprotective against oxidative damage.

The serum globulins are separated into alpha, beta and gamma types.

Some growth factors also induce differentiation of specific cell types. For example, peptide hormones that induce differentiation include melanotropin for melanocytes, thyrotropin for the thyroid, erthropoeietin for erythoblasts, prolactin for mammary epithelium, and insulin for mammary epithelium. Cytokines that induce differentiation include NGF for neurons, glia maturation factor for glial cells, epimorphin for kidney epithelium, CNTF for type 2 astrocytes, HGF for hepatocytes and kidney TGFβ for melanocytes and bronchial epithelium.

Some of the growth factors in serum are listed below. Many are also described in the above ECM section. Growth factors are polypeptides with mitogenic activity (amongst many other functions) such as EGF (epidermal growth factor), FGF (fibroblast growth factor), PDGF (platelet derived growth factor), insulin growth factors (IGF I, II) and IGF binding proteins. Growth factors overlap in function with polypeptides made by cells of the endocrine system, such as growth hormone from the pituitary gland. For example, the hormones insulin and progesterone, and the growth factors adiponectin (acts on adipose tissue) and leptin are endocrine factors.

Transport proteins can be hormone binding or growth factor binding proteins are also important for proper delivery or for proper sequestration of various hormones so that the hormone can act or not act on the target cells. Thyroxine binding globulin binds thryoxine. Transcortin binds cortisol and other steroid hormones. Other transport proteins can carry nutrients. Transcobalamin is the main transport protein for vitamin B 12. B12 is needed for the immune B cell response and cell energy metabolism. Transferrin, a glycoprotein, transports iron from the blood to receptors on cells. The major iron transport protein in serum binds to a specific membrane receptor CD71. It is needed for growth of cells in vitro. It can be considered a growth factor since proliferating cells express high numbers of receptors and transferrin binding is needed for DNA synthesis. Besides its role in iron transport, transferrin acts as a cytokine with functions unrelated to iron transport. It can act as a cell proliferator of immune and other cell types in vitro. Normal transferrin contains sialic acid residues. Ferritin is an iron-containing protein, serving as a source of stored iron and prevents iron cytotoxicity.

Growth factors are usually polypeptides often as large as 100 amino acids. Growth factors are absorbed to the cell surface, attaching to specific cell surface proteins (growth factor receptors). In the presence of growth factors acting as mitogens, cells pass through G1, S, G2 and M phases, doubling in size and then dividing. Serum growth factors are needed to stimulate the first ⅔ of G1, thereafter the cells divide around the cycle. Without serum growth factors, cells exit the cycle into a resting phase, G0, which is reversible upon addition of serum growth factors. A short description of growth factors include the following:

BDNF (brain-derived neurotrophic factor) promotes differentiation of stem cells into muscle and blood vessel cells, as does BEGF-A.

FGFs (fibroblast growth factors) have a central 140 aa core and a strong affinity for heparin. Many FGFs stimulate the growth of fibroblasts and other cell types. FGF 1 is important for wound healing, angiogenesis and mitogenesis of many cell types.

Hematopoietic Cell Growth Factors are a family of hematopoietic regulators that support proliferation and differentiation of blood cells of different lineages. Erythropoietin and colony-stimulating factors belong to this family. Erthropoietin can be used to increase wound healing of skin and other tissues. The growth factor increases production of red blood cells. G-CSF (granulocyte colony-stimulating factor) facilitates formation of granulocytes in bone marrow. G-CSF is made by fibroblasts, endothelial cells, macrophages, T, B and mast cells in response to cytokine, immune or inflammatory stimuli and synergistically acts with IL-3 in stimulating bone marrow cells. GM-CSF is a growth factor for granulocyte-macrophage, erythroid, megakaryocyte, and eosinophil progenitor cells. It is a survival factor and activator for granulocytes, monoctyes, macrophages and eosinophils. GM-CSF (granulocyte macrophage-colony stimulating factor) is a growth factor for hematopoietic cells. GM-CSF stimulates production of leukocytes and initiates hematopoiesis. GM-CSF also induces endothelial cells to migrate and proliferate. GM-CSF proliferates tumor cells.

Insulin-like growth factors or somatomedins are made by the liver and many cell types such as fibroblasts. Their release into the blood is stimulated by somatotropin.

PDGF is a connective tissue mitogen, such as for fibroblasts and intimal smooth muscle proliferation. It induces vasoconstriction, chemotaxis and activates intracellular enzymes.

TGF (transforming growth factor[s]) are polypeptides that induce various cells to alter their phenotype.

TGF-α is a 5.5 kDa polypeptide made in the liver that shares ⅓ of its 50 amino acid sequence with EGF, epidermal growth factor. TGF-α stimulates cell growth of (proliferation of epidermal and epithelial cells) and promotes capillary formation. TGF-α can induce anchorage independent cell growth and loss of contact inhibition.

TGF-β has five (1-5) subtypes that are all structurally similar in the C-terminal region of the protein and all have similar functions in their regulation of cellular growth and differentiation. TGF-β regulates growth depending on the cell type and the presence or absence of other growth factors. It regulates the deposition of extracellular matrix (e.g. fibronectin, chondroitin/dermatin sulfate proteoglycans, collagen and glycosaminoglycans), protease inhibitors and cell attachment to the extracellular matrix. TGF-β increases wound healing and induces granulation tissue. It increases the proliferation of osteoblasts and chondrocytes. It differentiates fibroblasts into myofibroblasts. It blocks bone marrow cell proliferation and interferon α induced activation of natural killer cells, decreases IL-2 induced proliferation of T lymphocytes, inhibits T cell precursor differentiation into cytotoxic T lymphocytes and reverses macrophage activation by preventing the development of cytotoxic activity and superoxide anion formation needed for antimicrobial effects. It can diminish MHC class II molecule expression. It also decreases Fcε receptor expression in allergic reactions. Thus, TGF-β has potential value as an immunosuppressant in tissue and organ transplantation. It is also used as an anti-inflammatory agent since it inhibits the growth of both T and B cells. Thus, it can be used to dampen an immune response, increase ECM deposition and inhibit degradation of the ECM.

TNF α (tumor necrosis factor α or cachectin) is a cytotoxic 157 amino acid residue monokine produced by macrophages, monocytes, T lymphocytes, B lymphocytes, NK cells and other cell types when stimulated with bacterial endotoxin or other microbial products. TNF α is involved in inflammation, wound healing and tissue remodeling, can induce septic shock, hemorrhagic nercrosis of tissue, organ failure and cachexia. TNF α increases leukocyte recruitment, induces angiogenesis and promotes fibroblast proliferation.

TNF β is a 25 kD protein that stimulates fibroblast proliferation, kills tumor cells in culture, and simulates most of TNFα actions. It is the mediator in which cytolytic T cells, lymphokine-activated killer cells, natural killer cells and helper-killer T cells induce fatal injury to their targets.

VEGF (vascular endothelial growth factor or vasculotropin), has the isoforms A, B, C, D. VEGF is a mitogen for vascular endothelium and promotes angiogenesis. VEGF can improve the blood supply of the cell implantation site.

Immunoglobulins are present at about 20 mg/ml in serum. IgG forms antibodies against soluble antigens, represents 73% of normal immunoglobulins and protects the body fluids. IgE mediates reaginic hypersensitivity and peaks after the third day after exposure to allergen. IgA forms antibodies against pathogens entering via the gastrointestinal and respiratory tracts, is the predominant immunoglobulin in body fluids, forms 19% of immunoglobulins and protects the body surface (most is synthesized beneath mucous membranes). IgM are natural antibodies aginst gram negative organisms and forms the early antibody response and protects the bloodstream. Immunoglobulin is a mature B cell product synthesized in response to stimulation by an antigen. Antibody molecules are immunoglobulins of defined specificity produced by plasma cells. IgM, IgG and IgA are the 3 predominant classes, whereas IgD and IgE comprise less than 1% of total immunoglobulins. The immunoglobulin superfamily members participate in the immune response and show similarities in structure to immunoglobulins. The family includes CD 2, 3, 4, 7, 8, 28, T cell receptor, MHC class I and II, leukocyte function associated antigen 3 (LFA-3), the IgG receptor and many other proteins.

Metal-binding proteins include haptoglobin, hemopexin, ceruloplasmin, superoxide dismutase, ferritin, and transferrin.

Negative acute phase reactants include albumin- pre-albumin, transferrin, apoA1, ApoA11, α2-HS glycoprotein, inter-α-trypsin inhibitor, and histidine-rich glycoprotein.

Other proteins present are α1-acid glycoprotein, heme oxygenase, mannose-binding protein, leukocyte protein 1, lipoprotein (a), and lipopolysaccharide-binding protein.

Laminins are basement membrane glycoproteins, 820 kDa, that facilitate cell attachment, migration and differentiation. Laminins are produced by macrophages, endothelial cells, epithelial cells and Schwann cells and promote cell attachment to basement membrane components collagen type IV, heparin and glycosminoglycans.

Lipoproteins (high density and low density) and very low density, chylomicrons, apoA1, apolipoprotein A-II and apolipoprotein B are used in cholesterol and fatty acid transport. Apolipoprotein E binds low-density lipids and high-density cholesterol esters. Lipoproteins can increase cell proliferation of various cell types, such as mesangial cells.

Lymphokines are immune cell produced cytokines that facilitate cell proliferation, growth and differentiation such as IL-2, IL-3 and γ interferon.

Macroglobulins belong to the IgG class. The 820 and 900 kDa IgMs are both α2 macroglobulins.

Microglobulin is a globulin or its fragment with a molecular weight of 40 kDa or less. β2-microglobulin is a MHC class I molecule.

Plasminogen is the inactive precursor of the proteolytic enzyme plasmin. It is a β globulin present in tissue, body fluids and plasma. Plasmin, is a 90 kDa enzyme that hydrolyzes fibrin and facilitates the dissolution of intravascular blood clots. It is involved in coagulation, fibrinolysis, inflammation and stimulates B cell proliferation. Plasmin can facilitate the escape of cells from contact inhibition in culture and thus may be used to increase fibroblast cell proliferation in vitro. Plasminogen activator is an enzyme produced by macrophages and converts plasminogen to plasmin.

Senescent cell antigen is a neoantigen appearing on old red blood cells that binds IgG autoantibodies. It is also found on lymphocytes, platelets, neutrophils, adult human liver and embryonic renal cells (in culture).

Serum spreading factors are the 65 and 75 kD glycoproteins that facilitate the adherence of cells and their ability to spread, proliferate and differentiate (e.g. vitronectin).

Substance P is a tachykinin that can induce inflammation (e.g. in joints) when released at local sites. It facilitates the synthesis of IL-1, IL-6 and TNF-α by monocytes.

Suppressin is a 63 kDa protein produced by the pituitary gland and lymphocytes to negatively regulate cell growth, inhibiting lymphocyte proliferation. It is more effective on T cells than B cells. It has properties similar to TGF-β, but it is structurally different.

Thrombomodulin (TM), a transmembrane protein with multiple EGF extracellular domains expressed on endothelial cells and present in plasma, contains one chondroitin/dermatan sulpfate chain that binds to thrombin and is a cofactor for activated protein C. TM decreases thrombin acitivity and by protein C activation, inactivates factors Va and VIIIa and regulates leukocyte activation, reducing organ injury. Thus TM decreases coagulation and inflammation processes. Proteins C and S are physiological anticoagulants.

Thymic hormones, thymic humoral factor(s) (THF) are soluble peptides made by thymic epithelial cells in the thymus such as thymosins, thymopoietin(thymin), serum thymic factor, thymopentin, etc. and govern the differentiation and function of lymphocytes. Thymulin is a nonapeptide in serum and thymus and enhances T lymphocyte activity.

Tissue factor is involved in coagulation and present as a transmembrane receptor in endothelial cells and other cell types and can exist as an extracellular protein as well (e.g. atherosclerotic plaque).

Tuftsin is a tetrapeptide that enhances phagocytosis and is derived from a leukokinin globulin derived substance.

Ubiquitin is a 7 kDa protein found free in the blood or bound to cytoplasmic, nuclear or membrane proteins and marks proteins for degradation.

VCAM-1 (vascular cell adhesion molecule 1) is found on activated bone marrow fibroblasts, other fibroblasts, myoblasts, tissue macrophages, dendritic cells and activated endothelial cells. It facilitates the binding of lymphocytes and monocytes to these cells for the immune response.

Vitronectin (complement S protein, serum spreading factor, somatomedin B, epibolin, VTN, VN). Vitronectin is a 75 kDa (that can be enzymatically cleaved to a 65 kDa and 10 kDa form) cell adhesion glycoprotein in the serum and appears in the basement membrane and ECM. The protein combines with coagulation, complement, fibronolytic proteins and with C5b67 complex to block its insertion into lipid membranes. Vitronectin is the major cell-attachment protein in cell culture serum. The first 44 amino acid sequences is identical to somatomedin B found in the serum. The protein has binding domains for heparin, collagen, plasminogen, PAI-1 (plasminogen activator inhibitor I), C9 and perforin. Vitronectin is associated with C5b-9 and the thrombin-antithrombin complex, serving as a scavenger. This opsonization by vitronectin may be useful to rid the injection site of blood clots. Plasma VN regulates coagulation, fibrinolysis, complement activation, extracellular anchoring or attachment and cell proliferation, spreading, migration and adhesion. All of these features can be used in the invention. VN inhibits fibrinolysis by mediating the interaction of type 1 plasminogen activator inhibitor (PAI-1) with fibrin. VN binds PAI-1 in the ECM and in serum. VN associates with fibrin during coagulation and thus regulates hemostasis and inflammation. VN consists of the N-terminal end that contains PAI-1 and urokinase binding sites, an RGD sequence that binds a number of different integrins, a string of acidic amino acids that bind thrombin-anti-thrombin III complexes, and a collagen binding site. The C-terminal end contains binding sites for glycosaminoglycan, PAI-1 and heparin. uPAR can promote adhesion to vitronectin via a high affinity binding site on uPAR.

Von Willebrand Factor is an essential multimeric glycoprotein to stop bleeding after injury. The protein is present in blood, inside platelets, endothelial cells, and the subendothelial extracellular matrix of vessel walls. It contains collagen, heparin, factor VIII and GPIb (platelet glycoprotein) binding domains. The protein mediates platelet adhesion and thrombin formation at the site of injury. The factor is useful in the invention to limit bleeding at the site of the injection.

Wound healing factors include nerve β-NGF, NT-3 and L1 (a distroglycan). Dermal wounds utilize PDGFs (e.g. BB, AB), VEGFs 121, 165, Ang1, ECM proteins, and others. Nonunion bone defects utilize BMP-2, IGF-1 and PTH1-34.

All serum proteins that have cell binding sites can be used for cell adhesion in vivo and in vitro. The binding sites can be the RGD domain as well as other known domains or sites that are included but not limited to the examples given above. Proteins with binding sites for other proteins that assist in adhesion to limit migration of the injected protein or cells can be used. Similarly, other functions such as nutrient delivery, transport protein, protease inhibitor, apoptosis inhibitor, anoikis inhibitor, amongst others can encompass those serum proteins demonstrating such properties.

Hormones, most growth factors, cytokines, chemokines, many ECM proteins and enzymes exist in the serum. Converting enzymes for the pro form or precursor form of protin in the serum produce active protein. More detail on the proteins are listed in the cell growth, proliferation and other sections to follow.

To treat defects, serum proteins can range in concentration from more than 0% to less than 100% w/w when used in conjunction with cells and more than 0% to 100% w/w when used alone.

Procoagulants

The coagulation system is a cascade of interactions comprising at least twelve serum proteins that result in the generation of fibrin. Blood clotting cascade factors, referred to as procoagulants herein, have been described in the scientific literature. Such factors may be combined with materials as described herein, e.g., cells or proteins. Procoagulants can be useful to stop any bleeding due to implantation (e.g. injections) of cells or proteins. Procoagulants can be useful as cell mitogens for increased cell number. Without being bound to a particular theory, it is believed that these factors can trigger responses from the patient or the implanted cells that are useful for the implant's success, as described below.

Blood coagulation represents a series of reactions in which plasma zymogens are converted into active enzymes resulting in a fibrin clot. The coagulation system includes Factors (F) and activated factors (a): factors I (fibrinogen), II (prothrombin), IIa (thrombin), III, IV, V, Va, VII, VIIa, VIII, VIIIa, IX, IXa, X, Xa, XI, XIa, XII (Hageman factor), XIIa, XIII, XIIIa, prekallikrein, and high-molecular weight kininogen. The His rich domain of the light chain of kininogen can be involved in the clotting process. Factor VIIa and the other clotting proteins can be used to control bleeding at the implantation site.

The extrinsic and intrinsic coagulation pathways converge into the common pathway. In the cell model, tissue factor or extrinsic pathway of coagulation, the initiation phase starts upon injury, with tissue factor (TF) from subendothelial tissue binding FVII and FVIIa. The TF and FVIIa complex proteolytically activates FX and FIX. FXa with cofactor FVa activate plasma FV as prothrombin (FII) is cleaved to thrombin. The next step, in the priming phase, thrombin activates platelets at the site of injury to release FV from their granules. Thrombin activates the released FV to FVa and FVIII to FVIIIa, bound to von Willebrand factor and these factors and FXI bind to activated platelets for thrombin activation to FXIa in a positive feedback loop. In the propagation phase, phospholipids act as cofactor for activating FVa-FXa (prothrombinase) and FVIIIa-FIXa complexes that increase thrombin and FXa formation. FXIa on platelet surface activates FIX to produce more FVIIIa-FIXa. Thrombin cleaves fibrinogen to fibrin polymer and activates FXIII to FXIIIa. The soluble fibrin interacts with FXIIIa to form a thrombus (crosslinked fibrin network). FXIII is a transglutaminase that crosslinks fibrin and other proteins resulting in improved clot strength and resistance to fibrinolysis. In the contact activation pathway or intrinsic pathway the trigger is the autoactivation of factor XII to its active serine protease form (factor XIIa) on surfaces (e.g. negatively charged). The pathway is optimal in the presence of two other contact activation proteins, plasma prekallikrein and high-molecular-weight kininogen. These factors and FXI, are involved mainly in in vitro coagulation. FXIIa activates the prekallikrein—kininogen complex to produce kallikrein that activates more FXII. FXI is activated also by FXIIa thus activating FIX. When FIXa activates FX, the two pathways coverge, since FX is used in the TF-pathway.

Thrombin, a multifunctional serine protease, has a central role in blood coagulation by converting fibrinogen into fibrin clot. Thrombin stimulates fibroblasts to produce procollagen by activation proteolytically of the thrombin receptor PAR-1 (protease-activated receptor 1). Thrombin is a potent mitogen for mesenchymal cells such as fibroblasts, smooth muscle cells and endothelial cells. The autocrine release of PDGF forms AA and AB by thrombin increases cell proliferation. Thrombin increases cell interactions when bound to the ECM, and can be useful in wound healing. Thrombin increases release of IL-1, -6 and -8 from many cells including fibroblasts, endothelial cells and vascular cells. Thrombin is a chemoattractant for fibroblasts. Thrombin, Factor VIla and PAR-1 receptor agonist induce CTGF and IGFBP 10 (cyr61). Thrombin also increases wound contraction through differentiation of fibroblasts into smooth muscle myofibroblasts. Thrombin regulates thrombospondin-1 in endothelial cells. In certain disease states such as atherosclerosis, restenosis and glomerulonephritis, ECM bound thrombin interacts with cells to produce excess cell proliferation and ECM deposition. Thrombin stimulates ECM production in cells (e.g. fibroblasts, smooth muscle cells, epithelial cells) such as procollagen and fibronectin, mainly by PAR-1 activation. Thrombin remodels nascent ECM. Thrombin regulates certain MMPs by activating their latent forms. Many of thrombin's actions can be mediated by proteins that activate the PAR-1 receptor (or use of soluble PAR-1) and subsequent signaling pathway(s). Inclusion of thrombin in vitro and in vivo can enhance cell proliferation and ECM synthesis. Thrombin can limit bleeding at the site of injection.

Coagulation proteases can be mitogens for fibroblasts and other cell types. For example, factors VIIa, Xa, XIIIa (but not IXa) are fibroblast mitogens. Factor Xa is a fibroblast mitogen by binding effector-cell protease receptor-1 on fibroblasts and subsequent autocrine release of PDGF in which PAR-1 is the signaling receptor. Factor VII activating protease (FSAP) activates prourokinase. Inhibition of FSAP assists in coagulation. Thrombin is the main protease in coagulation. Many coagulation proteases can serve as mitogens for cell proliferation of fibroblasts and other cell types in vitro and in the implantate in vivo. Stimulation of PAR receptors by other proteins can also increase ECM production by cells (e.g. fibroblasts).

TF-FVIIa complexes result in cell migration, production of cytokines, angiogenesis, chemotaxis and cell survival. Small peptides and proteins released during coagulation cascades effect cellular immune responses.

Inhibitors or anticoagulants of the coagulation process are: The tissue factor pathway inhibitor (TFPI). TFPI is produced by endothelial cells that bind FXa and forms a complex with TF and FVIIa. It has two active sites for FXa and FVIIa. Thrombomodulin (TM) serves as an inhibitor by binding to thrombin and its transmembrane receptor on cells (e.g. endothelial cells). The TM-thrombin complex also inactivates FVa and FVIIIa through protein C activation and the presence of cofactor protein S. Complement C1-esterase inhibitor is an inactivator of C1 proteins that bind kallikrein, FXIa and FXIIa. The main inhibitor of thrombin is antithrombin (AT), a member of the serpin family of serine protease inhibitors. AT also inhibits FXa, FXIa, FXIIa and the other clotting factors in the intrinsic and common pathways. Antithrombin effectiveness is increased in the presence of GAGs or proteoglycans containing heparan sulfate or heparin. It is the major anticoagulant mechanism of heparin action. Another thrombin inhibitor is the recombinant protein bivalirudin. The extracellular domains of PAR-1 and -4 receptors can antagonize thrombin receptor signaling and platelet activation. Pepducins are cell penetrating palmitolylated peptides based on the third intracellular loop of several G protein receptors. Pepducins inhibit by targeting the intracellular surface of the receptor. PAR-1 antagonists can be used to negate thrombin induced platelet activation and counter restenosis following invasive coronary intervention and neointimal formation following vascular injury. Protein Z-dependent protease inhibitor (ZPI) is a heparin-independent inhibitor of factor Xa. Protein Z complexes with ZPI in the plasma for effectiveness. Protein C, a plasma glycoprotein made in the liver, becomes an anticoagulant when it is activated by thrombin and interacts with TM located on endothelial cell surfaces. This is a second type of inhibiton by TM of the clotting process. Protein C acts as an anticoagulant by degrading membrane-bound FV (Va) and VIII(VIIIa). Protein S is an inhibitor by accelerating protein C's action. Hirudin is an anticoagulant peptide from leech salivary glands. Annexin V and Kunitz protease inhibitors are anticoagulants, as well as other protease inhibitors to the coagulation proteases.

The fibrinolytic system dissolves the fibrin clot and includes: plasminogen, its conversion by plasminogen activators (the serine protease tissue-type plasminogen activator[t-PA] and urokinase-type plasminogen activator[u-PA]) to plasmin, and the plasmin conversion of the fibrin clot into fibrin degradation products and fibrin factor VII activating protease (activator of fibrinolysis). Endothelial cells secrete tissue-type plasminogen activator (t-PA) which cleaves proenzyme plasminogen to plasmin. t-PA is inhibited by plasminogen activator inhibitor type-1 (PAI-1) in the absence of fibrin. In the presence of fibrin, t-PA and plasminogen bind to fibrin. Another pathway uses urokinase type plasminogen activator (u-PA). FXIIa, kallikrein and plasmin (feedback loop) activate u-PA.

Inhibitors to the fibrinolytic system are plasminogen activator inhibitors (PAI-1, PAI-2). The plasmin inhibitor α₂-antiplasmin (AP) is secreted by the liver to form a plasmin-anti-plasmin complex. Fibrinolysis is also inhibited by thrombin via thrombin activated fibrinolysis inhibitor (TAFI) that is enhanced by TM-thrombin formation. TAFI acts by degrading the binding sites for t-PA and plasminogen on fibrin. uPA is a mitogen for cells including fibroblasts and smooth muscle. Plasminogen activators can serve as mitogens for cell proliferation of fibroblasts and other cell types in vitro and in the implantate in vivo. Plasmin, a serine protease, dissolves fibrin clots and is converted from single-chain plasminogen. Plasmin consists of heavy chain A and light chain B. Heavy chain A contains 5 kringle domains and the fragment containing the first 4 kringle domains is an angiogenesis inhibitor called angiostatin. Plasmin can facilitate the escape of cells from contact inhibition in culture and thus may be used to increase cell proliferation (e.g. fibroblasts) in vitro. Plasmin can assist in the removal of clots formed from the injection site of this invention. Plasmin can increase cell proliferation in vivo via its action on implantate clot degradation products.

Kallikrein is an enzyme, present in the plasma and in tissue and glandular secretions, that cleaves kininogens to generate bradykinin. Kallikrein can activate the intrinsic mechanism of blood coagulation. Bradykinin has an effect on pain receptors, smooth muscle and a chemotactic effect on neutrophils. Bradykinin is a nonapeptide inducing vasodilation and increasing capillary permeability. Kallikrein causes the release of renin and synthesis of kinins that influence the immune system, urinary sodium secretion and act as powerful vasodilators. The kallikrein-kinin system consists of vasopressive peptides that control blood pressure through maintenance of regional blood flow and the excretion of water and electrolytes. Kallikrein inhibitors exist in the serum.

Prekallikrein generates kallikrein which then can activate the intrinsic mechanism of blood coagulation. Kininases are plasma enzymes that degrade kinins to inactive peptides. Kininase I degrades kinins whereas kininase II cleaves kinins and liberates angiotensin II from angiotensin I. Kininogens, which are plasma α-2 globulins, are precursors of kinins. Kinins exert potent vasomotor effects, causing vasodilation of most vessels in the body and promoting vascular permeability. Vasodilation can be useful to deliver blood nutrients and growth factors to the implantate site.

Hemostasis promotes blood fluidity under normal circumstances. Hemostasis consists of plasma proteins (the coagulation and fibrinolytic factors), the vessel wall itself and platelets.

Inflammation.

Coagulation and inflammation are integrated. Inflammation is a protective response for vascular tissue mediated by humoral and cellular interaction of several pathways that result in production of cell adhesion proteins, thrombin generation, complement activation and cytokine release and production. The plasmin/plasminogen activator system is important for the protease network associated with inflammation. CRP and fibrinogen are some of the markers of inflammation. Inflammation induces thrombin generation via cytokine-activated mononuclear cells that express tissue factor. Thrombin receptor activation on endothelial cells and leukocytes produces and releases inflammatory and chemotactic cytokines such as IL-1, IL-6, IL-8, MCP-1 and cell adhesion molecules such as P and E-selectins and ICAM-1. Proteolytically active tissue factor-FVIIa complex leads to PDGF-BB stimulated chemotaxis and monocyte production of IL-8 and TNF-α. Thrombin is a chemoattractant for monocytes and a platelet activator which then releases granule contents (PDGF), express P-selectin, CD40 ligand and gpIIb/IIIa on the cell surface. Fibrinogen binds activated platelets to leukocytes that lead to tissue factor production and cytokine release. Selectins and integrins assist endothelial (P and E selectins) transmigration in which leukocytes (L-selectins) transmigrate across the endothelium into the site of inflammation. The transmigration is dependent on ICAM-1 and PECAM (platelet/endothelial cell adhesion molecule-1). Proinflammatory proteins that include chemokines and growth factors direct leukocytes to the inflammation site. Examples of proinflammatory proteins are immune complexes, oxidized LDL, TNFα, IL-8, MCP-1 (monocyte chemoattractant protein-1), PDGF (BB), C-reactive protein (CRP), and formyl-Met-Leu-Phe (fMLP). Chemokine SDF-1 (stromal derived factor) and cytokine TGFβ, made by stromal cells such as fibroblasts, endothelial and dendritic cells, and CXCR4 chemokine can promote inflammation. The transcription factor NF-κB, controlled by growth factors, regulates many proinflammatory genes and proteins. Inhibitors of NF-κB or AP-1 activities, such as PPARα, PPARγ, ERα and LXR (liver X receptor), block inflammation. Proteins that counteract NF-κB or AP-1 can promote inflammation. Fibronectin, collagen (e.g. type III) and other ECM proteins provide matrices for cell adhesion and migration during the early phases of tissue repair and angiogenesis that help regulate inflammation.

Examples of inflammatory molecules are the cytokines IL-α, IL-β, IL-6, TNFα, F2-isoprostane, complement proteins, interferons, colony-stimulating factors, many chemokines, certain growth factors, amongst others.

Advantages of inflammation for the invention is that there is increased blood flow, chemoattraction of desired cell types, the effect is transient and limits the area of protein's action to the implantante site, tissue remodeling and repair, enhancement of immune cell entry to the site as well as other beneficial proteins, growth factors, hormones, ECM proteins, etc. are delivered. Chemokines and cytokines attract other immune cells that promote ECM production if desired.

Transient inflammation promotes a host of beneficial events including enhanced blood flow, nutrient and hormonal delivery and thus is preferred to establish seeding of cells, as well as the metabolism, survival and proliferation of implanted cells. It is also beneficial for the cells already present in situ in the tissue.

Bloodflow

Bloodflow increase in the area of the implantation can be beneficial to the defect treatment. Increased delivery of nutrients, growth factors, hormones, survival factors and many other useful functions can be obtained as described in the invention. Thus the addition of cells or macromolecules such as proteins, hormones, growth factors, cytokines, chemokines, ECM proteins, serum proteins, immunogenic proteins and other proteins and molecules that increase the bloodflow is desired. This includes proteins that locally increase vasodilation, angiogenesis, inflammation, coagulation, complement reactions and immune responses. Separate sections throughout the document describe these bloodflow processes. Also, those cell types, proteins and molecules that affect these processes are detailed throughout the document. Furthermore, other cell types, proteins and molecules that increase the bloodflow but not described are included in which an ordinary artisan in the field would recognize. Additionally, other treatments known in the art, such as physical or mechanical therapy such as ultrasound or agents that create heat or vasodilation are amongst many other available therapies to increase bloodflow in the implantate area.

Blood vessel diameter increases as blood vessels relax during vasodilation, thereby increasing tissue perfusion. Inpaired vasodilation includes a decrease in nitric oxide production and an increased vasoconstriction (e.g. endothelin-1). These events can be predominant in the elderly. Angiopoietin enhances vascular enlargement and blood flow. Improved bloodflow increases initial innate immune responses as well as the adaptive immune response. Increased tissue repair is enhanced. Proteins that increase nitric oxide production or primed endothelial cells can be used to vasodilate.

Angiogenesis requires a protein matrix for endothelial cells to attach onto, migrate and invade. Thus ECM proteins support endothelial networks and their behavior. Cell attachment is mainly mediated by the integrins. MMPs secreted by endothelial cells and supporting cells during migration and invasion, regulate the proteolytically degradation of the ECM. The supporting cells include fibroblasts and mural cells, which are adjacent cells (e.g. pericytes, smooth muscle cells) to endothelial cells in the microvasculature.

Angiogenesis is promoted by growth factors such as VEGF and its isoforms (eg 121 and 165), Angiogenin 1, matrix adhesion factors L1 and ephrin B2. The matricellular proteins tenascin, osteonectin, TSP-1 and -2 mainly regulate endothelial behavior. TSPs and osteonectin are anti-angiogenic. The matricellular proteins regulate the balance between pro-angiogenesis (e.g. VEGF) and anti-angiogenesis (e.g. angiostatin, PEDF). Tie receptors are expressed on endothelial and hematopoietic progenitor cells playing roles in angiogenesis, vasculogenesis and hematopoiesis. Tie-1 is involved in endothelial cell differentiation and its maintenance of endothelium integrity. Tie-2 has angiopoietin-1 and -2 as ligands and is involved in angiogenesis.

After injury, angiogenesis occurs during the formation of granulation tissue in the wound bed. High vascularization in tissue promotes the migration of needed cell types for tissue integrity and remodeling, such as that occurs with keratinocytes and fibroblasts wound healing. Growth factor receptor tyrosine kinases have central roles in angiogenesis and vasculogenesis.

The aged have less capillary density in tissues and it takes longer to make new vessels and to repair tissues. This is accompanied by reduced concentrations of angiogenic growth factors and ECM (e.g. collagen deposition), and more TSP-2 activity.

Angiogenic growth factors can be used and include VEGF, PDGF, FGF2, TGF-β, and steroid hormones (which enhance synthesis and function of angiogenic growth factors such as VEGF). TNFα induces PDGF signalling and enhancement of angiogenesis in endothelial cells. Delays or absence of influx or function of inflammatory cells that increase the delivery of cytokines inhibit angiogenesis. Thus proteins that chemoattract or activate cytokine producing cells or inclusion of cytokine producing cells can be used to assist angiogenesis.

Chemoattractants

The migration of specific cell types into the area of the implantation can be beneficial to the defect treatment. The migration of specific cell types can be productive in eliciting the production of ECM proteins and survival factors, removing clotted blood, amongst other desired functions described in this invention. Thus the addition of cells or macromolecules such as proteins, hormone, cytokine, chemokines, immunogenic proteins, serum protein, ECM proteins and other proteins and molecules that attract specific cell types is desired. This includes proteins secreted by added cells or proteins that are added to the implantate that signal other cell types to migrate to the implantate area. For example, the addition of a growth factor to connective tissue in the skin can attract fibroblasts in situ to migrate to the implantate area. Some of the proteins that locally increase vasodilation, angiogenesis, inflammation, coagulation, complement reactions and immune responses can serve as chemoattractants or as a source of cell migration. For example, after injury increased angiogenesis can promote keratinocye and fibroblast migration to the wound bed. Separate sections throughout the document describe those proteins, molecules and cell types that affect these processes are detailed. Furthermore, other proteins, molecules, and cell types that increase the bloodflow but not described here are included in which an ordinary artisan in the field would recognize.

Connective tissue growth factor and thrombin are examples of a chemoattractant for fibroblasts.

Transport Proteins

Proteins that are often required as carriers for minerals, fatty acids, growth factors, cytokines, hormones and many other molecules are referred to herein as transport proteins. Transport proteins include, for example, albumin as a carrier for lipids, minerals and globulins and transferrin that binds iron, making it less toxic but bioavailable. Serum contains a variety of transport proteins. Many of these transport proteins are multi-functional and also serve other physiologic and regulatory pathway roles.

Albumin is the principal protein of serum, regulates osmotic pressure, binds anions, and also helps to keep blood from leaking out of blood vessels. Albumin is important for tissue growth and healing. Albumin, like many other serum proteins, is made in the liver. It is used as an immunogen in studies. It functions as a transport protein for fatty acids, bilirubin, hormone, growth factor, vitamins and other large anions, selected hormones (e.g. cortisol, thyroxine), and many drugs. Albumin bound bilirubin is cytoprotective against oxidative damage. The serum globulins and albumin carry hormones and other substances. Pre-albumin (transthyretin) is a serum carrier protein.

Certain globulin proteins are also made by the liver while others are formed by the immune system. Some globulins are transport proteins that transport metals, such as iron or copper in the blood, and help fight infection. Some of the globulin proteins are acute reaction proteins (ARP), α-1 antitrypsin, haptoglobin, ceruloplasmin, CRP, C3, α-1 acid glycoprotein, CRP-beta-gamma, haptoglobin-alpha₂, AFP, steroid binding proteins (such as cortisol binding protein deliver steroids for cell growth and proliferation), TBG, immunoglobulins IgG, IgM, IgD, IgG, IgA, alpha-2 macroglobulin, beta lipoprotein, and the components of complement.

The globulins are separated into alpha, beta and gamma types. Alpha-1 globulins include α-1 antitrypsin, thryoxine binding globulin (T3, T4, retinol, RT3U). Alpha-2 globulins include haptoglobin, ceruloplasmin, HDL and α2-macroglobulin. Beta globulins include transferrin, plasminogen and beta-lipoproteins (LDL). Gamma globulins contain the immunoglobulins M, G, and A. Globulins is an obsolete term for immunoglobulins. γ globulin has the slowest mobility, β globulin is next slowest, followed by α-2 and α-1 globulin toward the anode during electrophoresis at neutral pH and thus most cationic of the serum globulins. All migrate behind albumin. Originally globulins were characterized by their solubility, e.g. (β Euglobulin), a water insoluble globulin that is salt soluble and is part of the electrophoretic globulins. immunoglobulins classes are IgM, IgG, IgA, IgD and IgE.

Hormone binding and growth factor binding proteins are needed to transport hormones and growth factors in the blood and extracellular fluid to their target cell receptors. Such transport results in cell survival, increased ECM synthesis, apoptosis or anti-apoptosis, cell proliferation, promotion of cell adhesion, etc. Many of these binding proteins are also multi-functional physiological and regulatory pathway proteins. A few hormones circulate dissolved in the blood, but most are carried in the blood bound to soluble plasma proteins. Hormone and growth factor binding proteins (HBPs) are in extracellular fluids such as blood. Examples of such transport proteins are: Androgen binding protein (ABP) transports testosterone. Gonadal steroid binding globulin (GBG) transports testosterone and dihydrotesterone. Human growth hormone binding protein (GHBP, 237 aa protein), also known as serum binding protein, transports human growth hormone. Insulin-like growth factors are transported by insulin-like growth factor-binding proteins (IGFBPs 1-10). Transthryetin (T4 binding protein, thyroid-binding pre-albumin) is a binding protein for thyroid (thyroxine) hormones, vitamin A, retinols, sequesters toxic β-amyloid and is involved in homeostasis. Thyroxine-binding globulin and albumin also transports these substances. Thyroxine-binding globulin is the primary carrier for thyroxine and triiodothryonine in serum. Retinoid binding proteins (RBPs) bind retinoids such as vitamin A. LBP (lipopolysaccharide-binding protein), made by hepatocytes as a 58 kda glycoprotein, is a member of the lipid-binding proteins family that includes BP (bactericidal/permeability increasing protein). LBP increases in the serum during the acute phase response, catalytically transfers LPS to HDL increasing the LPS detoxification, functions in phospholipid transport along with soluble CD14, promotes the LPS induced immune response and induces IL-8 secretion. Lipocalins are extracellular carriers of lipophilic molecules and interact with cell surface receptors and proteases. Cortisol binding protein deliver steroids for cell growth and proliferation, Corticotrophin-releasing hormone-binding protein (CRHBP), albumin, plasma binding proteins for steroid (steroid binding globulins) and corticosteroid binding protein transport these steroids and assist in their action, sex hormone-binding globulin (SHBG), vitamin D-binding proteins (VDBPs), TGF-β binding proteins, BMP binding proteins, PLTP (phospholipids ester transfer protein and CETP (cholesterol ester transfer protein)), mannose binding protein, complement binding proteins, growth factor binding proteins for FGF, HB-FGF, latent TGFβ binding protein (LTBP), NGF and heparin binding protein are but a few of the many other globulins and proteins present in serum that are specific for the particular hormones, growth factors, cytokines, nutrients, trace elements and others listed in the invention. Some of the carrier proteins are very specific for their substrate while others, such as albumin, show broad specificities and and lower binding affinities for substrates. Albumin and other serum proteins can deliver active forms of hormones and other factors. For example, NGF (nerve growth factor) binds to carrier proteins in the serum.

Hormone binding proteins (HBPs) can serve roles beyond carrier hormone proteins and can exist as multifunctional regulatory proteins acting not only at the receptor level but also intracellular level, including nuclear. They influence cell proliferation, differentiation, survival, apoptosis, migration, spreading, cell size, etc. For example VDBP improves host defense and SHBG is an intermediate in sex steroid signaling. Some factors use more than one type of binding protein for transport and action. For example, DHEA is a precursor for the estrogenic and androgenic steroids. Circulating DHEA is bound by corticoid steroid binding globulin (CBG), albumin and SHBG. The bioavailable form of testosterone includes the free steroid and the albumin-bound form. The IGFBPs have separate growth factor actions independent of the ligand IGF. HBP (heparin binding protein), is similar to serine proteases in stucuture but lacks protease activity and is important as a paracrine in causing intercellular gaps on endothelial cells and allowing leukocyte intravasation. Lipocalins are a family of extracellular ligand-binding proteins having tight specificity for small hydrophobic molecules. They function in nutrient transport and protease interactions. Examples are plasma retinol-binding protein precursor (PRBP), bilin-bnding protein precursor (BBP), β-lactoglobulin precursor and proteinase inhibitor 12 with serine-type endopetidase inhibitor activity (e.g. pancreatic trypsin inhibitor, tissue factor pathway inhibitor).

Nutrients are carried by ECM, serum and fluid binding proteins to or into cells. This results in enhanced energy metabolism, cell survival, growth and proliferation, etc. Cholesterol and fatty acids are carried by albumin and by specific lipoproteins. Transferrin and ferritin carry iron. Apotransferrin is the non-heme form and holotransferrin is the heme form. Ceruplasmin transports copper. Glucose transport protein deposits sugar into cells. Lipoproteins (HDL, LDL, VLDL, apoA1, apolipoprotein A-II, apolipoprotein B) transport cholesterol and fatty acids. Fatty acid binding protein (FABP) transports fatty acids. Transcobalamin is the main transport protein for vitamin B 12. Many other nutrient transport proteins exist. Transport proteins (e.g., albumin), can extend the half life of drugs, proteins, and other molecules.

Serum proteins and transport proteins can also serve in cell implantation and in cell culture as an immediate nutrient source for cell survival, proliferation, differentiation, amongst other functions.

Growth Factors and Cytokines

Many types of growth factors exist in the ECM and in serum. Growth factors are produced by specific cell types and tissues. Growth factors are often mitogenic for the cells they target. Growth factors also are involved in differentiation, ECM synthesis or degradation, protease and protease inhibitor production, chemoattraction, metabolism, amongst other functions. Multiple growth factors can in tandem or singly can act on a biological function. A number of growth factors exist such as TGF-α, TGF-β, PDGF, FGF, EGF and IGF.

Examples are listed below, in the section on serum proteins and throughout the document. Growth factors may be added with cells to a tissue at a defect site. Extracellular matrix molecules that bind to growth factors, e.g., heparan sulfate proteoglycans, may advantageously be added to serve as a reservoir for the factors.

Growth factors and cytokines are present in the ECM and serum. Some of the factors are Endothelial growth factors, EGF, HGF, neuregulins, PDGF, IGF-1, IGF-II, FGFs, interleukins, interferons, TGFs, NGFs, neuroleukin, GRP, CSF-1, G-CSF, TNFs, EGFs, VEGFs, and many others.

To treat defects, ECM, growth factors, cytokines, chemokines, hormones, serum proteins and other proteins can vary in concentration from >0% to 100% if used alone and >0% to <100% if part of the cell composition.

Cell Growth and Proliferation—Growth Factors, Cytoidnes, Chemokines, Hormones

Growth factors, cytokines, chemokines and hormones are proteins or endocrines best known for enhancing cell proliferation and growth but also have roles in differentiation, apoptosis, cell survival, cell adhesion, cell spreading, cell migration, proteolysis, angiogenesis, tissue morphogenesis, homeostasis and regeneration, wound healing, ECM production, cancer processes, amongst others. These factors function is cell type specific ways. Cells numbers are not only determined by cell proliferation, apoptosis, proteolysis, survival and other processes, but cell numbers are also controlled by inhibitory factors that inhibit proliferation, apoptosis, proteolysis, survival, amongst others. Cell culture or implantation of cells with these cell type specific factors in addition to fimctions listed above can promote seeding and metabolism, thereby ensuring cell survival and optimizing treatment.

Hormones in general have low redundancy with few biological actions (low pleiotropy) while cytokines and growth factors often display high redundancy covered by different proteins with multiple actions (high pleiotropy). Growth factors, cytokines, chemokines and hormones, though made differently (endocrine, exocrine, paracrine, autocrine) or in different protein type or size class, are often used in the same context with respect to their actions on cells. Thus EPO, for example, is an endocrine (thus hormone) but is also classified as a growth factor or cytokine. Some cytokines are also listed as chemokines or growth factors. Thus for the purpose of the invention the classifications can be interchanged.

Growth factors, cytokines and polypeptide hormones share many similarities including structure similarity and mechanism of action. All repesent proteins released from one cell that influence other cells, in minute quantities, via binding to high affinitiy specific receptors that are generally on the cell surface. The proteins bind to specific cell surface receptors that in turn initiate signallng pathways and some of the receptors and ligands share distinct structural homologies. Also many share intracellular signaling components in which the activated cell surface receptor transmits its message to the cell nucleus. Some of the ligand-receptor complexes also translocate directly to the nucleaus and acts on transcription factors. Many activities of growth factors, hormones and cytokines are determined by interactions with ECM, transport proteins and serum proteins. The activities are expressed by binding to cell transmembrane receptors that are part of signalling pathways. For example, transmembrane receptors can be G proteins linked to membrane bound phospholipase C (PLC). Receptor activation cleaves PIP2 (phosphatidylinositol 4,5 bisphosphate) to form diacyglycerol (DAG) and D-myo-inositol-1,4,5-triphopsphate (IP3). IP3 binds to the endoplasmic reticulum to release calcium stores which in turn activates calmodulin. DAG, calcium/calmodulin and activated PKC (protein kinase C) trigger a protein kinase cascade that regulates many aspects of cell function and gene transcription including the mobilization of calmodulin kinase II that directly activates transcription factors. Most polypeptide hormones and some growth factor and cytokines bind receptors linked to other G-proteins that are associated with the enzyme adenylate cyclase (AC). The enzyme generates cAMP which activates protein kinase A (PKA) and triggers a protein kinase cascade. Other growth factors and cytokines activate protein tyrosine kinases (PTKs) (e.g. JAK kinase) which triggers a protein kinase cascade ultimately controlling gene expression specifying specific cell functions.

Some of the hormones and growth factor supplements used in cell culture include aldosterone, dexamethasone, hydrocortisone, testosterone, dihydrotestosterone, β estradiol, thyroxine, triiodo-L-thyronine, thyrotropin-releasing hormone, luteinizing hormone releasing hormone, progesterone, insulin, glucagons, prostaglandins D2, E1, E2, F2, linoleic acid, somatostatin, growth hormone, thrombin, and transferrin.

Many of the growth factors are members of families or superfamilies such as TNF, EGF, FGF, IGF, VEGF, PDGF, Hedgehog, TGF-β superfamily, proteoglycans and regulators, Wnt-related proteins, or are other growth factors such as SCF, Flt-3 and M-CSF. Individual members and some functions are listed below.

The EGF (epidermal growth factor) family members use the ErbB1-4 receptor tyrosine kinases to regulate cell proliferation, differentitation, motility, apoptosis, development, wound healing, amongst other functions. The EGF ligand members can be mitogens. All members have at least one EGF structural unit in their extracellular domain wherein the proteins are synthesized as transmembrane precursors and play a role in stimulation of adjacent cells. The precursors are often cleaved to soluble, mature proteins.

Some of the EGF members follow. EGF, 6 kDa, is made by platelets and keratinocytes, among other cell types, is present in urine, serum and submaxillary gland. EGF is a membrane bound precursor containing EGF structural units in the extracellular domain. The mature sequence is soluble. EGF targets all three germ layers including the cell types of fibroblasts, epithelial cells, glial cells and endothelial cells. It promotes cell proliferation and differentiation of mesenchymal cells such as fibroblasts, chondrocytes, prostate, vascular, epithelial, endothelial and epidermal (keratinocytes) cells. EGF induces epithelial development, angiogenesis, inhibits gastric acid secretion and promotes wound healing. EGF stimulates ECM metalloproteinases (e.g. collagenase, stromelysin). EGF is synergistic with IGF-1 and TGF-β. Heparin binding EGF (HB-EGF) is a fibroblast, keratinocyte and smooth muscle cell mitogen, induces autocrine release of FGF-2 by fibroblasts and is made by many cell types including monocytes, macrophages, vascular endothelial cells and aortic smooth muscle cells. HB-EGF is a transmembrane protein with EGF motifs in the extracellular domain that is cleaved to a soluble protein. It binds to the EGF receptor as does EGF, TGFα and AR (amphiregulin).

The EGF family of mitogens also include TGFα, AR and other regulins, SDGF (rat schwanoma-derived growth factor), VGF (vaccinia growth factor), ligands for the HER2/erbB2/neu receptor, epigen and betacellulin. TGF-α is involved in cell-cell adhesion and its expression is widespread. NRG is a member of the heregulin (neuregulin) family comprised of multiple secreted or membrane-bound isoforms made from a single heregulin gene through alternative splicing. All members share an EGF-like domain which activate the erbB family of tyrosine kinase receptors. NRG1 is expressed in the nervous system and has no transmembrane domain or cytoplasmic tail. Heregulins are mitogenic for epithelial, tumor and Schwann cells. Neuregulins are glycoproteins NRG-1 to -4 that through alternative splicing encode more than 14 soluble or transmembrane proteins. The extracellular domain, contain EGF-like domains for binding to ErbB3 or ErbB4 receptor tyrosine kinases. The transmembrane isoforms can be proteolytically cleaved, releasing soluble growth factors. NRG isoforms are of 3 types: Type 1 (heregulin) and type II (glial growth factor) have an Ig-like domain N-terminal to the EGF domain and type III (sensory and motor neuron-derived factor) instead has a cysteine rich domain. NRGs promote differentiation and development of Schwann cells from neural crest stem cells and help establish the oligodendroglial lineage. NRG-1 stimulates proliferation of cells. Neuregulins (NRGs) include neuregulin-3, NRG1 isoforms GGF2 and SMDF, NRG1-α and β1. Other regulins that are members of the EGF family include epiregulin. Epigen acts on epithelium. Amphiregulin is made as a transmembrane precursor and the soluble form is released by protease cleavage. It is expressed in epithelial cells of colon, reast, ovary, kidney, stomach carcinoma cells and others. It stimulates the proliferation of keratinocytes, epithelial cells and fibroblasts. Betacellulin (BTC), a member of the EGF family, is made as a transmembrane precursor and has one or more EGF motifs in the extracellular domain. Soluble forms are generated by proteolytic cleavage. BTC is a heparin binding protein. Betacellulin is expressed in most tissues. BTC binds to the EGF receptor and is mitogenic for fibroblasts, epithelial and vascular smooth muscle cells. EGF can synergize with IGF-1 and TGFβ.

Binding to the EGF receptor (e.g. calmodulin) can increase fibroblast cell proliferation through the down regulation of the Ras/Raf/MEK/ERK pathway that is a common feature of cell proliferation in many systems. Thus factors that alter this pathway can control cell proliferation. Sustained activation of this pathway can lead to senescence or apoptosis of fibroblasts and other cell types.

The 23 some FGF (fibroblast growth factor) family members use the FGF R1-R5 receptors and act on mesodermal, endothelial cells and neuroectodermal origin cells that function in cell growth, migration, proliferation, survival, shape, motility, metabolic regulation, tissue repair, wound healing, apoptosis, angiogenesis, embryonic development, pattern formation and neurotrophic effects such as myelation, oligodendrocyte development and nerve regeneration. The receptors are a family of type 1 transmembrane tyrosine kinases. Unlike other growth factors, FGFs act in concert with heparin or heparin sulfate proteoglycan (HSPG) to activate FGF receptors. The members are regulated by FGF binding proteins such as FGFBP (FGF binding protein), a low affinity heparin binding protein that binds FGF acidic and basic non-covalently in a reversible manner. FGFBPs share ten conserved cysteine residues that form five intracellular disulfide bridges forming the know structure needed for receptor binding and biological action. FGFs are expressed in all mesodermal and many cells of neuroectodermal, ectodermal and endodermal and embryonic origin including fibroblasts, endothelial cells, macrophages, astrocytes, oligodendrocytes, neruoblasts, keratinocytes, osteoblasts, intestinal columnar epithelial cells, pituitary basophils and acidopils, smooth muscle cells, and melanocytes. The FGFs are mitogenic peptides. FGF acidic is FGF1 (˜16 kDa) and FGF basic is FGF2 (˜18 kDa). FGF 1 and 2 act on a range of mesoderm and ectoderm derived cells including fibroblasts, smooth muscle cell, vascular endothelial cells and glial cells. FGF2 is a fibroblast, endothelial, epithelial (retinal pigmented epithelium), stromal cell (e.g. bone marrow) mitogen. FGF 2 inhibits apoptosis of many different cell types such as epithelial, endothelial, fibroblasts, smooth muscle, retinal pigmented epithelial and neuronal cells. FGF2 promotes hematopoietic cell development, and adherent stromal cell layer formation. FGF2 regulates the transcription and activity of multiple other genes and thus is involved in cell proliferation, differentiation and survival of almost all organ systems. FGF2 is chemotactic for endothelial cells and induces neuron differentiation, survival and regeneration. FGF2 plays a role in angiogenesis, wound healing, tissue repair, embryonic development, differentiation and neuronal function. FGF1 is also known as β-ECGF or β-endothelial cell growth factor. Astroglial growth factors (AGF-1, -2), produced in brain tissue, are members of FGF-1 and mitogenic for astroglia. FGF3 and FGF4 are involved in embryonic development, are mitogenic for fibroblasts and endothelial cells, are morphogens and promote angiogenesis. FGF5 and FGF6 are morphogens and mitogenic for fibroblasts and endothelial cells. FGF5 is a survival factor for spinal motor neurons. FGF5 is associated with neurons. It is a neurotrophic factor of skeletal muscle, is involved in myoblast differentiation during cell migration, and plays a role in angiogenesis. The members of the FGF family do not need to stimulate fibroblast growth. At least seven FGF polypeptides are potent regulators of cell proliferation, differentiation and function. FGF 7 (keratinocyte growth factor) is made by stromal cells (e.g. fibroblasts), but not epithelial, and stimulates the proliferation, differentiation and cytoprotection of epithelial cells, including skin keratinocytes and epithelial (prostate, alveolar, intestinal) cells. FGF 9 is a glial activating factor and a steroid regulated mitogen and survival factor for nerve (glial cells, oligodendrocyte astrocyte progenitor cells) cells and mesenchymal cells (fibroblasts), and acts in an autocrine and paracrine fashion. FGF 10 is involved in wound healing and is a mitogen for epithelial (urothelial cells) and epidermal cells, but not fibroblasts. FGF 17 is involved in arteries and bone development and proliferation of fibroblasts. FGF 23 prevents osteomalacia. FGF8 b and c are isoforms of FGF 8, which can be induced by androgens and can proliferate carcinoma cells. FGFs also promote osteoprogenitor cell proliferation, osteogenesis, eye development and retinal cell rescue.

IGFs (insulin-like growth factors). IGF-1 (somatomedin C or A, ˜7 kDa) and IGF-2 (multiplication stimulating activity or MSA, ˜7 kDa) belong to the family that are structurally homologous to proinsulin. These factors are expressed in many tissues (e.g. liver, lung) and cell types (e.g. fibroblasts) in vivo and in vitro. IGFs have autocrine, paracrine and endocrine functions. The IGF-1 receptor is expressed in all cell types and tissues. IGFs target the cells of mesenchymal origin and binds to most cell types. Cell proliferation, differentiation, metabolism, wound healing and apoptosis are some of the functions of IGF growth factors. IGF (e.g. IGF-1) mediates the growth-promoting activities of growth hormone and is mitogenic for fibroblasts, osteoblasts, smooth muscle cells, lymphocytes, chondrocytes, neuroglial cells, erythroid progenitors, amongst others and made in the liver. IGF-2 has many similar activities to IGF-1 and stimulate fetal development. Mystique is an IGFI regulated PDZ-LIM doman protein that promotes cell attachment and migration via cell adhesion to collagen and fibronectin.

IGF binding proteins (IGFBP) modulate the activities of IGF factors and also have intrinsic bioactivity. They are present in many tissues, body fluids and serum. Glycosylation, phosphorylation and proteolysis of IGFBPs modifies their affinity to IGF. The IGFBP family contains IGFBP1-6, IFBP-7, NOV/CCN3, Endocan, CTGF/CCN2 and ALS. ALS (Acid Labile Subunit) is made by the liver, binds IGFBP-3 or -5 and complexes with IGF1 or 2 in the serum, thereby increasing the half-life of the IGF/IGFBP complexes in the circulation. CTGF/CCN2 is connective tissue growth factor. Endocan, is a dermatan sulfate proteoglycan expressed by endothelial cells in the kidney vasculature and alveolar walls of the lung. IL-1β, LPS, TNF-α increase endocan and IFN-γ decreases endocan expression. Endocan inhibits immune cell binding to ICAM-1. IGFBPs can inhibit or enhance IGF actions. Proteolysis of IGFBP decreases IGF affinity resulting in release of IGF for binding to cell receptors. The kallikrein, cathepsin and MMP proteases cleave IFGBPs 2-6 with different specificity. Some IFGBPs have there own bioactivity, such as IFGBP10 (cyr61, CNN1), an inducer of angiogenesis and fibroblast proliferation or IGFBP5 that alter mineral and ECM deposition in bone. IFGBPs have proteases associated with them, such as IFGBP-3 protease. IGFBP-3 is the major IGF binding protein in serum, is present in the alpha granules of platelets and in non-parenchymal liver cells. IGFBP-3 binds IGF-1 and -2. It inhibits FSH (follicle stimulating hormone). PDGF, EGF, vasopressin and bombesin stimulate fibroblast synthesis of IGFBP-3 and in skin fibroblasts it is stimulated by TGFβ. IGFBP-1 contains a RGD integrin receptor recognition sequence and is expressed in most tissues, abundantly in liver, kidneys, serum and fluid. Corticosteroids and insulin regulate the levels of IGFBP-1. IGFBP-2 has highest expression in the central nervous system and binds preferentially to IGF-2. IGFBP-5 is produced by fibroblasts, myoblasts, osteoblasts, amongst others, is the predominant IGFBP in bone extracts and has a strong affinity for hydroxyapatite allowing for its binding to bone cells. Binding to ECM protects it from proteolysis and enhances IGF activity, while the soluble IGFBP-5 is cleaved to an inactive fragment. IGFBP-6 is found predominantly in serum and CSF and is present in fibroblasts, ovarian cells and prostatic cells.

PDGF (platelet derived growth factor, 31 kDa dimer) and VEGF (vascular endothelial growth factor) family members are mitogenic for many cell types, are angiogenic and have roles in wound healing, tumor formation, and embryonic development. The members have an 80-90 amino acid sequence with conserved cysteine residues. PDGF acts mainly on connective tissue. PDGF members are PDGF-AA, AB, BB, receptors Rα and Rβ, PIGF and PIGF-2. PDGF is made by platelets, macrophages, monocytes, megakaryocytes, fibroblasts, smooth muscle cells, keratinocytes, transformed and endothelial cells. It is mitogenic (stimulates κB binding activity) and chemotactic for cells of mesenchymal and neuroectodermal origin such as fibroblasts, chondrocytes, smooth muscle and glial cells, certain endothelial and epithelial cells, neutrophils and mononuclear cells. PDGF is a major growth factor in fibroblasts and glia. It is important in the modification of ECM (e.g stimulation of collagen synthesis, collagenase and thrombospondin activity and secretion), neuron survival, regeneration and differentiation. PDGF stimulates neutrophil phagocytosis and granule release by neutrophils and monocytes and steroid synthesis by Leydig cells. PDGFRα and PDGFRβ are members of class III subfamily of receptor tyrosine kinases (RTK). Soluble PDGFRα is present on endothelial, mesothelial and oligodendrocyte progenitor cells and in plasma. Both receptors are antagonistic to PDGF by binding to the growth factor. PDGFRα binds all three PDGF isoforms while PDGFRβ binds PDGF-BB and AB, but not AA. Recombinant PDGF (becaplernin) can be used as a therapeutic. PDGF can synergize with EGF and IGF-1 for certain biological actions.

Other family members are VEGF-A, B, C, D and the neuropilins (e.g. 1 and 2). VEGF receptors are tyrosine kinases and are present on endothelial cells. VEGF has a central role in angiogenesis, acting as a mitogen on endothelial cells, their progenitors and monocytes. Akt, Src, focal adhesion kinase and calcineurin pathways mediate the multiple VEGF functions of cell survival, proliferation, migration, vascular permeability, tubulogenesis and gene expression. VEGF-D is expressed in lung, muscle, heart, and small intestine and is a ligand for VEGF receptors 2 and 3. It is expressed in lymphatic and endothelial cells. VEGF-D is involved in regulation of the growth and differentiation of lymphatic endothelium. EG-VEGF (endocrine gland-derived vascular endothelial growth factor) is an endothelial cell mitogen and chemotactic factor. EG-VEGF is a member of the prokineticin family of secreted proteins containing the knot structure. VEGF growth factors have isoforms such as VEGF 121 and 165. PD-ECGF (human platelet-derived endothelial cell growth factor) is a mitogen for some cell types and for others a growth inhibitor. It is produced by fibroblasts, smooth muscle cells, platelets, amongst other cell types and present in liver, lung, spleen, lymph nodes, lymphocytes and astrocytes. It has chemotactic and angiogenic activity. It is an endothelial cell mitogen and involved in neuronal viability and glial cytostasis. P/GF (placenta growth factor) is a member of the VEGF family. It is expressed in umbilical vein endothelial cells, placenta, carcinoma cells and is associated with angiogenesis. It is mitogenic for monocytes, endothelial cell and progenitors. It binds to Flt-1. VEGF R1, R2, R3 is one of five tyrosine kinase receptors (RTKs) that is restricted to endothelial cells. The others are Flt-1, Flk-1, Flt-4, Tie-1 and -2. All RTKs have central roles in angiogenesis and vasculogenesis. VEGF R1, R2, R3 promote endothelial cell proliferation. Soluble VEGF R can be used to antagonize VEGF action.

The TGF-β superfamily is involved in cell proliferation, migration, differentiation, morphogenesis and many other functions. The superfamily consists of over 30 proteins arranged into the subfamilies of bone morphogenetic proteins (BMPs), growth differentiation factors (GDFs), activins, inhibins, GDNF (glial cell-derived neurotrophic factor) ligands, TGF-β members, and other ligands such as Lefty, Nodal and MIS (Mullerian Inhibiting Substance)/AMH. The members are secreted C-terminal segments of the protein and has 6 to 7 cysteine residues conserved to form the knot structure that gives receptor specificity and biological function. TGFs are members of the EGF family. TGFs are produced in most adult and many embryonic tissues and many cell types in culture. TGFs are synthesized as transmembrane precursors and contain one or several EGF motifs in the extracellular domain. Soluble forms of these cytokines are released by proteolytic cleavage of the transmembrane protein. TGFβ subfamily members include TGFβ (˜25 kDa dimer), β1, 1.2, 2, 3, 5, latent TGF-β1, β bp (binding protein) 1, and LAP (TGF-β1). TGF-β generally is stimulatory (e.g. mitogenic) for mesenchymal cells and inhibitory of epithelial or neuroectodermal cells. TGFβ is an inhibitor or stimulator of apoptosis depending on the many cell types. TGFα is made by platelets, macrophages, keratinocytes, transformed cells, tumors, embryonic tissue, pituitary, brain, and activates neutrophils, stimulates angiogenesis, osteogenesis. TGFα is mitogenic for many cells including fibroblasts, keratinocytes and osteoprogenitor cells. TGFα mediates cell-cell adhesion, juxtracrine stimulation of adjacent cells, induces epithelial development, promotes angiogenesis, and stimulates keratinocyte migration. TGFα mediates its effects by binding to EGF receptors and thus display similarities to EGF activities. The ligand binding activates the receptor tyrosine kinase. TGFβ is made by platelets, macrophages, lymphocytes and is mitogenic for many cell types including fibroblasts. TGFβ inhibits keratinocyte proliferation and induces squamous differentiation. TGFβ stimulates fibroplasias, bone formation and angiogenesis. TGFs can stimulate proliferation and promote multiple cellular responses. The downstream signaling of TGF-β binding to its serine/threonine kinase receptor is performed by Smad family members which ultimately activates Smad 2 or 3 to complex with Smad 4 for translocation to the nucleus for gene expression regulation. Suppression of the Smads by other hormones or growth factors such as insulin inhibit TGF-β induced apoptosis. TGFβ1 and β2 is in highest concentration in platelets and bone but is produced by many cell types in lower concentration. TGFβ3 is mainly in cells of mesenchymal origin and TGFβ4 is in chondrocytes. TGFβ 1, 2, 3 are mitogenic for mesenchymal derived cells and inhibits proliferation of hepatocytes, keratinocytes and many epithelial, T and B cells. TGFβ production is stimulated by IL-2, EGF, PDGF, TGFβ1, estrogen and wounds. The production is inhibited by androgens. TGFβ is secreted by cells in an inactive complex by non-covalently interaction with the latency associated peptide (LAP). This complex often is bound to an additional protein, the latent TGFβ binding protein (LTBP), forming the large latent TGFβ complex. These complexes are formed to tightly control the activitiy of TGFβ. LAP can combine with the other TGFβ forms as well. Thus LAP is a neutralizer of TGFβ activity. TGFβ2, TGFβ1.2 and TGFβ5 inhibit IL-4 dependent proliferation of cells. Receptors are present on almost all cell types and the effect of TGFβ depends on the cell type and growth conditions. Three sizes of receptors are made by most cell types for TGFβ. Type III, 250-350 kDa, is a proteoglycan that exists in both membrane bound and soluble forms, binds TGFβ1, β2 and β3 but is not involved in signal transduction. Membrane bound type II receptor binds TGFβ1, β3, β5 and TGFβ2. Membrane bound type I receptor needs type II receptor presence to bind TGF β. Soluble type II receptor can be a TGFβ antagonist. Soluble type III receptor binds TGFβ2 with the highest affinity and other TGFβ isoforms with lower affinities and displays antagonistic TGFβ2 activities. Soluble receptors are secreted by certain cell types.

BMPs include family members 2, 3, 3b, 4, 5, 6, 7 and 8. There are over 20 related BMPs. BMPs are involved in bone and cartilage formation, tissue morphogenesis and embryogenesis in which BMPs regulate growth, differentiation, chemotaxis and apoptosis of many cell types such as mesenchymal, epithelial, hematopoietic and neuronal cells. The myostatin member of BMPs can inhibit myoblast proliferation and increase muscle cell size. The GDF members include GDF-1 to -15 and are members of the BMP family. GDF-5 regulates myogenesis, chondrogenesis, bone morphogenesis and survival and differentiation of neurons. GDF-6 (BMP-13) is involved in myogenesis, chondrogenesis, bone morphogenesis, neuron survival and differentiation.

Neuropilins (Npns) are transmembrane type I receptors that bind class II secreted members of the semaphorin family, often involved in repulsive axon guidance. Npns are made by endothelial and tumor cells and are receptors for VEGF₁₆₅. Neurturin (NTN) promotes survival and outgrowth of neurons. Neurturin is a member of the GDNF (glial cell line-derived neurotrophic factor) family which includes as members artemin, neurturin, persephin and GDNF. Artemin promotes neuron survival, development and growth, including dopaminergic and sympathetic neurons. GDNF promotes neuron outgrowth and proliferation. GDNF promotes survival of neurons (motorneurons, midbrain dopaminergic neurons, Purkinje cells, sympathetic neurons) and is expressed by skeletal muscle cells, pinealocytes, neurons, Schwann cells, astrocytes and Sertoli cells. Activin family members induce mesoderm, bone remodeling, hematopoeiesis, neural cell differentiation, morphogenesis and are involved in reproduction. It stimulates FSH secretion. Members are activin A, B, C, AB, and inhibins A and B. Other members of the TGFβ superfamily are Lefty A and B. Inhibin is a FSH suppressing protein.

Protein regulators and inhibitors of the TGFβ superfamily members include amnionless, BAMBI/NMA, Chordin, Chordin-like 1 and 2, CRIM1, Cripto, Crossveinless-2, Cryptic, decorin, FLRG, Follistatin, Follistatin-like 1, GASP-1 and 2, NCAM-1, noggin, Smad 1, 4, 5, 7, 8, SOST, latent TGFβ bp1, TMEFF1 and 2, vasorin and the Cerberus/DAN family. The Cerberus/DNA family consists of BMP antagonists and are the secreted glycoprotein members Caronte, DAN, Cerberus, gremlin/DRM, Cer1 (cerberus-related), Dante and PRDC (protein related to DAN and Cerberus). Chordin is a secreted glycoprotein that is a BMP antagonist. Cryptic is involved in mesoderm differentiation. Along with Cripto these proteins are part of the EGF-CFC family of signaling proteins. Decorin, a small secreted chondroitin/dermatan sulfate proteoglycan is involved in ECM assembly and suppresses tumor cell line growth through activation of EGF receptor. Follistatin-related gene protein (FLRG) is upregulated by TGFβ and activin by Smad proteins. Follistatin originally was shown to be a follicle-stimulating hormone inhbiitng substance. It is an activin binding antagonist. GASPs (growth and differentiation factor-associated serum proteins) are protease inhibitors due to the follistatin, WAP, Kunitz and Netrin protease inhibitor domains. Noggin is expressed in skin, skeletal muscle, lung, central nervous system and other adult peripheral tissues and is a BMP binding protein that antagonizes BMP bioactivities. SOST (sclerostin) is expressed in osteoclasts and is involved in bone development.

The TNF superfamily consists of members TNFSFs (tumor necrosis factor superfamily) 1-18. Some are better known as TNFβ (TNSF1, lymphotoxin), TNFα (TNFSF2, cachetin), CD40 ligand (TNFSF5), Fas ligand (TNFSF 6), CD27 ligand (TNFSF 7), CD30 ligand (TNFSF 8), TWEAK (TNFSF 12), APRIL (TNFSF13), BAFF/BLyS (TNFSF 13B), LIGHT (TNFSF 14), VEGI (TNFSF15) and GITR ligand (TNFSF 18). Many of the TNFSFs are involved in apoptosis. Others, such as TNFα and TNFβ, can spur on cell proliferation of specific cell types (e.g. fibroblasts, osteoclasts, PMN cells). TNFα (cachetin) is produced by astrocytes, endothelial cells, smooth muscle cells, transformed cells, LAK cells, monocytes, macrophages, lymphocytes, neutrophils and NK cells, amongst others. TNFα occurs in biologically active membrane or soluble forms. TNFα and β mediate inflammatory responses, cytotoxicity (1.e. vascular endothelial cells), tumor growth, host defense, immune responses and can induce apoptosis.

TNFα production is stimulated by TNF, IL-1, IL-2, GM-CSF, M-CSF and inhibited by IFNα, IFNβ, TGFβ, IL-4, -6, -10, -11, -13 and GM-CSF. TNF1sR (receptor), TNFRSF1A or TNF R2 are soluble TNF receptors that contain the soluble extracellular domain of the TNF receptor. Soluble TNF receptors in serum can neutralize the activities of TNF. For example, TNFR-p60 Type B and TNFR-p80 Type A can bind TNFα and TNFβ. Soluble receptors can act as a reservoir of TNF also. TNFRs are made by many cell types, including mesenchymal types such as adipose cells, fibroblasts and muscle cells, immune cells and others. TNFα elevates levels of soluble TNFα receptors, IL-6, IL-1 receptor antagonist, and C-reactive protein. TNFβ, a 25 kDa glycoprotein, is expressed in activated T and B cells. TNFβ uses the receptor TNFRSF3 inducing NFκB activity, apoptosis, growth arrest, tumor cytoxicity and chemokine production and is involved in controlling cellular immune functions and lymphoid organogenesis. CD30 (TNFRSF8) is expressed on virus infected T and B cells, activated normal T and B cells, epithelial cells, monocytes and granulocytes. Receptor binding of CD30 ligand mediates cell proliferation, activation, differentiation and apoptosis. RANK (TNFRSF11A) receptor is widely expressed with highest levels present in adrenal gland, small intestine, thymus, liver, colon, skeletal muscle and dendritic cells. It inhibits TRANCE induced osteoclast differentiation. It is induced by IL-4 and TNF-β in peripheral blood T lymphocytes. TRANCE, RANK ligand, OPGL and ODF (osteoclast differentiation factor) are the ligands for RANK receptor. RANK results in T cell growth, dendritic activities, osteoclastogenesis and lymph node organogenesis. Osteoprotegerin receptor (OPGR, TNFRSF11B) is produced by many cells including fibroblasts and inhibits osteoclast development. OPG is a soluble TNF receptor which binds RANK ligand and is a decoy receptor to balance the effects of RANK ligand. TRAIL (TNF-related apoptosis-inducing ligand or TNFSF10) is a type II transmembrane protein and is expressed in many cell types and tissues. TRAIL receptors consist of 2 decoy receptors (TRAIL R3, 4) that antagonize TRAIL induced apoptosis and 2 receptors (TRAIL R1, 2) that transduce the apoptotic signals. OPG ligand (TRANCE, RANKL) and TRAIL ligand interact with OPGR and have roles in apoptosis, immune system and osteoclastogenesis. These ligands also bind TRAIL receptors 1-4. HVEM (Herpesvirus entry mediator) is a TNF receptor-like type I membrane protein and a member of the TNF/NGF receptor superfamily. Fas (CD95 or TNFRSF6) is expressed in liver, heart, lung, kidney, thymus, etc. Membrane and soluble forms exist. Fas ligand is a type II membrane protein that modulates immune response by apoptosis to maintain homeostasis and immune privilege. It is a chemoattractant for neutrophils and is proinflammatory. The membrane precursor is cleaved by metalloproteinase to generate soluble Fas ligand, which may inhibit the potent cytotoxicity of membrane bound Fas. HVEM can inhibit apoptosis. LIGHT (is homologous to lymphotoxins, exhibits inducible expression and competes with HSV glycoprotein D for HVEM. LIGHT is a type II membrane protein. LIGHT is produced by T cells, binds to LTβR (lymphotoxin beta receptor) and a decoy receptor (TR6), and can induce apoptosis in tumor cells that is enhanced by IFNγ. TNFSF8 (CD30L) is a type II membrane protein that through its CD30 or TNFRSF8 type I transmembrane receptor induces cell proliferation, activation, differentiation and apoptosis in immune cells and other cell types. GITR (glucocorticoid-induced TNF receptor, TNFRSF18) is a type I transmembrane protein expressed in peripheral blood T cells, thymus, bone marrow, spleen and lymph nodes. It modulates T cell functions and prevents T cells from TCR apoptosis. GITR ligand (TNFSF18) is expressed in endothelial cells. In general TNF can be mitogenic for specific normal cells but initiates apoptosis in transformed cells and specific cell types.

The TNF receptor (TNFR) transduces regulatory signals into the cell. The TNF receptors are all type I transmembrane glycoproteins with an extracellular domain containing cysteine-rich motifs. Soluble receptors shed by protease cleavage or alternate splicing can serve to concentrate the active TNF ligand. Most of the receptors regulate cell viability. FasR and TNFR type I contain a DD (cytoplasmic death domain) to signal apoptosis. Other receptors such as TNFR type II, lymphotoxin-β receptor (LT-βR) and CD30 signal apoptosis without having a DD domain. Some complex proteins to TNF receptors have the DD domain, including TRADD, FADD, RIP, MADD, and RAIDD. TNFR1 (TNFR-A, TNFR p55, TNFR p60, CD120a) binds TNFα, TNFβ or LT-α, associates with TRADD-FADD, TRAF-2, SODD, TANK, RAIDD, GCK and RIP, sheds soluble forms, is widely expressed and functions in apoptosis and inflammation. TNFR2 (TNFR-B, TNFR p75, TNFR p80, CD120b) binds TNFα, TNFβ, LT-α, complexes with TRAF-1 and -2, TRIP, sheds soluble forms, is widely expressed including the hemapoietic system, and functions in apoptosis and inflammation. LT-βR (TNFrrp) binds LT-α 1/β2, LIGHT, complexes with TFAF-5, has broad expression and is involved in apoptosis and lymph node development. Fas receptor (Apo1, CD95) binds Fas ligand, contains a DD, complexes with FADD, Daxx, FAF, has an alternate spliced soluble form, is expressed in lymhocytes and many tissues, and functions in apoptosis and immune privilege. CD27 (Tp55) binds CD27 ligand, complexes with TRAF-2 and -5, sheds soluble forms, is expressed in resting T cells and is involved in costimulation. CD30 (Ki-1) binds CD30 or CD153 ligand, complexes with TRAF-1, -2, -3, -5, is expressed in hematopoietic systems and Hodgkin's lymphoma, and functions in apoptosis and negative selection. CD40 binds CD40 ligand, TBAM, TRAP, complexes with TRAF-2, 3, 5, 6, sheds a soluble form, is expressed in T and B cells and carcinomas for cell survival and isotype switch. RANK (TRANCE R, ODF R) binds RANK ligand (OPGL), TRANCE, complexes with TRAF-2, -3, -4, -6, has broad expression and is involved in cell survival, bone mass regulation and lymph node development. OPG (OCIF) is a secreted soluble receptor that binds RANKL, is widely expressed, and is involved in bone mass regulation and lymph node development.

Hedgehog family members are involved in neurogenesis, bone formation, hematopoiesis and gonad development. Sonic, desert, and Indian hedgehog members can be regulated by Gas1 and Hip. Indian and sonic hedgehogs play a role in embryonic and eye development and retinal cell rescue. Sonic has a role in development of tissues such as hair, whisker, tooth, bone and foregut. It regulates the stem cell fates of neural and hematopoeitic lineages.

Wnts are key modulators of embryonic development, important in stem cell organization, maintenance, tissue differentiation, cell adhesion, migration, cancer induction, amongst other functions. Wnts are present in many cell types. At least 19 members of Wnt ligands are secreted glycoproteins including Wnts 1, 2, 2b, 3, 3a, 4, 5a, 5b, 6, 7a, 7b, 8a-d, 9a, 9b, 10a, 10b, 11, 12, 13, 14, 14b, 15 and 16. Wnt-3a induces myocyte aggregation, adhesion by cadherin-beta-catenin stabilization in muscle and is involved in BMP-2 chondrogenic differentiation. Wnt 3a and 4 is involved in wound healing wherein fibroblasts are surrounded by fibrin degradation products. Wnt 3 is expressed in premedullary cells of the hair follicle and enamel epithelium. Wnt related proteins are beta-catenin, GSK-3, Kermen-1, 2, LRP-1,-6, ROR 1, 2, WISP-1/CCN4. LRP-6 (low-density lipoprotein receptor-related protein-6} is a co-receptor with the Frizzled protein in the Wnt signaling pathway that stabilizes beta-catenin. The Dickkopf family of proteins interact with LRP-6. The Frizzled family of proteins, 1-10, are receptors for Wnt proteins that contain a conserved extracellular cysteine-rich domain. These receptors are present in fibroblasts, myofibroblasts, smooth muscle cells, and many other cell types. Frizzled related proteins are sFRPs 1-4 and MFRP. The Dickkopf family of proteins, 1-4, are secreted proteins (soluble receptors) that regulate Wnt signaling. Other soluble receptors in Wnt signaling are Norrin, WISE, WIF, Cerberus and sFRP (secreted Frizzled-related Proteins, secreted apoptosis-related proteins) members. Wnt inhibitors include Soggy-1 and WIF-1.

Proteoglycans can serve as growth factors. This family includes the members aggrecan, biglycan, decorin, endocan, endorepellin, glypicans (e.g. 2, 3, 6), mimecan and testicans (e.g. 1, 2, 3). These members can bind growth factor receptors determining activation or inhibition of receptor biological actions. Aggrecan macromolecules bind non-covalently via link protein to a single chain of hyaluronic acid. Decorin activates EGF receptor and is involved in ECM assembly. Glypican 3 is involved in the regulation of many signaling pathways such as IGF, FGF, BMP and Wnt. Testicans and extracellular multi-domain chondroitin sulfate proteoglycans modulate cell attachment in vitro and suppresses acitivity of lysosomal proteases like cathepsin L, and MT1 and MT3 MMP.

Other growth factors include: HGF (hepatocyte growth factor) a multifunctional growth factor made by fibroblasts, hepatocytes and present in the plasma. HGF stimulates epithelial cells to undergo tubulogenesis (e.g. epithelial cells in collagen gels) and cell growth and motility. It is a mitogen for hepatocytes, keratinocytes, melanocytes, endothelial and epithelial cells. HGF promotes epithelial and vascular endothelial dissociation of cell colonies in culture by stimulation of cell migration. HGF is an adipocytokine. HGF propeptide is cleaved by an extracellular serum protease into an active form. HGF inhibits TGFβ action, such as the transdifferentiation of fibroblasts into myofibroblasts. HGF mediates epithelial-mesenchymal interactions in tooth morphogenesis. HGF, through its c-Met tyrosine kinase receptor, is involved in cell migration, cell growth, cell motility, cancer invasion and metastasis in tumor cells. HGF has potent angiogenic activity. Thrombopoietin a ligand for the Mpl protooncogene receptor which regulates thromobopoiesis and megekaryocytopoiesis.

NGF (nerve growth factor, 26 kDa dimer) contains α, β, and γ subunits. The α and γ subunits are members of the kallikrein family of serine proteases. The β subunit represents β-NGF that has the biological activities of NGF including neurotropic activities, chemotaxis, immune regulation, differentiation, and neuronal development in sympathetic and peripheral nervous system. It is a trophic factor in basal forebrain for cholinergic neurons, targets neurons in vivo, induces differentiation and survival of neuronal cells in culture and has mitogenic properties for various cell types. NGF enhances the outgrowth and survival of nerve cells in vivo. The α and γ subunits of NGF are members of the kallikrein family of serine proteases. NGF binds to carrier proteins in the serum. NGF is produced by many tissues including the submaxillary gland. NGFR, is a type I transmembrane receptor that is part of the TNF receptor family. It is widely expressed in tissues on both neuronal and non-neuronal cells. Soluble NGFR, containing the extracellular domain of the membrane receptor, is present in serum, fluids, and can antagonize NGF by binding the cytokine. NGFR regulates cell migration, gene expression and apoptosis. p75 NGFR (NGFR) is expressed in the nervous system and binds NGF, BDGF, NT3 and NT4.

Neurotrophins (NTs) are members of the NGF family of neurotthrphic factors needed for differentiation and survival of specific neuronal (hippocampal, cholinergic, motor) cells. NTs (NT-3) are found in skin, skeletal muscle, placenta, heart, hippocampus, cerebellum, amongst other tissues. BDNF, NT-4/5 and TrkB are other neurotrophins. TrkB inhibits BDNF induced cell proliferation. BDNF is needed for differentiation and survival of specific neurons and it is present in cerebellum, hippocampus, fetal eye, placenta, pituitary gland, heart, lung, skeletal muscle and spinal cord. The ligand binds TrkB tyrosine kinase receptor. It stimulates substantia dopaminergic neurons, hippocampal neurons, neural crest sensory neurons, basal forebrain cholinergic neurons and retinal ganglial cells.

Connective tissue growth factor (CTGF, CCN2, insulin-like growth factor binding protein-related protein 2), 38 kDA, is a fibroblast, chondrocyte and vascular endothelial cell mitogen and chemoattractant. CTGF stimulates ECM production, such as collagen deposition (e.g. in skin). Excess CTGF is involved in tissue fibrosis. CTGF can be useful in anti-fibrotic therapy when there is excessive coagulation proteases and TGFβ present. CTGF is an angiogenic factor. CTGF mediates TGFβ induced collagen synthesis.

Hepassocin, NOV/CCN3 and progranulin are growth factors. Angiopoietins (e.g. Ang-1, 2, 3/4) are agonists and antagonists of the Tie-2 receptor tyrosine kinase and modulator of angiogenesis. MSP (macrophage stimulating protein or hepatocyte growth factor-like protein[HGFI] or scatter factor-2 [SF2]) is a member of the HGF growth factor family. MSP prevents epithelial cell anoikis. MSP proliferates keratinocytes, affects macrophage cell migraton and shape, bone resorption by osteoclast-like cells, inhibits IFN or LPS induced iNOS expresson in macrophages, and is a chemoattractant for macrophages. MSP binds to RON/STK, a tyrosine kinase receptor that is present on macrophages, keratinocytes, vascular endothelium, epithelial cells, neurons, and lymphocytes. Flt-3 ligand synergizes with a variety of hematopoietic cytokines that stimulate growth and differentiation of hematopoietic progenitors and proliferation of pro-B cells. Flt-3 is found in various tissues including the reproductive, nervous and hemoatopoietic. The transmembrane protein form can be proteolysed into a soluble form that acts as an antogonist to Flt-3 ligand. M-CSF is produced by a number of cells including fibroblasts, epithelial cells, bone marrow stromal cells, astrocytes, keratinocytes, osteoblasts, renal mesangial cells, macrophages, monocytes, B cells, T cells, mast cells and endothelial cells. M-CSF is involved in macrophage progenitor proliferation and differentiation. SCT (stem cell factor) plays a role in melanogenesis, early hematopoiesis, gametogenesis, immature and mature cell proliferation (e.g. mast cells, melanoblasts, bone marrow cells) and is expressed in progenitor cells (e.g. hematopoietic, B, T cell), mast, germ and glial cells, melanocytes, neurons, kidney, lung, gut and placenta cells. LDGF (leukemia-derived growth factor) is produced by immune cells. Leiomyoma-derived growth factor is a mitogen for smooth muscle like cells. Leukocyte-derived growth factor (e.g. LDGF-3) is a major fibroblast mitogen produced by macrophages in culture after lipopolysaccharide activation. The protein is a precursor for other cytokine and chemokine factors such as PBP (platelet basic protein), CTAP-3 (connective tissue activating protein-3), β thromboglobulin, and NAP-2 (Neutrophil-activating protein-2). Neuregulins (NGR1, 2, 3) are a family of peptides that stimulate the erb-2 receptor (e.g. phosphorylation) and influence muscle cell proliferation. Neuroleukin is a neuronal growth factor and lymphokine produced by T cells (lectin stimulated) and induces immunoglobulin secretion. Neurotrophic factors include nerve growth factor, brain-derived neurtrophic factor, ciliary neurotrophic factor, glial cell line-derived neurotrophic factor, IL-6 and FGF-2. Peptide YY is a growth factor for intestinal epithelium. Platelet factor 4 or its 24 carboxy terminal fragment binds to FGF, inhibiting its mitogenic activity and acting as an inhibitor of the MAP kinase pathway (includes Raf, MEK1/2, ERK1/2) that controls cell proliferation and survival of tumor cells.

Endothelial growth factors are soluble mitogens made by a variety of organs and are a mixture of two single chain polypeptides that have affinity to heparin. The factors are mitogenic and chemotactic, stimulate endothelial cells to grow and are related to acidic and basic FGF.

M-CSF (macrophage colony stimulating factor) is present in serum, urine, and other fluids. M-CSF is made by fibroblasts, activated macrophages, secretory epithelial cells, bone marrow stromal cells, cytokine and LPS activated endothelial cells. M-CSF is mitogenic for macrophages, enhances macrophages to kill tumor cells, regulates cytokine and inflammatory factors release from macrophages and differentiates osteoclasts. G-CSF (granulocyte-macrophage colony-stimulating factor), a 15-30 kDa glycoprotein, is produced by many cell types such as activated T lymphocytes, fibroblasts, endothelial cells and macrophages. It stimulates the proliferation of neutrophilic, eosinophilic granulocytes and macrophages and initiates proliferation of bone marrow precursor cells, erythroid and megakaryocyte precursors. TNFα, IFNγ and endotoxin stimulate its production by monocytes and macrophages. LPS, IL-1 or TNFα stimulation of fibroblasts, endothelial cells, bone marrow stromal cells and astrocytes causes the secretion G-CSF. It is involved in inflammation and repair and maintenance of steady state hematopoieses. CSF-1 (colony-stimulating factor or M-CSF, macrophage colony-stimulating factor) is a 14-21 kDa homodimer glycoprotein produced by many cell types such as fibroblasts, endothelial cells, monocytes and macrophages. It stimulates the proliferation and differentiation of bone marrow progenitor cells to form macrophages and is needed for monocyte and macrophage survival. Gly-His-Lys is a growth factor for fibroblasts, kidney cells, eosinophils and hepatoma cells.

EPO (erythropoietin) is made by the kidney and regulates erythropoiesis by stimulating proliferation and differentiation of erythroid progenitor cells. Its receptor is a type I transmembrane protein and a soluble cleaved product is present in the plasma.

Receptor tyrosine kinases (e.g. growth factor) can activate the MAPK signaling pathway that controls proliferation, differentiation and motility among other cell functions. For example, osteoblast differentiation and bone formation is activated through the Cbfa1 transcription factors by the MAPK pathway. The MAPK pathway can by stimulated by ECM signals, osteogenic growth factors such as BMPs and FGF-2 and by parathyroid hormone, amongst other growth factors and molecular signals.

Many ECM and serum proteins can be considered growth factors or required for growth factor action. Proteoglycans sequester growth factors and their release dictates the growth factor action. Many cell types such as fibroblasts, epithelial and smooth muscle, NK and T cells, macrophages, osteoclasts respond to cytokine, growth factors or inflammatory mediators. Cell adhesion can trigger ligand-independent activation of growth factor receptors resulting in the biological action of these receptors. Growth factors can induce adhesion molecules to promote adhesion-independent signals. Cell adhesion proteins interact with receptors that signal pathways of cell behavior. For example, OPN (Osteopontin) is a RDG containing glycoprotein that due to the RGD domain binds to integrins α₅β₁, α₅β₃ and α₅β₅. OPN also has a non-RGD interaction with CD44 and integrins α₈β₁ or α₉β₁. Through these receptor interactions, OPN is chemotactic for macrophages, smooth muscle, endothelial and glial cells.

As demonstrated above, growth factors are involved in a number of processes that include cell adhesion, cell migration, cell proliferation, apoptosis, anoilds, proteolysis, differentitation, ECM synthesis and degradation, wound healing, amongst others.

Cytokines

Cytokines are extracellular short-range polypeptide or small protein mediators with a wide range of action. Growth factors and cytokines terminology is often used interchangeably and share common pathways in many instances. There is overlap with some chemokines as well. Immune cells and epithelial cells as well as other cell types such as fibroblasts are involved in producing cytolcines. Cytokines are expressed by a variety of cells in response to infection, inflammation, lymphokines (cytokines produced by immune cells), coagulation, bacterial endotoxins, etc. Cytokines are also involved in other immune and non-immune functions and physiological processes, since immune cells pervade all tissues including connective tissue. Thus cytokines produced by cells, immune cells attracted by the cytokines, and other cells affected by the cytokines have effects on the ECM and other components in tissue. Most cytokines are secreted, although some may be expressed in the cell membrane and many are present in the ECM and serum. Cytokines bind to specific receptors on the target cell membrane which is linked to intracellular transduction and second messenger signalling pathways. For example, IL-2, 4, 7, 15 and 21 are involved in T cell growth, and TNF, IL-1, IL-6, IFNγ in inflammation and IL-4, IL-10, TGFβ in inhibition of inflammation.

Interferons and interleukins are some of the primary cytokines These and other cytokines not described under the growth factor section are listed below and throughout the text.

Interferons are not themselves viricidal. They are a group of immunoregulatory proteins made by T lymphocytes, fibroblasts and other cell types following stimulation by viruses, antigens, mitogens, double-stranded DNA, or lectins. The interferons have antiviral properties and immunoregulatory functions by enhancing the ability of immune cells such as macrophages to destroy tumor cells, viruses and bacteria. Interferon α (IFN α), 20-25 kDa comprise glycoproteins synthesized by most cell types including macrophages and B cells. This class of interferon is able to prevent the replication of viruses, is antiproliferative, is pyrogenic, stimulates natural killer cells, enhances the expression of class I MHC antigens and immunoregulates through alteration of antibody responsiveness. Interferon β (IFN β or fibroblast interferon) is a 25-35 kDa glycoprotein produced by fibroblasts and activated T cells, among other cell types and prevents replication of viruses. It can induce the differentiation of keratinocytes. IFN-α and IFN-β production is induced by viruses, growth factors, cytokines and ds RNA. These IFNs induce differentiation and inhibit the proliferation of a number of cell types as well as transformed or tumor cell lines.

Interferon γ (IFN γ) is a cytokine, lymphokine, a 21 to 24 kDa homodimer protein produced by activated T lymphocytes and natural killer cells. It has antiproliferative, proinflammatory, immunoregulatory and antiviral properties. IFN γ decreases synthesis of collagen by fibroblasts. It is an activator of mononuclear phagocytes and macrophages, increasing the ability to destroy intracellular microorganisms and tumor cells. It causes many cell types to express class II MHC molecules and also increases expression of class I. IFN γ facilitates differentiation and maturation of both B and T lymphocytes, enhances secretion of immunoglobulins by B cells, inhibits osteoclast activation and induces MHC class I and II antigens and cytokine production. It activates natural killer cells, neutrophils and vascular endothelial cells. IFNγ receptor is found on almost all cell types and is related to the IL-10 receptor. β and γ interferons enhance expression of MHC molecules, β2-microglobulin, cytokine receptors for TNF, IL-1, IL-2, and colony stimulating factor in a variety of cell types. Interferons in general are anti-growth.

Interleukins are a group of cytokines made by lymphocytes, monocytes and other select cells. Interleukins promote growth of T cells, B cells and hematopoietic stem cells in addition to other biological functions. Interleukins are soluble factors that enhance cell proliferation and differentiation, DNA synthesis, secretion of other active molecules and responds to immune and inflammatory stimuli. They stimulate leukocyte and other cell type growth related activities.

There are more than 32 members of the interleukin family. Many of the interleukins assist the immune response by proliferation of immune cells and secretion of immune factors including interleukins. Some of the known interleukins and their known sources, targets and functions are:

IL-1 represents two proteins IL-1α and IL-β. IL-1α, ˜17 kDa, is a pleiotropic factor made by a variety of cells. IL-1α targets B, T and DC cells and monocytes. It stimulates T, B and NK cells, microglia, astroglia, and modulates neuronal electrophysiology. IL-1β, ˜17 kDa, is made by a variety of cells and targets B and T cells and monocytes. As a pleiotropic factor it stimulates many cell types and is a central mediator of inflammation. IL-1 stimulates proliferation of fibroblasts, T and B cells, helper T cells, hepatocytes, macrophages, chondrocytes, endothelial cells, epithelial cells, additional lymphocytes and other cell types. Inflammation stimulates the production of IL-1 by macrophages, osteoblasts, monocytes, keratinocytes, hepatocytes, fibroblasts, glia (oligodendroglia, astrocytes, microglia), Kupffer cells, epithelial cells (thymic, salivary gland), amongst other cell types. IL-1 stimulates B-cell function, fever, IL-2 production and synthesis of collagenase. IL-1 is made by activated mononuclear phagocytes that have been stimulated by ribopolysaccharide or by interaction with CD4+ T lymphocytes. IL-1β is processed by interleukin 1β-converting enzyme (ICE). Both IL-α and β are pro-inflammatory cytokines that act on many cell types with a variety of biological actions. The IL-1β pathway is inhibited by TGF-β, IL-10, -13 and IFNα. IL-1 is mitogenic for keratinocytes, fibroblasts, stimulates IL-2 production and stimulates B-cell function.

A number of receptors are available to interact with IL-1. IL-1 has two general types of receptors, a type I transmembrane, present predominantly on fibroblasts, endothelial cells and T cells, that mediates the IL-1 biological responses. Type II transmembrane and soluble receptors act as a decoy receptor to prevent IL-1 binding to its type I receptor. Type II is present on B lymphocytes, neutrophils, monocytes, leukocytes and endothelial cells. An IL-1 receptor accessory can heterodimerize with Type I receptor in IL-1α or β presence, but not with IL-1rα, to conduct IL-1 biological processes. Soluble receptor I is an antagonist of IL-1 action. IL-1R4 has two forms, a transmembrane type I protein (ST2L) and a soluble protein (ST2). ST2 is deposited in the ECM and is involved in cell adhesion. IL-1 receptor 6 (R6) is expressed on fibroblasts, endothelial cells, keratinocytes, monocytes, kidney, epithelial cells (lung), brain vasculature and testis. IL-1R6 mediates activation of transcription factor NF-KB by IL-1 F9 (IL-1 H1) and this action is antagonized by IL-1 F5. IL-1RAcP, 60 kDa, is made by many cells, complexes with IL-1R Type 1 and IL1-α or IL1-β. IL-1RA, 17 kDa, is made by fibroblasts, macrophages, monocytes and neutrophils, inhibiting the release of IL-1, the secretion of IL-2, the expression of IL-2 receptors, and the stimulation of PGE2.

IL-2, 15 kDa, is made from activated lymphocytes (e.g. activated T cells), targets T, B NK and LAK cells, monocytes and oligodendrocytes. IL-2 proliferates activated lymphocytes T, NK and B and tumor-infiltrating lymphocyte. IL-2 matures these cells to become cytotoxic to kill target cells, and is involved in tumor surveillance. IL-2 activates neutrophils, induces IFNγ, TNFα, β from blood mononuclear cells, IL-2 receptors on T cells, c-myc RNA and transferrin receptor. IL-Rα binds IL-2, activates T and B cells and the immune system. Glucocorticoids and CTLA-4 inhibit IL-2 production.

IL-3 (multi-CSF), a 15-28 kDa glycoprotein, is made by activated T cells (antigen or mitogen stimulated), monocytes, keratinocytes, NK, mast cells, endothelial cells, neurons, astrocytes, epithelial cells (thymic) and targets hematopoietic progenitor cells into many lineages, functions in hematopoiesis and pre-B cell development and self-renewal, and survival and differentiation of multipotential stem cells. IL-3 is a chemoattractant for eosinophils. IL-3 also stimulates the proliferation and differentiation of pluripotent hematopoietic stem cells and various lineage committed progenitors such as granulocytes and macrophages, regulates the activity of mast cells, eosinophils, macrophages and basophils. IL-3 receptors are present on bone marrow progenitors, macrophages, mast cells, eosinophils, megakaryocytes, basophils and other myeloid cells.

IL-4, a 13-20 kDA glycoprotein, is made by helper and activated T cells, mast cells and basophils. IL-4 targets B and T cells, monocytes, macrophages, mast cells, endothelial cells, erythroid progenitors, fibroblasts, amongst others, and targets the proliferation of B cells and its secretion of Ig, promotes production of IgG and IgE, regulates T helper subset, matures mast cells, stimulates proliferation of specific cells and has anti-tumor effects. The alternate splice variant IL-4δ-2 is an IL-4 antagonist.

IL-5, a 22-40 kDa glycoprotein, is made by T cells, mast cells and eosinophils and targets eosinophils, basophils and B cells by regulating the proliferation, differentiation and activation of these cells and enhances IgM and IgA production. The IL-5 receptor is a transmembrane protein. The soluble receptor α binds IL-5 and can be an IL-5 antagonist.

IL-6, a 21-28 kDa glycoprotein (when complexed in serum with α2-microglobulin, 42-45 kDa), is made by fibroblasts, endothelial cells, hepatocytes, keratinocytes, astrocytes, vascular endothelial cells, tumor cells, bone marrow, muscle fibers, T and B cells, stimulated monocytes and macrophages. It is present in serum, milk and the synovial fluid of rheumatoid arthritis patients. IL-6 targets peripheral blood monocytes, T and activated B cells, nerve cells, fibroblasts, hepatocytes and epithelial cells. IL-6 is involved in inflammation, T cell and neuron proliferation, B cell to plasma cell maturation, trophoblast development, immune response, host defense, hematopoiesis, keratinocyte differentiation and acute phase reaction mediation. IL-6 can stimulate production of anti-inflammatory cytokines such as IL-1rα and IL-10 and inhibits the production of the proinflammatory cytokine TNF-α. IL-6 production is upregulated by mitogenic or antigenic stimulation, LPS, calcium, IL-1, IL-2, IFN, TNF, PDGF, viruses and inhibited by IL-4 and IL-13. Gp130 is a membrane bound protein that is proteolytically cleaved to soluble forms that are present in serum inhibiting OSM and CNTF activities. Gp130 is a common signal transducing receptor component used by the IL-6 family members (e.g. LIF, OSM, CNTF). Binding of IL-6 or IL-11 to soluble or membrane bound gp130 triggers signal transduction.

IL-7, a ˜15-25 kDa glycoprotein, is made by keratinocytes, bone marrow and thymus stromal cells, targeting T and B progenitor cell proliferation and megakaryocytes. IL-7 is involved in immature and mature T cell and thymocyte growth, megakaryocyte and pre-B cell development, and proliferation of thymocytes and lymphocytes. IL-7 transmembrane and soluble forms of receptors are present on pre-B and T cells and bone marrow cells. IL-8 (CSCL8), 8 kDa, is made by many cell types including fibroblasts, endothelial cells, keratinocytes, hepatocytes, chondrocytes, tumor cells, T cells, neutrophils and activated monocytes. Upon proinflammatory stimuli such as LPS, viruses, TNF and IL-1, IL-8 targets T and B lymphocytes, neutrophils, monocytes, NK cells, basophils, eosinophils, hematopoietic stem cells, fibroblasts, endothelial and smooth muscle cells, keratinocytes and melanoma cells. IL-8 is involved in neutrophil activation, chemoattraction, pro-inflammatory reactions, and with cell adhesion molecules. IL-8 is a chemotactic factor for neutrophils, basophils and T cells. CD11, enhances neutrophil adherence to endothelial cells and subendothelial ECM, is a co-mitogen for keratinocytes, a growth factor for melanoma cells and has angiogenic activity. IL-4, -10, TGFβ, glucocorticoids, and vitamin D3 inhibits IL-8 production. IL-9, is made by activated Th2 (T helper 2) or Hodgkin's lymphoma cells, targets T and progenitor erythroid cells, promotes B cell growth with IL-4 and mast cell growth with IL-3. IL-9 promotes T cell survival, stimulates proliferation of specific immune cells such as T-helper cells, megakaryocytes and mast cells, and has transmembrane and soluble receptors present in serum, T cells, neutrophils, mast cells and macrophages. IL-10, 18 kDa, is made by Th2, keratinocytes, B1, activated CD8 and CD4 cells. IL-10 targets fibroblasts, macrophages, granulocytes, eosinophils, mast and B cells and is a chemoattractant for CD8 T cells. IL-10 promotes B cell viability, enhances proliferation of immune cells such as mast cells, thymocytes and B cells, increases Ig secretion, enhances the ability of antigen-presentation and down regulates Class II MHC expression, suppresses activation and the cytotoxicity of monocytes, macrophages and T cells. IL-11, 23 kDa, is made by bone marrow stromal and mesenchymal cells, targets hematopoietic stem cells, stimulates erythropoiesis and liver acute-phase protein activity, stimulates T-cell dependent maturation of B cells (e.g. IgG production), increases cycling rates of bone marrow-derived progenitor cells, inhibits the differentiation of pre-adipocytes as an adipogenesis inhibitory factor (AGIF) and is involved in megakaryocytopoiesis. IL-11 is a mitogen for IL-6 responsive cells and other cells and uses the IL-6 signal transducer gp130 for signal transduction (as does LIF, OSM and CNTF). IL-11 stimulates T cell dependent development of specific immunoglobulin secreting B cells. IL-11 Rα is a receptor that is a membrane and cleaved soluble receptor. The receptor is present in all tissues and in cells expressing the gp130 protein, during embroninc development and totipotent and differentiating embryonic stem cells. IL-12 p70, 70 kDa heterodimer, is made by macrophages, dendritic cells and monocytes and targets, T, B and NK cells. IL-12 regulates cell-mediated immune responses, stimulates NK activity, induces IFN-γ production, enhances cell proliferation and cytotoxicity of NK and T cells and induces Th1 responses. IL-12 p40 homodimer is made by macrophages, monocytes and dendritic cells, targets macrophages and is involved in macrophage chemotaxis, proinflammation and is an antagonist to IL-12 p70. IL-12 activates T cell and NK cell growth and induces IFN-γ. IL-13, 12.5 kDA, is made by NK and mast cells, activated CD 8, Th CD4 and Th2 cells. IL-13 targets B cells and the monocyte lineage, inhibits macrophage cytotoxic activity, suppresses inflammatory cytokine expression, upregulates IL-1RA expression, induces CD23 expression on B cells, modulates differentiation of monocytes and macrophages and enhances expression of CD72 and class II MHC gene expression. IL-14, is made by T cells and B cell lines after PHA stimulation, targets B cells, inhibits secretion of immunoglobulin and is a mitogen for activated B cells. IL-15, 14-15 kDa, is made by fetal astocytes, fibroblasts, epithelial cells, adherent peripheral blood mononuclear cells and microglia in response to IL-1β, IFN-γ or TNF-α. IL-15 targets monocytes, NK cells, and is similar in activities to IL-2 including T, B and NK cell stimulation activities, NK cell maturation, T cell mediated immune response, cytolytic cell generation and LAK cell activity in vitro. IL-15 is a mitogen for immune cells. IL-15 binds to its receptor IL-15Rα that is expressed in T, B and non-lymphoid cells. Binding of IL-15 to its receptor inhibits TNFa mediated apoptosis in fibroblasts by competition with TNFR1 for TRAF2 binding. IL-16 (lymphocyte chemoattractant factor), is made by fibroblasts, epithelial and mast cells, eosinophils and activated CD8+ cells. IL-16 targets T cells, macrophages, eosinophils, cells in the thymus and lymph nodes, spleen leukocytes, cerebellum and bone marrow. IL-16 suppresses HIV replication and chemoattracts CD4+ T cells. IL-17, 15-25 kDa, is made by CD4+ memory T cells and targets fibroblasts, stromal cells, endothelial and epithelial cells, and is involved in angiogenesis and neutrophil maturation. IL-17 induces IL-6 and IL-8 production and the surface expression of ICAM-1 in fibroblasts, activates NF-κB and co-stimulates T cell proliferation. IL-17R is present in all cells and tissues. IL-18, 24 kDa, is made by many cell types including activated macrophages, keratinocytes, epithelial cells (intestine), osteoblasts, adrenal cortex cells and Kupffer cells. IL-18 targets T cells, is a pro-inflammatory cytokine, induces IFN-γ production by T and NK cells and GM-CSF in peripheral blood mononuclear cells and induces T helper type I cytokines, IL-2, GM-CSF and IFNγ in T cells. IL-18 also enhances Fas ligand production by Th1 cells and has a role in angiogenesis. IL-18 Rα is a member of the IL-1 family, shares immunoregulatory functions with IL-12 and is expressed in many tissues including spleen, lung, liver, heart, intestine, prostate, thymus and leukocytes. IL-19, is made by activated monocytes and B cells and targets activated T cells and monocytes. IL-19 induces IL-6 and TNF-α by monocytes, induces apoptosis, reactive oxygen species production by monocytes, induces IL-4, IL-5, IL-10, IL-13 production by activated T cells and is involved in asthma. IL-20 is made by monocytes and keratinocytes. IL-20 targets keratinocytes, regulates keratinocyte differentiaon, proliferation, and functioning. IL-21 is made by activated CD4 T cells, targets dendritic, T, B and NK cells, stimulates proliferation of bone marrow progenitor cells, and B cell proliferation with CD40, and stimulates T and NK cells. IL-22, 25 kDa, is made by NK and CD4+ Th1 cells, T and mast cells, is pro-inflammatory and induces acute phase protein synthesis. IL-23, is made by activated dendritic cells, targets T cells, is pro-inflammatory, induces memory T cell proliferation and induces IFN-γ production by naïve and memory T cells. IL-24, 35 kDa, is made by monocytes, melanocytes, fibroblasts, breast epithelium and vascular smooth muscle cells, B, naive T and NK cells and induces apoptosis (e.g. cancer cells). IL-24 induces IL-6 and TNF-α in monocytes and differentiation in megakaryocytes. IL-25, is made by Th2 cells, bone marrow stromal cells, induces serum IgG, IgE and eosinophil production and inflammation, is involved in the proliferation of lymphocytes, and mediates effects though induction of IL-4, IL-5 and IL-13. IL-26, 36 kDa, is made by T, NK and Th1 cells, targets epithelial cells and induces secretion of IL-8, IL-10 and CD54 expression. IL-27, is made by mature dendritic cells, targets NK and naïve CD4+ T cells, induces proliferation of naïve CD4+ T cells and is an activator of Th1 responses. IL-28A, IL-28B and IL-29 are made by dendritic cells and induced by virus infection or dsRNA. They target most tissues but brain and spinal cord, upregulates class I MHC and have anti-viral activity. IL-30 is made by activated APC (antigen presenting cells), targets NK and naïve CD4+ T cells. IL-31 is made by activated T cells, targets epithelial cells and activated monocytes and is involved in allergic reactions and dermatitis. IL-32 is made by activated T and NK cells, mitogen activated lymphocytes, IFN-γ treated epithelial cells and targets macrophages, is inflammatory, and induces TNF-α, IL-8 and MIP-2 production.

Interleukins that promote apoptosis can by neutralized to prevent cell apopotosis. Tissue damage can be caused by interleukins such as IL-6, IL-8, IL-1α and IL-1β. IL-11 can be used to promote pre-adipocytes expansion in situ and in vitro. IL-13 can reduce cytokines in inflammation thus reducing reaction to immunogenic agents.

Other cytokines include the Mer, Axl and Dtk receptor tyrosine kinases whose extracellular portion contains Ig-like domains and two fibronectin III domains. They are found in cell adhesion molecules (e.g. neural) and in receptor tyrosine phosphatases. One ligand for these receptors is the vitamin K-dependent growth-arrest-specific protein (Gas 6), related to anticoagulation factor protein S. Binding of Gas 6 induces receptor autophosphorylation leading to cell proliferation, migration and apoptosis prevention.

Secreted MIF inhibits macrophage migration, as does IL-4 and IFNγ and other cytokines, and has a role in inflammatory responses.

Oncostatin M (OSM) binds LIFR (receptor) and is mitogenic for fibroblasts, stimulates plasminogen activator acitivity and regulates IL-6 production in endothelial cells, and stimulates LDL uptake and LDL receptor production. OSM, in the presence of glucocorticoids, can induce differentiation. Gp130, a signal transducing component of IL-6, LIF and CNTF receptor complexes binds OSM without transducing OSM signals.

CNTF (ciliary neurotrophic factor) is a survival factor for neuronal cell types such as hippocampal, sympathetic ganglion, embryonic motor and dorsal root ganglion sensory neurons. The polypeptide is mitogenic for specific cell types and shares gp130 as the signal transducing subunit in their receptor complexes with IL-6, IL-11, LIF and OSM. All have four helix bundles. CNTFRα is restricted to the central and peripheral nervous systems. Soluble receptor can be released from skeletal muscle from peripheral nerve injury and it is present in cerebrospinal fluid.

Pleiotrophin (PTN) is a heparin-binding developmentally regulated cytokine. It is mitogenic for fibroblasts, endothelial and epithelial cells.

Stem Cell Factor (SCF, mast cell growth factor[MGF], steel-factor[SLF]) stimulates proliferation and maturation of mast cells, and is involved in melanogenesis, hematopoiesis, gametogenesis and nervous system development. SCF promotes pluripotent hematopoietic stem cell maturation. SCF synergistically interacts with many growth factors such as IL-1, -3, -6, -7 and Epo, inducing myeloid, lymphoid and erythroid lineage colony formation. SCR is produced by fibroblasts, endothelial cells, bone marrow and Sertoli cells. SCF receptor is expressed in gut, kidney, lung, placenta, glial cells, neurons, melanocytes, germ, mast, tumor and hematopoietic, B and T progenitor cells. SCFR (receptor) can be proteolytically cleaved into a soluble form present in plasma and is a SCF antagonist.

LIF (Leukemia inhibitory factor) binds to its receptor consisting of two membrane glycoproteins (LIF Rα and gp130). LIF and its receptor mediate the effects of oncostatin M, cardiotrophin-1 and CNTF (ciliary neurotrophic factor). LIFRα is a type I membrane protein with a 789 aa extracellular domain that contains two cytokine receptor domains and three fibronectin type III repeats. Soluble LIFRα is present in plasma and has LIF antagonistic activity. LIF is mitogenic for stem cells, hepatocytes, hematopoietic cells, and carcinoma cells. LIF inhibits embryonic stem cell differentiation.

Cardiotrophin-1 (CT-1) is a member of the family consisting of IL-6, IL-11, LIF, OSM and CNTF. It is expressed in heart, skeletal muscle, liver, lung, kidney and other tissues. It is mitogenic for many cell types.

DCC (deleted in colorectal cancer) is a tumor suppressor protein that is a type I transmembrane with an extracellular domain containing four Ig like and six fibronectin type III like repeats. DCC is a receptor for the netrins for axon guidance. DCC, a caspase substrate, promotes apoptosis unless bound by netrins.

DNAM-1 is a type I transmembrane glycoprotein that is expressed on T and NK cells and macrophages. DNAM-1 is a signal transducing adhesion molecule that is involved in the adhesion of tumor cells to CTL and NK cells and mediates the cell's cytotoxicity, dependent on the PKC pathway activated.

Ties (tyrosine kinase with Ig and EGF homology domains 1) comprise a receptor tyrosine kinase (RTK) that contain 2 immunoglobulin motifs flanked by 3 EGF-like motifs, followed by 3 fibronectin type III-like repeats in the extracellular domain of the transmembrane protein. These receptors are expressed on endothelial and hematopoietic progenitor cells playing roles in angiogenesis, vasculogenesis and hematopoiesis. Tie-1 is involved in endothelial cell differentiation and its maintenance of endothelium integrity. Tie-2 has angiopoietin-1 and -2 as ligands and is involved in angiogenesis. Angiopoietin-1 (Ang1) and -2 (Ang2) are secreted ligands involved in angiogenesis and maintenance of the adult vasculature. Ang 2 can be an antagonist to Ang 1 and Tie-2.

TPO (thrombopoietin) is the ligand for the c-Mpl proto-oncogene receptor and regulates megakaryocytopoiesis and thrombopoiesis. It can serve as a mitogen for some cell types.

uPA (urokinase-type plasminogen activator) is the ligand for the receptor uPAR, a serine protease needed for cell migration and causing tissue destruction. uPAR localizes uPA protease activity and initiates the signal transduction process to activate protein tyrosine kinases, gene expression, chemotaxis and cell adhesion. uPAR can suppress normal integrin adhesive function and promote adhesion to vitronectin via a high affinity binding site on uPAR. An alternate spliced variant of uPAR produces a secreted soluble form. The urokinase receptor derived peptide SRSRY can promote adhesion to vitronectin.

Angiogenin is present in plasma has high as 120 ng/ml. It is involved in angiogenesis and is an endothelial cell mitogen. Angiogenin supports endothelial and fibroblast cell adhesion and spreading. Angioarrestin is an anti-angiogenic protein with tumor-inhibiting properties.

B7-1 and -2 ligands and CD28 and CTLA-4 receptors costimulate pathways that regulate T and B cell responses. B7-1 is expressed on activated B and T cells and macrophages. B7-2 is expressed on dendritic cells, Langerhan's, memory B, germinal center B and peripheral blood dendritic cells, monocytes and can be induced by IFNγ. CD28/B7 interaction prevents T cell apoptosis by upregulation of bcl-X_{L}. CD4 is a type I membrane glycoprotein or soluble receptor expressed in thymocytes and T cells and is a co-receptor of HIV entry that binds the gp120 protein. CD6 is involved in T cell activation and is an adhesion receptor, mitogenic for T cells, binds ALCAM, the activated leukocyte cell adhesion molecule and is expressed in B cells, T cells, neuronal cells and thymocytes. CD14 is expressed on monocytes and macrophages.

Chemokines

In general, chemokines, ˜8 to 14 kDa, are soluble cytokines that activate or chemoattract leukocytes through G-protein coupled receptors. Chemokines are also involved in other immune and non-immune functions and physiological processes, since immune cells pervade all tissues including connective tissue. Thus chemokines produced mainly by immune cells, the immune cells attracted by the chemokines, and other cells affected by the chemokines have effects on the ECM and other components in tissue. HIV uses chemokine receptors to enter host cells. Chemokines have roles in inflammation, infectious disease, and normal and pathologic immune responses. Inflammation triggers include infection, allergen, autoantigen, alloantigen, tumor, etc.

Other cells than immune cells make chemokines, such as fibroblasts, epithelial urothelial and smooth muscle cells. The transition from innate immunity to acquired immune response involves signals that activate tissue macrophages and fibroblasts producing chemokines that recruit additional imflammatory cells. Dendritic cells mature and migrate with the specific antigen to draining lymph nodes during an acquired immune response.

Many chemokines are under the regulation of IL-1 and TNF. There are more than 18 cytokine receptors, grouped into 4 subfamilies that bind the 4 major subfamilies of chemokines (CXC, CC, CX3C, and C) in which there are more than 50 members. Two main groups of chemokines exist. One group is the inflammatory chemokines that are induced by inflammatory stimuli which recruit leukocytes. The other group, the homeostatic chemokines are constitutively expressed in tissues and certain cell types to support the development and maintenance (homeostasis) of the immune and hematopoietic systems. Tumor cell produced chemokines can be autocrine or paracrine growth factors providing survival signals. Production of inflammatory chemokines by tumor cells and stromal cells recruit leukocytes and play a role in invasion and metastasis. Chemokines can bind GAGs, such as heparan (HS), chondroitin sulfate (CS) or the proteoglycans (PGs) containing these GAGs, promote or retard presentation of chemokines to their receptors. HSPG promotes chemokine delivery whereas CSPG (versican) attenuates chemokine binding thus downregulating integrin mediated cell adhesion of cells, such as occurs in secondary lymphoid tissue. MMPs degrade chemokines. Cell migration and cell proliferation that is needed for specific cell type differentiation, such as in thymocyte differentiation, involve chemokines and ECM changes.

Some of the chemokines are:

CCL1 (TCA-3) is a member of CC beta family and induces chemotaxis in immune cells. CCL2 (MCP-1) displays chemotaxis for immune cells such as monocytes or basophils and is induced by PDGF in cells such as fibroblasts. CCL2 generates superoxide anions, regulates adhesion molecule and cytokine production in monocytes, and activates and enhances histamine release from basophils. CCL2 has roles in leukocyte accumulation at lesion sites, inflammation and other disease states including atherosclerosis and delayed hypersensitivity reactons. CCL2 binds to CCR1. MIPs (macrophage inflammatory proteins 1 to 3) are present in T and B cells and monocytes after antigen or mitogen stimulation. They are chemoattractants for immune cells such as monocytes and eosinophils and induce histamine secretion from basophils. CCL3 (MIP-1α, 70 aa) and CCL4 (MIP-1β, 69 aa) are produced by macrophages, T and B cells and monocytes after antigen or mitogen stimulation. Both chemokines are inflammatory proteins, monocyte chemoattractants, inhibitors of hematopoietic stem cell proliferation, and have adhesive effects on lymphocytes. CCL5 (RANTES) is expressed in T but not B cells, fibroblasts (e.g. synovial), renal tubular epithelium and tumor cells. RANTES has a role in mediating immune and inflammatory process, chemotaxis on monocytes and esoinophils through thrombin stimulated platelets. CCL6 is expressed in monocytes, neutrophils, T cells and is induced by GM-CSF or IL-4. CCL6 is a chemoattractant for monocytes. CCL7 (MCP-3 or MARC) is a monocyte, eosinophil and T-lymphocyte chemoattractant. MCPs-1 to -3 induce histamine secretion from basophils. CCL10 (interferon γ inducible protein 10) is induced by IFNγ, LPS, IL-1β, TNFα, IL-12 and viruses in monocytes, fibroblasts, endothelial cells, keratinocytes, osteoblasts, astrocytes, smooth muscle cells, splenocytes and activated T lymphocytes. CCL10 is an inhibitor of angiogenesis, has an antitumor effect that is thymus dependent and is a chemoattractant for T cells and others. Its receptor is highly expressed in IL-2 activated T cells. CCL11 (eotaxin) is an eosinophil chemoattractant. CCL12 (SDF-1α, stromal cell derived factor I α) is a chemoattractant for T cells and monocytes and is an inhibitor of infection by HIV-1. SDF-1α and SDF-1β are mitogenic for stromal cell dependent pre-B cells. SDF is made in a number of cells including fibroblasts. CCL12 is a ligand for CSCR4. CCL13 (MCP-4) is produced by endothelial, epithelial (bronchial, type II alveolar) cells, lymphocytes, macrophages, amongst others. CCL14 is present in plasma and various tissues such as muscle, liver, gut, bone marrow and spleen. It promotes chemotaxis of T cells, monocytes, eosinophils and inhibits HIV-1 M-tropic infection. Plasmin or uPA mediates CCL14a propeptide conversion to active peptide. CCL15 (MIP-1δ, leukotactin-1) is made by T, B, NK and dendritic cells and monocytes. It is chemotactic for T cells, eosinophils, monocytes and suppresses colony formation by granulocyte-macrophage, erythroid and multipotent progenitor cells. CCL16 (HCC-4) is expressed in liver and is a lymphocyte and monocyte chemoattractant. CCL17 is expressed in thymus, lung, small intestine, colon and peripheral blood mononuclear cells. It is chemotactic for T cells and is a ligand for CCR-4 present on T cells. CCL20 (MIP3α) is chemotactic for lymphocytes, inhibits proliferation of myeloid progenitors and is a ligand for CCR-6 present on cord blood precursors (dendritic cells). CCL19 (MIP3β) is chemotactic for lymphocytes, a ligand for CCR-7 present on lymphoid tissues, B and T cells, and is down regulated by anti-inflammatroy IL-10. Midkine, a 15 kDa heparin-binding molecule produced by endothelial cells, astrocytes and epithelial cells (renal tubule and Wilms' kidney tumor) is present in Alzheimer's disease senile plaques. Midkine has a role in epithelial-mesenchymal interactions and nervous system development, such as neuronal outgrowth. CCL21 (6Ckine) is a CC chemokine made in lymphoid tissues and is a chemoattractant for lymphocytes such as T cells and thymocytes, but not for monocytes. CCL22 (macrophage-derived chemokine, MDC) is expressed in macrophages, monocytes and dendritic cells and is an immune cell chemoattractant. CCL23 (myeloid progenitor inhibitory factor, MPIF-1) is present in bone marrow, lung, liver, amongst others, is a ligand for CCR1 and is a chemoattractant and activator of dendritic cells, monocytes, and osteoclast precursors. CCL24 (eotaxin-2) is a chemoattractant for eosinophils, basophils and resting T cells. CCL25 (thymus-expressed chemokine or TECK) is a CC chemokine expressed by dendritic cells in the thymus and small intestine. CCL25 is chemotactic for activated macrophages, dendritic cells and thymocytes. CCL26 (Eotaxin-3) is produced in vascular endothelial cells, heart and ovary. It induces chemotaxis. CCL28 is expressed in epithelial cells and is a chemoattractant.

CXCL1 (GROα) activates immune cells such as neutrophils, monocytes, T lymphocytes, basophils, B cells and other cell types such as fibroblasts, melanocytes, endothelial and melanoma cells. It is made by normal cells during growth stimulation and in tumorigenic cells. GRO is induced by serum, PDGF, and inflammatory mediators (IL-1, TNF) in fibroblasts, monocytes, melanocytes and epithelial cells. The three GRO proteins are neutrophil attractants and activators (basophils also). They bind IL-8 receptor type B. CXCL5 is an epithelial cell derived neutrophil activating peptide produced in monocytes and neutrophils and is induced by proinflammatory cytokines IL-1 and TNF in fibroblasts (e.g. pulmonary), endothelial and epithelial cells. It is a neutrophil attractant and activator. CSCL6 (granulocyte chemotactic protein-2, GCP-2) is a neutrophil chemoattractant and produced by LPS induction of fibroblasts. CXCL7 (NAP-2) binds to CSCR-2, activating and chemoattracting neutrophils and basophils. CSCL10 is induced by IFNs α, β, γ and LPS in astrocytes, microglia and macrophages. It is present also in T cells, splenocytes, keratinocytes, astrocytes, smooth muscle cells and osteoblasts. It is a chemoattractant for T cells, an inhibitor of angiogenesis and has anti-tumor effects. CXCL13 is a B lymphocyte chemoattractant. CX3CL1 (fractalkine) is membrane bound and cleaved to a soluble form. It is upregulated in endothelial cells and microglia by inflammation. It is chemotactic for T cells, monocytes, neutrophils and promotes leukocyte adhesion.

XCL1 (lymphotactin) has chemotactic activity for NK cells and lymphocytes. CINCs (cytokine-induced neutrophil chemoattractants) are a group of CXC chemokines that are neutrophil attractants and activators. CINCs play a role in neutrophil infiltration into inflammatory sites and are neutrophil chemoattractants. CINCs are made by fibroblasts, macrophages and in granulation tissue. IP-10 targets monocytes, T and NK cells, TIL (tumor infiltrating lymphocytes), hematopoietic stem cells and endothelial cells. PF-4 targets neutrophils, monocytes, mast cells, eosinophils, hematopoietic stem cells, fibroblasts, endothelial and tumor cells. SPF-1 targets neutrophils, monocytes, T lymphocytes, and hematopoietic stem cells. MIG targets T lymphocytes and TILs. ENA (epithelial cell-derived neutrophil-activating peptide) is a member of the CSC subfamily of chemokines. It activate neutrophils, chemotaxis and elastase release. KC is a member of the CXC subfamily and a neutrophil attractant and activator. KC plays a role in inflammation and monocyte arrest on atherosclerotic endothelium and has a role in Alzheimer's disease. LIX (LPS induced CSC chemokine) is produced by epithelial cells and fibroblasts that are stimulated with LPS or other agents. It is downregulated by dexamethasone. It is a chemoattractant and activator for neutrophils and binds the CSCR2 receptor. MAG (myelin associated glycoprotein) is a type I transmembrane glycoprotein with 5 Ig-like domains in the extracellular domain. MAG is an adhesion protein as part of the immunoglobulin sialoadhesin superfamily. It is expressed on Schwann cells and myelinating oligodendrocytes. It has a role in the interaction between axons and myelin. Soluble MAG is present in the plasma and tissues and can contribute to inhibition of neuron regeneration after injury. Viral CMV UL146 and 147 proteins are similar in sequence to CXC chemokines and induce chemotaxis and degranulation of neutrophils. Viral MCV type II chemokine like protein inhibits monocyte chemotaxis.

Chemokines effect their actions by binding to receptors on specific cell types. Some of the interactions are:

Polymorphonuclear cells express CCR1 binding MIP-1α, RANTES and MCP-3, and CCR8 binding ligand 309. B cells express CCR7 binding MIP-3b/ELC. Macrophages express receptors CCR1 binding MIP-1α, RANTES and MCP-3, CCR2 binding MCP-1 to -4, CCR5 binding RANTES, MIP-1α, and MIP-10 and CCR8 binding ligand 309. Eosinophils express receptors CCR1 binding MCP-3, 4, MIP-1α, RANTES, CCR2 binding MCP-3, 4, eotaxin-1, RANTES, and CCR3 binding eotaxin, MCP-3, -4 and RANTES. Basophils express CCR2 for MCP-1 to -5, CCR3 for MCP-3, -4, eotaxin-1, -2, RANTES, and CCR4 binding TARC. Monocytes express receptors CCR1 binding MCP-3, -4, MIP-1α, RANTES, CCR2 binding MCP-1 to -5, CCR5 binding MIP-1α, MIP-1β, RANTES, and CCR8 binding I-309. MDC, HCC-1, TECK are additional chemokines acting on monocytes. Activated T cells express receptor CCR1 binding MCP-3, -4, MIP-1α, RANTES, CCR2 binding MCP-1 to -5, CCR4 binding TARC, CCR5 binding MIP-1α, MIP-1β, RANTES, CCR7 binding MIP-3β, CX3CR3 binding IP-10, MIG, I-TAC. Activated T cells use the chemokines PARC, SLC and exodus-2 also. Resting T cell express receptor CCR3 binding eotaxin, MCP-3, -4, RANTES, CCR6 binding MIP3α/LARC, CCR8 binding ligand 309. Additional chemokines acting on resting T cells are PARC, DC-CK1, lymphotactin and SDF-1. Dendritic cells express CCR1 binding MCP-3, -4, MIP-1α, RANTES, CCR2 binding MCP-1 to -5, CCR3 binding MCP-3, -4, eotaxin1, 2, RANTES, CCR4 binding TARC, CCR5 binding MIP-1α, MIP-1β, RANTES, CCR6 binding MIP-3α, and CXCR4 binding SDF-1. Other chemokines acting on dendritic cells are MDC and TECK. Neutrophils express CSCR1 binding IL-8 and GCP-2, CSCR2 binding IL-8, GCP-2, GRO-α,-β,-γ and ENA-78. Other chemokines acting on neutrophils are NAP-2 and LIX. Natural killer cells express CCR2 binding MCP-1 to -5, CCR5 binding MIP-1α, MIP-1β, RANTES, CX3CR1 binding fractalkine and CXCR3 binding IP-10, MIG and I-TAC.

Immune cells, chemokines and cytokines are involved in a number of disease states including:

Neutrophils and IL-8, GRO-α,β,γ, and ENA-78 are involved in inflammatory disease, such as acute respiratory distress syndrome. Neutrophils and IL-8, ENA-78 are involved in bacterial pneumonia. Eosinophils and MCP-1, -4, T cells and MIP-1α, monocytes and eotaxin and basophils and RANTES are involved in asthma infiltrates. T cell, monocyte infiltrates and IP-10 are involved in sarcoidois. Monocytes, neutrophils and MIP-1α, MCP-1, IL-8, ENA-78 are involved in rheumatoid arthritis. Monocytes, neutrophils and MIP-1β are involved in osteoarthritis. Monocytes and MCP-1, T cell and RANTES, neutrophil and IP-10 are involved in glomerulonephritis. T cell and MCP-1 and neutrophil and IP-10, MIG, GRO-β, IL-8 are involved in psoriasis. Monocytes and MCP-1, neutrophil and MIP-1α, T cells and eotaxin, eosinophils and IP-10 and IL-8 are involved in inflammatory bowel disease. T cell and MCP-1 to -4, and monocyte and IP-10 are involved in atherosclerosis. T cell and MCP-1 and monocyte and IP-10 are involved in viral meningitis, while neutrophils and IL-8 and monocytes and GRO-α, MCP-1, MIP-1α and 1β are involved in bacterial meningitis.

Some major receptor category, receptor type and ligand binding chains for growth factors, cytokines or chemokines are:

The hematopoietin domain receptor category: 1) IL-6 receptor type for IL-12 binding to (IL-12Rβ1 and β2 chains), leptin binding to (leptin R dimer) and G-CSF binding to (G-CSF R dimer); 2) IL-6 and gp130 shared receptor types for IL-6 binding to (IL-6rα and gp130 chains), IL-11 binding to (IL-Rα and gp130 chains), OSM binding to (OSMRα or LIFRα and gp130 chains), LIF binding to (LIFRα and gp130 chains), CNTF binding to (CNTFRα, LIFRα and gp130 chains); 3) GH monomeric receptor type for EPO binding to (EPO-R chain), TPO binding to (TPO-R or c-Mlp chain), growth hormone (GH) binding to (GH R chain), prolactin binding to (PRL R chain); 4) IL-2 shared γ chain receptor type for IL-2 binding to (IL-Rα, IL-Rβ, and γc chains), IL-4 binding to (IL-4Rα and γc chains), IL-7 binding to (IL-7Rα and γc chains), IL-9 binding to (IL-9Rα and γc chains), IL-13 binding to (IL-13α and IL-4α chains), IL-15 binding to (IL-15Rα, IL-2Rβ and γc chains); 5) IL-3 shared β chain receptor type for IL-3 binding to (IL-3Rα and βc chains), IL-5 binding to (IL-5Rα and βc chains), GM-CSF binding to (GM-CSFRα and βc chains).

The Class II cytokine receptor category: Heterodimeric interferon receptor type for IL-10 binding to (IL-10R1 and IL-10R2 chains), IFNγ binding to (IFNGR1 and IFNGR2), and IFNα/β binding to (IFNAR1 and IFNAR2).

Phosphotyrosine kinase (PTK) receptor category: 1) class I (cysteine) receptor type for EGF binding to (EGF R chain), TGFα binding to (EGF R chain), amphiregulin binding to (EGF R chain), HB-EGF binding to (EGF R chain), BTC binding to (EGF R or ErbB4 R chain), HRGs binding to (ErbB2, ErbB3 or ErbB4 chain), GGF binding to (ErbB2, ErbB3, or ErbB4 chain); 2) class II (cysteine) receptor type for insulin binding to (insulin R, IGF-I R, or IGF-II R chain), IGF-I binding to (IGFI R chain) and IGF-II binding to (IGFI R, IGFII R, or insulin R chain); 3) class III (Ig) receptor type for CSF-1 binding to (M-CSF R chain), SCF binding to (c-Kit R chain), Flk-2L binding to (Flk-2 R chain), PDGF-A binding to (PDGFα and PDGFβ R chain) and PDGF-B binding to (PDGFα and PDGFβ R chain), VEGFs binding to (VEGFR-1, -2, or -3 chain), PIGF binding to (VEGFR-1 chain); 4) class IV (Ig, heparin) receptor type for FGFs binding to (FGF R-1, -2, -3, -4 chain); 5) class V (cysteine) receptor type for NGF binding to (TrkA or p75NGF R chain), BDNF binding to (TrkB or p75NGF R chain), NT-3 binding to (TrkC or p75NGF R chain), NT-4 binding to (TrkB or p75NGF R); 6) class VI (c-Met) receptor type for HGF binding to (HRG-R (c-Met) chain).

Serine/threonine kinase receptor category: 1) TGFβ, class I, II, III receptors type for TGFβ-1 to -5 binding to (TGF-βR type I, II and III chains); 2) TGFβ, class I, II receptor type for activin, inhibin, BMPs binding to TGFβR/BMPRs types I and II chains.

TNF receptor category: TNF receptor type for TNFα binding to (p75TNF R, p55 TNF R chain), TNFβ binding to (p75TNF R, p55 TNF R chain, LTR), CD40 ligand binding to (CD40 R chain), CD27 ligand binding to (CD27 R chain), Fas ligand binding to (Fas R chain), RANK ligand binding to (RANK R, OPG R chain).

Ig-like receptor category: Ig-like receptor type for IL-1α binding to (IL-1R chain), IL-1β binding to (IL-1R chain), and IL-18 binding to (IL-18 R chain).

Serp.7 transmembrane G protein coupled receptor category: 1) C-X-C cytokine receptor type for IL-8, GRO, MIP-2, NAP binding to CXC(α) chemokine receptors; 2) C-C cytokine receptor type for MCP-1-3, RANTES, MIP-1 binding to the chain of CC(β) chemokine receptors.

Hormones

There are four main types of hormones: 1) peptides, protein and modified amino acid hormones 2) steroid hormones 3) tyrosine or amine-derived hormones and 4) fatty acid derivatives. Peptide and amine hormones are water soluble, circulating freely for a very limited amount of time and before being degraded. Protein hormones can have binding proteins to transport to target cells. Steroid and thyroid hormones are lipid soluble and carried by plasma bound proteins in the blood with long plasma half-lives.

Hormones can be autocrine, paracrine or endocrine in nature, although endocrine actions predominate. In autocrine action, the cell signals itself by a chemical it synthesizes and can occur in the cell cytoplasm or at the receptor on the cell surface. Paracrine signals diffuse from one cell and interact with receptors on nearby cells, such as the case with inflammatory cytokines and synaptic neurotransmitters. Endocrine signals are chemical secreted into the blood and carried by blood and tissues to the target cells. Hormones in all three mechanisms, just as the case with growth factors, cytokines and chemokines, are present in the serum and ECM and in some cases other fluids in the body (nervous system, lymph).

Hormones to be used in tissues (e.g. connective) for a number of tissue repair or augmentation of defects are listed below. Most hormones are well-known and their modes of action are known to those versed in the art. Hormones and growth factors are interchanged in terminology at times. For example, EPO is an endocrine hormone but is often classified as a growth factor.

Hormones, as with the growth factors, cytokines and chemokines, can be added to specific cell types in vitro and in vivo to inhibit apopotosis, anoikis and protease activity, increase ECM production, increase cell adhesion, cell spreading, cell migration, cell proliferation, promote differentiation, enhance metabolism for optimal survival and cell activity, and regenerate tissue. These attributes can be used to treat tissue defects.

A few hormones circulate dissolved in the blood, but most are carried in the blood bound to soluble plasma proteins. Hormone and growth factor binding proteins (HBPs) are in extracellular fluids such as blood.

Many of the hormones are: endothelin-1 (a potent endogenous vascocontrictor and smooth-muscle mitogen), thyroid-stimulating hormone (TSH, 201 aa protein), follicle-stimulating hormone (FSH, 204 aa protein), luteinizing hormone (LH, 204 aa protein), luteinizing hormone releasing hormone, prolactin (PRL, 198 aa protein), growth hormone (GH, 191 aa protein), adrenocorticotropic hormone (ACTH, 39 aa peptide), antidiuretic hormone (ADH, vasopressin, 9 aa peptide), oxytocin (9 aa peptide), thyrotropin-releasing hormone (TRH, 3 aa peptide), gonadotropin-releasing hormone (GnRH, 10 aa peptide) acts on the pituitary gland controlling amounts of many different types of hormones including sex steroids estrogens and androgens, a synthetic analogue of GnRG is the triptorelin peptide (Pyr-His-Trp-Ser-Tyr-D-Trp-Leu-Arg-Pro-Gly-NH2), growth hormone-releasing hormone (GHRH, 40 aa peptide), corticotrophin-releasing hormone (CRH, 41 aa peptide), somatostatin (14 and 28 aa peptide) inhibits release of growth hormone and gastro-entero-pancreatic peptide hormones, melanocyte stimulating hormone (MSH), dopamine (tyrosine derivative), melatonin (tryptophan derivative), thyroxine (tetra-iodinethyronine or T4, triiodinethyronine or T3, tyrosine derivatives), calcitonin (32 aa peptide), parathyroid hormone (PTH, 84 aa protein), steroids such as glucocorticoids (cortisol, corticosterone), mineralocorticoids (aldosterone), androgens (testosterone, dihydrotestosterone), estrogens (estradiol, estratriol, estrone), progesterone, androstenedione, pregnenolone, dehydroepiandrosterone (DHEA), DHEA-S, androstenediol, 7-keto DHEA, human chorionic gonadotropin (HCG), adrenaline (epinephrine, tyrosine derivative), noradrenaline (norepinephrine, tyrosine derivative), insulin (51 aa protein), glucagon (29 aa protein), amylin (37 aa protein), glucagon like peptide (GLP-1) has cytokine activity promoting differentiation, tissue regeneration, and cytoprotection, erythropoietin (EPO, 166 aa protein), calcitrol (steroid derivative), calciferol (vitamin D3), atrial-naturetic peptide (ANP, 28, 32 aa peptides), gastrin (14 aa peptide), secretin (27 aa peptide), cholecystokinin (CCK, 8 aa peptide), neuropeptide Y (36 aa peptide), neurophysins, orexins, ghrelin (28 aa peptide), PYY3-36 (34 aa peptide), insulin-like growth factors (IGF-1, 70 aa protein), angiotensinogen (485 aa protein), thrombopoietin (332 aa protein), leptin (167 aa protein), adiponectin (117 aa protein), renin-angiotensin system, retinoids, proliferin, calcitonin, serum gonadotropin, placental growth hormone (PGH), prolactin, buserelin, goserelin, leuprorelin, pineal peptides (epithalmin) and hormones, and angiotensins (e.g. 1-7). Other protein and peptide hormones are also classified as growth factors.

Leptin is made by adipose tissue, involved in hematopoiesis, body weight, reproduction and cell proliferation of specific cells. The leptin receptor (OB-R), is a type I cytokine transmembrane protein that includes a soluble form containing the extracellular domain. OBR is present in the hypothalamus and many other tissues including lung, kidney, progenitor hematopoietic cells, and the chorid plexus. The soluble OB-R can be an antagonist to leptin.

Insulin stimulates glucose and amino acid uptake for cell metabolism and growth. The hormone promotes glyconeogenesis, fatty acid synthesis and transport, amongst many other actions.

Prolactin (PRL) is made by the anterior pituitary, placenta, brain, fibroblasts (e.g. dermal), uterus, deciduas, T, B, NK and breast cancer cells. It is a lactogenic hormone and involved in reproductive function and immuoregulation as a proliferative growth factor secreted by PBMC. Prolactin stimulates PBMC to make IFNγ. Prolactin receptor is a type I transmembme glycoprotein present in hypothalamus, liver, kidney, ovaries, testis, prostrate, seminal vesicles, neutrophils, macrophages, monocytes, CD34+ progenitor, NK, T and B cells.

GRP (gastrin releasing peptide or bombesin) is a 3 kD polypeptide from neural and endocrine cells that targets fibroblasts, smooth muscle cells, neurons and small-cell lung carcinoma cells.

Atrial natriuretic peptide (ANP) is made in response to increased blood volume and venous pressure within the atria. ANP causes vasodilation of peripheral and renal blood vessels.

Hormone replacement for men with testosterone can reduce serum levels of pro-inflammatory cytokines (IL-1β and TNFα, VCAM-1 in endothelial cells, prothrombotic PAI-1 and fibrinogen, LDL, body mass index and visceral fat mass. This hormone replacement can increase antiiflammatory cytokines (IL-10), vascular endothelial and smooth muscle cell proliferation, vasodilation, and insulin sensitivity. Hormone replacement therapy for women with endogenous hormones can also be anti-atherosclerotic.

Free fatty acid derivatives such as eicosanoids are hormones. HETE (hydroperoxyeicosatetranoic acid), the leukotrienes such as B4, B4 R1 and cysteinyl leukotriene, the prostaglandins including PGE 1, 2, PGF1a, PGJ2, and thromboxanes such as A2 and B2, are eicosanoids. Some of the functions eicosanoids are involved in are: HETE suppreses rennin production, stimulates insulin secretion, induces cell adhesion (tumor cells) and endothelial cell retraction, induces angiotensin II induced aldoserone production, and is chemotactic for leukocytes (polymorphonuclear and smooth muscle cells (vascular). Cysteinyl leukotriene is an inflammation mediator, causes smooth muscle contraction and increases capillary permeability. PGE1 increases cell proliferation (e.g. vascular smooth muscle cells), is involved in vasodilation, platelet aggregation and insulin-like effects. PGE2 has action in vasodilation, regulation of renal hemodynamics, sodium excretion, bone resorption, thermoregulation and anti- and pro-inflammatory responses. PGF1a regulates water and electrolyte excretion and is a vaso- and bronchoconstrictor. PGJ2 is involved in adipogenesis. Thromboxane A2 is involved in platelet aggregation, vasoconstriction and reproduction while B2 is a marker for cirrhosis of the liver and thrombosis diseases.

Metabolites and related family hormones to the above hormones can be used. Inhibitors to these in situ hormones where indicated can be used. Many of these hormones and growth factors change with age in concentration. Addition of specific hormones to the cells in vitro or in the cell implantate affect a number of cell parameters similar to growth factors, cytokines and chemokines including proliferation, adhesion, migration, spreading, survival, apoptosis, differentiation, among the many other parameters already mentioned in the invention and are useful to treat defects.

As in previous sections, inhibition of unwanted hormone, growth factor, cytokine, chemokine action can be done by use of antibodies to the receptors or ligand, natural inhibitors, binding proteins, antisense oligonucleotides, interfering RNAs and soluble receptors. The use of these factors in a profashion can be done by the use of the natural, pro or precursor forms, recombinant, fragment, domain, or binding protein forms and synthesized protein or peptides representing the active properties of the growth factor, cytokine, chemokine or hormone. Other strategies using these factors are available for use as well.

Cell Proliferation—Mitogens

Mitogens stimulate cell division. Cell proliferation is increased by mitogen pathways and inhibited by growth inhibitor pathways (such as the p53 pathway).

Molecules, mainly protein, that enhance or stimulate cell proliferation in vitro and in vivo can be used in the invention. This includes known growth factors, cytokines, chemokines, and hormones acting in autocrine, paracrine and endocrine fashions, ECM and serum proteins that affect the cell cycle. For example, ECM proteins can regulate specific protein expression during cell proliferation by stimulation of mitogen-activated protein kinases downstream of integrin activation. Mitogenic cascades can take place in tissues by specific pathways such as: hormone receptor-adenylate cyclase-cDMAP protein kinase, hormone receptor-tyrosine protein kinase, and hormone receptor-phospholipase C pathways. The receptor tyrosine kinase consists of 2 branches, those growth factors that can proliferate and suppress differentiation like EGF and those growth factors that are either mitogenic or are needed for proliferation by other factors without being mitogenic by themselves, such as FGF, insulin or IGF-1. Many signaling pathways exist for mitogenesis.

Mitogens stimulate a wide variety of cells. Thus, PDGF acts on fibroblasts, smooth muscle cells, neuroglial amongst others, EGF acts on epidermal, epithelial and nonepithelial cells, erthropoeitin primarily induces red blood cell precursors and TFG-β stiumlates cell some cell types and inhibits others.

Examples of some of the mitogens for some of the cells used in the invention are given below. More complete examples are given through out the text and are known in the art.

Pre-adipocytes are stimulated to proliferate by serum, EGF, heparin, hydrocortisone and IL-11. Serum is inhibitory to pre-adipocyte differentiation. Differentiation proceeds in the presence of insulin, dexamethasone, L-thyroxine and d-biotin. Differentiated adipocytes have a nutrition medium containing serum, EGF, heparin and hydrocortisone. Adipocytes produce TGF-β, IGF-1, IL-8, IL-6, angiopoietin-like 4/PGAR, TNF-α, M-CSF, VEGF, leptin, resistin, ASP (acylation stimulating protein), and adiponectin extracellularly. ACRp30 (adiponectin apM1) accounts for 0.01% of total plasma protein and can induce apoptosis of immune cells. Adiponectin is an insulin-sensitizing peptide.

Epidermal cell proliferation is stimulated by the growth factors EGF, HB-EGF, TGFα, βFGF, βNGF, FGFs (FGF-7 and 10), the interleukins, pituitary hormones and other immune cytokines. These cells produce IL-1α that release IL-6 from keratinocyte and stimulates their growth. Keratinocytes make also IL-3, IL-4, IL-8 and GM-CSF. Keratinocytes convert testosterone to 5 alpha dihydrotestosterone. Androgens and vitamin A are mitogenic for keratinocytes. Keratinocyte-derived factors regulate proliferation and differentiation of epidermal melanocytes. Mitogens for keratinocytes include IL-8, βNGF, HGF, amphiregulin, KGF (FGF7), HB-EGF, pituitary hormones, EGF, TGFα, insulin, hydrocortisone, transferrin and epinephrine. Keratinocyte growth inhibitors include TGFβ, IFNα, γ, TNF and the polypeptides chalones made by suprabasal cells.

Mitogens for melanocytes include HGF, FGF6, cholera toxin, phorbol esters (TPA, PMA), hypothalamic hormones, FGF2, hydrocortisone and leukotriene C₄

Epithelial cells are stimulated to proliferate by ECM protein CYR61, pleiotrophin, heregulin, βNGF, EGF, FGF2, FGF10, HGF, amphiregulin, betacellulin, KGF, pituitary hormones, Peptide YY, prolactin, insulin, hydrocortisone insulin, glucocorticoid (e.g. hydrocortisone), cholera toxin, pituitary hormones, triiodo-L-thyronine, transferrin and retinoic acid. Serum and androgens can inhibit proliferation of epithelial cells.

Endothelial cells are stimulated to proliferate by ECM-1, ECM protein CYR61, pleiotrophin, βNGF, EGF, FGF2, FGF4, FGF5, FGF6, FGF10, VEGF, EG-VEGF, PD-ECGF, HGF, betacellulin, GM-CSF, IL-1, pituitary hormones, serum, heparin, hydrocortisone, IGF-1 (long R3), pituitary hormones, angiogenin, fetuin, apo transferrin (low iron) or holo transferrin (iron saturated).

Smooth muscle cells are stimulated to proliferate by serum, EGF, FGF2 and insulin.

Skeletal muscle cells are stimulated to proliferate by serum, fetuin, EGF, FGF2, insulin and dexamethasone. Skeletal muscle cells differentiate in the presence of insulin.

Fibroblast proliferation is stimulated by TGFα, TGFβ, TNF, IL-1, PDGF (AA, AB), CTGF, thrombin, coagulation proteases, blood coagulation Factor Xa, VIIa, and XIIIa, fibrinogen, soluble partially degraded fibrinogen, EGF, HB-EGF, FGFs (e.g. FGF-2, 4, 5, 6, 9, 17), IGF (e.g. IGF-1), insulin, various interleukins (e.g. IL-1), MDGF (leukocyte-derived growth factor, LDGF-3), angiotensin II, endothelin-1, urokinase-type plasminogen activator (uPA), CYR61, oncostatin M, pleiotrophin, leukemia inhibitory factor, amphiregulin and betacellulin. Dermal papilla fibroblasts are stimulated to proliferate by pituitary hormones. Non-protein factors can be used in the invention. For example, asiaticoside, a triterpene glycoside, increases cell proliferation, including fibroblasts such as dermal fibroblasts.

Certain lectins induce mitogenic activity such as concanavalin A, pokeweed lectin, a variety of agglutins such as leucoagglutinin PHA-L and phytohemagglutinin PHA-P.

The cell cycle is characterized by 4 sequential phases, the G1 to S (DNA replication) to G2 to M (mitosis, cytokinesis) phases. The G1 and G2 phases allow the cells to adjust to internal and external environments before committing to the major S and M phases. G1, especially can delay cell division if the environmental signals are not favorable. In fact, a G0 phase can be reached, a specialized resting phase in which cells can remain in for long periods before resuming cell proliferation. After reaching a commitment point in G1 the cells go on to the S phase.

Most of the events of the cell cycle are initiated by cyclin-Cdk (cyclin dependent kinase) activities. During the G1 phase Cdk activity is low due to Cdk inhibitors (CKIs), cyclin proteolysis and decreased cyclin gene transcription. An increase in G1 and G1/S-Cdks overcome the inhibitors in late G1 and this activates S-Cdk which in turn phosphorylates proteins at DNA replication origins triggering DNA synthesis. After S phase, M phase Cdk is activated leading to mitosis. Maturation promoting factor is a protein kinase that drives the G2/M phase transition. M-Cdk is inactivated by cyclin proteolysis ending the M phase and the start of cytokinesis. Thus the cell cycle is controlled at various checkpoints by inhibitory mechanisms, DNA repair and extracellular conditions.

Proteins, mainly enzymes, control the cell cycle. Cdks (cyclin dependent kinases) are a family of protein kinases that change activities as the cell progresses through the cycle. Cyclins are a main regulator of the Cdks by binding the Cdks and altering their activity by cyclic changing concentrations of cyclin throughout the cell cycle. The activation of the cyclin-Cdk complexes triggers cell cycle events. There are 4 classes of cyclins: the G1/S cyclins (cyclin E) that bind Cdks (Cdk2) at the end of G1, committing the cell to DNA replication. The S-cyclins (e.g. cyclin A) bind Cdks (Cdk2) during S phase and is needed for DNA replication initiation. The M cyclins (e.g. cyclin B) bind Cdks (e.g. Cdk1) and promotes mitosis. The G1 cyclins (cyclin D) bind Cdks (e.g. Cdk4, Cdk6) and promote passage of the cells through the restriction point in late G1. Full activation of the cyclin-Cdk complex is performed by CAK (Cdk activating kinase). Cyclin-Cdk complexes can be inhibited by phosphorylation by the Wee1 kinase and their activity increased by a phosphatase Cdc25. Also, Cdk inhibitor proteins (CKIs) regulate cyclin-Cdk complexes. The cyclins are proteolyzed by a ubiquitin-dependent mechanism as are many other intracellular proteins. The rate-limiting step is catalzyed by the ubiquitin ligases. Cdk activity is controlled in G1 by Hct1 activation, an increase in p27 protein and repression of cyclin gene transcription. E2F activates S phase gene expression by binding to many genes that encode proteins needed for S phase entry (G1/S cyclins and S cyclins). Rb, unphosphorylated retinoblastoma protein, inhibits cell cycle progression by binding E2F.

G1 checkpoint blocks progression into S phase by inhibiting activation of G1/S-Cdk and S-Cdk complexes. p53, a major gene regulatory protein produced for example when DNA damage occurs, increases transcription of other genes such as p21, a CKI protein that binds to G1/s-Cdk and S-Cdk and inhibits their activities. Mdm2, binds p53 acting as a ubiquitin ligase that targets it for proteolysis and controls the levels of p53. Other CKIs are p27 that suppress G1/S-Cdk and S-Cdk and p16 that suppresses G1-Cdk in G1. Some of the ubiquitin ligases and their activators are SCF, APC, Cdc20 and Hct1.

Various kinase pathways are involved in proliferation, including JNK, p38 protein kinases, ERK (extracellular signal-regulated kinase) and MAPK (mitogen activated protein kinases), a superfamily of kinases. Receptor tyrosine kinases (e.g. growth factor) can activate the MAPK signaling pathway that controls proliferation, differentiation and motility among other cell functions. A number of mitogen-activated protein kinases are involved including MAPK1-15, MAP2K1-7, MAP2K1IP1, MAP2K1P1, MAP3K1-15, MAP3K7IP1, MAP3K7IP2, MAP4K1-K5, MAPK6PS1-6, MAPK8IP1-P3, MAPK8IPP, MAPKAP1 and MAPKAPK2-K5. Sprys (e.g. dSpry) are ligand induced feedback inhibitors of a number of growth factor receptors. Inhibition of FGF and VEGF receptor activation in endothelial cells for example, occur with EGF stimulated cells. Sprys enhance MAPK activation. Maturation promoting factor is a protein kinase that drives the mitotic and meiotic cycles. Cyclins are regulatory proteins that function in the cell cycle to activate maturation promoting factor by complexing with p34cdc2, the catalytic subunit of maturation promoting factor. Cyclin dependent kinases promote cell proliferation. Rb, the retinoblastoma tumor suppressor pathway has a critical role in the control of cellular proliferation by modulating E2F activities. E2F1-3 and the E2F family of factors act as transcriptional activators for progression through the G1/S transition. pRB (retinoblastoma protein), p130, p107, p27Kip1, p19Ink4d and other cyclin-dependent kinase inhibitors can cause cell cycle arrest and thus inhibitors to these proteins can increase cell proliferation. Regulation of the Ras/Raf/MEK/ERK pathway, via receptor binding, is a common feature of cell proliferation in many systems. Thus factors that alter this pathway can control cell proliferation.

RelB is a member of the NF-κB/Rel family of transcriptional regulators and present in fibroblasts, hepatocytes, immune cells, and other cell types and skin, brain, kidney, intestinal and other tissue types. RelB is present but inactive (bound to IκB) in quiescent fibroblasts (and other cell types). RelB and NF-κB is activated by increasing DNA binding activity by the presence of PDGF, TNF-α, phorbol esters or serum. Agents that promote intracellular cAMP levels, such as PDGF, traverse the G0/G1 cell cycle phase. RelB promotes cell proliferation. PDGF initiates and maintains cell cycle traverse in both quiescent and cycling cells. NFκB family of transcription factors consists of RelA, Rel B, c-Rel, p100, p105, NFκB1 and NFκB2. The pathway produces several growth factors, cytokines, chemokines and receptors and anti-apoptotic proteins that stimulate cell proliferation in specific cell types. In some cell types in tandem with several stimuli the pathway can lead to apoptosis.

Oncogenes, protooncogenes in general increase and tumor suppressors decrease mitogenicity.

Paracrine, autocrine or endocrine action by growth factor, cytokines, chemokines or hormones, present in the serum, ECM and tissue fluids can inhibit specific cell proliferation. For example, TGF-β is cytostatic to many epithelial cells. The most common inhibitors act at the G1 level of the cell cycle. Proteins that counteract the inhibitors or act by entering the cells back into the cell cycle are useful for cell proliferation of cells.

Primarily extracellular proteins such as growth factors, ECM and serum proteins that control various intracellular protein activities to regulate the cell cycle are useful in the invention. Some of the intracellular proteins that modulate the cell cycle and can be controlled by extracellular proteins include those listed below:

The classes of cyclins include cyclins D1, D2, G2, H, I, G1/S-specific cyclins D3, C, E, and G2/mitotic-specific cyclins A, B1, and G1. The oncogenes and tumor suppressors include p53 tumor antigen, p21, MDM2-like p53-binding protein or MDMX, p33ING1, WAF1 or wild-type p53 acitvated fragment 1, SDI1, CAP20, retinoblastoma-associagted protein 1 or RB1, RB2 or RBL2 retinoblastoma-like protein 2, 130 kDa retinoblastoma-associated protein, CHOP or C/EBP homologous protein and jun-B, N-myc proto-oncogene, c-myc proto-oncogene, c-myc-binding protein MM-1, prefolding 5, rafl proto-oncogene, GRB-IR/GRB10, B-raf proto-oncogene or RAFB1, CDC42 GTPase-activating protein, Abl interactor 2 or Abl-2, and Abl binding protein 3. The DNA polymerases, replication factors, and topoisomerases include of proliferating cyclic nuclear antigen or PCNA, cyclin, replication factors C 36 kDa, C 37 kDa, C 38 kDa, C 40 kDa, single-stranded DNA binding protein, replication protein A 70-kDa. The DNA synthesis, recombination and repair proteins include ubiquitin-protein ligase, ubiquitin-conjugating enzyme E2A, ubiquitin carrier protein, HR6A, ataxia telangiectasea mutated protein, DNA damage-inducible transcript 1 and 3, and RAD23A. The chromatin proteins, histone acetytransferases, deacetylases, transcription proteins and factors including activators and repressors include CAF1 p48 subunit, retinoblastoma-binding protein 4, RBAP48, msi1 protein homolog, RBP2 retinoblastoma binding protein, RBQ1 retinoblastoma binding protein, RBQ3, RBBP3, serum response factor and binding protein, PRB-binding protein E2F1, E2F transcription factors 3, 5, p73, PURA or purine-rich element-binding protein A and single-stranded DNA-binding protein alpha or PUR-alpha and transcription factor DP2. The CDK inhibitors include cyclin-dependent kinase inhibitor 2A or CDKN2A, other CDKNs 2B, 2D, 1A, 1C, p57, KIP2, p19-INK4D, INK4A, and wee1+homdog. The kinase activators and inhibitors include CMM2, MLM, multiple tumor suppressors or MTSs 1, 2, CIP-1 or CDK-interacting protein 1. The intracellular kinase network members include CMM3 or cutaneous malignant melanoma protein 3, PSK-J3, PSSALRE, PLSTIRE, PITALRE, KKIALRE, CDK-activating kinase 1 or CAK 1, serine/threonine kinase 1 or STK1, K35, cell division protein kinase 9, cell division cycle protein 2-like 4 or CDC2L4, p21 activated kinase 1 or PAK1, PCTK1, 2 or PCTAIRE protein kinase 1, 2, CDC2-related protein kinase, cholinesterase-related cell division controller or CHED, MAP kinase or MAPK, mitogen-activated protein kinase kinases or MAPKKs, MAPKKKs, p38, p38β, 8, 9, 10, 11, extracellular signal-regulated kinases or ERK 1, 3, 5, ERK3 related protein, p21 activated kinase 2 or PAK2, hPAK65, protein kinase B or PKB, glycogen synthase kinase 3 alpha or GSK3A and protein kinase B. The intracellular protein phosphatases include M-phase inducer phosphatase 1, 2 or MPI 1, 2. The intracellular transducers, effectors, and modulators, cytoskeleton and motility proteins and cell cycle-regulating kinases include DRTF1 polypeptide 1, cell division control protein 2 homolog or CDC2, cyclin-dependent kinases or CDKs1, 2, 4, 5, 6, 7, 8, 9, 10, cell division protein kinases 2, 4, 5, 6, 7, 8, 9, 10, CDC-like kinase or CLK1, 2, 3, cyclin-dependent kinase-like or CDKL1, polo-like kinase or PLK, cell division cycle protein 2-like 5 or CDC2L5, mitogen-activated protein kinase/ERKkinase or MEK 1, 2, 5, 6, stress-activated protein kinase kinase 3 or SAPKK3, mitogen-activated protein kinases such as MAPK 3, 4, 6, 7, 12, MAPK/ERK kinase kinase 3 or MEKK3, MAX-interacting protein 2 or MX12 and p34 protein kinase. The apoptosis associated proteins include GADD45 or growth arrest and DNA damage-inducible protein and GADD153. The death kinases include akt1 proto-oncogene and rac alpha serine/threonine kinase. The stress response proteins include stress-activated protein, or SAP, SAP kinase, jun N-terminal kinases or JNK 1, 2, 3A2. The GTP/GDP exchangers, GTPase activity modulators, G proteins, other cell cycle proteins include CDC6-related protein, CDC 10 protein homolog, CDC 16HS, CDC27HS protein, CDC37 homolog, PBR3, cyclin-dependent kinase 5 activator regulatory subunit 1 or 2 or CDK5R2 or 1, neuronal CDK5 activator or NCK5A, the isoform NCK5AI, cell division cycle 25 homolog A, B, C, HU2 or CDC25, E2F dimerization partner 1, 2 or TFDP1, 2, DRTF1 polypeptide 1 or DP1, RBP1 isoform I and II, RBQ retinoblastoma binding protein, RBQ-3, p53-dependent cell growth regulator CGR19, GAS1 or growth arrest-specific protein 1, NEDD5 protein homolog, DIFF6, KIAA0158 and ubiquitin. The G proteins include RAC1 or ras-related C3 botulinum toxin substrate 1, ras-like protein TC25, CDC42 homolog, and G25K GTP-binding protein.

Differentiation

In general as differentiation progresses, cell proliferation is reduced and eventually stopped. Differentiation culminates the expansion of non-differentiated cell types into cell types with a desired phenotype to specify appropriate tissue function to treat defects. Differentiation of precursor cells can occur in cell culture with proper addition of inducers. Upon implantation, differentiation can occur depending on the in situ environmental cues in the cells and ECM. Introduction of proteins and molecules with the implantate can effectively differentiate cells since the appropriate spatial (e.g. 3 dimensional ECM) and temporal environmental cues are already present in the tissue. These cues include cell-cell, cell-ECM and three dimensional interactions with cells. Physiological inducers of differentiation can be ECM proteins, serum proteins, hormones, cytokines, chemokines, growth factors, other macromolecules, small molecules, amongst others. Factors can also be used to maintain differentiation of the current cell type. For example, MIP-1α can maintain the stem cell phenotype while LIF prevents embryonic stem cell differention or TGFβ prevent alveolar type II differentiation.

Examples of soluble inducers of differentiation include: HGF and kidney cells (e.g. tubule formation), HGF and hepatocytes, KGF and keratinocytes and prostatic epithelial cells, growth factors and hormones and embryonic cells, melanotropin and melanocytes, thryotropin and thyroid cells, insulin, prolactin, TGFβ and epithelium, TGFβ and melanocytes, βNGF and neurons, glia maturation factor and glial cells, IFNγ and neuroblastoma, CNTF and astrocytes, EPO and erythroblasts, G-CSF, GM-CSF, IL-1, IL-6 and hematopoietic cells, calcium and keratinocytes, vitamin D and monocytes and osteoblasts, retinoids and endothelium, epithelium and cancer cells, hydrocortisone and hepatocytes, and epithelium and glia. Other examples are provided throughout the text and that which is present in the art.

Apoptosis Inhibiting Factors

Apoptosis

Loss of inappropriate cell numbers in a tissue causes tissue defects. Loss of cells are promoted by apoptosis.

Apoptosis is the intracellular programmed death of cells that is initiated by specific “death” signals. Cells require signals from other cells not only to grow and proliferate, but to survive. Without survival factors cells die by apoptosis. A good example is the competition between nerve cells for survival factors secreted by the target cells they contact during the development of the nervous system. Other cells in tissues are thought to be controlled in a similar fashion by survival signals produced by proximal or neighboring cells. Survival factors usually bind to cell-surface receptors, as does mitogens and growth factors for cell proliferation and cell growth.

Apoptotic cells undergo a programmed series of morphological changes including cytoskeletal disorganization, chromatin condensation and fragmentation (internucleosmal fragmentation), membrane blebbing, ultimate cell breakup and engulfment by surrounding cells and immune cells (phagocytes). Apoptotic markers are increases in enzyme activities such as caspase 3 activity, poly(ADP-ribose) polymerase (PARP) cleavage, decreased cellular metabolism, compromised membrane permeability, and cleavage of nuclear envelope proteins (lamins). Necrotic cells in contrast are characterized by nuclear, cytoplasmic and lysosomal membranes resulting in the cell swelling and breakage. Inhibition of apoptosis can lead to immunogenicity of the cells.

Proteins that promote apoptosis can do so through a number of pathways, including the intrinsic or mitochondrial and extrinsic or cytoplasmic pathways. Chemicals and radiation, such as chemo or radiotherapy in cancer treatments, for example initiate the intrinsic pathway. The primary pathway, the intrinsic pathway, is initiated in which the major organelle involved is the mitochondrion. Cytochrome c and Smac/DIABLO are released from the mitochondrion. Bcl-2 family members inhibit the activation and BH3only/Bax family members initiate the activation of release. Cytochrome c allows a conformational change in the cytosolic adapter molecule, Apaf-1 then permits the recruitment and oligomerization of caspase-9. Caspase-9 becomes activated. Thus caspase activation begins the apoptotic pathway. The caspases are synthesized as inactive proenzymes that are proteolyzed to form the active caspases: caspase-1 (ICE, interleukin-1β converting enzyme), caspase-2 (ICH-1), caspase-3 (CPP32, Yama, apopain), caspase-4 (TX, ICH-2, ICErel-II), caspase-5 (ICErel-III), caspase-6 (Mch2), caspase-7 (Mch3, ICE-LAP3, CMH-1), caspase-8 (MACH, FLICE, Mch5), caspase-9 (ICE-LAP6, Mch6), caspase-10 (Mch4, FLICE2), and caspase DRONC. Substrates for caspases besides other caspases, include SREBP, AP-24, D4-GDI, DFF, Lamins, PARP, MMPs, amongst others.

Caspase activation in most cells requires permeabilization of the OMM (outer mitochondrial membrane). Bax is a mitochondrial outer membrane, channel forming protein that leads to the permeability transition and release of cytochrome c into the cytosol. Cytochrome c acts as a cofactor with a host of factors for the recruitment of caspase 9 and its interaction with APAF-1 (apoptotic protease activation factor 1), resulting in cleavage and activation. Other factors include Smac/DIABLO (second mitochondria derived activator of caspase/direct IAP binding protein with low pI), which blocks the function of IAP (inhibitor of apoptosis protein), a protein that inhibits the activated caspases. OMM permeabilization initiates the whole caspase cascade, culminating in cell death. Members of the Bcl-2 family regulate OMM permeabilization.

The extrinsic pathway is initiated by death-receptor ligands. For example, Fas ligand binding to its receptor Fas or TRAIL ligand binding to its receptor initiates apoptosis. These interactions recruit procaspase-8 which then triggers caspase-8 oligomerization and autoproteolytic activation by adapter molecules FADD/Mort1. TNF-α, through TRADD, RIP, RAIDD and pro-caspase 2 activate effector caspases 3, 6 and 7. TNF-α and FAS ligand induce procaspase 8 to effect caspases and to a lesser degree through BID, the intrinsic pathway. Clustering of cellular receptors often is a first step in the signal transduction pathways that result in apoptosis.

Apoptosis is promoted by activator pathways, such as the p53 pathway or tumor necrosis factor or TNF/neuronal growth factor that binds cell surface death receptors. Apoptosis is inhibited by survival factor or anti-apoptosis pathways.

Proteins involved in proapoptosis include, but are not limited to, the ligands, ligand receptors, adaptor proteins, proteases (e.g. caspases), amongst others. Soluble TRAIL, Fas, and TNFα are some of the prominent ligands. Examples of ligands include the TNF ligand superfamily of TNF-β (TNFSF1, lymphotoxin β, LT-β), TNFα (TNFSF2, LT-α,), βNGF (nerve growth factor), BDNF, NTs-3 and -4, OX40L, TNFSF9 (4-IBB), CD30 (TNFSF8), CD27 (TNFSF7), CD40 (TNFSF5), CD95 or Fas (TNFSF6), TRAIL (TNFSF10, Tumor Necrosis Factor-related Apoptosis-inducing Ligand, Apo-2), TNFSF11A (RANK), TNFSF11B (OPG), TNFSF12 (TWEAK), TNFSF13 (APRIL), TNFSF13B (BAFF/BLyS), TNFSF14 (LIGHT), TNFSF15 (VEGI), TNFSF18 (GITR), FASα, IL-18, other interleukins, and TRANCE (TNFSF11, TNF-related activation-induced cytokine). The receptors for apoptotic ligands include the TNF/NGF (nerve growth factor) receptor family of death receptors of tumor necrosis factor receptor-1 (TNFR1), TNF RI (TNFRSF1A), TNFRII (TNFRSF1B), TNFRSF3 (LTβR), TNFRSF5 (CD40), TNFRSF6 (CD95, Apo-1/Fas), TNFRSF7 (CD27), TNFRSF8 (CD30), TNFRSF9 (4-1BB), TNFRSF11A (RANK, receptor activator of NF-κB), TNFRSF11B (osteoprotegerin, a soluble secreted protein), TNFRSF14 (HVEM), NGFR (p75 neurotrophin R), OX40, ATAR, TRAMP, TACI and TRAIL-receptors. Receptors that activate TRAIL and TRANCE signal pathways are (TNFRSF10A), (TNFRSF10B), (TNFRSF10C), (TNFRSF10D), DR3, DR4 and DR5. The mitochondrial proteins include cytochrome C, the intracellular Bcl-2 family of proteins such as BID, Biml, HRK, Bcl-w, Bik, Bcl-X, Bcl-X_{L}, Bcl-X_{S}, Bfl-1, Bax, Nip-3, Blk, Bok/Mtd, Bak, Bad, BCL2L13, BCL-2, BCL-10, A1, Smac/DIABLO, and MCL-1. Proteases include the procaspases (e.g. 3, 8, 9), the initiator caspases 2, 8, 9, the effector caspases 3, 6, 7 and caspases 1 (ICE), 4, 5, 10, 11, 12, 13, 14-20, tBid (a caspase 8 truncated form of the Bcl2 related protein Bid), APAF-1 (apoptotic protease activation factor 1) and the extracellular granzymes B, A and C. The adaptors include Fas associated death domain protein (FADD), CIDE (cell death inducing DFFA like Effector Proteins), CIDE-B, TRAF2, TRAF6, TRAF4, TRAF1, RIP, I-TRAF, Flash, Apaf-1, DAP-kinase2, Myd88, CRADD, TRAF6, Bar and Trip. Other proteins, sone of which are in the above classes, include GADD45, p53, cardiac/Rip2, Chk2, RAD 53, Mdm2, IAP-2, BCL-10, CIDE-A, RPA, Hus1, p63, p33, Rb (retinoblastoma protein), β-amyloid and fragments such as 1-40, 1-42 and 1-43 amino acids, DFF40, DFFA, Chk1, Nod/CARD4, Apollon/Bruce, FAF, DAXX, RAIDD, BH3 proteins, MADD, FAP, jun, NOP30, ATM, and perforin. Other apoptosis-related proteins include NIK, lkk1, Ikk2, Ikk3, IkB, NF-κB, TACI, NF-AT, Ras, Raf, MEK, ERK, ELK1, ASK1, MKK3, MKK6, p38, Atf2, Rac1, Pak, MEKK, NFκB, JNKKs, JNK, Jun, c-myc, N-myc, tumor suppressor genes, p53 (overexpression induces apoptosis), p33, p21, p300, Rb, β-amyloid, acinus, A1 (member of Bcl-2 family), ASC (apoptosis-associated speck-like protein), ASK-1 (Apoptosis signaling regulating kinase-1), BAFF, BAR, Bcl-10, Bcl-xs, Bim, Blys, Bnip3L, CAD (caspase activated deoxyribonuclease), CARD9, CARD11, DAP kinase 2 (Death Associated Protein), DEDAF, DNA fragmentation factor, DRAK (DAP kinase related apoptosis inducing protein kinase), endonuclease G, DNase, NAC, Pak-2, PKCδ, RICK (regulates Fas-induced apoptosis), cell cycle regulatory kinases (e.g. Cdk2, MAP kinase), p400, E1A, and surface calreticulin.

TNF-α is a potent pro-inflammatory and pro-apoptotic mediator. The cytokine activates a number of transcription factors including the forkhead box class-O 1 (FOXO1, also known as FKHR). FOXO3 or FKHR-L1 and FOXO4 or AFX modulate apoptosis through gene expression. An example of transcription factor involvement in apoptosis is Akt, a protein kinase, which phosphorylates FKHRL-1 causing FKHRL-1 to be sequestered in the cytoplasm. When cells are starved of growth factors, FKHRL-1 returns to its unphosphorylated state, enters the nucleus, transcribes FAS ligand, triggering cell death.

Hormone withdrawal such as glucocorticoids from thymocytes or serum from fibroblasts can lead to apoptosis.

Proteins can be apoptotic or anti-apoptotic depending on cell types or protein member interactions. For example, the intracellular Bcl-2 family members can have both pro or anti-apoptotic properties. Bax homodimers are apoptotic whereas Bax heterodimers with Bcl-2 or Bcl-X_{L} block cell death. Bad heterodimer with Bcl-2 promotes cell death. Bcl-2, and its close homologues BCl-X_{L} and Bcl-w have four BH domains (BH 1-4). The BH3 domain is required for the pro-apoptotic activity of not only these, but also of Bax and Bak. Certain receptor type activation, such as PAR-1 activation, can induce or inhibit apoptosis on many cell types, including fibroblasts, neuronal, endothelial, epithelial and tumor cells, depending on the dose of thrombin (natural agonist) or synthetic receptor activators. Proteins that alter the Ras/Raf/MEK/ERK pathway, a common feature of cell proliferation in many systems, can control cell proliferation and inhibit apoptosis. Activation of mitogen-activated protein kinases controlling cell proliferation can inhibit apoptosis (e.g. in fibroblasts). Sustained activation of this pathway can lead to apoptosis of fibroblasts and other cell types. The transcription factor NFκB can induce pro or anti-apoptotic genes and proteins depending on cell type.

Among the typical proteins known to promote cell survival through anti-apoptosis are: the IAPs (inhibitor of apoptosis proteins) that include survivin, IAP-1, XIAP, NAIP, DIAP1, c-FLIP, cIAP, cIAP-1, cIAP-2, CrmA, ARC, IEX-1L, Bcl-2, BIRC5, CASPER, BAG-1, Bax, Bcl-6, usurpin, ICAD, livin (baculoviral IAP repeat containing protein 7, a caspase inhibitor), Protein C and Protein A20. Survivin is expressed in human cancers, of the colon, bladder, brain, lung, skin and others. Inhibitors to anti-apoptotic proteins or proapoptotic proteins can be used to eliminate cancerous cells and maintain normal cells.

The serpin-like cowpox protein CrmA (cytokine response modifier A) and baculovirus p35 protein inhibit TNF and CD95 (FAS) induced apoptosis. P35 inhibits apoptosis triggered by many signal transduction pathways, but CrmA inhibits mostly caspases 1 and 8. Tetrapeptide sequence inhibitors incldue DEVD for caspases 3, 7 and 10 and WEHD for caspases 1, 4 and 5. VAD modified as a fluromethylketone can react nonspecifically with other proteins but is active against most caspases. When the following tetrapeptides are coupled with aldehydes they are potent inhibitors of the caspases: VEID for caspases 6, 7, 8, 1; YVAD for caspases 1, 4; LETD for caspases 8; LEHD for caspases 4, 5, 9. Other inhibitors that can be synthesized with a trapping group, that are irreversible and non-toxic include fluoromethly ketone (FMK), WEHD for caspase 1, VDVAD for caspase 2, DEVD for caspase 3, YVAD for caspase 4, VEID for caspase 6, IETD for caspase 8, LEHD for caspase 9, AEVD for caspase 10, and LEED for caspase 13. Additonal inhibitors are Ac-YVAD-CHO for caspase 1, Ac-DEVD-CHO for caspase 3, Ac-VEID-CHO for caspase 6, Ac-IETD-CHO for caspase 8, and pan inhibitors that include VKD and VAD sequences.

Growth factors, transcription factors, kinases, decoy receptors, ECM and serum proteins, amongst other proteins can inhibit apoptosis. Transcription factor proteins that inhibit caspases or other proapoptosis proteins, and inhibitors of the death domain contained in the TNF receptor and other apoptotic receptors are useful. Other examples are the agonist binding of the AFP receptor (α-fetoprotein receptor) and the PDGF signaling of protein kinase B (AKT) which phosphorylates and inactivates proteins in apoptosis via activation of transcription factor NF-κB. Fibroblast growth factors such as FGF-2 or FGF-9 inhibit apoptosis of many different cell types such as epithelial, endothelial, fibroblasts, smooth muscle and neuronal cells. Binding of IL-15 to its receptor IL-15 Rα, inhibits TNFα mediated apoptosis in fibroblasts. IGF deters apoptosis. The NFκB pathway codes for proteins in anti-apoptosis in certain cell types. Decoy receptors like TRAIL decoy receptors DCR-1 to -5 lack the death domain needed for apoptosis pathways. Death domains of receptors can be bound by SODD to inhibit apoptosis. Inhibitors to the adaptor proteins that contain death effector domains or caspase activation recruitment domains or procaspases at the membrane surface that are activated by proteolysis can be useful to inhibit apoptosis.

Soluble receptors can be used to inhibit apoptosis. For example, TRANCE a member of the TNF family, binds the secreted receptor protein osteoprotegerin in which it serves as a decoy receptor. TRANCE and TRAIL are ligands for osteoprotegerin. TRANCE is also a ligand for RANK. RANK can activate NF-κB. Soluble RANK can inhibit TRANCE induced activity by competitively binding TRANCE. Similarly, decoy TRAIL receptors can be used in the same capacity. Thus soluble receptors can inhibit ligand binding to the appropriate receptors which inhibits the apoptosis pathway. Many of the apoptosis receptors can exist in soluble forms naturally as well as by recombinant DNA means. Proteins that inhibit TNF-α or other growth factor, hormone or signalling proteins can inhibit apoptosis. Thus antibodies to known proapoptotic proteins, including ligands or transmembrane receptors, or ECM or serum proteins that interact and neutralize propapoptic proteins can be used. Antibodies monoclonal, polyclonal, fusion proteins such as Fc, among others can be used to inhibit the activity of apoptotic proteins. Soluble receptors that block ligand activity and decoy receptors that antagonize ligand binding induced apoptosis, such as for TRAIL ligands and other macromolecules that bind ligands, can be used to inhibit the activity of these apoptotic proteins. Also inhibitors of proteases such as caspases blocks apoptosis. Protease inhibitors of procaspases can block apoptosis. Blocking peptides or peptides that compete against apoptosis proteins can be used. Inhibitors to various parts of the apoptotic signaling pathways can be used.

AGEs (advance glycation end-products) promote apoptosis through interaction with the RAGE receptor that ultimately reduces ECM formation. Antibodies to RAGE inhibit binding of proteins that are AGEs. Higher rates of fibroblast apoptosis is observed in aging tissues, poor wound healing, diabetic tissues and inflammation. The higher rates of apoptosis parallels the formation of AGEs in these tissues. The RAGE receptor is a member of the immunoglobulin superfamily. Addition of RAGE soluble receptor, extracellular portion of the receptor, the peptides containing the AGEs binding site, antibodies to the AGEs or to the RAGE receptor can be used to bind and remove AGEs and negate RAGE signaling. A similar strategy can be used with other ligands and receptors for apoptosis.

Inhibitors to apoptosis will allow survival of the cells in vivo. All proteins and substances that inhibit apoptosis can be used in the invention. Proteins or molecules that inhibit apoptosis can act by interaction with proapoptotic proteins and receptors or by acting as an anti-apoptotic factor itself. These antiapoptotic factors can be used singly or in combination with implanted cells for the invention. This includes proteins that control different parts of the signaling pathway for production of antiapoptotic activities.

Proteins and other molecules can be added to the cell implantate and/or cells grown in vitro to suppress the programmed death of the cells or apoptosis. These anti-apoptotic proteins and substances promote the survival of cells after implantation and is necessary to optimize the effect of the cells. Examples of some of these anti-apoptotic agents are given above. Pan caspase inhibitors to the ligands such as antibody to FAS or TNF or other ligands that promote apoptosis with cells can be used. Blocking antibodies to the receptors for the extrinsic pathway to apoptosis can be used. Inclusion of antisense, siRNAs and other intracellular agents to prevent the production of apoptotic proteins can be used.

Similar strategies for the use of promoting apoptosis can be used in the scenario wherein overproliferating cells (e.g. cancer cells, fibrosis producing cells) need to be removed.

Anoikis

There is a high rate of apoptosis during cell culture, cell isolation, cryopreservation, and engraftment, thus compromising cell transplantation or implantation. Apoptosis occurring due to cell detachment from the extracellular matrix is a phenomenon termed “anoikis.” Anoikis is the apoptotic response induced in normal cells by inadequate or inappropriate adhesion to substrate. All the features that characterize apoptosis, including nuclear fragmentation and membrane blebbing, are observed during anoikis. Anoikis was observed initially after disruption of the interactions between normal epithelial cells and extracellular matrix. Cells are critically dependent upon cell-matrix adhesion for growth and survival. Thus, the removal of extracellular or serum substrata results in the death of cells.

In the multicellular organism, cells do not exist in isolation but associate with neighbouring cells and the extracellular environment. The ECM (extracellular matrix) is a part of this environment and serves in part as the physical scaffold on which the cells adhere. The ECM also provides cells with information regarding their context within a tissue or organ, information required for proliferation, migration, differentiation and survival. Most cell-ECM interactions depend on integrins, transmembrane heterodimeric receptors for ECM proteins that associate with a large number of proteins on the cytoplasmic face of the plasma membrane, forming cell-ECM adhesion complexes (focal complexes and focal adhesions). These complexes provide a structural link between the ECM and the cytoskeleton, and act as a scaffold for signalling molecules. Signals, such as the adhesion-activated tyrosine kinases (e.g. pp125FAK), are propagated from cell-ECM adhesion complexes, activate a number of well-characterized pathways, many of which play a role in the suppression of anoikis. Spreading out from pp125FAK is a web of signalling networks, including the mitogen-activated protein kinases, PI3K (phosphoinositide 3-kinase), Src, and others. Protein kinase signaling pathways control anoikis both positively and negatively.

In an intact organism anoikis ensures that cells are unable to survive in an inappropriate location. Anoikis thus is apoptosis caused by cell isolation or cells in suspension. Anoikis is induced by loss of cell adhesion or inappropriate cell adhesion. Adhesion on the extracellular matrix is important to determine whether a cell is in the correct location and to delete displaced cells by apoptosis. The list of ECM proteins and serum proteins in this invention can provide benefit by preventing anoikis and thus promoting the survival of the cell implantate as well as for increased cell yields in vitro. Anoikis can be avoided by the inclusion of cell produced ECM, undegraded ECM which contain cell binding sites, partially degraded ECM that still contain cell binding protein domains to the cell receptors, individual or combined ECM consitutuents, fragments of ECM with cell binding sites intact, recombinant or man-made protein sequences containing cell binding and ECM binding sites to stabilize the cells from substrata withdrawal done in vitro. Thus cell adhesion proteins are anti-anoikis proteins. Disintegrins are pro-anoikis proteins. Proteins can induce anoikis through cell retraction and detachment and thus inhibitors to these proteins (e.g. binding proteins or antibodies) can be used in the invention to prevent anoikis by this mechanism.

Withdrawal of serum, growth factors cytokines, or cell mitogens can cause anoikis. The presence of these same factors can prevent cell death by anoikis. For example, IGF-1 protects fibroblasts from anoikis and HGF (hepatocyte growth factor) protects hepatocytes. TFF-3 (trefoil factor-2), a peptide secreted by intestinal goblet cells, has been shown to induce resistance to anoikis and TrkB, a neurotrophic tyrosine kinase receptor, is a potent and specific suppressor of caspase-associated anoikis of non-malignant epithelial cells. Some proteins widely used as tumor markers, such as human carcinoembryonic antigen (CEA), are upregulated in many types of human cancers and have been shown to inhibit cell death by anoikis. These proteins may promote metastatic processes by blocking the tissue architecture surveillance mechanism monitoring adherence and anchorage to their substrates. Caveolin-1 inhibits growth, anoikis and invasiveness in breast cancer cells, transformation of epithelial cells with oncogenes such as v-src, v-Ha-Ras, treatment with phorbol esters or exposure to migratory factors such as HGF (hepatocyte growth factor) all decrease the susceptibility to detachment-induced apoptosis. The expression of some genes involved in cell death, for example TRAIL, has been shown to be suppressed by anchorage, which can provide a mechanism to prevent apoptosis of cells that would otherwise experience anoikis. Providing substrata for the cell implantate or cell culture can be accomplished by adding ECM or serum proteins.

Anoikis can be suppressed by integrins through the focal adhesion kinase activity. Phosphatidylinositol 3-kinase/Akt and mitogen-activated protein kinase may mediate this suppression. Stress-activated protein kinase/Jun amino-terminal kinase pathway promotes anoikis. Bcl-2 and related proteins also may participate in anoikis. The characterization of focal adhesion proteins such as pp125FAK has revealed that multiple pathways connect adhesion to the suppression of apoptosis. Anokis can be resisted through G1/S cell cycle arrest for example by factors that act through the Erk-mediated bim suppression pathway. Thus the presence of ligand-integrin interactions can prevent anoikis.

ECM or serum proteins, such as fibronectin and vitronectin, through their integrin receptors, is critical for transducing survival signals in various cell types. The process of anoikis involves cellular integrins and components of the extracellular matrix. Fibronectin and vitronectin amongst other cell adhesion proteins provide survival signals for many cell types through the RGD as well as ancillary domains to bind the ECM such as the heparin binding domain in fibronectin. Thus binding to ECM or serum components can be used prevent anoikis. Cell adhesion enhances proliferation of cells onto a substrata and prevents anoikis. Proteins that increase proliferation by cell adhesion can be used in the invention.

The tripeptide (Arg-Gly-Asp) RGD, is an important cell adhesion motif. It is contained in a number of extracellular and serum proteins. The adhesion receptors of the integrin family are involved in preventing anoikis and enhancing cell growth, proliferation, migration and differentiation and are formed by noncovalent associated α and β subunits. Activating antibodies can activate the receptors as well as ligands with an RGD sequence such as fibronectin, vitronectin, and fibrinogen. Activating antibodies such as TS2/16, 8A2, TASC, 9EG7, 12G10 and HUTS can activate the integrins so that only a RGD motif need be present instead of synergistic sites on a protein, such as fibronectin, to bind. Also integrins that may bind one type of cell adhesion site can now bind other sites. For example, α4β1 integrin that binds the LDV sequence, when activated with antibodies will also bind the RGD site of fibronectin. Activating antibodies can also change the specificity of the ligand, e.g. TS2/16 induces the added interaction of the collagen receptor α2β1 with laminin. Activation of the receptors to proteins that normally prevent anoikis can be done with antibodies.

Protease Inhibitors

Protease inhibitors specifically inhibit the action of certain proteases. Some inhibitors are narrowly directed to one or a few proteases, while others (e.g., aprotinin, α2-macroglobulin) act more generally. Protease inhibitors may be added with cells and/or other factors described herein, e.g., extracellular matrix molecules and cell adhesion proteins, some of which also serve as protease inhibitors. Protease inhibitors can be used in vitro as well to control protease activity in culture and handling of the cells (e.g. trypsin cell dissociaton from the culture). Protease inhibitors, reduce the rate of proteolytic destruction of proteins and may advantageously be used to slow destruction of implanted materials, e.g., cells or proteins. Without being bound to a particular theory, the protease inhibitors can help the implanted materials persist through an initial burst of proteolytic activity caused by the introduction of the materials.

Many protease inhibitors are known and can often be found or created when inhibition of a particular protease is desirable. In some embodiments, a particular extracellular matrix molecule is chosen for introduction to a patient and a protease inhibitor that inhibits proteases that attack that extracellular matrix are chosen. For example, a plasmin inhibitor is combined with fibrin, a TIMP is combined with collagen, or a particular TIMP is combined with aggrecans. In use, the desired extracellular matrix molecule is chosen and then an inhibitor of proteases that attack that extracellular matrix molecule is chosen and combined with the extracellular matrix molecule for introduction into a patient. Also, substrates (e.g. a particular extracellular matrix molecule) or receptors of proteases also can serve as inhibitors to the protease via mechanisms such as competitive binding. Competitive, substrate and non-competitive inhibitors are the most common mechanisms of inhibition.

Protease inhibitors can also be used to quench protease activity (e.g. trypsin) in cell culture processes. For example, inhibitors quench proteases after protease release of cells during passaging and/or harvesting of the cells In addition, protease inhibitors can be used to prevent further protease digestion of cells in vivo after cell implantation by inclusion in the cell implantate.

Proteases can reduce cell adhesion to other cells and to extracellular matrix, and can destabilize the extracellular matrix by degradation of the proteins present in the extracellular matrix and enzymes such as transglutaminase or lysyl oxidase needed for crosslinking and stabilization of extracellular matrix proteins. Proteases also can activate by cleavage of certain growth factors and cytokines. Two general classes of proteases that degrade ECM are the matrix metalloproteases which usually depend on bound Ca++ or Zn++ and the serine proteases that have a highly reactive serine in their active site. Some of the controls for modulating or inhibiting proteolysis can be by local activation of precursors such as plasminogen precursor conversion to plasmin by plasminogen activators via tissue-type plasminogen activator; by absorption through cell receptors such as bound urokinase-type plasminogen activator (uPA) that is on the leading edge of migrating cells; by the secretion of inhibitors such as the TIMPs; by the presence of excess substrate to quench protease activity; by the presence of specific domains of protease inhibitors such as the follistatin, WAP, Kunitz and Netrin protease inhibitor domains in proteins such as GASPs (growth and differentiation factor-associated serum proteins) or the presence of inhibitor domains singly or in combination; by growth factors and other proteins that control protease production or acitivity, protease inhibitor production or activity, receptor binding acitivity to proteases or their signaling pathways; by use of select proteases that degrade specific proteases; by use of proteins that are modified (e.g. naturally by native cells, by recombinant DNA means, chemically, etc.) to protect against proteolysis such as proteins that are glycosylated; amongst other means of protease inhibition.

Below are listed some of the proteases, their substrates and protease inhibitors such as α1-antitrypsin, α1-antichymotrypsin, α2-antiplasmin, heparin cofactor II, plasminogen activator inhibitor 1, the proteases matrix metalloproteinases (MMPs), and the tissue inhibitors of metalloproteinases (TIMPs).

Four main general classes of proteases are the metalloproteases, serine proteases, aspartic and cysteine proteases. Most are endopeptidases and some are exopeptidases. Many proteases and protease inhibitors are present in plasma and the ECM. General inhibitors of all protease classes can be the presence of the protease substrates in high concentrations, binding proteins such as soluble protease receptors or domains of the protease substrates and the plasma protein α2-macroglobulin. Many specific protease inhibitors are available for a protease class, subclass or individual protease.

Metalloproteases can be secreted as MMPs, pappalysins, BMP-1, ADAMTSs, can be membrane-bound as MT-MMPs, ADAMs, ACEs, neprilysins or can be cytosolic as THOP1 and insulysin. Most are endopeptidases in contrast to aminopeptidases or exopeptidases. MMPs (matrix metalloproteinases) degrade ECM protein and process various biological molecules. MMPs have the pro-metalloprotease and hemopexin-like domains. MMPs are synthesized as inactive precursors containing a prodomain that is removed by proteolysis to form active MMP molecules by cell and serum proteases.

MMPs and some of their ECM substrates are listed below. Specific inhibitors of MMPs are the TIMPs. Regulation of MMPs can also be through proenzyme activation of the MMPs. MMPs can activate other MMPs and proteins by cleavage of the pro-forms. Many cell types including keratinoctyes, fibroblasts, osteoblasts, immune and endothelial cells produce MMPs.

MMP1 (fibroblast collagenase, collagenase-1) m.w. 54,000, cleaves the ECM structural substrates collagens I, II, III, VII, VIII, X, gelatin (non-triple helical collagen), other ECM substrates proteoglycans, versican, perlecan, aggrecan, proteoglycan link protein, tenascin-C, entactin, casein, serpins, and non-structural ECM component substrates ovostatin, L-selectin, IL-1β, MMP-2, -9, α1-antichymotrypsin, α1-antitrypsin/α1-proteinase inhibitor, IGFBP-3, IGFBP-5, SDF-1 and TNF-α peptide (recombinant). Plasmin, plasminogen activator and cell proteases activate MMP1. MMP2 (gelatinase A, collagenase), m.w. 74,000, is broadly expressed, cleaves denatured collagens, collagens I, IV, V, VII, X, XI, XIV, gelatin, fibronectin, aggrecan, versican, proteoglycan link protein, elastin, MBP, osteonectin, laminin-1, -5, and non-structural ECM component substrates MMP-1, -9, -13, IGFBP-3, -5, IL-1β, TGF-β, FGF receptor1, and TNF-α peptide (recombinant). It contains fibronectin type II repeats and a collagen binding region. MMP3 (stromelysin-1, proteoglycanase, transin), expressed by epithelial cells and carcinomas, degrades collagens I, II, III, IV, V, IX, X, gelatin, denatured collagens, fibronectin, aggrecan, perlecan, versican, proteoglycan link protein, proteoglycans, decorin, elastin, laminin, osteonectin, entactin, MBP, casein, and non-structural ECM component substrates ovastatin, plasminogen, α₁-proteinase fibrinogen, α₁-antichymotrypsin, L-selectin, pro IL-1β, IL-β, IGFBP-3, pro-MMP-1, -8, -9, MMP-7, -8, -9, -13, MMP-2/TIMP-2, pro-HB-EGF, pro-TNFα and SDF-1. MMP3 activates pro-MMP1, pro-MMP8, progelatinase B (pro-MMP9) and is induced by fibronectin fragment 45. Plasmin activates MMP3 by cleavage of proMMP3. MMP7 (matrilysin, neutrophil collagenase), m.w. 30,000, degrades denatured collagen, gelatin, collagen types I, III, IV, V, X, fibronectin, decorin, aggrecan, proteoglycan link protein, elastin, laminin, entactin, casein and non-structural substrates plasminogen, transferrin, syndecan, MBP, β4-integrin, MMP-1, -2, -9, MMP9/TIMP-1, pro-MMP-2, -7, pro-TNFα, defensin, E-cadherin, Fas ligand and insulin. MMP7 activates procollagenases. MMP8 (neutrophil collagenase-2), m.w. 53,000, degrades collagens type I, II, III, V, VII, VIII, X, gelatin, fibronectin, laminin, entactin, aggrecan and non-structural ECM component substrates α2-antiplasmin and pro-MMP-8. MMP9 (gelatinase B), m.w. 78,000, is a major protease in the ECM that is induced by cytokines, growth factors and mitogens. Produced by mesenchymal cells, alveolar macrophages, granulocytes and tumor cells, MMP9 plays a major role in tumor metastasis, basement membrane turnover, and osteoclastic resorption. MMP-9 degrades collagens I, IV, V, VII, X, XIV, gelatin, fibronectin, aggrecan, versican, proteoglycan link protein, elastin, entactin, osteonectin, MBP, and non-structural ECM substrates IL-1β, plasminogen, TGFβ, pro-TNFα, CXCL5, IL 2-receptor, and SDF1. MMP9 has stromelysin cleavage sites and fibronectin type II repeats. MMP10 (stromelysin-2), m.w. 54,000, expressed by epithelial cells and carcinomas, cleaves denatured collagens I, III, IV, V, collagen types III, IV, and V, gelatin, elastin, fibronectin, laminin, entactin, aggrecan, casein and the non-structural ECM component substrates pro-MMP-1, -8, -10 and MMP-1, -8. MMP11 (stromelysin-3), m.w. 55,000, degrades a variety of extracellular structural matrix components including fibrillar collagens, gelatin, laminin, casein and non-structural ECM component substrates α₁-antitrypsin, α₁-proteinase inhibitor, and IGFBP-1. MMP11 is expressed in many tissues and cell types including fibroblasts, stromal cells and carcinomas. MMP11 cleaves serine protease inhibitors. MMP12 (macrophage metalloelastase), m.w. 54,000, expressed in macrophages and stromal cells, degrades collagen IV, gelatin, fibronectin, vitronectin, soluble and insoluble elastin, laminin, entactin, fibrin, casein, MBP, and the non-structural ECM substrates fibrinogen and plasminogen. MMP12 plays a role in tissue remodeling. MMP13 (collagenase-3), m.w. 54,000, degrades collagens I, II, III, IV, V, IX, X, XI, XIV, gelatin, osteonectin, aggrecan, perlecan, laminin, large tenascin C, fibronectin, recombinant fibronectin fragments and the non-structural ECM component substrates plasminogen, plasminogen activator 2, pro-MMP-9, -13 and SDF-1. TNFα and IL-1β or α increase MMP-13 expression, for example in dermal fibroblasts, as does FN fragments 45 and 70. MMP-14 (MT1-MMP) degrades collagens I, II, III, gelatin, fibronectin, vitronectin, entactin, proteoglycans, aggrecan, dermatan sulphate proteoglycan, perlecan, tenascin, laminin, fibrin, casein, COMP (cartilage oligomeric matrix protein) and the non-structural ECM substrates tissue transglutaminase, SDF-1, CD44, gC1qR, αᵥβ₃ integrin, pro-TNFα, pro-MMPs-2, -13 and MMPs-2, -13. MMP-15 (MT2-MMP) degrades the ECM substrates collagen types I, II, III, gelatin, fibronectin, vitronectin, entactin, aggrecan, perlecan, laminin, tenascin, and the non-structural ECM substrates tissue transglutaminase, pro-MMP-2, -13 and MMP-2. MMP-16 (MT3-MMP) cleaves collagen I, III, gelatin, fibronectin, vitronectin, aggrecan, perlecan, laminin, casein and the non-structural ECM substrates pro-MMP-2, -13 and MMP-2. MMP-17 (MT4-MMP) cleaves gelatin, fibronectin, and fibrin. MMP-18 (Xenopus collagenase-4) cleaves collagen I. MMP-19 (RASI) cleaves collagen I, IV, gelatin, fibronectin, aggrecan, laminin, entactin, tenascin-c, COMP, casein. MMP-20 (enamelysin) cleaves amelogenin, aggrecan and COMP. MMP-21 (Xenopus MMP, MMP23A) cleaves the non-structural ECM substrate α₁-antitrypsin. MMP-22 (MMP-23B) is also known as chicken MMP. MMP-23 (cysteine array matrix metalloproteinase or CA-MMP) cleaves gelatin. MMP-24 (MT5-MMP) cleaves gelatin, fibronectin, chondroitin and dernatan sulfphate proteoglycans, but not collagen I or laminin. MMP-25 (MT6-MMP or leukolysin) cleaves collagen IV, gelatin and the non-structural ECM substrate pro-gelatinase A. MTMMPs contain a furin (Golgi-associated MMP activator) recognition domain. MMP-26 (matrilysin-2, endometase) cleaves collagen IV, gelatin, fibronectin, casein and the non-structural ECM substrates fibrinogen, α₁ and β₁-proteinase inhibitors. MMP-28 (epilysin) cleaves casein. MMP-27 and other MMPs exist as well. MMPs-4, -5, -6, and -29 are redundant in humans and no longer in use.

TIMPs or tissue inhibitor of metalloproteinases, regulate the activation and proteolytic activity of the MMPs. The TIMP mechanism is to block MMP activity by binding to the zinc binding active site domain. TIMPs are 21-28 kDa proteins produced by a number of cells including keratinoctyes, fibroblasts, osteoblasts and endothelial cells as soluble proteins. TIMPs have roles in development, cell-growth regulation, cancer cell invasion and metastasis, erthyroid potentiation and degenerative diseases. TIMP1, m.w. 23,000, is present in fibroblasts and other cell types, binds tightly to proMMP9 and its expression is regulated by cytokines and growth factors. TIMP 2 has a m.w. 24,000, binds tightly to proMMP2 and is constitutively expressed. TIMP 3, m.w. 24,000, is present in retina and choroids and other tissues, is an inhibitor of angiogenesis and is regulated in a cell cycle dependent manner. It inhibits MMP1, stromelysin-1, gelatinases A and B. TIMP 4 has a m.w. 26,000, is expressed highest in heart and may inhibit tumor invasion. TIMPs are inhibitors to many of the ADAMs, ADAMTSs and other proteases described below. MMPs can also be regulated at the transcriptional, pro-enzyme activation and storage levels (e.g. MMP-7, -8, -9 can be controlled by release from stores in cells). Structural regions of MMPs are the prodomains that contain the PRCGVPD motif and maintains zymogen (precursor) latency. Molecules and proteins that interact with this motif can keep the MMPs in an inactive form. The active site is a zinc-binding domain that includes the HEXGHXXGXXH sequence motif and molecules that disrupt or bind this motif can inactivate the protease. The hemopexin domain is present in all MMPs but MMP7 and promotes interaction with substrate. Molecules that compete with or bind to this domain can retard or inactive the MMP. Collagenases, including bacterial sources, can be quenched with α2-macroglobulin as well as with specific collagenase inhibitors α1-antitrypsin present in serum or ovostatin from egg white.

The secreted metalloproteases ADAMTSs are a subset of ADAMS proteases and contain a thrombospondin (TS) domain, in addition to the pro, metalloprotease, disintegrin-like and cysteine-rich extracellular domains. ADAMTS 1-13 include ADAMTS1, a protease associated with the ECM, cleaves aggrecan, binds α2-macroglobulin, suppresses FGF2 induced vascularization and VEGF induced angiogenesis. The protease is involved in normal growth, acute inflammation, mineralized nodule and bone formation and organ morphology. ADAMTS2 is a procollagen I and II N-proteinase involved in collagen biosynthesis and fiber formation. ADAMTS4 (aggrecanase-1) cleaves aggrecan and brevican. ADMATS5 (aggrecanase-2) cleaves aggrecan. ADAMTS8 inhibits vascularization and angiogenesis. ADAMST9 is expressed in dendritic cells. ADAMTS13 cleaves von Willebrand factor. ADAMTS2 and ADAMS14 cleave procollagens.

ADAMs (A Disintegrin and Metalloprotease), are 80 to 120 kDa membrane bound proteases, that are involved in cell-cell interactions, development and other processes. ADAMs are part of the 40 plus member family and have diverse functions. Many ADAMs are type I transmembrane proteins and contain the extracellular domains of ADAMTS in addition to extracellular EGF, transmembrane (TM) and cytosolic domains. ADAMs can be alternately spliced before the TM domain to produce soluble secreted forms such as ADAMs 11, 12, 17, 28 and others. ADAM 9 cleaves the insulin B chain. ADAM 10 cleaves the extracellular domain of membrane bound Notch receptor, proTNFα and type IV collagen. ADAM12 cleaves α2 macroglobulin, binds to and cleaves IGFB3, binds to α actinin-2, is involved in adhesion and migration of cells (e.g. neural) and assists in myoblast fusion. ADAM15 contains a RGD domain and functions as an adhesion molecule that interacts with α₅β₃ integrin. ADAM17 (TACE or TNFα converting enzyme) processes TNF ligand and receptor generating soluble TNF receptor that is involved in inflammation. ADAM17 processes L-selectin, TRANCE, HER4 JM-a, Notch 1 receptor, and contains a secretase activity. ADAM19 is involved in osteoblast differentiation.

The disintegrin domain, present in ADAMS shows sequence similarity to snake venom peptides generated from repolysin precursors. Disintegrin domains have a RGD integrin binding site and bind platelet integrin αIIb/β3. Most ADAMS do not have a RGD site in the distintegrin domain but still bind integrins. For example the ECD motif of the ADAM2 disintegrin domain or ADAM12 supports cell-cell interaction. The pro domain of ADAMs, consist of about 200 amino acids, contains a cysteine switch motif that keeps ECM MMPs and reprolysins in a latent form. Zinc activates these latent MMPs and reprolysins. The cysteine switch in ADAMs prevents autocatalysis and MMP and reprolysin degradation.

A zinc binding site is present in all ADAMSTS whereas not all ADAMS possess this site. Reprolysins are part of the metzincins family. Zincins contain metalloproteases and the zinc metalloproteases are proteinases or peptidases that all need Zn for catalysis.

BMP-1 (procollagen C-proteinase) is a zinc protease of the astacin family. BMP-1 cleaves ECM precursor proteins into mature proteins such as collagens, biglycan, laminin 5, dentin matrix protein-1, lysyl oxidase, etc. Pappalysins (pregnancy-associated plasma proteins A1 and A2) cleave proteins such as the A1 form that cleaves IGFBP-4 and -5 releasing bioactive IGF.

ACE (angiotensin converting enzyme) and ACE-2, are membrane proteins that regulate the renin-angiotensin system, maintaining blood pressure homeostasis and fluid salt balance. ACE is involved in immunity and ACE-2 in heart function, as a negative regulator of RAS. ACE-2 cleaves angiotensins I and II. Soluble serum and fluid forms of ACE are formed by secretase (sheddase) action. ACE degrades amyloid β-peptide (Aβ), retards its aggregation, deposition, fibril formation and inhibits amyloid cytotoxicity. Aminopeptidase (APN) is a transmembrane protein with an extracellular Zn metalloprotease domain. It is widely expressed in tissues and inactivates proteins by cleaving N-terminal amino acids from peptides. It is involved in cell adhesion, metastasis, and antigen processing and presentation. Aminopeptidase PILS is a Zn metalloprotease and promotes TNFR1 and IL-6 receptor ectodomain cleavage. PILS is involved in antigen presentation and hypertension. Methionine aminopeptidase (MAP) removes initiator Met residue from nascent proteins. Carnosine dipeptidase 1 (serum camosinase) degrades carnosine, homocarnosine and related peptides.

ECEs are members of the 8 member neprilysin (NEP) family and consist of zinc type II transmembrane proteases with a large ectodomain. The NEP family also includes PEX, XCE, DINE, Kell and NEP-like proteins. Soluble forms of NEPs exist. ECE-1 and ECE-2 cleave endothelin-1, bradykinin, neurotensin, angiontensin I, substance P, dynorphin B, proenkephalin-derived peptides (e.g. peptide E, BAM 18 and 22, PEN-LEN an endogenous inhbitor of prohormone convertase 1), amongst other bioactive peptides. Kell cleaves endothelins. Neprilysin cleaves enkephalins, circulating arial natriuretic peptides, and amyloid β peptide. NEP2 cleaves tachykinins and enkephalins. EMMPRIN, another transmembrane protease, has two Ig extracellular domains. It interacts with integrins, caveolin-1 and MCT1, among others, and induces extracellular metalloprotease activity, such as MMPs-1, -2-3 and -9 production.

General inhibitors of these metalloproteases are the protease substrates in high concentrations and α2-macroglobulin. Other regulators include Lipocalin-2, the TIMPs-1, -2, -3, -4, testicans 1-3, RECK, and PCPE. RECK inhibits MMP-9 and lipocalins are inhibitors of cysteine proteases. Lipocalins are a family of extracellular ligand-binding proteins having tight specificity for small hydrophobic molecules. They function in protease interactions, for example with proteinase inhibitor 12 and with serine-type endopetidase inhibitor activity (e.g. pancreatic trypsin inhibitor, tissue factor pathway inhibitor). Testicans are extracellular multi-domain chondroitin sulfate proteoglycans, highly expressed in the brain, modulates cell attachment and neurite outgrowth in vitro. Testican 1 and 3 inhibit MT (membrane type) 1-MMP and MT3-MMP activities and testican 2 suppresses the inhibitory activity of other testican family members.

Serine proteases are involved in a number of biological processes including coagulation, and complement. The members of this class of proteases include trypsin, chymotrypsin, elastase, proteinase K, angiostatin, complement components (C1r, C1s, C2), complement factor D, MASPs, cathepsin A, coagulation factors II (thrombin), VII, X, XI, granzymes such as B, D, G, H, kallikreins such as isoforms 3-8, 10, 11, 14, 15, plasma kallikrein, plasminogen, uPA, proteinase K, tryptases such as isoforms α, β-1, γ-1, 5, TSP50, HGF activator, HTRA, furin, corin, DPP6, DPPIV, spinesin and marapsins.

The classical complement pathway is triggered by C1, a complex of recognition protein C1q and two serine proteases, C1r and C1s. After C1 recognition C1r autoactivates and then activates C1s which cleaves the substrates C4 and C2. C1 cleaves C2 into two chains C2A and C2b. C2a contains a von Willebrand Factor domain and a serine protease domain, while C2B contains 3 Sushi domains. Complement factor D (adipsin) is the initial proteolytic step in the alternative pathway of complement and cleaves factor B in complex with C3.3. It is regulated by reversible conformational changes. Complement MASP3 is a member of the MASPs that are involved in the mannan-binding lectin (MBL) complement pathway.

Thrombin precursor, ˜62 kDa, is processed into several forms of α, β, and γ thrombin. Thrombin cleaves fibrinogen to fibrin, activates coagulation factors V, VII, VIII, XIII, and complexes with protein C and thrombomodulin. Thrombin activates platelets and through protease-activated receptors (PARs) regulates signaling pathways. Coagulation factor VII binds to tissue factor (TF). Coagulation factor X activates thrombin. Factor X is activated by both intrinsic and extrinsic pathways to factor Xa. Factor XI is complexed with kininogen and converts into XIa by contact with blood coagulates or by thrombin mediated activation on the platelet surface. XIa then converts factor IX into IXa which then activates factor X into Xa. Xa mediates thrombin activation. uPA (u-plasminogen activator, urokinase) converts plasminogen to plasmin.

The kallikrein (KLK) family has more than 15 members (KLK1-15). KLK3 is known as PSA (prostate specific antigen). KLK4 is known as enamel matrix serine protease 1. KLK5 is found in skin, brain and breast and is a stratum comeum tryptic enzyme. KLK5 digests ECM proteins collagen types I, II, III, IV, fibronectin, and laminin. KLK5 regulates the binding of plasminogen activator inhibitior 1 to vitronectin. KLK5 is involved in tumor progession, especially invasion and angiogenesis.

Granyzme serine proteases are found in cytotoxic T lymphocytes and natural killer cell granules.

Tryptases have trypsin-like specificity and together with chymases and cathepsin G, these proteases are mediators of inflammatory and allergic responses via mast cells. Tryptase β-1 (mast cell protease 7) shows anticoagulant activity via fibrinogen degradation. Trypsin has substrate specificity on positively charge lysine and arginine side chains. Many of these cleavage sites are present on ECM proteins.

Elastase is made by a variety of cell types including immune and pancreatic cells, is present in blood and acts on elastin and a number of other proteins (e.g. aggrecan).

Spinesins are type II transmembrane serine proteases. Marapsins are produced in the pancreas. Plasma HGF activator cleaves the single chain HGF precursor into the active heterodimer. Thrombin activates the circulating inactive HGF activator zymogen. HTRAs, such as HTRA2, remove IAP mediated inhibition of caspase activity by the BIR domain binding and also serves as a serine protease. Furin is a member of the proprotein convertase family in the subtilisin superfamily of serine proteases. Cathepsin A is a lysosomal carboxypeptidase. Enteropeptidase activates pancreatic proteases by cleaving trypsinogen to trypsin which then activates chymotrypsin, carboxypeptidases and elastases. Dipeptidyl peptidase IV (DPPIV) cleaves dipeptides from the N-terminus of oligo and polypeptides. It is involved in cleavage of chemokines such as SDF-1 α, MDC, procalcitonin, has a role in T cell-activating molecule and is a cofactor for HIV entry. It is present in on the surface of many cell types and present in soluble form in the serum and other body fluids.

Most serine proteases are regulated by activation of zymogens or inactivation by inhibitor binding. Serpins have more than 35 members (e.g. A1, A5, B5, C1, D1, E1[PAI-1], E2, F1, F2, G1[C1 inhibitor], I2) that bind the protease active site of serine proteases as well as non-protease proteins. The binding covalently traps the protease. Serpins are involved in blood coagulation, inflammation, immunity, angiogenesis, cancer and reproduction. uPAR (u-plasminogen activator receptor) is a transmembrane protein that binds uPA through its extracellular domain. Plasminogen kringle 5 (one of five kringle domains in plasminogen heavy chain A) inhibit serine proteases. Coagulation factor III (tissue factor) is a binding protein or receptor for coagulation factor VII. Ecotin is a general inhibitor of serine proteases including trypsin, chymotrypsin, elastase, factors Xa, XIIa, plasma kallikrein, granzyme B and uPA. EPR-1 or effector cell protease receptor-1 is a transmembrane glycoprotein receptor for factor Xa. GASP-1 (growth and differentiation factor associated serum protein-1) contains WAP, follistatin, immunoglobulin, kunitz and netrin domains. WAP, follistatin, and netrin domains are involved in protease inhibition. GASP-1 inhbits GDFs 8 and 11. Netrins are part of the laminin-related family of axon-guidance molecules and found in neurons, Schwann cells, osteoclast and fibroblasts. Trappins include elafin (elastase specific inhibitor), also known as skin-derived anti-leucoproteinase. Trappins include SLPI (secretory leukocyte protease inhibitor) found in fluids and an inhibitor of neutrophil proteases, elastase, cathepsin G, chymotrypsin, trypsin, amongst others. HAIs (HGF activator inhibitors, HAIs-1, 2, 2A, 2B), are transmembrane type I proteins, and suppress HGFA. Soluble forms of HAIs are formed by ectodomain shedding. Other serine protease inhibitors include aprotinin (potent inhibitor of e.g. trypsin, plasmin, kallikrein), α1-antitrypsin, plasminogen activator inhibitor-1, EPCR, leupeptin, antipain, chymostatin, elastatin, kallikrein inhibitor, soybean trypsin inhibitor, TFPI-2, hirudin, bikunin and members of the I-α-I family and members of the Kunitz, Kazal and STI-Kunitz families. Soybean trypsin and kallikrein inhibitors inhibit the proteolytic but not the elasteolytic activity of elastase. C1 esterase inhibitor interferes with the initiating component of the complement cascade. α1-chymotrypsin is an inhibitor for chymotrypsin. α1-antitrypsin (A1AT) is a serum glycoprotein that inhibits trypsin, chymotrypsin, and elastase, among other proteases. Amyloid protein can be an elastase inhibitor. Ovomucoid, derived from egg whites, inhibits certain elastases, trypsins and chymotrypsins. IαI (inter-alpha-inhibitor), basic pancreatic trypsin inhibitor and lima bean trypsin inhibitor inhibit plasmin.

Potent trypsin inhibitors include α1-antitrypsin, aprotinin, trypstatin, soybean trypsin inhibitor, lima bean trypsin inhibitor, basic pancreatic trypsin inhibitor (Kunitz), and ovostatin and ovomucoid from egg white. Trypsin digestion can be used to free cells from the ECM in order to harvest and passage cells in cell culture. Trypsin cleaves proteins at the positively charged lysine and arginine side chains and trypsin inhibitors can be used to stop trypsin damage to the cells.

Aspartic proteases contain the members: BACE-1, BACE-2, Presinilin-1, Presilin-2, Cathepsin D, Cathepsin E, β and γ secretases. BACEs (Beta-site APP-Cleaving Enzymes) are membrane bound members of the pepsin family, widely expressed and cleave amyloid precursor protein (APP) (e.g. Alzheimer's disease). BACE-2 has α in addition to β secretase activity. Cathepsin D is a lysosomal member of the pepsin family, Cathepsin E is an intracellular member. The D member degrades proteins in lysosomes and is involved in antigenic presentation of peptides. Secretases cleave the membrane proximal domains of various growth factors, cytokines, receptors, cell adhesion molecules, and ectoenzymes. Active γ secretase include presenilins (transmembrane), nicastrin, Aph-1, Pen-2. Amyloid β, a component of plaques in Alzeheimer's, is cleaved from APP by β and γ secretases. Cathepsins are lysosomal proteases. General inhibitors, pepstatin and the peptide VdLPFFVdL are effective inhibitors on this class of proteases.

The cysteine proteases consist of two families, the cytosolic, asparatic specific caspases involved in apoptosis and the lysosomal cathepsins involved in protein degradation. Some of these proteases are caspases-1 to -13, and the primarily lysosomal cathepsins 1, 3, 6, B, C, F, H, L, O, S, V, X, cathepsin-like proteases, legumain, papain and separase. Caspases are produced as latent zymogens and activated by autoproteolysis or by other proteases, including other caspases. The three functional groups are the cytokine activated caspases-1, -4, -5, -13; apoptosis initiation caspases-2, -8, -9, -10; and the apoptosis execution caspases-3, -6, and -7. Caspases are stimulated by APAF1, CFLAR or FLIP, NOL3 or ARC, amongst others.

Caspase inhibitors are LAP family members that include NAIP, cIAP-1, cIAP-2, XIAP, survivin (binds to caspases 3, 7 or 9) and livin (inhibits caspase-9). DAIBLO and Omi regulate IAP activity. Additional inhibitors of cysteine proteases include the cystatins A, B, C, D, E/M, F, H, H2, S, SA, SN, Fetuin A and B, HPRG, kininogen, kininostatin, lipocalin-1, aprotinin and α2-macroglobulin. Cystatin A and B are intracellular inhibitors for cysteine proteases of the papain family. Cystatin C is present in tissues and body fluids and inhibits lysosomal proteases. Cystatin E/M is also a substrate for transglutaminases and needed for viability and formation of cornified layers of the epidermis and hair follicles. Cystatin F is produced by hematopoietic cells. Plasma glycoprotein kininogen is processed into heavy and light chains and the release of active peptide bradykinin. The His rich domain of the light chain is associated with clotting activity. Plasma kallikrein cleaves kininogen into bradykinin and Hka. Domain 5 of HKa, called kininostatin, displays anti-angiogenic activity. Aprotinin inhibits tissue and plasma kallikrein. Lipocalins are extracellular carriers of lipophilic molecules and interact with cell surface receptors and proteases.

Other classes of protease include proteasome multicatalytic endopeptidase, acid proteases such as rennin and HIV protease, ubiquitin-proteasome and mitochondrial proteases. Naturally occurring protease inhibitors exist for these classes.

A general non-specific inhibitor of all classes of proteases is α2-macroglobulin, a human serum glycoprotein has sequence similar to complement components C3, 4, 5. It contains four identical subunits of 180 kDa each, has a broad range of specificity. The irreversible protease inhibitor inhibits proteases by a trapping mechanism. The trapped protease loses its ability to be active against large substrates.

Other non-specific and general inhibitors of the α-macroglobulin complement family found in plasma include α1-macroglobulin and α1-inhibitor III. α1-macroglobulin, a 725 Kd glycoprotein that inhibits proteolysis of the extracellular processes resulting from clotting, fibrinolysis and proteinases of inflammation. Both proteases are obtained from rat.

Protease inhibitors can be categorized into the low-molecular weight inhibitors (LMWIs) and naturally occurring inhibitor of proteins of which many examples are given above. LMWIs, most of which are toxic, are synthetic or from bacteria or fungi that irreversibly modify an amino acid in the protease active site. These include phenylmethane sulfonyl fluoride (PMSF), amstatin, antipain, APMSE, bestatin, benzamidine, chymostatin, 3,4,-dichloroisocoumarin, DFP, E-64, elastatinal, leupeptin, pepstatin, diportin A and B, 1,10-phenanthroline, phosphoramidon, TLCK, and TPCK. Some of these small molecules or bioactive peptides inhibit exopeptidases as well.

General or specific protein inhibitors can be used. Fragments, domains, motifs and other forms of the inhibitors can be utilized. For example, the follistatin, WAP, Kunitz and Netrin protease inhibitor domains can be effective against the proteases that their naturally occurring protein inhibitors (e.g. GASP) are. Furthermore, factors (e.g. proteins, growth factors) that modulate signaling pathways of protease activity and protease inhibitor activity can be used.

Macromolecules (Proteins) Present During Aging

A decline in the cell population of tissue can promote physiological aging, functional and morphological deficits in the tissue, all of which are classified as tissue defects.

Macromolecules such as ECM, serum and other proteins can have altered expression, activity and structure due to aging.

AGEs (advanced glycation end-products) are formed in tissues. For example, collagen and all other ECM and serum proteins become non-enzymatically glycated with age. Sugar adducts are often bound to lysine and hydroxylysine residues. AGEs affect many cell functions and protein functions including ligand binding, interactions with other macromolecules, increase in immunogenicity of the protein, decrease in protease susceptibility of the protein, increase in the crosslinking of the protein within polypetides of the protein, intra and inter-cellular crosslinking to proteins, increase trapping of non-glycoslyated proteins like LDL and immunoglobulins, increase aberrant cell proliferation such as in fibroblasts and smooth muscle cells, increase in ECM components, amongst other deleterious actions. AGEs interact with many proteins including serum proteins such as hemoglobin β₂-microglobulin, ECM proteins like collagen, elastin, β-amyloid, etc., increases cytokine production such as TNFα and the transcription factor pathway NF-κB, increases inflammation of a tissue and increases apoptosis of cells. Many AGEs exist in serum including glycated proteins.

AGEs (advance glycation end-products) promote apoptosis through interaction with the RAGE receptor that ultimately reduces ECM formation. Antibodies to RAGE inhibit binding of proteins that are AGEs. AGEs also induce NF-κB activity without affecting apoptosis. Higher rates of fibroblast apoptosis is observed in aging tissues, poor wound healing, diabetic tissues and inflammation. Control of apoptosis may increase efficacy of treatments for these tissues. The higher rates of apoptosis parallels the formation of AGEs in these tissues. The RAGE receptor is a member of the immunoglobulin superfamily. Addition of RAGEs soluble receptor, the extracellular portion of the receptor or the peptides containing the AGEs binding site, can be used to bind and remove AGEs from serum and ECM. Camosine can prevent formation of AGEs. ALT-711 and other crosslink breakers can remove AGEs. Anti-oxidant sources, including proteins can be added to prevent AGEs formation or oxidation of cells and proteins. For example, superoxide dismutase (e.g. SOD-3) is present in the ECM and serum and protects cells.

The amyloids, in particular β amyloid, increase with age in serum and tissues. Specific proteins or molecules can neutralize amyloid β. Angiotensin converting enzyme degrades amyloid β-peptide (Aβ), retards its aggregation, deposition, fibril formation and inhibits amyloid cytotoxicity. Neprilysin and other proteases can cleave amyloid β peptide. Inhibitors to amyloid precursor conversion, such as inhibitors to β and γ secretase, can prevent amyloid β peptide formation. Glycation can cause the formation of amyloid.

ECM proteins can decrease in quantity as tissues age. Fibrillar collagen, the primary structural protein, is reduced (except in the heart) in aged tissues. Fibronectin is decreased in aged tissue and wounds of aged organisms. Many other ECM proteins are down-regulated in aging tissues. Basement membrane proteins and other ECM proteins can be increased in various pathologies such as diabetes and atherosclerosis.

Proteins obtained from different aged sources other than the age the cells are obtained from can be used. Younger serum can be used singly or in tandem with cells or younger ECM proteins or other proteins can be used singly or in tandem with cells to treat defects. Cells from different aged sources and proteins produced by these cells can be used to treat defects, including autologous cells that have been chronologically stored.

Growth factors, cytokines, chemokines, hormones, ECM and serum proteins can change quantitiatively and qualitatively with age. Incubation of cells in vitro and in vivo with the proper type, form and concentration of these factors or hormones can be used to augment the cell survival, behavior and proliferation of the invention. For example, with skin fibroblasts, estrogen and progesterone suppress ECM degradation by inhibiting metalloproteinases, and estrogen increases ECM synthesis such as for collagen, hyaluronic acid, GAGs, and specific proteoglycans. Skin thickness can be maintained or improved. Additionally keratinocyte proliferation is increased by these steroids, while estrogen suppresses apoptosis preventing epidermal atrophy. In wound healing, estrogen stimulates macrophages to produce NGF, GM-SCF production in keratinocytes, βFGF and TGF-beta 1 production in fibroblasts that leads to enhanced wound re-innervation, re-epithelialization and granulation tissue formation. In aged tissues (e.g. skin) there is excess protease activity compared to structural ECM made. TIMP-1 inhibits MMPs 1, 2, and 3 which degrade collagen, elastin and other ECM components. Other TIMPs inhibit additional MMPs, thus preserving ECM. UV aging of tissues such as skin also involve cytokines TGFα and IL-1β, decreased fibrillin, increased MMP-1, -2 and -9, and altered synthesis of tropoelastin, collagen and TIMPs. Addition of tissue inhibitors can prevent degradation of cell made ECM in the implantate.

Certain hormone concentrations and factors can change with age. Growth hormone, IGF-1, DHEA, sex steroids and a number of others decrease in quantity in the elderly. Increased concentration of hormones can be used in cell culture or can be incubated with cells in the implantate. Hormones and factors can singly or in combination, with cells or without cells, be used in the implantate to correct tissue defects.

Cell Senescence-Telomeres, Cell Quiescence-Serum Withdrawal

Three constraints to grow somatic cells in good numbers are cell quiescence, cell senescence and cell-cell contact inhibition. Cell quiescence occurs when serum free media is employed or when serum is withdrawn, causing a cessation of cell proliferation in which the cells are locked in the G0 cell cycle phase until induced with serum into the G1 to S phase. Cell-cell contact inhibition occurs when cells in vitro become confluent and proliferation ceases until the cells are re-seeded at a lower density. This inhibition of cell proliferation can be due to loss of serum factors for growth.

Cell senescence occurs when the genetically dictated replicative lifespan limits the number of somatic cell numbers and cells remain in the G1 cell cycle phase permanently. Often fibroblasts have been studied and these cells reach their lifespan usually between 40 and 80 population doublings. Irradiation, oxidative stress and intrinsic factors can bring cells to senescence by triggering the activation of tumor suppressor proteins such as p53, Rb, and p16/INK4A. Intrinsically telomere shortening is responsible for senescence.

Senescence can be stopped and immortality ofreplicative lifespan can be accomplished by viral transformation with viral genes from Epstein-Barr virus, simian virus 40 T antigen, adenovirus E1A nd E1B, or human papillmarvirus E6 and E7. In a preferred embodiment exogenous expression of hTERT (telomerase reverse transciptase) is employed to maintain or regain telomere lengths in cells.

Many of the proteins involved in these processes are present as cell cycle proteins. As inhibitors of cell proliferation, in which the cell cycle is in the G1 phase, it is within the invention to override the inhibitors of cell quiescence or senescence with proteins that either quench the inhibitory proteins or activate the cell cycle proteins to push the cell cycle into the S phase and beyond.

Cycling cells proliferate in the presence of growth factors, such as present in serum. Withdrawal causes a reversible exit into the G0 phase of the cell cycle.

TGF-β, retinoids, p53, histone acetylase inhibitors, p38, p27, p19, p16, p14, p21, and pRb are protein checkpoints that can trigger senescence. p53 and pRb represent major pathways that maintain the senescence phenotype and telomere pathways are an escape from senescence. p53 is produced upon DNA damage and telomere shortening can represent DNA damage. p53 produces p21 which inhibits cyclin dependent kinases.

Cell growth arrest in the G1 phase of the cell cycle during quiescence and senescence involve the cyclin-dependent kinase inhibitors (CDKIs). The CDKIs of the CIP/KIP family p21^{CIP1}, p27^{KIP1} and p57^{KIP2} and the INK4 family p15^{INK4b}, p16^{INK4a}, p18^{INK4c}, p19^{INK4d}, pRb, p107, p130 and p53 are also involved in the growth arrest. In quiescent cells p21, p53 are expressed. Mitogens can down-regulate p21 and p53 inhibitors and induce expression of ID1 and 2, c-myc, c-fos, cdk 4 and 5, cyclins A, C, D1, E, c-H-RAS, JUNB, c-JUN, CDK 4, 5, 6, CDC2, CyclinE-CDK2 kinase, PCNA, Histones, DHFR, TS, TK, E2F-1, RNR, and phosphorylated pRB. Mitogens can downregulate p21 and p16 CDK inhibitors. In senescent cells a similar profile is observed except mitogens can not induce ID1, 2 c-FOS, Cyclin E-CDK2 kinase, cylin A, CDCD2, E2F-1, RNR, Histones, PCNA, DHFR and pRB remains unphosphorylated. Mitogens can not downregulate p21 and p16 inhibitors.

Senescent cells produce increases in collagenase, stromolysin, plasminogen activator, plasmin, and TIMPs activity, amongst others. In general there is a reduced structural ECM synthesis and increase in protease activity. It is important not to reach senescence because cells will not proliferate in vitro. However, it is also important not to approach cell senescence during the expansion process. Otherwise adequate cell numbers will be difficult or impossible to reach for implantation. In addition the cells will have an altered phenotype which can cause damage to the implantated tissue (less ECM synthesis, excess protease activity). Furthermore, the cells can be rejected due to an altered gene expression profile and inappropriate protein production recognized by the immune system.

Premature cell senescence can occur by DNA damage, oxidative stress, excess proliferation or culture shock in which the cell culture conditions are changed and the cell do not adapt (e.g. feeder layers to plastic surfaces for cell growth). Insulin like growth factor I can extend in vitro replicative life span of cells (e.g.skeletal muscle satellite cells). Other such factors that can enhance the G1/S phase can as well. Overexpression of oncogenes such as Ras or Raf, or in a preferred embodiment the addition of specific growth factors or specific ECM constiutients such as fibronectin and cell adhesion proteins can be used to maintain proliferation of cells.

Senescence can be overcome by inhibition of the retinoblastoma (pRB) and p53 tumor suppressor pathways until telomere shortening triggers crisis. Endogenous expression of TERT (telomerase reverse transcriptase) at any replicative stage of the cells will render the cells immortal with respect to proliferation. Uncapped telomeres triggers cell cycle arrest or apoptosis or genetic instability. Telomere erosion may represent a form of DNA damage that sets into action the CDKIs. Telomeres are tandem repeats (TTAGGG/CCCTAA)ₙ located at the ends of linear eukaryotic chromosomes in which the length successively decreases (50-200 base pairs) with each population doubling. Telomeres protect damage and fusion of chromosome ends, allow chromosome replication and position the chromosome within the nucleus. Maintaining telomerase activity by transfection of cells in vitro with telomerase cDNA, the tert cDNA or other telomere factors can be part of the invention. DNA repair related enzymes and telomere binding proteins can be used including telomere/DNA repair complexes and associated proteins such as TRF-2, TRF-1, Rad51D, Mre11/Rad50/Nbs, DNAPKcs, Ku70/80, Wrn, POT1, PIP1, TIN2, hRAP1, Blm, ERCC1/XPF. Control of telomerase can be a therapeutic for cancer cells or to ensure proper somatic cells divide sufficiently for therapeutic amounts of cells for use in the invention. Senescence is resistant to mitogens but can be overcome by induction of downsteam oncogenes such as cMyc and E1A, cyclin E1, and those acting downstream of p16. Viral proteins reactivate terminally differentiated cells (e.g. T-viral oncoprotein or E1A in skeletal muscle differentiated cells) or inactivation of p53 or SV40 re-enters senescent cells into S phase of the cell cycle. Inhibitors to tumor suppressor genes and proteins such as p21, pRb and p53 can be used in the invention. Prevention of senescence or reversal by anti-senescence strategies are part of the invention. This preferably is done by addition of TERT or factors that increase telomerase activity resulting in telomere preservation or addition in the culture of cells. This manipulation can be performed at any time prior to implantation of the cells.

Cell contact inhibition can be overcome by reseeding at a lower density or addition of serum or serum factors. Without re-seeding, serum or serum factors can allow the 3 dimensional aggregation and formation of cells in vitro. Thus for tissue or organ synthesis, without introducing scaffolds, a natural 3 dimensional array of tissue components and cells can be formed in vitro. Additionally, cultures of such an array can permit cells more readied for implantation since the cells will be primed for the natural tissue environment in vivo.

Addition of Molecules and Proteins to the Cells

Molecules and proteins can be added to the cells prior to implantation. Molecules and proteins can be added as part of the co-injectate or composition of cells introduced into the subject or for the in vitro expansion of the cells. The purpose of the addition of molecules or proteins can be to maintain or improve the effect of the cells or the defect itself. Cell seeding, cell adhesion to the site of implantation, cell migration, survival, proliferation, nutrition, metabolism, differentiation and growth of the cells are some of the beneficial properties the addition of molecules or proteins can have on the cells. The molecules or proteins can optionally be immunogenic. Proteins and other molecules in serum may optionally be immunogenic and also provide important activities to the treatment of the defect and/or to the culturing of the cells. Accordingly, the various proteins and other factors that are described herein may optionally be immunogenic and may be used as part of the compositions and methods described herein, for example, as materials introduced into cell culture of introduced with cells into the patient. And, for example, the proteins or other factors can be part of the cell culture medium or serum used to grow the cells. Factors from the cell culture medium may be left with the cells and introduced intot the patient, or, alternatively, factors from the culture medium may be added to the cells for introduction into patient.

In some embodiments, cultured cells are collected by mechanical, physical or chemical means, e.g., by scraping, vibration, peeling, trypsinization, pressure or use of a chelating agent. The cells may be centrifuged, washed, and resuspended in a physiological solution, culture medium, or osmotically balanced preparation. The collection of cells may be incubated with factors by adding the factors to the solution that contains the cells. The factor is kept in contact with the cells for a predetermined amount of time. The amount of time allows the factor to interact with the cells and achieve the desired degree of incorporation onto or into the cells. The cells may be incubated in, e.g., a warm bath or incubator.

The cells may be incubated with an effective amount of absorbable proteins. The proteins are added to a collection of cells and, when added in a concentration commensurate to the number and concentration of cells, absorb to the surface of the cells. The proteins may then specifically interact with cell surface receptors that are available on the cells. The specific interaction provides signals to the cells to achieve a desired effect either in the collection of cells prior to implantation or after introduction into the patient. Absorbable proteins are thus effectively bound only to a cell in the collection and not to other surfaces or materials. A protein that is bound only to a cell can be internalized or degraded by the cell. This absorbability is often advantageous because the degradation, internalization of the protein or signal transduction elicited is often a key aspect of regulating the cell-to-protein interaction. In contrast, a protein that is part of a tissue, in a matrix, or adsorbed to a surface is hindered from being absorbed onto or into a cell. Cell-absorbable is a term that refers to an absorbable protein that specifically binds a receptor on a cell and is bound only to a cell. A cell-absorbable protein, by definition, is not a protein in a matrix or tissue. An effective amount refers to an amount that is sufficient to cause a significant portion of the cells to respond. The intent of treating the cells with the protein is to produce a desired effect in the cells, so that a sufficient number of cells and cell receptors must be exposed to the factor to produce a result. An effective amount is thus easily distinguishable from, among other things, an incidental or trace-amount exposure to a factor.

A collection of cells for introduction into a patient has certain characteristics that distinguish it from groups of cells in a cell culture or in a patient. Collection for introduction into a patient requires, for instance, careful sterile technique, collection of a suitable number and concentration of cells, use of carefully selected reagents that are free of unintended side effects, e.g., using appropriate sera, growth factors, and other ingredients. In contrast, culturing of cells can be expected to involved lower concentrations of cells for passaging or analytical purposes, use of different reagents, and use of different devices. Further, ordinary artisans can distinguish cultured cells from cells that are native to a patient, e.g., by use of biochemical markers or visualization of the morphology of the cells and tissues containing the cells.

The proteins listed and their respective family members are also included in certain embodiments of the invention. Proteins described herein can be alternatively spliced and thus exhibiting different characterizations and abilities. The majority of proteins are alternatively spliced as shown by the Human Genome Project and one versed in the art can incorporate these alternate spliced versions into the invention. Functional fragments, domains, motifs and sequences inherent to the proteins can be used, amongst others mentioned throughout the text and known in the art.

Additionally, polymers of amino acids or other chemical compositions can be used in conjunction with the cells of the invention. Many of the serum proteins and ECM proteins and other protein factors act through receptors to conduct the signaling pathway. Many of these receptors are transmembrane proteins. Receptors, especially soluble versions of receptors can be used to trigger the intended signaling pathways or to inhibit the natural receptor pathway by binding the appropriate biological ligand. Factors that control the various signaling pathways or proteins involved in the signaling pathways can be used that are described in the text.

Devices

The composition of the invention can be delivered using a device that is a hypodermic syringe, laparoscope, or other means depending on the defect and location of the tissue. For example, for repairing a dermal defect in a subject, a hypodermic syringe would have a syringe chamber, a position disposed therein, and an orifice communicating with the chamber and a suspension comprising the cells (such as papillary, reticular, fascia fibroblasts, pre-adipocytes, adipocytes, myoblasts, myofibroblasts, other fibroblast types, other cell types or a combination thereof). In a preferred embodiment the cells are from the subject and contain proteins that can be immunogenic or from the cell culture medium (e.g. serum-derived). A pharmaceutically acceptable carrier solution in which the suspension is disposed in the chamber and a hypodermic needle is affixed to the orifice. A similar situation prevails for laparascopic injections with these and other cells into different tissues. Other means of protein and/or cell delivery to tissue can occur by chemical means such as a penetrating agent, vasodilator, by physical means such as absorption, spraying, ultrasound, ballistic delivery, amongst other means.

Treatment of Defects and In Vivo Tests in Human Patients

This application includes materials and methods for the implantation of cells and/or macromolecules (or molecules) such as proteins into tissue defects from conditions associated with aging. One useful purpose of the treatment can be to increase a tissue's elasticity, which often declines with age. A tissue is a collection of cells that together perform a specific function in a body. Many tissues exist in a body, e.g., dermis, lung, neural, kidney, organs, muscle, fascial, cormective, bone. Processes are described herein that are useful to change, modify and/or restore the morphology of a tissue including many tissues affected by hypertrophy, atrophy, or dystrophy. Other embodiments are directed to repair of these and other defects by augmentation of existing tissue with cells and/or proteins to provide additional tissue structure and/or unction.

Methods and compositions are described for treating other tissue defects. The defects include those set forth in U.S. patent application Ser. No. 09/632,581 (filed Aug. 3, 2000) and Ser. No. 10/129,180 (filed May 3, 2002), which further provide detailed explanations of techniques for treatment of those defects. Defects include, but are not limited to, urological sphincter defects resulting in urinary incontinence, fecal incontinence due to anal sphincter degeneration or defects, ureteral orifice degeneration or defects causing vesicoureteral reflux, and gastroesophageal sphincter defects such as gastroesophageal reflux. Skin defects include wrinkles or rhytids, depressed scar or other cutaneous depression, stretch marks, hypoplasia of the lip, prominent nasolabial fold, prominent melolabial fold, acne vulgaris scar, post-rhinoplasty irregularity, hypotrophic scar, hypertrophic scar (e.g., keloids), scars due to injury, vaccination, surgery, amongst other causes, cellulite, skin laxness, aging skin, skin thinning and need for skin augmentation. An inclusive, but not exclusive list of defects include breast tissue deficiency, wounds and bums, hernias, periodontal disease and disorders, tendon, muscle and ligament tears, baldness and tissue mass adjustment.

In general, methods of practicing augmentation and repair may include placing cells and other compositions as described herein into the tissue at or near the defect that is to be treated or site of augmentation. The cells may be in singlet state, meaning that at least about three-fourths of the cells are not attached to other cells. The cells may be separated from each other, meaning that at least about half the number of the cells are not attached to other cells when injected. The cells may be partially separated from each other meaning that at least about half of the number of the cells are in groups of about ten cells or less. Or the cells may be attached to each other, meaning that at least about half the number of cells are in groups of about fifty cells or more. The cells may be, e.g., in a sheet, e.g., as lifted off of a cell culture flask or roller bottle, or in a three-dimensional matrix. The manner and exact placement of the cells depends on the defect to be treated or desired augmentation, and is generally related to the structure and function of the tissue.

Particular Embodiments and Additional Aspects of the Invention

Particular embodiments of the invention include: (A) A method or composition comprising an in vitro preparation of autologous cells and an immunogenic cell-absorbable protein; (B) A composition or method of treating a defect in a patient comprising: expanding a culture of autologous cells in vitro to form cultured cells, collecting the cultured cells for introduction into the patient, and depositing the cultured cells with a cell culture medium serum-derived protein at or near the defect in the patient; (C) A composition or method of treating a defect in a patient comprising: expanding a culture of autologous cells in vitro, collecting the cells for introduction into the patient, incubating the cells with an effective amount of an immunogenic cell-absorbable protein to bind the protein exclusively to the cells, wherein the protein specifically interacts with cell surface receptors on the cells, and depositing the cells at or near the defect in the patient to repair or augment a tissue at or near the defect; (D) A method or composition comprising an in vitro preparation of autologous cells and an immunogenic cell-absorbable protein immunogenic relative to an individual that contributed the autologous cells; (E) A composition or method of treating a defect in a patient comprising: expanding a culture of autologous cells in vitro and depositing the cells with a predetermined apoptosis inhibiting protein at or near the defect in the patient to repair or augment a tissue at or near the defect; (F) A composition or method of treating a defect in a patient comprising: expanding a culture of autologous cells in vitro and preparing a mixture that comprises the cells and a purified absorbable serum protein, and depositing the mixture at or near the defect to repair or augment a tissue at or near the defect; (G) A composition or method of treating a defect in a patient comprising: expanding a culture of autologous cells in vitro and depositing a mixture comprising the cells and a predetermined protease inhibiting factor at or near at or near the defect in the patient to repair or augment a tissue at or near the defect; (H) A composition or method of treating a tissue in a patient comprising expanding a culture of autologous cells in vitro and implanting the autologous cells at or near a tissue defect to treat the tissue for a deficiency caused by aging; (I) A composition or method of treating a defect in a patient comprising depositing a cell adhesion mediating protein at or near the defect in the patient to repair or augment a tissue at or near the defect; (J) A composition or method of treating a defect in a patient comprising: expanding a culture of autologous cells in vitro and incubating the autologous cells in a nongellable physiological solution that further comprises an absorbable immunogenic protein and depositing a mixture of the cells and the protein at or near the defect in the patient to repair or augment a tissue at or near the defect; (K) A composition or method of treating a defect in a patient comprising: expanding a culture of autologous cells in vitro, collecting the cells for introduction into the patient and depositing a mixture of the cells and a protein at or near the defect in the patient to repair or augment a tissue at or near the defect; (L) A comoposition or method of treating a patient comprising culturing non-autologous cells in autologous serum and introducing the non-autologous cells into the patient; (M) A composition or method of treating a defect in a patient comprising: expanding a culture of autologous cells in vitro to form cultured cells, collecting the cultured cells for introduction into the patient, and depositing the cultured cells with a serum-derived protein at or near the defect in the patient; (N) A composition or method of treating a tissue defect in a patient comprising placing mammalian cells at or near the tissue defect; (O) A composition or method of treating a tissue defect in a patient comprising placing mammalian cells and an immunogenic protein at or near a tissue defect in the subject; (P) A composition or method of treating a patient comprising using whole blood, fractionated blood, plasma, and/or serum from a donor younger than the patient to expand a culture of autologous cells in vitro for implantion at or near a tissue defect; e.g., to treat the tissue for a deficiency caused by aging, (Q) A composition or method of treating a patient comprising implanting whole blood, fractionated blood, plasma, and/or serum from a donor younger than the patient into the patient in combination with a culture of cells expanded from autologous cells, (R) A composition or method of treating a patient comprising using cells and/or whole blood, and/or fractionated blood and/or plasma, and/or serum from a donor younger than the patient at or near the tissue defect, e.g., to treat the tissue for a deficiency caused by aging, (S) A composition or method of treating a patient comprising using cells and/or whole blood, and/or fractionated blood and/or plasma, and/or serum from a donor younger than the patient, e.g., to treat a tissue, tissues or the entire body for a deficiency caused by, for example, aging, and (T) A composition or method of treating a patient comprising using cells and/or whole blood, fractionated blood, plasma, and/or serum from a donor younger than the patient, e.g. to treat the tissue, tissues or the entire body for a deficiency caused by, for example, aging.

Features, steps, or other elements of (A)-(T), above, may optionally be directed to one or more of the following elements indicated herein by roman numerals, in any self-consistent combination, including subcombination: (i) wherein the protein is: a recombinant protein, a soluble protein, an insoluble protein, an extracellular matrix molecule a serum protein, a growth factor, a hormone, a cytokine, a chemokine or a cell adhesion protein; (ii) wherein the protein is non-autologous; (iii) wherein the protein is used during the culture of the cells or is added to the cells after culturing of the cells is completed; (iv) wherein the protein contains a cell binding site or further contains an ECM binding site; (v) wherein the protein is a proteoglycan, fibronectin, vitronectin, chondronectin, laminin, tenascin, fibrinogen, fibrin, fibulin, von Willebrand's factor, aggrecan, or elastin; (vi) further comprising a protein that provides additional elasticity to the tissue; (via) wherein the protein provides additional elasticity to the tissue (vii) wherein the defect is chosen from the group consisting of a rhytid, stretch mark, depressed scar, cutaneous depression, hypoplasia of the lip, wrinkle, prominent nasolabial fold, prominent melolabial fold, and scarring from acne vulgaris; (viii) wherein the defect is chosen from the group consisting of skin laxness, skin thinning, hypertrophic scars, keloids, wound, burn, hernia, breast deficiency, ligament tear, tendon tear, muscle tear, baldness, a periodontal disorder, a periodontal disease, and sphincter structure deficiency; (ix) wherein the defect is a deficiency caused by aging chosen from the group consisting of tissue dysfunction, tissue dystrophy, laxness, thinning, loss of elasticity, altered protein profile, diminished tissue mass, decreased amounts of extracellular matrix, decreased proteoglycan, decreased tissue turgor, decreased tissue moisture, increased amounts of protease activity loss of cell numbers, or loss of progenitor or stem cells; (x) wherein the protein is a proteoglycan chosen from the group consisting of, agrin, bamacan, brain enriched hyaluronan, biglycan, brevican, decorin, fibromodulin, keratocan, lumican, neurocan, perlecan, syndecan, heparan sulfate proteoglycan, and versican; (xi) wherein the protein is an apoptosis inhibiting protein, an anoikis inhibiting protein, an angiogenesis protein, a vasodilator protein, a pro-inflammatory protein, a filler or augmenting protein, a differentiation protein, a cell mitogen, a promoter of extracellular matrix production, a chemoattractant, a cell culture medium serum-derived protein, a procoagulation protein, a transport protein, or a protease inhibiting factor; (xii) further comprising introducing an apoptosis inhibiting protein with the cells into the defect; (xiii) further comprising introducing an anoikis inhibiting protein with the cells into the defect; (xiv) further comprising introducing a protease inhibiting factor with the cells into the defect; (xv) further comprising introducing a transport protein with the cells at or near the defect; (xvi) further comprising introducing a procoagulation protein with the cells at or near the defect; (xvii) further comprising introducing a cell culture medium serum-derived protein with the cells at or near the defect; (xviii) further comprising introducing a chemoattractant with the cells at or near the defect; (xix) further comprising introducing a promoter of extracellular matrix production with the cells at or near the defect; (xx) further comprising introducing a cell mitogen with the cells at or near the defect; (xxi) further comprising introducing a differentiation protein with the cells at or near the defect; (xxii) further comprising introducing a filler or augmenting protein with the cells at or near the defect; (xxiii) further comprising introducing a pro-inflammatory protein with the cells at or near the defect; (xxiv) further comprising introducing a vasodilator protein with the cells at or near the defect; (xxv) further comprising introducing an angiogenesis protein with the cells at or near the defect; (xxvi) wherein the autologous cells comprise papillary fibroblasts, reticular fibroblasts, fascia fibroblasts, preadipocytes, or adipocytes; (xxvii) wherein the autologous cells comprise dermal fibroblasts; (xxviii) wherein the autologous cells comprise lamina propria fibroblasts, stromal fibroblasts, myofibroblasts, derma papilla fibroblasts, muscle cells, smooth muscle cells, skeletal muscle cells, striated muscle cells, myoblasts, epithelial cells, endothelial cells or epidermal cells; (xxix) wherein the autologous cells are mesenchymal cells, nondifferentiated mesenchymal cells, or stem cells; (xxx) wherein the protein is autologous or non-autologous; (xxxi) wherein the protein is used during the culture of the cells or is added to the cells after culturing of the cells is completed; (xxxii) wherein the protein contains a cell binding site or contains an ECM binding site; (xxxiii) wherein the protein is a cell mitogen, a differentiation protein, a filler or augmenting protein, a pro-inflammatory protein, a vasodilator protein, an angiogenesis protein, a chemoattractant, a vasodilator, a promoter of ECM production, a cell proliferation protein, a differentiation protein, or a cell culture medium serum-derived protein; (xxxiv) wherein the autologous cells are chosen from the group consisting of papillary fibroblasts, reticular fibroblasts, fascia fibroblasts, preadipocytes, and adipocytes; (xxxv) wherein the autologous cells comprise dermal fibroblasts; (xxxvi) wherein the autologous cells comprise lamina propria fibroblasts, stromal fibroblasts, myofibroblasts, derma papilla fibroblasts, muscle cells, smooth muscle cells, skeletal muscle cells, striated muscle cells, myoblasts, epithelial cells, endothelial cells or epidermal cells; (xxxvii) wherein the autologous cells are mesenchymal cells, nondifferentiated mesenchymal cells, or stem cells; (xxxvii) wherein the protein is an amount of soluble or absorbable extracellular matrix molecule effective to inhibit anoikis; (xxxviii) wherein the extracellular matrix molecule is an (effective) amount of fibronectin or vitronectin effective to inhibit apoptosis or anoiki; (xxxix) wherein the protein is an inhibitor of tumor necrosis factor, Fas, βNGF, RANK, TRAIL, RAGE receptor or apoptosis receptors; (xl) wherein the apoptosis receptor inhibitors are PDGF, IGF, FGFs, IL-15, decoy receptors, soluble receptors, or antibodies to the apoptosis receptors; (xli) further comprising introducing at or near the defect, with the cells, an extracellular matrix molecule; (xlii), wherein the extracellular matrix molecule is produced in vitro by the autologous cells; (xliii) further comprising introducing at or near the defect, with the cells, a proteoglycan chosen from the group consisting of, agrin, bamacan, brain enriched hyaluronan, biglycan, brevican, decorin, fibromodulin, heparan sulfate proteoglycan, keratocan, lumican, neurocan, perlecan, syndecan, and versican.; (xliv) further comprising introducing a protease inhibiting factor at or near the defect; (xlv) wherein the tissue used as a source of cells or the tissue having the defect is fascia, connective, papillary tissue, reticular tissue, lamina propria, adipose, tendon, or ligament; (xlvi) wherein the tissue used as a source of cells or the tissue having the defect is dermis, stroma, hair follicle region, dermal papilla, epidermal tissue, epithelial tissue, or muscle tissue; (xlvii) wherein the autologous cells comprise dermal fibroblasts, papillary fibroblasts, reticular fibroblasts, fascia fibroblasts, preadipocytes, or adipocytes; (xlviii) wherein the autologous cells comprise lamina propria fibroblasts, stromal fibroblasts, myofibroblasts, derma papilla fibroblasts, muscle cells, smooth muscle cells, skeletal muscle cells, striated muscle cells, myoblasts, epithelial cells, or epidermal cells; (xlix) wherein the autologous cells comprise mesenchymal cells or nondifferentiated mesenchymal cells; (l) further comprising combining with the apoptosis inhibiting protein at least one of: an anoikis inhibiting protein, an angiogenesis protein, a vasodilator protein, a pro-inflammatory protein, a filler or augmenting protein, a differentiation protein, a cell mitogen, a promoter of extracellular matrix production, a chemoattractant, a cell culture medium serum-derived protein, a procoagulation protein, a transport protein, or a protease inhibiting factor; (li) wherein the autologous cells are cultured with the serum protein; (lii) wherein the serum protein is part of a mixture of serum proteins; (liii) (the mixture) further comprising a cell culture media factor that specifically binds the serum protein; (liv) wherein the serum protein is albumin and the cell culture media factor is a lipid; (lv) wherein the serum protein is ferritin and the cell culture media factor is iron; (lvi), wherein the serum protein is a transport protein; (xlvii) wherein the serum protein is a transport protein and binds to a serum growth factor, cytokine, chemokine or hormone; (lviii) wherein the serum protein is transferrin, transcobalamin, high density lipoprotein, low density lipoprotein, ceruloplasmin, or a hormone binding protein; (lix) further comprising introducing at or near the defect, with the cells, an extracellular matrix molecule; (lx) wherein the extracellular matrix molecule is produced in vitro by the autologous cell; (lxi) (the mixture) further comprising an apoptosis inhibiting protein, an anoikis inhibiting protein, an angiogenesis protein, a vasodilator protein, a pro-inflammatory protein, a filler or augmenting protein, a differentiation protein, a cell mitogen, a promoter of extracellular matrix production, a chemoattractant, a cell culture medium serum-derived protein, a procoagulation protein, a transport protein, or a protease inhibiting factor; (lii) wherein the serum protein is an apoptosis inhibiting protein, an anoikis inhibiting protein, an angiogenesis protein, a vasodilator protein, a pro-inflammatory protein, a filler or augmenting protein, a differentiation protein, a cell mitogen, a promoter of extracellular matrix production, a chemoattractant, a cell culture medium serum-derived protein, a procoagulation protein, a transport protein, or a protease inhibiting factor; (liii), wherein the tissue with the defect or the tissue used as a cell source is fascia, connective, papillary tissue, reticular tissue, lamina propria, adipose, tendon, or ligament; (liv) wherein the protease inhibiting factor is a matrix metalloproteinase inhibitor, tissue inhibitor of metalloproteinase, alpha1 anti-trypsin, soybean tryspin inhibitor or alpha2-macroglobulin; (lv) wherein the protein is: a soluble protein, an insoluble protein, in a gel, an extracellular matrix molecule, a serum protein, a growth factor, a hormone, a cytokine, or a cell adhesion protein; (lvi) wherein the protein is immunogenic; (lvii) wherein the protein is used during the culture of the cells or is added to the cells after culturing of the cells is completed; (lviii) wherein the protein contains a cell binding site or further contains an ECM binding site; and/or (lix) wherein an immunogenic protein, serum-derived protein, serum protein, or other protein is present at a concentration of, e.g., more than 0.1% or 0.15-20%.

Further embodiments are contemplated for use alone or in combination as aspects of certain embodiments of the invention; these include embodiments L, M, N, O, P, Q, R, S, and T, already described, above. These embodiments may be combined, as appropriate, with the following elements (which are also available for combination with embodiments A-K): (lx) wherein the (non-autologous) cells are stem cells, umbilical cord cells, somatic nuclear transfer cells, embryonic stem cells or adult stem cells; (lxi) treating a patient with cells comprising culturing the cells in human serum, wherein the cells are optinally mammalian cells, stem cells, embryonic stem cells, umbilical cord stem cells, fetal stem cells, somatic nuclear transfer stem cells, adult stem cells, autologous stem cells, autologous cells, or nonautologous cells, and optionally introducing the cells into the patient to treat a defect; (lxii) culturing cells in a human serum taken from a person that is younger than patient that receives the cells; (lxiii) culturing cells in human serum taken from a person that is not an adult, with the cells optionally being mammalian cells, stem cells, embryonic stem cells, umbilical cord stem cells, fetal stem cells, somatic nuclear transfer stem cells, adult stem cells, autologous stem cells, autologous cells, or nonautologous cells; (lxiv) wherein serum from a family member is used to culture the cells; (lxv) culturing cells in umbilical cord serum; (lxvi) culturing cells in human fetal serum; (lxvii) treating a tissue defect in a patient comprising placing mammalian cells at or near the tissue defect wherein the mammalian cells are optionally autologous cells; (lxviii) wherein the autologous cells are younger than the cells of the patient when the patient receives the cells; (lxix) wherein the mammalian cells or serum are histocompatible with the subject; (lxx) wherein the mammailian cells are from a family member of the patient; (lxxi) wherein the family member is younger than the patient; (lxxii) wherein the mammalian cells are obtained from young adult, pre-adolescent, neonatal, fetal, or embryonic tissue; (lxxiii) wherein cells and/or serum younger than the subject is used to treat a tissue defect; (lxxiv) treating a tissue defect in a patient comprising placing mammalian cells and an immunogenic protein at or near a tissue defect in the subject, wherein the mammalian cells are optionally autologous cells; and (lxxv) wherein the cells and/or serum introduced into the patient are derived from a donor that is a genetically related family member.

All patents, patent applications, publications, journal articles, and publications mentioned herein are hereby incorporated by reference herein to the extent that the incorporated subject matter is not contradictory with the explicit disclosure herein.

1. A composition comprising an in vitro preparation of autologous cells and an immunogenic cell-absorbable protein immunogenic relative to an individual that contributed the autologous cells.

2. The composition of claim 1, wherein the protein is:
a recombinant protein, a soluble protein, an insoluble protein, an extracellular matrix molecule a serum protein, a growth factor, a hormone, a cytokine, a chemokine or a cell adhesion protein.

3. The composition of claim 1, wherein the protein is non-autologous.

4. The composition of claim 1, wherein the protein is used during the culture of the cells or is added to the cells after culturing of the cells is completed.

5. The composition of claim 1, wherein the protein contains a cell binding site or contains an ECM binding site.

6. The composition of claim 1, wherein the protein is a proteoglycan, fibronectin, vitronectin, chondronectin, laminin, tenascin, fibrinogen, fibrin, fibulin, von Willebrand's factor, aggrecan, or elastin.

7. The composition of claim 1, further comprising a protein that provides additional elasticity to the tissue.

8. The composition of claim 1, wherein the protein that provides additional elasticity to the tissue.

9. The composition of claim 1, wherein the protein is a proteoglycan chosen from the group consisting of, agrin, bamacan, brain enriched hyaluronan, biglycan, brevican, decorin, fibromodulin, keratocan, lumican, neurocan, perlecan, syndecan, heparan sulfate proteoglycan, and versican.

10. The composition of claim 1, further comprising an apoptosis inhibiting protein, an anoikis inhibiting protein, a protease inhibiting factor, a transport protein, a procoagulation protein, a cell mitogen, a differentiation protein, a filler or augmenting protein, a pro-inflammatory protein, a vasodilator protein, an angiogenesis protein, a chemoattractant, a vasodilator, a promoter of ECM production, a cell proliferation protein, a differentiation protein, or a cell culture medium serum-derived protein.

11. The composition of claim 1, wherein the protein is an apoptosis inhibiting protein, an anoikis inhibiting protein, an angiogenesis protein, a vasodilator protein, a pro-inflammatory protein, a filler or augmenting protein, a differentiation protein, a cell mitogen, a promoter of extracellular matrix production, a chemoattractant, a cell culture medium serum-derived protein, a procoagulation protein, a transport protein, or a protease inhibiting factor.

12. The composition of claim 1, wherein the autologous cells are chosen from the group consisting of papillary fibroblasts, reticular fibroblasts, fascia fibroblasts, preadipocytes, and adipocytes.

13. The composition of claim 1, wherein the autologous cells comprise denial fibroblasts.

14. The composition of claim 1, wherein the autologous cells comprise lamina propria fibroblasts, stromal fibroblasts, myofibroblasts, derma papilla fibroblasts, muscle cells, smooth muscle cells, skeletal muscle cells, striated muscle cells, myoblasts, epithelial cells, endothelial cells or epidermal cells.

15. The composition of claim 1, wherein the autologous cells comprise mesenchymal cells, nondifferentiated mesenchymal cells, or stem cells.

16. A method of treating a defect in a patient comprising:
expanding a culture of autologous cells in vitro to form cultured cells, collecting the cultured cells for introduction into the patient, and depositing the cultured cells with a serum-derived protein at or near the defect in the patient.

17. The method of claim 16, wherein the protein is present as a component of serum.

18. The method of claim 16, wherein the serum-derived protein has not been previously exposed to the cells.

19. The method of claim 16, wherein the serum-derived protein bas been previously used in culture medium used to culture the cells and was thereby exposed to the cultured cells.

20. The method of claim 16, wherein the protein is found in serum and is produced by recombinant techniques.

21. The method of claim 16, wherein the protein is an immunogenic protein.

22. The method of claim 16, wherein the protein is present at a concentration of more than 0.1% v/v or a concentration of more than 0.1% w/w relative to the concentration of the protein in a cell culture medium last used to culture the cultured cells.

23. The method of claim 16, wherein the protein is a soluble proteins an insoluble protein, in a gel, an extracellular matrix molecule, a serum protein, a growth factor, a hormone, a cytokine, or a cell adhesion protein.

24. The method of claim 16, wherein the protein is non-autologous.

25. The method of claim 16, wherein the protein contains a cell binding site or contains an ECM binding site.

26. The method of claim 16, wherein the protein is a proteoglycan, fibronectin, vitronectin, chondronectin, laminin, tenascin, fibrinogen, fibrin, fibulin, von Willebrand's factor, aggrecan, or elastin.

27. The method of claim 16, further comprising mixing a protein that provides additional elasticity to the tissue with the cells and the serum-derived protein.

28. The method of claim 16, wherein the protein provides additional elasticity to the tissue.

29. The method of claim 16, wherein the defect is chosen from the group consisting of a rhytid, stretch mark, depressed scar, cutaneous depression, hypoplasia of the lip, wrinkle, prominent nasolabial fold, prominent melolabial fold, and scarring from acne vulgaris.

30. The method of claim 16, wherein the defect is chosen from the group consisting of skin laxness, skin thinning, hypertrophic scars, wound, burn, hernia, breast deficiency, ligament tear, tendon tear, muscle tear, baldness, a periodontal disorder, a periodontal disease, and sphincter structure deficiency.

31. The method of claim 16, wherein the protein is a proteoglycan chosen from the group consisting of, agrin, bamacan, brain enriched hyaluronan, biglycan, brevican, decorin, fibromodulin, keratocan, lumican, neurocan, perlecan, syndecan, heparan sulfate proteoglycan and versican.

32. The method of claim 16, further comprising, in a mixture with the serum-derived protein and the cultured cells, a protein that is an apoptosis inhibiting protein, and anoikis inhibiting protein, an angiogenesis protein, a vasodilator protein, a pro-inflammatory protein, a filler or augmenting protein, a differentiation protein, a cell mitogen, a promoter of extracellular matrix production, a chemoattractant, a cell culture medium serum-derived protein, a procoagulation protein, a transport protein, or a protease inhibiting factor.

33. The method of claim 16, wherein the protein is an apoptosis inhibiting protein, and anoikis inhibiting protein, an angiogenesis protein, a vasodilator protein, a pro-inflammatory protein, a filler or augmenting protein, a differentiation protein, a cell mitogen, a promoter of extracellular matrix production, a chemoattractant, a cell culture medium serum-derived protein, a procoagulation protein, a transport protein, or a protease inhibiting factor.

34. The method of claim 16, wherein the autologous cells comprise papillary fibroblasts, reticular fibroblasts, fascia fibroblasts, preadipocytes, or adipocytes.

35. The method of claim 16, wherein the autologous cells comprise lamina propria fibroblasts, stromal fibroblasts, myofibroblasts, derma papilla fibroblasts, muscle cells, smooth muscle cells, skeletal muscle cells, striated muscle cells, myoblasts, epithelial cells, or epidermal cells.

36. The method of claim 16, wherein the autologous cells comprise mesenchymal cells, nondifferentiated mesenchymal cells, or stem cells.

37. A method of treating a defect in a patient comprising:
expanding a culture of autologous cells in vitro, collecting the cells for introduction into the patient, incubating the cells with an effective amount of an immunogenic cell-absorbable protein, and depositing the cells at or near the defect in the patient to repair or augment a tissue at or near the defect.

38. The method of claim 37, wherein the protein is:
a recombinant protein, a soluble protein, an insoluble protein, an extracellular matrix molecule a serum protein, a growth factor, a hormone, a cytokine, a chemokine or a cell adhesion protein.

39. The method of claim 37, wherein the protein is non-autologous.

40. The method of claim 37, wherein the protein is used during the culture of the cells or is added to the cells after culturing of the cells is completed.

41. The method of claim 37, wherein the protein contains a cell binding site or contains an ECM binding site.

42. The method of claim 37, wherein the protein is a proteoglycan, fibronectin, vitronectin, chondronectin, laminin, tenascin, fibrinogen, fibrin, fibulin, von Willebrand's factor, aggrecan, or elastin.

43. The method of claim 37, further comprising a protein that provides additional elasticity to the tissue.

44. The method of claim 37, wherein the protein provides additional elasticity to the tissue.

45. The method of claim 37, wherein the protein is a proteoglycan chosen from the group consisting of, agrin, bamacan, brain enriched hyaluronan, biglycan, brevican, decorin, fibromodulin, keratocan, lumican, neurocan, perlecan, syndecan, heparan sulfate proteoglycan, and versican.

46. The method of claim 37, further comprising an apoptosis inhibiting protein, an anoikis inhabiting protein, a protease inhibiting factor, a transport protein, a procoagulation protein, a cell mitogen, a differentiation protein, a filler or augmenting protein, a pro-inflammatory protein, a vasodilator protein, an angiogenesis protein, a chemoattractant, a vasodilator, a promoter of ECM production, a cell proliferation protein, a differentiation protein, or a cell culture medium serum-derived protein.

47. The method of claim 37, wherein the protein is an apoptosis inhibiting protein, an anoikis inhibiting protein, an angiogenesis protein, a vasodilator protein, a pro-inflammatory protein, a filler or augmenting protein, a differentiation protein, a cell mitogen, a promoter of extracellular matrix production, a chemoattractant, a cell culture medium serum-derived protein, a procoagulation protein, a transport protein, or a protease inhibiting factor.

48. The method of claim 37, wherein the autologous cells are chosen from the group consisting of papillary fibroblasts, reticular fibroblasts, fascia fibroblasts, preadipocytes, and adipocytes.

49. The method of claim 37, wherein the autologous cells comprise dermal fibroblasts.

50. The method of claim 37, wherein the autologous cells comprise lamina propria fibroblasts, stromal fibroblasts, myofibroblasts, derma papilla fibroblasts, muscle cells, smooth muscle cells, skeletal muscle cells, striated muscle cells, myoblasts, epithelial cells, endothelial cells or epidermal cells.

51. The method of claim 37, wherein the autologous cells comprise mesenchymal cells, nondifferentiated mesenchymal cells, or stem cells.
